(19) United States
(12) Patent Application Publication
(10) Pub. No.: US 2021/0214727 A1
(43) Pub. Date: Jul. 15, 2021

(54) ENHANCED OLIGONUCLEOTIDES FOR INHIBITING SCN9A EXPRESSION

(71) Applicant: Hoffmann-La Roche Inc., Little Falls, NJ (US)

(72) Inventors: Lykke PEDERSEN, Copenhagen NV (DK); Søren V. RASMUSSEN, Espergærde (DK)

(21) Appl. No.: 17/127,099

(22) Filed: Dec. 18, 2020

(30) Foreign Application Priority Data
Dec. 20, 2019 (EP) 19218780.5

(51) Int. Cl. C12N 15/113

(52) U.S. Cl. CPC C12N 15/113; C12N 2310/11

(57) ABSTRACT

The present invention relates to antisense oligonucleotides that are capable of modulating expression of SCN9A in a target cell. The antisense oligonucleotides hybridize to SCN9A mRNA. The present invention further relates to conjugates of the antisense oligonucleotide and pharmaceutical compositions and methods for treatment of or prevention of pain, such as peripheral pain.

### SEQUENCE LISTING

The present application contains a Sequence Listing which has been submitted electronically in ASCII format and is hereby incorporated by reference in its entirety. Said ASCII copy, created on Dec. 14, 2020, is named 51551-002002_Sequence_Listing_12.14.20_ST25 and is 399,285 bytes in size.

### FIELD OF THE INVENTION

The present invention relates to antisense oligonucleotides (ASOs) that are complementary to human SCN9A, for use in the inhibition of expression of SCN9A nucleic acid. SCN9A encodes the voltage-gated sodium channel Naᵥ1.7. Inhibition of SCN9A expression is useful in the prevention or the treatment of pain.

### BACKGROUND

Voltage-gated sodium channels (Nays) play essential roles in excitable tissues, with their activation and opening resulting in the initial phase of the action potential. The cycling of Nays through open, closed and inactivated states, and their closely choreographed relationships with the activities of other ion channels lead to exquisite control of intracellular ion concentrations.

Naᵥ1.7 is a voltage activated ion channel expressed almost exclusively in the small cell peripheral sensory nerves. Mice with a conditional knock-out of Naᵥ1.7 in sensory neurons displayed an antinociceptive phenotype (Nassar et al., 2004, Proc Natl Acad Sci USA. 2004 Aug. 24; 101(34):12706-11). The role of Naᵥ1.7 in pain sensation in humans was demonstrated by association between the spontaneous pain syndrome inherited erythromelalgia (IEM) (Yang et al., J Med Genet. 2004; 41(3):171-4) and paroxysmal extreme pain disorder (PEPD) (Fertleman et al., J Neurol Neurosurg Psychiatry. 2006 November; 77(11):1294-5) and gain of function mutation in Naᵥ1.7 of these patients (Cummins et al., J Neurosci. 2004; 24(38):8232-8236). Further support for Naᵥ1.7 was generated by identification of loss of function mutations that resulted in congenital insensitivity to pain (Cox et al., Nature AAA. 2006; 7121:894-8). These findings led to a number of small molecule drug discovery programs for identification of Naᵥ1.7 modulators, however it appears that finding good compounds with high selectivity and good PK/PD properties have been challenging.

US2016024208 discloses human antibodies to Naᵥ1.7.

WO02083945 refers to synthetic oligonucleotides with antisense sequence to specific regions of SCN5A and optionally also SCN9A for use in the treatment of breast cancer.

US2007/212685 refers to methods of identifying analgesic agents and mentions that specific compounds which will modulate the gene expression or gene transcript levels in a cell of SCN9A include antisense nucleic acids.

US2010273857A refers to methods, sequences and nucleic acid molecules used to treat pain via locally administering siRNA molecules that suppress the expression of amino acid sequences that encode for Naᵥ1.7 channels or that otherwise inhibit the function of Nav1.7 channels, and reports that local suppression of Naᵥ1.7 channel levels and/or function will occur in the peripheral sensory neurons of the dorsal root ganglia.

WO12162732 relates to novel screening assays for modulating sodium channels, particularly voltage-gated sodium channels.

KR20110087436 discloses an SCN9A antisense oligonucleotide.

Mohan et al., discloses antisense oligonucleotides targeting Naᵥ1.7, and characterize the pharmacodynamic activity of ASOs in spinal cord and dorsal root ganglia (DRG) in rodents (Pain (2018) Volume 159-Number 1, p 139-149).

WO18051175 discloses SCN9A antisense peptide nucleic acid oligonucleotides targeting a part of the human SCN9A pre-mRNA. The peptide nucleic acid derivatives potently induce splice variants of the SCN9A mRNA in cells and are useful to treat pains or conditions involving Naᵥ1.7 activity.

WO19243430 discloses LNA gapmer antisense oligonucleotides targeting SCN9A.

There is therefore a need for antisense oligonucleotides therapeutics which are effective in inhibiting expression of voltage-gated sodium ion channel encoding nucleic acids, such as SCN9A in humans, such as for the prevention or treatment of pain.

### Objective of the Invention

The present invention identifies novel oligonucleotides which are capable of inhibiting the expression of SCN9A and may be used in medicine, such as for the prevention or treatment of pain, such as an analgesic. The compounds of the present invention may be used in the prevention or treatment of peripheral pain.

### SUMMARY OF THE INVENTION

The present invention provides antisense oligonucleotides, which are complementary to, and are capable of inhibiting the expression of, a SCN9A nucleic acid, and for their use in medicine.

The invention provides for an antisense oligonucleotide which is complementary to, such as fully complementary to a region of the human SCN9A pre-mRNA (as illustrated in SEQ ID NO: 1), selected from nucleotides 97704-97732, 103232-103259, 151831-151847, and 151949-152006, of SEQ ID NO: 1.

The antisense oligonucleotide of the invention is typically 12-24 nucleotides in length, and comprises a contiguous nucleotide sequence of at least 12 nucleotides which is complementary to, such as fully complementary to a region of the human SCN9A pre-mRNA (as illustrated in SEQ ID NO: 1), selected from nucleotides 97704-97732, 103232-103259, 151831-151847, and 151949-152006, of SEQ ID NO: 1.

The invention provides for an antisense oligonucleotide 10 to 30 nucleotides in length, which comprises a contiguous nucleotide sequence of 10 to 30 nucleotides in length, wherein the contiguous nucleotide sequence is 100% identical to a sequence selected from the group consisting of SEQ ID NOs: 28-52; or at least 14 contiguous nucleotides thereof.

The invention provides for an antisense oligonucleotide 10 to 30 nucleotides in length, which comprises a contiguous nucleotide sequence of 10 to 30 nucleotides in length, wherein the contiguous nucleotide sequence is 100% identical to a sequence selected from the group consisting of SEQ ID NOs: 28-52, or at least 15 contiguous nucleotides thereof.

The invention provides for an antisense oligonucleotide 10 to 30 nucleotides in length, which comprises a contiguous nucleotide sequence of 10 to 30 nucleotides in length, wherein the contiguous nucleotide sequence is 100% identical to a sequence selected from the group consisting of SEQ ID NOs: 28-52, or at least 16 contiguous nucleotides thereof.

The invention provides for an antisense oligonucleotide, which comprises a contiguous nucleotide sequence, which is 100% identical to a sequence selected from the group consisting of SEQ ID NOs: 28, 29, 30, 31, 32, 33, 34, 35, 36, 37, 38, and 39, or at least 14 contiguous nucleotides thereof.

The invention provides for an antisense oligonucleotide, which comprises a contiguous nucleotide sequence selected from the group consisting of SEQ ID NOs: 28, 29, 30, 31, 32, 33, 34, 35, 36, 37, 38, and 39.

The invention provides for an antisense oligonucleotide, which comprises a contiguous nucleotide sequence, which is 100% identical to SEQ ID NO: 29, or at least 14, 15, 16 or 17 contiguous nucleotides thereof.

The invention provides for an antisense oligonucleotide, which comprises a contiguous nucleotide sequence, which is 100% identical to SEQ ID NO: 31, or at least 14, 15, 16, 17 or 18 contiguous nucleotides thereof.

The invention provides for an antisense oligonucleotide, which comprises a contiguous nucleotide sequence, which is 100% identical to SEQ ID NO: 33, or at least 14, 15, 16, 17, 18 or 19 contiguous nucleotides thereof.

The invention provides for an antisense oligonucleotide, which comprises a contiguous nucleotide sequence, which is 100% identical to SEQ ID NO: 39, or at least 14, 15, 16, 17 or 18 contiguous nucleotides thereof.

The invention provides for an antisense oligonucleotide, which comprises a contiguous nucleotide sequence, which is 100% identical to SEQ ID NO: 47, or at least 14, 15, 16, 17 or 18 contiguous nucleotides thereof.

The invention provides for an antisense oligonucleotide, which comprises a contiguous nucleotide sequence, which is 100% identical to SEQ ID NO: 48, or at least 14, 15, 16, 17 or 18 contiguous nucleotides thereof.

The invention provides for an antisense oligonucleotide, which comprises the contiguous nucleotide of a compound selected from the group consisting of compound ID Nos #29_15, 29_10, 29_22, 39_6, 39_1, 39_2, 39_7, 31_1, 31_3, 31_4, 31_5, 33_1, 33_2, 33_3, 47_1, 48_8, 29_24, 29_25, 29_26, 39_9, 39_10, 48_10, 29_35, 29_34, 39_17, 39_18, 39_19, 39_20, and 29_11.

In some embodiments, the antisense oligonucleotide is not an antisense oligonucleotide selected from the group consisting of compound ID Nos, 29_33, 39_13, 48_9, 29_33, 39_13, 48_9, 29_33, 39_13 and 48_9.

The invention provides for an antisense oligonucleotide selected from the group listed in Table 1, or a pharmaceutically acceptable salt thereof.

The invention provides for an antisense oligonucleotide selected from the group listed in Table 3, or a pharmaceutically acceptable salt thereof.

The invention provides for the antisense oligonucleotide of FIG. 1, or a pharmaceutically acceptable salt thereof. The invention provides for the antisense oligonucleotide of Compound ID Number 29_15, or a pharmaceutically acceptable salt thereof.

The invention provides for the antisense oligonucleotide of FIG. 2, or a pharmaceutically acceptable salt thereof. The invention provides for the antisense oligonucleotide of Compound ID Number 29_10, or a pharmaceutically acceptable salt thereof.

The invention provides for the antisense oligonucleotide of FIG. 3, or a pharmaceutically acceptable salt thereof. The invention provides for the antisense oligonucleotide of Compound ID Number 29_22, or a pharmaceutically acceptable salt thereof.

The invention provides for the antisense oligonucleotide of FIG. 4, or a pharmaceutically acceptable salt thereof. The invention provides for the antisense oligonucleotide of Compound ID Number 39_6, or a pharmaceutically acceptable salt thereof.

The invention provides for the antisense oligonucleotide of FIG. 5, or a pharmaceutically acceptable salt thereof. The invention provides for the antisense oligonucleotide of Compound ID Number 39_1, or a pharmaceutically acceptable salt thereof.

The invention provides for the antisense oligonucleotide of FIG. 6, or a pharmaceutically acceptable salt thereof. The invention provides for the antisense oligonucleotide of Compound ID Number 39_2, or a pharmaceutically acceptable salt thereof.

The invention provides for the antisense oligonucleotide of FIG. 7, or a pharmaceutically acceptable salt thereof. The invention provides for the antisense oligonucleotide of Compound ID Number 39_7, or a pharmaceutically acceptable salt thereof.

The invention provides for the antisense oligonucleotide of FIG. 8, or a pharmaceutically acceptable salt thereof. The invention provides for the antisense oligonucleotide of Compound ID Number 31_1, or a pharmaceutically acceptable salt thereof.

The invention provides for the antisense oligonucleotide of FIG. 9, or a pharmaceutically acceptable salt thereof. The invention provides for the antisense oligonucleotide of Compound ID Number 31_3, or a pharmaceutically acceptable salt thereof.

The invention provides for the antisense oligonucleotide of FIG. 10, or a pharmaceutically acceptable salt thereof. The invention provides for the antisense oligonucleotide of Compound ID Number 31_4, or a pharmaceutically acceptable salt thereof.

The invention provides for the antisense oligonucleotide of FIG. 11, or a pharmaceutically acceptable salt thereof. The invention provides for the antisense oligonucleotide of Compound ID Number 31_5, or a pharmaceutically acceptable salt thereof.

The invention provides for the antisense oligonucleotide of FIG. 12, or a pharmaceutically acceptable salt thereof. The invention provides for the antisense oligonucleotide of Compound ID Number 33_1, or a pharmaceutically acceptable salt thereof.

The invention provides for the antisense oligonucleotide of FIG. 13, or a pharmaceutically acceptable salt thereof. The invention provides for the antisense oligonucleotide of Compound ID Number 33_2, or a pharmaceutically acceptable salt thereof.

The invention provides for the antisense oligonucleotide of FIG. 14, or a pharmaceutically acceptable salt thereof. The invention provides for the antisense oligonucleotide of Compound ID Number 33_3, or a pharmaceutically acceptable salt thereof.

The invention provides for the antisense oligonucleotide of FIG. 15, or a pharmaceutically acceptable salt thereof. The invention provides for the antisense oligonucleotide of Compound ID Number 47_1, or a pharmaceutically acceptable salt thereof.

The invention provides for the antisense oligonucleotide of FIG. 16, or a pharmaceutically acceptable salt thereof. The invention provides for the antisense oligonucleotide of Compound ID Number 48_8, or a pharmaceutically acceptable salt thereof.

The invention provides for the antisense oligonucleotide of FIG. 17, or a pharmaceutically acceptable salt thereof. The invention provides for the antisense oligonucleotide of Compound ID Number 29_24, or a pharmaceutically acceptable salt thereof.

The invention provides for the antisense oligonucleotide of FIG. 18, or a pharmaceutically acceptable salt thereof. The invention provides for the antisense oligonucleotide of Compound ID Number 29_25, or a pharmaceutically acceptable salt thereof.

The invention provides for the antisense oligonucleotide of FIG. 19, or a pharmaceutically acceptable salt thereof. The invention provides for the antisense oligonucleotide of Compound ID Number 29_26, or a pharmaceutically acceptable salt thereof.

The invention provides for the antisense oligonucleotide of FIG. 20, or a pharmaceutically acceptable salt thereof. The invention provides for the antisense oligonucleotide of Compound ID Number 39_9, or a pharmaceutically acceptable salt thereof.

The invention provides for the antisense oligonucleotide of FIG. 21, or a pharmaceutically acceptable salt thereof. The invention provides for the antisense oligonucleotide of Compound ID Number 39_10, or a pharmaceutically acceptable salt thereof.

The invention provides for the antisense oligonucleotide of FIG. 22, or a pharmaceutically acceptable salt thereof. The invention provides for the antisense oligonucleotide of Compound ID Number 48_10, or a pharmaceutically acceptable salt thereof.

The invention provides for the antisense oligonucleotide of FIG. 23, or a pharmaceutically acceptable salt thereof. The invention provides for the antisense oligonucleotide of Compound ID Number 29_35, or a pharmaceutically acceptable salt thereof.

The invention provides for the antisense oligonucleotide of FIG. 24, or a pharmaceutically acceptable salt thereof. The invention provides for the antisense oligonucleotide of Compound ID Number 29_34, or a pharmaceutically acceptable salt thereof.

The invention provides for the antisense oligonucleotide of FIG. 25, or a pharmaceutically acceptable salt thereof. The invention provides for the antisense oligonucleotide of Compound ID Number 39_17, or a pharmaceutically acceptable salt thereof.

The invention provides for the antisense oligonucleotide of FIG. 26, or a pharmaceutically acceptable salt thereof. The invention provides for the antisense oligonucleotide of Compound ID Number 39_18, or a pharmaceutically acceptable salt thereof.

The invention provides for the antisense oligonucleotide of FIG. 27, or a pharmaceutically acceptable salt thereof. The invention provides for the antisense oligonucleotide of Compound ID Number 39_19, or a pharmaceutically acceptable salt thereof.

The invention provides for the antisense oligonucleotide of FIG. 28, or a pharmaceutically acceptable salt thereof. The invention provides for the antisense oligonucleotide of Compound ID Number 39_20, or a pharmaceutically acceptable salt thereof.

The invention provides for the antisense oligonucleotide of FIG. 29, or a pharmaceutically acceptable salt thereof. The invention provides for the antisense oligonucleotide of Compound ID Number 29_11, or a pharmaceutically acceptable salt thereof.

TABLE 1
Compound Table - HELM Annotation Format

| SEQ | Compound | | Exemplary |
|---|---|---|---|
| ID | ID | | compound - |
| Number | Number # | HELM Annotation Written 5′ - 3′. | see FIG. No: |
| 29 | 29_15 | [LR](G)[sP].[LR](T)[sP].[LR](T)[sP].[dR](T)[sP].[LR](T)[sP]. | 1 |
| | | [LR](A)[sP].[dR](A)[sP].[dR](T)[sP].[dR](A)[sP].[dR](C)[sP]. | |
| | | [dR](C)[sP].[dR](A)[sP].[dR](T)[sP].[dR](T)[sP].[dR](T)[sP]. | |
| | | [LR]([5meC])[sP].[LR](A) | |
| 29 | 29_10 | [LR](G)[sP].[LR](T)[sP].[dR](T)[sP].[dR](T)[sP].[LR](T)[sP]. | 2 |
| | | [LR](A)[sP].[dR](A)[sP].[dR](T)[sP].[dR](A)[sP].[dR](C)[sP]. | |
| | | [dR](C)[sP].[dR](A)[sP].[dR](T)[sP].[dR](T)[sP].[LR](T)[sP]. | |
| | | [LR]([5meC])[sP].[LR](A) | |
| 29 | 29_22 | [LR](G)[sP].[LR](T)[sP].[dR](T)[sP].[dR](T)[sP].[dR](T)[sP]. | 3 |
| | | [LR](A)[sP].[dR](A)[sP].[dR](T)[sP].[dR](A)[sP].[dR](C)[sP]. | |
| | | [dR](C)[sP].[dR](A)[sP].[dR](T)[sP].[LR](T)[sP].[LR](T)[sP]. | |
| | | [LR]([5meC])[sP].[LR](A) | |
| 39 | 39_6 | [LR](T)[sP].[LR](T)[sP].[LR]([5meC])[sP].[LR](A)[sP].[dR](C)[sP]. | 4 |
| | | [dR](A)[sP].[dR](T)[sP].[dR](A)[sP].[dR](A)[sP].[dR](T)[sP]. | |
| | | [dR](T)[sP].[dR](T)[sP].[dR](A)[sP].[dR](T)[sP].[dR](T)[sP]. | |
| | | [dR](C)[sP]. [LR]([5meC])[sP].[LR]([5meC]) | |
| 39 | 39_1 | [LR](T)[sP].[LR](T)[sP].[dR](C)[sP].[dR](A)[sP].[dR](C)[sP]. | 5 |
| | | [dR](A)[sP].[dR](T)[sP].[dR](A)[sP].[dR](A)[sP].[dR](T)[sP]. | |
| | | [dR](T)[sP].[dR](T)[sP].[LR](A)[sP].[LR](T)[sP].[dR](T)[sP]. | |
| | | [dR](C)[sP].[LR]([5meC])[sP].[LR]([5meC]) | |
| 39 | 39_2 | [LR](T)[sP].[LR](T)[sP].[dR](C)[sP].[dR](A)[sP].[dR](C)[sP]. | 6 |
| | | [dR](A)[sP].[dR](T)[sP].[dR](A)[sP].[dR](A)[sP].[dR](T)[sP]. | |
| | | [LR](T)[sP].[dR](T)[sP].[LR](A)[sP].[LR](T)[sP].[dR](T)[sP]. | |
| | | [dR](C)[sP].[LR]([5meC])[sP].[LR]([5meC]) | |
| 39 | 39_7 | [LR](T)[sP].[LR](T)[sP].[LR]([5meC])[sP].[dR](A)[sP].[dR](C)[sP]. | 7 |
| | | [dR](A)[sP].[dR](T)[sP].[dR](A)[sP].[dR](A)[sP].[dR](T)[sP]. | |
| | | [dR](T)[sP].[dR](T)[sP].[dR](A)[sP].[dR](T)[sP].[LR](T)[sP]. | |
| | | [dR](C)[sP].[LR]([5meC])[sP].[LR]([5meC]) | |
| 31 | 31_1 | [LR]([5meC])[sP].[LR](A)[sP].[dR](G)[sP].[dR](T)[sP].[LR](T)[sP]. | 8 |
| | | [dR](T)[sP].[dR](T)[sP].[dR](A)[sP].[dR](A)[sP].[dR](T)[sP]. | |
| | | [dR](A)[sP].[dR](C)[sP].[dR](C)[sP].[dR](A)[sP].[dR](T)[sP]. | |
| | | [LR](T)[sP].[LR](T)[sP].[LR]([5meC]) | |
| 31 | 31_3 | [LR]([5meC])[sP].[LR](A)[sP].[dR](G)[sP].[dR](T)[sP].[LR](T)[sP]. | 9 |
| | | [dR](T)[sP].[LR](T)[sP].[dR](A)[sP].[dR](A)[sP].[dR](T)[sP]. | |
| | | [dR](A)[sP].[dR](C)[sP].[dR](C)[sP].[dR](A)[sP].[dR](T)[sP]. | |
| | | [dR](T)[sP].[LR](T)[sP].[LR]([5meC]) | |
| 31 | 31_4 | [LR]([5meC])[sP].[LR](A)[sP].[dR](G)[sP].[LR](T)[sP].[dR](T)[sP]. | 10 |
| | | [dR](T)[sP].[LR](T)[sP].[dR](A)[sP].[dR](A)[sP].[dR](T)[sP]. | |
| | | [dR](A)[sP].[dR](C)[sP].[dR](C)[sP].[dR](A)[sP].[dR](T)[sP]. | |
| | | [dR](T)[sP].[LR](T)[sP].[LR]([5meC]) | |
| 31 | 31_5 | [LR]([5meC])[sP].[LR](A)[sP].[dR](G)[sP].[LR](T)[sP].[dR](T)[sP]. | 11 |
| | | [dR](T)[sP].[LR](T)[sP].[dR](A)[sP].[dR](A)[sP].[dR](T)[sP]. | |
| | | [dR](A)[sP].[dR](C)[sP].[dR](C)[sP].[dR](A)[sP].[LR](T)[sP]. | |
| | | [dR](T)[sP].[LR](T)[sP].[LR]([5meC]) | |
| 33 | 33_1 | [LR]([5meC])[sP].[LR](A)[sP].[dR](G)[sP].[dR](T)[sP].[LR](T)[sP]. | 12 |
| | | [dR](T)[sP].[dR](T)[sP].[LR](A)[sP].[dR](A)[sP].[dR](T)[sP]. | |
| | | [dR](A)[sP].[dR](C)[sP].[dR](C)[sP].[dR](A)[sP].[dR](T)[sP]. | |
| | | [LR](T)[sP].[dR](T)[sP].[LR]([5meC])[sP].[LR](A) | |
| 33 | 33_2 | [LR]([5meC])[sP].[LR](A)[sP].[dR](G)[sP].[LR](T)[sP].[dR](T)[sP]. | 13 |
| | | [dR](T)[sP].[dR](T)[sP].[dR](A)[sP].[dR](A)[sP].[dR](T)[sP]. | |
| | | [dR](A)[sP].[dR](C)[sP].[dR](C)[sP].[dR](A)[sP].[dR](T)[sP]. | |
| | | [LR](T)[sP].[dR](T)[sP].[LR]([5meC])[sP].[LR](A) | |
| 33 | 33_3 | [LR]([5meC])[sP].[LR](A)[sP].[dR](G)[sP].[dR](T)[sP].[LR](T)[sP]. | 14 |
| | | [dR](T)[sP].[dR](T)[sP].[dR](A)[sP].[dR](A)[sP].[dR](T)[sP]. | |
| | | [dR](A)[sP].[dR](C)[sP].[dR](C)[sP].[LR](A)[sP].[dR](T)[sP]. | |
| | | [LR](T)[sP].[dR](T)[sP].[LR]([5meC])[sP].[LR](A) | |
| 47 | 47_1 | [LR](T)[sP].[LR]([5meC])[sP].[dR](A)[sP].[LR]([5meC])[sP]. | 15 |
| | | [dR](A)[sP].[dR](T)[sP].[dR](A)[sP].[dR](A)[sP].[dR](T)[sP]. | |
| | | [dR](T)[sP].[dR](T)[sP].[dR](A)[sP].[dR](T)[sP].[dR](T)[sP]. | |
| | | [dR](C)[sP].[dR](C)[sP].[LR]([5meC])[sP].[LR](T) | |
| 48 | 48_8 | [LR]([5meC])[sP].[dR](T)[sP].[LR]([5meC])[sP].[dR](A)[sP]. | 16 |
| | | [dR](T)[sP].[dR](A)[sP].[dR](C)[sP].[dR](T)[sP].[dR](G)[sP]. | |
| | | [dR](C)[sP].[dR](T)[sP].[LR]([5meC])[sP].[dR](T)[sP].[dR](T)[sP]. | |
| | | [dR](T)[sP].[dR](C)[sP].[dR](T)[sP].[LR](A) | |
| 29 | 29_24 | [LR](G)[sP].[dR](T)[sP].[dR](T)[sP].[LR](T)[sP].[dR](T)[sP]. | 17 |
| | | [LR](A)[sP].[dR](A)[sP].[dR](T)[sP].[dR](A)[sP].[dR](C)[sP]. | |
| | | [dR](C)[sP].[dR](A)[sP].[dR](T)[sP].[dR](T)[sP].[LR](T)[sP]. | |
| | | [LR]([5meC])[sP].[LR](A) | |
| 29 | 29_25 | [LR](G)[sP].[dR](T)[sP].[dR](T)[sP].[LR](T)[sP].[LR](T)[sP]. | 18 |
| | | [mR](A)[sP].[dR](A)[sP].[dR](T)[sP].[dR](A)[sP].[dR](C)[sP]. | |
| | | [dR](C)[sP].[dR](A)[sP].[dR](T)[sP].[dR](T)[sP].[LR](T)[sP]. | |
| | | [LR]([5meC])[sP].[LR](A) | |
| 29 | 29_26 | [LR](G)[sP].[dR](T)[sP].[dR](T)[sP].[LR](T)[sP].[LR](T)[sP]. | 19 |
| | | [dR](A)[sP].[dR](A)[sP].[dR](T)[sP].[dR](A)[sP].[dR](C)[sP]. | |
| | | [dR](C)[sP].[dR](A)[sP].[dR](T)[sP].[dR](T)[sP].[LR](T)[sP]. | |
| | | [LR]([5meC])[sP].[LR](A) | |
| 39 | 39_9 | [LR](T)[sP].[dR](T)[sP].[LR]([5meC])[sP].[LR](A)[sP].[dR](C)[sP]. | 20 |
| | | [dR](A)[sP].[dR](T)[sP].[dR](A)[sP].[dR](A)[sP].[dR](T)[sP]. | |
| | | [dR](T)[sP].[dR](T)[sP].[dR](A)[sP].[dR](T)[sP].[LR](T)[sP]. | |
| | | [dR](C)[sP].[LR]([5meC])[sP].[LR]([5meC]) | |
| 39 | 39_10 | [LR](T)[sP].[dR](T)[sP].[LR]([5meC])[sP].[mR](A)[sP]. | 21 |
| | | [dR](C)[sP].[dR](A)[sP].[dR](T)[sP].[dR](A)[sP].[dR](A)[sP]. | |
| | | [dR](T)[sP].[dR](T)[sP].[dR](T)[sP].[dR](A)[sP].[dR](T)[sP]. | |
| | | [LR](T)[sP].[mR](C)[sP].[LR]([5meC])[sP].[LR]([5meC]) | |
| 48 | 48_10 | [LR]([5meC])[sP].[dR](T)[sP].[LR]([5meC])[sP].[mR](A)[sP]. | 22 |
| | | [dR](T)[sP].[dR](A)[sP].[dR](C)[sP].[dR](T)[sP].[dR](G)[sP]. | |
| | | [dR](C)[sP].[dR] (T)[sP].[LR]([5meC])[sP].[dR](T)[sP]. | |
| | | [dR](T)[sP].[mR](U)[sP].[dR](C)[sP].[LR](T)[sP].[LR](A) | |
| 29 | 29_35 | [LR](G)[sP].[dR](T)[sP].[dR](T)[sP].[LR](T)[sP].[mR](U)[sP]. | 23 |
| | | [LR](A)[sP].[dR](A)[sP].[dR](T)[sP].[dR](A)[sP].[dR](C)[sP]. | |
| | | [dR](C)[sP].[dR](A)[sP].[dR](T)[sP].[dR](T)[sP].[LR](T)[sP]. | |
| | | [LR]([5meC])[sP].[LR](A) | |
| 29 | 29_34 | [LR](G)[sP].[dR](T)[sP].[dR](T)[sP].[LR](T)[sP].[LR](T)[sP]. | 24 |
| | | [LR](A)[sP].[mR](A)[sP].[dR](T)[sP].[dR](A)[sP].[dR](C)[sP]. | |
| | | [dR](C)[sP].[dR](A)[sP].[dR](T)[sP].[mR](U)[sP].[LR](T)[sP]. | |
| | | [LR]([5meC])[sP].[LR](A) | |
| 39 | 39_17 | [LR](T)[sP].[mR](U)[sP].[LR]([5meC])[sP].[dR](A)[sP].[dR](C)[sP]. | 25 |
| | | [dR](A)[sP].[dR](T)[sP].[dR](A)[sP].[dR] (A)[sP].[dR](T)[sP]. | |
| | | [dR](T)[sP].[dR](T)[sP].[dR](A)[sP].[dR](T)[sP].[LR](T)[sP]. | |
| | | [mR](C)[sP].[LR]([5meC])[sP].[LR]([5meC]) | |
| 39 | 39_18 | [LR](T)[sP].[mR](U)[sP].[LR]([5meC])[sP].[mR](A)[sP].[dR](C)[sP]. | 26 |
| | | [dR](A)[sP].[dR](T)[sP].[dR](A)[sP].[dR](A)[sP].[dR](T)[sP]. | |
| | | [dR](T)[sP].[dR](T)[sP].[dR](A)[sP].[dR](T)[sP].[LR](T)[sP]. | |
| | | [dR](C)[sP].[LR]([5meC])[sP].[LR]([5meC]) | |
| 39 | 39_19 | [LR](T)[sP].[mR](U)[sP].[LR]([5meC])[sP].[mR](A)[sP].[dR](C)[sP]. | 27 |
| | | [dR](A)[sP].[dR](T)[sP].[dR](A)[sP].[dR](A)[sP].[dR](T)[sP]. | |
| | | [dR](T)[sP].[dR](T)[sP].[dR](A)[sP].[dR](T)[sP].[LR](T)[sP]. | |
| | | [mR](C)[sP].[LR]([5meC])[sP].[LR]([5meC]) | |
| 39 | 39_20 | [mR](U)[sP].[LR](T)[sP].[LR]([5meC])[sP].[dR](A)[sP].[dR](C)[sP]. | 28 |
| | | [dR](A)[sP].[dR](T)[sP].[dR](A)[sP].[dR](A)[sP].[dR](T)[sP]. | |
| | | [dR](T)[sP].[dR](T)[sP].[dR](A)[sP].[dR](T)[sP].[LR](T)[sP]. | |
| | | [dR](C)[sP].[LR]([5meC])[sP].[LR]([5meC]) | |
| 29 | 29_11 | [LR](G)[sP].[dR](T)[sP].[dR](T)[sP].[LR](T)[sP].[LR](T)[sP]. | 29 |
| | | [LR](A)[sP].[dR](A)[sP].[dR](T)[sP].[dR](A)[sP].[dR](C)[sP]. | |
| | | [dR](C)[sP].[dR](A)[sP].[dR](T)[sP].[dR](T)[sP].[LR](T)[sP]. | |
| | | [dR](C)[sP].[LR](A) | |

Helm Annotation Key:
[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC] is a beta-D-oxy-LNA 5-methyl cytosine nucleoside,
[dR](G) is a DNA guanine nucleoside,
[dR](T) is a DNA thymine nucleoside,
[dR](A) is a DNA adenine nucleoside,
[dR]([C] is a DNA cytosine nucleoside,
[mR](G) is a 2′-O-methyl RNA guanine nucleoside,
[mR](U) is a 2′-O-methyl RNA DNA uracil nucleoside,
[mR](A) is a 2′-O-methyl RNA DNA adenine nucleoside,
[mR]([C] is a 2′-O-methyl RNA DNA cytosine nucleoside,
[sP] is a phosphorothioate internucleoside linkage.

In some embodiments, the antisense oligonucleotide of the invention may comprise one or more conjugate groups, i.e. the antisense oligonucleotide may be an antisense oligonucleotide conjugate.

In some embodiments, the antisense oligonucleotide of the invention consists of the contiguous nucleotide sequence.

The invention provides pharmaceutical compositions comprising the antisense oligonucleotide of the invention and a pharmaceutically acceptable diluents, carriers, salts and/or adjuvants.

The invention provides for a pharmaceutically acceptable salt of the antisense oligonucleotide of the invention. In some embodiments, the pharmaceutically acceptable salt is selected from the group consisting of a sodium salt, a potassium salt and an ammonium salt.

The invention provides for a pharmaceutical solution of the antisense oligonucleotide of the invention, wherein the pharmaceutical solution comprises the antisense oligonucleotide of the invention and a pharmaceutically acceptable solvent, such as phosphate buffered saline.

The invention provides for the antisense oligonucleotide of the invention in solid powdered form, such as in the form of a lyophilized powder.

The invention provides for a conjugate comprising the antisense oligonucleotide according to the invention, and at least one conjugate moiety covalently attached to said antisense oligonucleotide.

The invention provides for a pharmaceutically acceptable salt of the antisense oligonucleotide of the invention, or the conjugate according to the invention.

The invention provides for a pharmaceutically acceptable salt of the antisense oligonucleotide according to the invention, wherein the pharmaceutically acceptable salt is a sodium or potassium salt.

The invention provides for a pharmaceutical composition comprising the antisense oligonucleotide of the invention or the conjugate of the invention, or the salt of the invention and a pharmaceutically acceptable diluent, solvent, carrier, salt and/or adjuvant.

The invention provides for a method for inhibiting SCN9A expression in a target cell, which is expressing SCN9A, said method comprising administering an antisense oligonucleotide of the invention or the conjugate of the invention, or the salt of the invention, or the composition of the invention in an effective amount to said cell. The method may be an in vivo method or an in vitro method.

The invention provides for a method for treating or preventing pain in a subject such as a human, who is suffering from or is likely to suffer pain, comprising administering a therapeutically or prophylactically effective amount of an antisense oligonucleotide of the invention or the conjugate of the invention, or the salt of the invention, or the composition of the invention, such as to prevent or alleviate the pain.

In some embodiments, the antisense oligonucleotide of the invention or the conjugate of the invention, or the salt of the invention, or the composition of the invention, is for the use in the treatment of chronic pain, neuropathic pain, inflammatory pain, or spontaneous pain.

In some embodiments, the antisense oligonucleotide of the invention or the conjugate of the invention, or the salt of the invention, or the composition of the invention, is for the use in the treatment of nociceptive pain.

In some embodiments, the antisense oligonucleotide of the invention or the conjugate of the invention, or the salt of the invention, or the composition of the invention, is for the use in the treatment of pain caused by or associated with a disorder selected from the group consisting of diabetic neuropathies, cancer, cranial neuralgia, postherpetic neuralgia and post-surgical neuralgia.

In some embodiments, the antisense oligonucleotide of the invention or the conjugate of the invention, or the salt of the invention, or the composition of the invention, is for the use in the treatment of pain caused by or associated with inherited erythromelalgia (EIM) or paroxysmal extreme pain disorder (PEPD) or trigeminal neuralgia.

In some embodiments, the antisense oligonucleotide of the invention or the conjugate of the invention, or the salt of the invention, or the composition of the invention, is for the use in the treatment of neurophathic pain, chronic pain, but also general treatment of nociceptive pain (e.g. decompression of a nerve), or neuropathic pain (e.g. diabetic neuropathy), visceral pain, or mixed pain.

In some embodiments, the antisense oligonucleotide of the invention or the conjugate of the invention, or the salt of the invention, or the composition of the invention, is for the use in the treatment of lower back pain, or inflammatory arthritis.

The invention provides the antisense oligonucleotide of the invention or the conjugate of the invention, or the composition or the salt of the invention for use in medicine.

In a further aspect, the invention provides methods for a method for inhibition of SCN9A expression in a target cell, which is expressing SCN9A, by administering an antisense oligonucleotide or composition of the invention in an effective amount to said cell. In a further aspect, the invention provides methods for in vivo or in vitro method for inhibition of SCN9A expression in a target cell, which is expressing SCN9A, by administering an antisense oligonucleotide or composition of the invention in an effective amount to said cell. The cell may for example be a human cell, such as a neuronal cell, such as a peripheral nerve cell, or a primary neuronal cell.

In a further aspect, the invention provides methods for treating or preventing a disease selected from the group consisting of or prevention of pain, such as peripheral pain comprising administering a therapeutically or prophylactically effective amount of the antisense oligonucleotide of the invention to a subject suffering from or susceptible to pain, such as peripheral pain.

In a further aspect, the invention provides the antisense oligonucleotide, the conjugate, or the pharmaceutical composition of the invention, for use in the manufacture of a medicament for the treatment or prevention of pain, such as peripheral pain.

In a further aspect, the invention provides the antisense oligonucleotide, the conjugate, or the pharmaceutical composition of the invention, for use in the manufacture of an analgesic.

The invention provides for the antisense oligonucleotide of the invention for use in the treatment of pain, such as peripheral pain.

The invention provides for the antisense oligonucleotide of the invention for use as an analgesic.

In a further aspect, the invention provides methods for treating or preventing pain comprising administering a therapeutically or prophylactically effective amount of the antisense oligonucleotide of the invention to a subject suffering from or susceptible to pain.

In a further aspect, the invention provides methods for treating or preventing peripheral pain comprising administering a therapeutically or prophylactically effective amount of the antisense oligonucleotide of the invention to a subject suffering from or susceptible to peripheral pain.

### SEQUENCE LISTING

The sequence listing submitted with this application is hereby incorporated by reference. The antisense oligonucleotide sequence motifs listed in the sequence listing are illustrated as DNA sequences. It will be noted that in some of the tested compounds disclosed herein 2′-O-methyl RNA nucleosides are used, with uracil in place or thymine bases.

### FIGURES

FIG. 1 Compound ID NO#29_15

FIG. 2 Compound ID NO#29_10

FIG. 3 Compound ID NO#29_22

FIG. 4 Compound ID NO#39_6

FIG. 5 Compound ID NO#39_1

FIG. 6 Compound ID NO#39_2

FIG. 7 Compound ID NO#39_7

FIG. 8 Compound ID NO#31_1

FIG. 9 Compound ID NO#31_3

FIG. 10 Compound ID NO#31_4

FIG. 11 Compound ID NO#31_5

FIG. 12 Compound ID NO#33_1

FIG. 13 Compound ID NO#33_2

FIG. 14 Compound ID NO#33_3

FIG. 15 Compound ID NO#47_1

FIG. 16 Compound ID NO#48_8

FIG. 17 Compound ID NO#29_24

FIG. 18 Compound ID NO#29_25

FIG. 19 Compound ID NO#29_26

FIG. 20 Compound ID NO#39_9

FIG. 21 Compound ID NO#39_10

FIG. 22 Compound ID NO#48_10

FIG. 23 Compound ID NO#29_35

FIG. 24 Compound ID NO#29_34

FIG. 25 Compound ID NO#39_17

FIG. 26 Compound ID NO#39_18

FIG. 27 Compound ID NO#39_19

FIG. 28 Compound ID NO#39_20

FIG. 29 Compound ID NO#29_11

FIG. 30. Low vs. high flushing volume. The chart shows exposure as a function of high (left) and low (right) flushing volume after 16 mg compound dosing. Each DRG data point was from pooling of two DRGs from one cynomolus (N=3). All data points were from cynomolgus monkeys sacrificed 14 days post a single, 16 mg dose of Compound ID NO#31_1.

FIG. 31. Dorsal root ganglia exposure as a function of days post-dosing. Each data point was the pooling of two DRGs from a cynomolgus monkey (N=3). All data were from the low flushing volume groups and a single dose of 16 mg of Compound ID NO#31_1.

### DEFINITIONS

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides such as 2′ sugar modified nucleosides. The oligonucleotide of the invention may comprise one or more modified internucleoside linkages, such as one or more phosphorothioate internucleoside linkages.

Antisense Oligonucleotides

The term “antisense oligonucleotide” as used herein is defined as oligonucleotides capable of modulating expression of a target gene by hybridizing to a target nucleic acid, in particular to a contiguous sequence on a target nucleic acid. Antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self-complementarity is less than 50% across of the full length of the oligonucleotide.

In some embodiments, the single stranded antisense oligonucleotide of the invention may not contain non modified RNA nucleosides.

Advantageously, the antisense oligonucleotide of the invention comprises one or more modified nucleosides or nucleotides, such as 2′ sugar modified nucleosides. Furthermore, it is advantageous that the nucleosides which are not modified are DNA nucleosides.

Contiguous Nucleotide Sequence

The term “contiguous nucleotide sequence” refers to the region of the antisense oligonucleotide which is complementary to the target nucleic acid. The term is used interchangeably herein with the term “contiguous nucleobase sequence” and the term “oligonucleotide motif sequence”. In some embodiments, all the nucleosides of the oligonucleotide constitute the contiguous nucleotide sequence.

In some embodiments, the oligonucleotide comprises the contiguous nucleotide sequence, such as a F-G-F′ gapmer region, and may optionally comprise further nucleotide(s), for example a nucleotide linker region which may be used to attach a functional group (e.g. a conjugate group) to the contiguous nucleotide sequence. The nucleotide linker region may or may not be complementary to the target nucleic acid. In some embodiments, the nucleobase sequence of the antisense oligonucleotide is the contiguous nucleotide sequence.

Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. Advantageously, one or more of the modified nucleosides of the antisense oligonucleotide of the invention comprise a modified sugar moiety. The term “modified nucleoside” may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

Modified Internucleoside Linkage

The term “modified internucleoside linkage” is defined as generally understood by the skilled person as linkages other than phosphodiester (PO) linkages, that covalently couples two nucleosides together. The oligonucleotides of the invention may therefore comprise one or more modified internucleoside linkages such as a one or more phosphorothioate internucleoside linkages, or one or more phoshporodithioate internucleoside linkages.

In some embodiments, at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments, all of the internucleoside linkages of the oligonucleotide, or contiguous nucleotide sequence thereof, are phosphorothioate.

In some advantageous embodiments, all the internucleoside linkages of the contiguous nucleotide sequence of the oligonucleotide are phosphorothioate, or all the internucleoside linkages of the oligonucleotide are phosphorothioate linkages.

It is recognized that, as disclosed in EP 2 742 135, antisense oligonucleotides may comprise other internucleoside linkages (other than phosphodiester, phosphorothioate and phosphorodithioate), for example alkyl phosphonate/methyl phosphonate internucleoside, which according to EP 2 742 135 may for example be tolerated in an otherwise DNA phosphorothioate the gap region.

Nucleobase

The term “nucleobase” includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments, the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function.

For example, in the exemplified oligonucleotides, the nucleobase moieties are selected from A, T, G, C, and 5-methyl cytosine. Optionally, for LNA gapmers, 5-methyl cytosine LNA nucleosides may be used.

Modified Oligonucleotide

The term “modified oligonucleotide” describes an oligonucleotide comprising one or more sugar-modified nucleosides and/or modified internucleoside linkages. The term chimeric” oligonucleotide is a term that has been used in the literature to describe oligonucleotides comprising sugar modified nucleosides and DNA nucleosides. The antisense oligonucleotide of the invention is advantageously a chimeric oligonucleotide.

Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

The term “fully complementary”, refers to 100% complementarity.

Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments, the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments, the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

The Target

The term “target” as used herein is used to refer to the human sodium voltage-gated channel alpha subunit 9 (SCN9A), and nucleic acids which encode for the human SCN9A, as illustrated herein as SEQ ID NO: 1. The SCN9A nucleic acid encodes the alpha subunit of a sodium channel called Naᵥ1.7.

Target Nucleic Acid

According to the present invention, the target nucleic acid is a nucleic acid which encodes the human SCN9A and may for example be a gene, a RNA, a mRNA, and pre-mRNA, a mature mRNA or a cDNA sequence. The target may therefore be referred to as an SCN9A target nucleic acid. For in vitro and in vivo use, a preferred target nucleic acid is the pre-mRNA or mRNA encoding SCN9A. If employing the oligonucleotide of the invention in research or diagnostics the target nucleic acid may be a cDNA or a synthetic nucleic acid derived from DNA or RNA.

A preferred target gene is the human SCN9A, for example the human SCN9A pre-mRNA (see genetic coordinates provided in Table 2, and as illustrated herein as SEQ ID NO: 1.

TABLE 2
Genome and assembly information for human and cyno SCN9A target gene

| | | | Genomic coordinates | | | |
|---|---|---|---|---|---|---|
| Species | Chr | Strand | Start | End | Assembly | Exemplary sequence |
| Human | 2 | Rev | 166195185 | 166375993 | GRCh38.p12 | SEQ ID NO: 1 |
| Cyno | 12 | Rev | 55049339 | 55256120 | Macaca_fascicularis_5 | SEQ ID NO: 2 |

In some embodiments, the target nucleic acid is a transcript variant of SEQ ID NO: 1—i.e. a transcript which is transcribed from the SCN9A gene encoded from the human chromosomal locus (coordinates are identified in Table 2).

Target Sequence

The term “target sequence” as used herein refers to a sequence of nucleotides present in the target nucleic acid which comprises the nucleobase sequence which is complementary to the antisense oligonucleotide of the invention. In some embodiments, the target sequence consists of a region on the target nucleic acid with a nucleobase sequence that is complementary to the contiguous nucleotide sequence of the antisense oligonucleotide of the invention. This region of the target nucleic acid may interchangeably be referred to as the target nucleotide sequence, target sequence or target region. In some embodiments, the target sequence is longer than the complementary sequence of a single antisense oligonucleotide, and may, for example represent a preferred region of the target nucleic acid which may be targeted by several antisense oligonucleotides of the invention.

In some embodiments, the antisense oligonucleotide of the invention, or the contiguous nucleotide sequence thereof, is complementary, such as fully complementary to a target sequence selected from nucleotides 97704-97732, 103232-103259, 151831-151847, and 151949-152006, of SEQ ID NO: 1.

The antisense oligonucleotide of the invention comprises a contiguous nucleotide sequence which is complementary to and hybridizes to the target nucleic acid, such as a target sequence described herein.

The target sequence to which the antisense oligonucleotide is complementary to generally comprises a contiguous nucleobases sequence of at least 10 nucleotides. The contiguous nucleotide sequence is between 10 to 30 nucleotides in length, such as 12 to 30, such as 14 to 20, such as 15 to 18 contiguous nucleotides in length, such as 15, 16, 17 contiguous nucleotides in length.

In some embodiments, the antisense oligonucleotide of the invention is fully complementary to the target sequence across the full length of the antisense oligonucleotide.

Target Cell

The term a “target cell” as used herein refers to a cell which is expressing the target nucleic acid. In some embodiments, the target cell may be in vivo or in vitro. In some embodiments, the target cell is a mammalian cell such as a rodent cell, such as a mouse cell or a rat cell, or a primate cell such as a monkey cell or a human cell.

Typically, the target cell expresses the SCN9A mRNA, such as the SCN9A pre-mRNA or SCN9A mature mRNA. For experimental evaluation a target cell may be used which expresses a nucleic acid which comprises a target sequence.

The poly A tail of SCN9A mRNA is typically disregarded for antisense oligonucleotide targeting. The antisense oligonucleotide of the invention is typically capable of inhibiting the expression of the SCN9A target nucleic acid in a cell which is expressing the SCN9A target nucleic acid (a target cell), for example either in vivo or in vitro.

The contiguous sequence of nucleobases of the antisense oligonucleotide of the invention is complementary, such as fully complementary to the SCN9A target nucleic acid, such as SEQ ID NO: 1, as measured across the length of the antisense oligonucleotide, optionally excluding nucleotide based linker regions which may link the antisense oligonucleotide to an optional functional group such as a conjugate, or other non-complementary terminal nucleotides (e.g. region D′ or D″). The target nucleic acid may for example be a messenger RNA, such as a mature mRNA or a pre-mRNA, which encodes SCN9A.

Naturally Occurring Variant

The term “naturally occurring variant” refers to variants of SCN9A gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the oligonucleotide, the oligonucleotide of the invention may therefore target the target nucleic acid and naturally occurring variants thereof.

In some embodiments, the naturally occurring variants have at least 95% such as at least 98% or at least 99% homology to a mammalian SCN9A target nucleic acid, such as a target nucleic acid selected form the group consisting of SEQ ID NO: 1. In some embodiments, the naturally occurring variants have at least 99% homology to the human SCN9A target nucleic acid of SEQ ID NO: 1.

Inhibition of Expression

The term “Inhibition of expression” as used herein is to be understood as an overall term for an oligonucleotide's ability to inhibit the amount or the activity of SCN9A in a target cell. Inhibition of activity may be determined by measuring the level of SCN9A pre-mRNA or SCN9A mRNA, or by measuring the level of SCN9A or SCN9A activity in a cell. Inhibition of expression may therefore be determined in vitro or in vivo. Inhibition of SCN9A expression may also be determined by measuring the Naᵥ1.7 activity or protein level.

Typically, inhibition of expression is determined by comparing the inhibition of activity due to the administration of an effective amount of the antisense oligonucleotide to the target cell and comparing that level to a reference level obtained from a target cell without administration of the antisense oligonucleotide (control experiment), or a known reference level (e.g. the level of expression prior to administration of the effective amount of the antisense oligonucleotide, or a predetermine or otherwise known expression level).

For example a control experiment may be an animal or person, or a target cell treated with a saline composition or a reference oligonucleotide (often a scrambled control).

The term inhibition or inhibit may also be referred as down-regulate, reduce, suppress, lessen, lower, the expression of SCN9A.

The inhibition of expression may occur e.g. by degradation of pre-mRNA or mRNA (e.g. using RNaseH recruiting oligonucleotides, such as gapmers).

High Affinity Modified Nucleosides

A high affinity modified nucleoside is a modified nucleotide which, when incorporated into the antisense oligonucleotide enhances the affinity of the antisense oligonucleotide for its complementary target, for example as measured by the melting temperature (Tm). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

Sugar Modifications

The antisense oligonucleotide of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance. Such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′—OH group naturally found in DNA and RNA nucleosides. Substituents may, for example be introduced at the 2′, 3′, 4′ or 5′ positions.

2′ Sugar Modified Nucleosides

A 2′ sugar modified nucleoside is a nucleoside which has a substituent other than H or —OH at the 2′ position (2′ substituted nucleoside) or comprises a 2′ linked biradical capable of forming a bridge between the 2′ carbon and a second carbon in the ribose ring, such as LNA (2′-4′ biradical bridged) nucleosides.

Indeed, much focus has been spent on developing 2′ sugar substituted nucleosides, and numerous 2′ substituted nucleosides have been found to have beneficial properties when incorporated into antisense oligonucleotides. For example, the 2′ modified sugar may provide enhanced binding affinity and/or increased nuclease resistance to the antisense oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

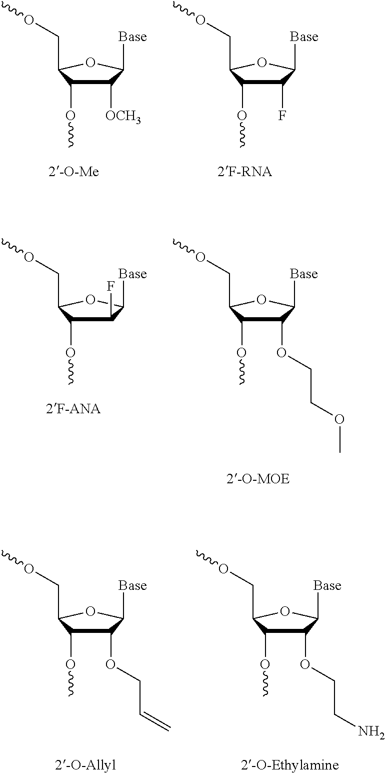

In relation to the present invention 2′ substituted sugar modified nucleosides does not include 2′ bridged nucleosides like LNA.

Locked Nucleic Acid Nucleosides (LNA Nucleoside)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an antisense oligonucleotide for a complementary RNA or DNA molecule. This can be routinely determined by measuring the melting temperature of the antisense oligonucleotide/complement duplex. Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

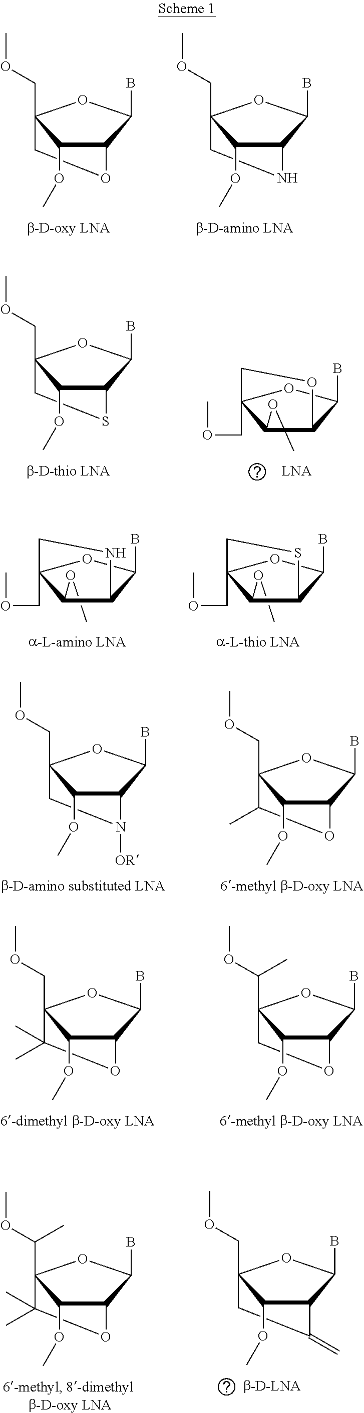

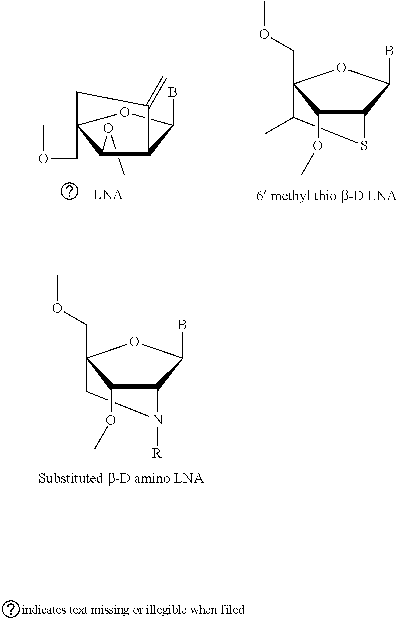

Particular LNA nucleosides are beta-D-oxy-LNA, 6′-methyl-beta-D-oxy LNA such as (S)-6′-methyl-beta-D-oxy-LNA (ScET) and ENA. A particularly advantageous LNA is beta-D-oxy-LNA.

Exemplary nucleosides, with HELM Annotation

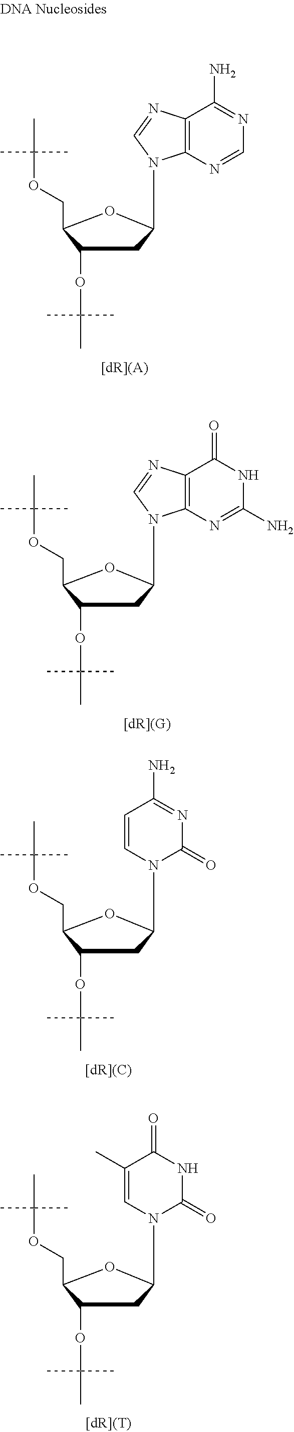

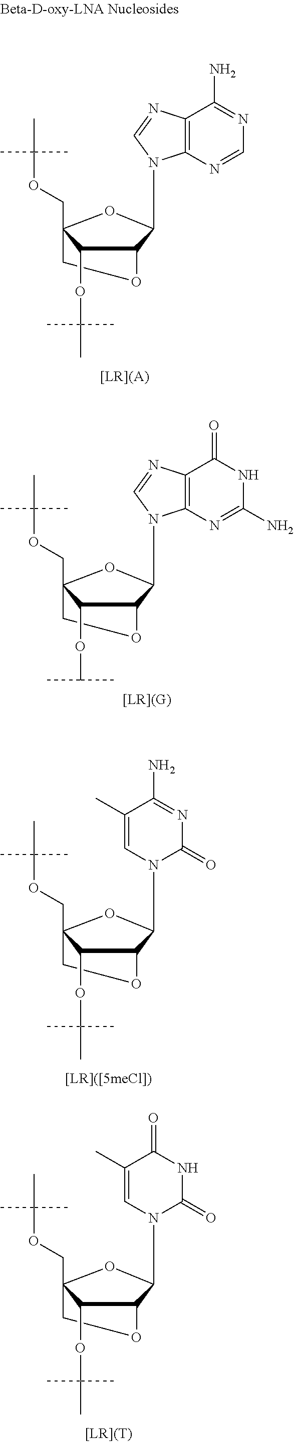

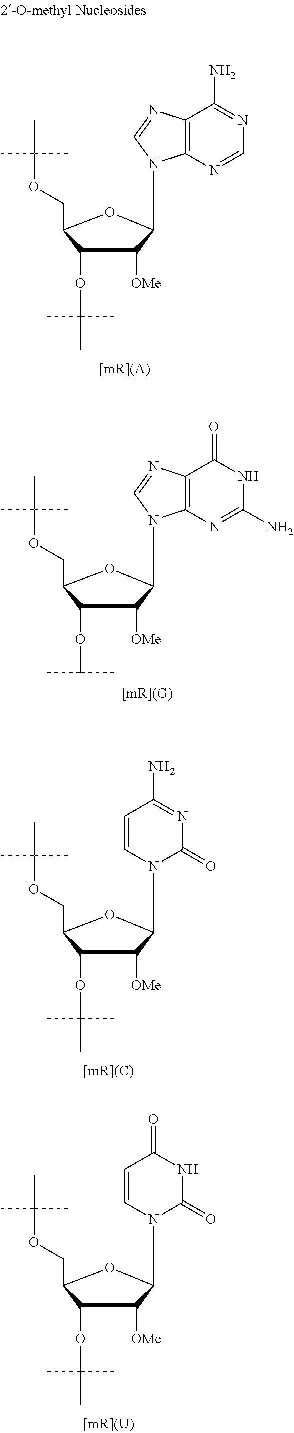

Exemplary Phosphorothioate Internucleoside Linkage with HELM Annotation

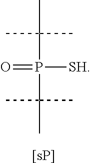

The dotted lines represent the covalent bond between each nucleoside and the 5′ or 3′ phosphorothioate internucleoside linkages. At the 5′ terminal nucleoside, the 5′ dotted lines represent a bond to a hydrogen atom (forming a 5′ terminal —OH group). At the 3′ terminal nucleoside, the 3′ dotted lines represent a bond to a hydrogen atom (forming a 3′ terminal —OH group).

RNase H Activity and Recruitment

The RNase H activity of an antisense oligonucleotide refers to its ability to recruit RNase H when in a duplex with a complementary RNA molecule. WO01/23613 provides in vitro methods for determining RNase H activity, which may be used to determine the ability to recruit RNase H. Typically an antisense oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a antisense oligonucleotide having the same base sequence as the modified oligonucleotide being tested, but containing only DNA monomers with phosphorothioate linkages between all monomers in the antisense oligonucleotide, and using the methodology provided by Example 91-95 of WO01/23613 (hereby incorporated by reference). For use in determining RNase H activity, recombinant human RNase H1 is available from Creative Biomart® (Recombinant Human RNASEH1 fused with His tag expressed in E. coli).

Gapmer

The antisense oligonucleotide of the invention, or contiguous nucleotide sequence thereof, may be a gapmer, also termed gapmer antisense oligonucleotide or gapmer designs. The gapmers are commonly used to inhibit a target nucleic acid via RNase H mediated degradation. A gapmer comprises at least three distinct structural regions a 5′-flank, a gap and a 3′-flank, F-G-F′ in the ‘5->3’ orientation. The “gap” region (G) comprises a stretch of contiguous DNA nucleotides which enable the gapmer to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the gapmer for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further be defined by having at least one sugar modified nucleoside at the end most distant from the gap region, i.e. at the 5′ end of the 5′ flank and at the 3′ end of the 3′ flank. Regions F-G-F′ form a contiguous nucleotide sequence. Antisense oligonucleotides of the invention, or the contiguous nucleotide sequence thereof, may comprise a gapmer region of formula F-G-F′. The overall length of the gapmer design F-G-F′ may be, for example 12 to 32 nucleosides, such as 13 to 24, such as 14 to 22 nucleosides, Such as from 14 to 17, such as 16 to 18 nucleosides. By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈

with the proviso that the overall length of the gapmer regions F-G-F′ is at least 12, such as at least 14 nucleotides in length.

In an aspect of the invention the antisense oligonucleotide or contiguous nucleotide sequence thereof consists of or comprises a gapmer of formula 5′-F-G-F′-3′, where region F and F′ independently comprise or consist of 1-8 nucleosides, of which 1-4 are 2′ sugar modified and defines the 5′ and 3′ end of the F and F′ region, and G is a region between 6 and 16 nucleosides which are capable of recruiting RNase H.

Regions F, G and F′ are further defined below and can be incorporated into the F-G-F′ formula.

LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments, the LNA gapmer is of formula: [LNA]1_5-[region G]-[LNA]1-5, wherein region G is or comprises a region of contiguous DNA nucleosides which are capable of recruiting RNase H.

MOE Gapmers

A MOE gapmers is a gapmer wherein regions F and F′ consist of MOE nucleosides. In some embodiments, the MOE gapmer is of design [MOE]₁₋₈-[Region G]₅₋₁₆-[MOE]₁₋₈, such as [MOE]₂₋₇-[Region G]₆₋₁₄-[MOE]₂₋₇, such as [MOE]₃₋₆-[Region G]₈₋₁₂-[MOE]₃₋₆, wherein region G is as defined in the Gapmer definition. MOE gapmers with a 5-10-5 design (MOE-DNA-MOE) have been widely used in the art.

Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleoside. In some embodiments, wherein at least one of region F and F′, or both region F and F′ comprise at least one LNA nucleoside, the remaining nucleosides of region F and F′ are independently selected from the group consisting of MOE and LNA. In some embodiments, wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

Alternating Flank Gapmers

Flanking regions may comprise both LNA and DNA nucleoside and are referred to as “alternating flanks” as they comprise an alternating motif of LNA-DNA-LNA nucleosides. Gapmers comprising such alternating flanks are referred to as “alternating flank gapmers”. “Alternative flank gapmers” are thus LNA gapmer oligonucleotides where at least one of the flanks (F or F′) comprises DNA in addition to the LNA nucleoside(s). In some embodiments, at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

Region D′ or D″ in an Antisense Oligonucleotide

The antisense oligonucleotide of the invention may In some embodiments, comprise or consist of the contiguous nucleotide sequence of the oligonucleotide which is complementary to the target nucleic acid, such as a gapmer region F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the antisense oligonucleotide and region D′ or D″ constitute a separate part of the antisense oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments, the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single antisense oligonucleotide.

In one embodiment, the antisense oligonucleotide of the invention comprises a region D′ and/or D″ in addition to the contiguous nucleotide sequence which constitutes the gapmer.

In some embodiments, the antisense oligonucleotide of the present invention can be represented by the following formulae:

F-G-F′; in particular F₁₋₈-G₅₋₁₆-F′₂₋₈

D′-F-G-F′, in particular D′₁₋₃-F₁₋₈-G₅₋₁₆-F′₂₋₈

F-G-F′-D″, in particular F₁₋₈-G₅₋₁₆-F′₂₋₈-D″₁₋₃

D′-F-G-F′-D″, in particular D′₁₋₃-F₁₋₈-G₅₋₁₆-F′₂₋₈-D″₁₋₃

In some embodiments, the internucleoside linkage positioned between region D′ and region F is a phosphodiester linkage. In some embodiments, the internucleoside linkage positioned between region F′ and region D″ is a phosphodiester linkage.

Conjugate

The term conjugate as used herein refers to an antisense oligonucleotide which is covalently linked to a non-nucleotide moiety (conjugate moiety or region C or third region). The conjugate moiety may be covalently linked to the antisense oligonucleotide, optionally via a linker group, such as region D′ or D″.

Antisense oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103.

In some embodiments, the non-nucleotide moiety (conjugate moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

Linkers

A linkage or linker is a connection between two atoms that links one chemical group or segment of interest to another chemical group or segment of interest via one or more covalent bonds. Conjugate moieties can be attached to the antisense oligonucleotide directly or through a linking moiety (e.g. linker or tether). Linkers serve to covalently connect a third region, e.g. a conjugate moiety (Region C), to a first region, e.g. an antisense oligonucleotide or contiguous nucleotide sequence complementary to the target nucleic acid (region A).

In some embodiments, of the invention the conjugate or antisense oligonucleotide conjugate of the invention may optionally, comprise a linker region (second region or region B and/or region Y) which is positioned between the antisense oligonucleotide or contiguous nucleotide sequence complementary to the target nucleic acid (region A or first region) and the conjugate moiety (region C or third region). Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment, the biocleavable linker is susceptible to Si nuclease cleavage. In some embodiments, the nuclease susceptible linker comprises between 1 and 5 nucleosides, such as DNA nucleoside(s) comprising at least two consecutive phosphodiester linkages. Phosphodiester containing biocleavable linkers are described in more detail in WO 2014/076195.

Region Y refers to linkers that are not necessarily biocleavable but primarily serve to covalently connect a conjugate moiety (region C or third region), to an antisense oligonucleotide (region A or first region). The region Y linkers may comprise a chain structure or an oligomer of repeating units such as ethylene glycol, amino acid units or amino alkyl groups. The antisense oligonucleotide conjugates of the present invention can be constructed of the following regional elements A-C, A-B-C, A-B-Y-C, A-Y-B-C or A-Y-C. In some embodiments, the linker (region Y) is an amino alkyl, such as a C2-C36 amino alkyl group, including, for example C6 to C12 amino alkyl groups. In some embodiments, the linker (region Y) is a C6 amino alkyl group. In some embodiments, the linker is NA.

Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, In some embodiments, be prophylactic.

### DETAILED DESCRIPTION OF THE INVENTION

The Antisense Oligonucleotides of the Invention

The antisense oligonucleotide of the invention is an antisense oligonucleotide, which targets SCN9A.

The Antisense Oligonucleotide

In some embodiments, the antisense oligonucleotide of the invention is capable of modulating the expression of the target by inhibiting or down-regulating it. Preferably, such modulation produces an inhibition of expression of at least 20% compared to the normal expression level of the target, more preferably at least 30%, at least 40%, at least 50% inhibition compared to the normal expression level of the target. In some embodiments, antisense oligonucleotides of the invention may be capable of inhibiting expression levels of SCN9A mRNA by at least 60% or 70% in vitro following application of 0.031 μM, 0.1 μM, and 0.4 μM antisense oligonucleotide to SK-N-AS cells. In some embodiments, antisense oligonucleotides of the invention may be capable of inhibiting expression levels of SCN9A protein by at least 50% in vitro following application of 0.031 μM, 0.1 μM, and 0.4 μM oligonucleotide to SK-N-AS cells. Suitably, the Examples provide assays which may be used to measure SCN9A RNA or protein inhibition (e.g. see Examples 1 and 3). In some embodiments, an antisense oligonucleotide of the invention can inhibit the expression level of the target RNA or protein in a cell with a half-maximal effective concentration (EC50) of no more than 1 μM, more preferably no more than 0.5 μM. For example, the antisense oligonucleotide may be capable of inhibiting the expression level of SCN9A mRNA (or protein) with an EC50 of no more than 0.3 μm, such as no more than 0.20 μM, such as no more than 0.15 μM, such as no more than 0.10 μM, such as no more than 0.08 μM, such as no more than 0.07 μM, such as no more than 0.06, 0.05, 0.04 or 0.03 μM, following application of the oligonucleotide to SK-N-AS cells. In some embodiments, the antisense oligonucleotide may be capable of inhibiting the expression level of SCN9A mRNA with an EC50 in the range of 0.03 μM to 0.15 μM, such as in the range of 0.05 to 0.10 μM, such as about 0.07 μM, in SK-N-AS cells. Suitably, this may be evaluated in the assay provided in Example 3.

An antisense oligonucleotide of the invention may also be characterized by a high selectivity for the target nucleic acid, e.g., the SCN9A mRNA. In some embodiments, the antisense oligonucleotide may, in a target cell that expresses a nucleic acid which comprises the target sequence, reduce the expression of few or no off-target nucleic acids, such no more than 20, such as no more than 15, such as no more than 12, such as no more than 10, such as no more than 8, 7, 6, 5, 4, 3, 2 or 1 off-target gene(s), or no off-target genes, e.g. when applied to the target cell at a concentration corresponding to about 50 times its EC50 in SK-N-AS cells. It is to be understood that “off-target gene” includes any gene or gene transcript that is not an SCN9A gene or gene transcript, e.g., mRNA, but whose expression is reduced by the antisense oligonucleotide. Preferably, when applied to a human neuronal cell, e.g., a human iCell GlutaNeuron (see Table 10), at a concentration of about 3 μM, the antisense oligonucleotide may reduce the expression of no more than 5, such as no more than 3, such as no more than 1 off-target gene(s), such a no off-target gene. A suitable assay for evaluating the selectivity of the antisense oligonucleotide is provided in Example 4. In some embodiments, an off-target gene may be defined as having reduced expression vs. control condition with adjusted p-value<0.05 when tested in the assay in Example 4, optionally also being among the top 1% predicted off-target genes based on binding affinity predictions or being able to bind to the corresponding unspliced transcript with 1 mismatch.

The target modulation is triggered by the hybridization between a contiguous nucleotide sequence of the antisense oligonucleotide and the target nucleic acid. In some embodiments, the antisense oligonucleotide of the invention comprises mismatches between the antisense oligonucleotide and the target nucleic acid. Despite mismatches, hybridization to the target nucleic acid may still be sufficient to show a desired modulation of SCN9A expression. Reduced binding affinity resulting from mismatches may advantageously be compensated by increased number of nucleotides in the antisense oligonucleotide and/or an increased number of modified nucleosides capable of increasing the binding affinity to the target, such as 2′ sugar modified nucleosides, including LNA, present within the antisense oligonucleotide sequence.

An aspect of the present invention relates to an antisense oligonucleotide, which comprises a contiguous nucleotide sequence of 10 to 30 nucleotides in length with at least 90% complementarity to SCN9A pre-mRNA, such as SEQ ID NO: 1, or a transcript variant derived therefrom.

In some embodiments, the antisense oligonucleotide comprises a contiguous sequence of 10 to 30 nucleotides in length, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence.

It is advantageous if the antisense oligonucleotide of the invention, or contiguous nucleotide sequence thereof is fully complementary (100% complementary) to a region of the target nucleic acid, or in some embodiments, may comprise one or two mismatches between the antisense oligonucleotide and the target nucleic acid.

In some embodiments, the antisense oligonucleotide comprises a contiguous nucleotide sequence of 10 to 30 nucleotides in length with at least 90% complementary, such as fully (or 100%) complementary, to a region of the target nucleic acid present in SEQ ID NO: 1 selected from the group consisting of selected from nucleotides 97704-97732, 103232-103259, 151831-151847, and 151949-152006, of SEQ ID NO: 1.

In some embodiments, the antisense oligonucleotide comprises a contiguous nucleotide sequence of 14 to 20 nucleotides in length which are fully (or 100%) complementary, to a region of the target nucleic acid present in SEQ ID NO: 1 selected from the group consisting of selected from nucleotides 97704-97732, 103232-103259, 151831-151847, and 151949-152006, of SEQ ID NO: 1.

In some embodiments, the antisense oligonucleotide comprises a contiguous nucleotide sequence of 15 nucleotides in length which are fully (or 100%) complementary, to a region of the target nucleic acid present in SEQ ID NO: 1 selected from the group consisting of selected from nucleotides 97704-97732, 103232-103259, 151831-151847, and 151949-152006, of SEQ ID NO: 1.

In some embodiments, the antisense oligonucleotide comprises a contiguous nucleotide sequence of 16 nucleotides in length which are fully (or 100%) complementary, to a region of the target nucleic acid present in SEQ ID NO: 1 selected from the group consisting of selected from nucleotides 97704-97732, 103232-103259, 151831-151847, and 151949-152006, of SEQ ID NO: 1.

In some embodiments, the antisense oligonucleotide comprises a contiguous nucleotide sequence of 17 nucleotides in length which are fully (or 100%) complementary, to a region of the target nucleic acid present in SEQ ID NO: 1 selected from the group consisting of selected from nucleotides 97704-97732, 103232-103259, 151831-151847, and 151949-152006, of SEQ ID NO: 1.

In some embodiments, the antisense oligonucleotide comprises a contiguous nucleotide sequence of 18 nucleotides in length which are fully (or 100%) complementary, to a region of the target nucleic acid present in SEQ ID NO: 1 selected from the group consisting of selected from nucleotides 97704-97732, 103232-103259, 151831-151847, and 151949-152006, of SEQ ID NO: 1.

In some embodiments, the antisense oligonucleotide comprises a contiguous nucleotide sequence of 19 nucleotides in length which are fully (or 100%) complementary, to a region of the target nucleic acid present in SEQ ID NO: 1 selected from the group consisting of selected from nucleotides 97704-97732, 103232-103259, 151831-151847, and 151949-152006, of SEQ ID NO: 1.

In some embodiments, the antisense oligonucleotide comprises a contiguous nucleotide sequence of 20 nucleotides in length which are fully (or 100%) complementary, to a region of the target nucleic acid present in SEQ ID NO: 1 selected from the group consisting of selected from nucleotides 97704-97732, 103232-103259, 151831-151847, and 151949-152006, of SEQ ID NO: 1.

In some embodiments, the antisense oligonucleotide comprises a contiguous nucleotide sequence of 21 nucleotides in length which are fully (or 100%) complementary, to a region of the target nucleic acid present in SEQ ID NO: 1 selected from the group consisting of selected from nucleotides 97704-97732, 103232-103259, 151831-151847, and 151949-152006, of SEQ ID NO: 1.

In some embodiments, the antisense oligonucleotide comprises a contiguous nucleotide sequence of 22 nucleotides in length which are fully (or 100%) complementary, to a region of the target nucleic acid present in SEQ ID NO: 1 selected from the group consisting of selected from nucleotides 97704-97732, 103232-103259, 151831-151847, and 151949-152006, of SEQ ID NO: 1.

In some embodiments, the antisense oligonucleotide comprises a contiguous nucleotide sequence, which is at least 90% complementary, such as at least 95% complementary to a region of the target nucleic acid selected from the group consisting of SEQ ID NO: 3, 4, 5, 6, 7, 8, 9, 10, 11, 12, 13, 14, 15, 16, 17, 18, 19, 20, 21, 22, 23, 24, 25, 26, and 27.

In some embodiments, the antisense oligonucleotide comprises a contiguous nucleotide sequence, which is fully (or 100%) complementary, to a region of the target nucleic acid selected from the group consisting of SEQ ID NO: 3, 4, 5, 6, 7, 8, 9, 10, 11, 12, 13, 14, 15, 16, 17, 18, 19, 20, 21, 22, 23, 24, 25, 26, and 27.

The antisense oligonucleotide of the invention comprises a contiguous nucleotide sequence, which is complementary to or hybridizes to a region of the target nucleic acid, such as a target sequence described herein.

The target nucleic acid sequence to which the therapeutic antisense oligonucleotide is complementary or hybridizes to generally comprises a stretch of contiguous nucleobases of at least 10 nucleotides. The contiguous nucleotide sequence is between 12 to 70 nucleotides, such as 12 to 50, such as 13 to 30, such as 14 to 25, such as 14 to 20 contiguous nucleotides.

In some embodiments, the antisense oligonucleotide of the invention or contiguous nucleotide sequence thereof, comprises or consists of 10 to 30 nucleotides in length, such as from 12 to 25, such as 11 to 22, such as from 12 to 20, such as from 14 to 18 or 14 to 16 contiguous nucleotides in length. In some embodiments, the antisense oligonucleotide or contiguous nucleotide sequence thereof comprises or consists of 22 or less nucleotides, such as 20 or less nucleotides, such as 18 or less nucleotides, such as 14, 15, 16 or 17 nucleotides. It is to be understood that any range given herein includes the range endpoints. Accordingly, if an antisense oligonucleotide is said to include from 10 to 30 nucleotides, both 10 and 30 nucleotides are included.

In some embodiments, the contiguous nucleotide sequence comprises or consists of 10, 11, 12, 13, 14, 15, 16, 17, 18, 19, 20, 21, 22, 23, 24, 25, 26, 27, 28, 29 or 30 contiguous nucleotides in length. In some embodiments, the antisense oligonucleotide or contiguous nucleotide sequence thereof comprises or consists of a sequence selected from SEQ ID NO: 28-52. In some embodiments, the contiguous nucleotide sequence comprises or consists of 12, 13, 14, 15, 16, 17, 18, 19, 20, 21, or 22, contiguous nucleotides in length.

In advantageous embodiments, the antisense oligonucleotide comprises one or more sugar modified nucleosides, such as one or more 2′ sugar modified nucleosides, such as one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid

(ANA), 2′-fluoro-ANA and LNA nucleosides. It is advantageous if one or more of the modified nucleoside(s) is a locked nucleic acid (LNA).

In some embodiments, the contiguous nucleotide sequence comprises LNA nucleosides.

In some embodiments, the contiguous nucleotide sequence comprises LNA nucleosides and DNA nucleosides.

In some embodiments, the contiguous nucleotide sequence comprises LNA nucleosides and DNA nucleosides and 2′-O-methyl RNA nucleosides.

In some embodiments, the contiguous nucleotide sequence comprises LNA nucleosides and DNA nucleosides and 2′-O-methyl RNA nucleosides, and the internucleoside linkages between each of the nucleosides of the contiguous nucleotide linkage are phosphorothioate internucleoside linkages.

In some embodiments, the contiguous nucleotide sequence comprises LNA nucleosides and DNA nucleosides and 2′-O-methyl RNA nucleosides, and the internucleoside linkages between each of the nucleosides of the contiguous nucleotide linkage are phosphorothioate internucleoside linkages.

In some embodiments, the contiguous nucleotide sequence comprises 2′-O-methoxyethyl (2′MOE) nucleosides.

In some embodiments, the contiguous nucleotide sequence comprises 2′-O-methoxyethyl (2′MOE) nucleosides and DNA nucleosides.

Advantageously, the 3′ most nucleoside of the antisense oligonucleotide, or contiguous nucleotide sequence thereof is a 2′sugar modified nucleoside.

Advantageously, the antisense oligonucleotide comprises at least one modified internucleoside linkage, such as phosphorothioate or phosphorodithioate.

In some embodiments, at least one internucleoside linkage in the contiguous nucleotide sequence is a phosphorothioate internucleoside linkages.

In some embodiments, at least one internucleoside linkage in the contiguous nucleotide sequence is a phosphorodithioate internucleoside linkages.

In some embodiments, at least one internucleoside linkage in the contiguous nucleotide sequence is a phosphodiester internucleoside linkages.

In some embodiments, all the internucleoside linkages within the contiguous nucleotide sequence are phosphorothioate internucleoside linkages.

In some embodiments, at least 75% the internucleoside linkages within the antisense oligonucleotide, or contiguous nucleotide sequence thereof, are phosphorothioate internucleoside linkages.

In some embodiments, all the internucleoside linkages within the antisense oligonucleotide, or contiguous nucleotide sequence thereof, are phosphorothioate internucleoside linkages.

In an advantageous embodiment of the invention the antisense oligonucleotide of the invention is capable of recruiting RNase H, such as RNase H1. In some embodiments, the antisense oligonucleotide of the invention, or the contiguous nucleotide sequence thereof is a gapmer.

In some embodiments, the antisense oligonucleotide, or contiguous nucleotide sequence thereof, consists or comprises a gapmer of formula 5′-F-G-F′-3′.

In some embodiments, region G consists of 6-16 DNA nucleosides.

In some embodiments, region F and F′ each comprise at least one LNA nucleoside.

TABLE 3
The invention provides the following antisense oligonucleotide compounds

| SEQ | | | | | Start |
|---|---|---|---|---|---|
| ID | | | CMP ID | | SEQ ID |
| NO: | Motif | Compound | NO | ΔG° | NO: 1 |
| 28 | CAAAGCTCGTGTAG | CAAagctcgtGTAG | 28_1 | -18.2 | 82676 |
| 29 | GTTTTAATACCATTTCA | GTTttaataccatTTCA | 29_1 | -19.1 | 97704 |
| 29 | GTTTTAATACCATTTCA | GTtTTaataccatTTCA | 29_2 | -19.8 | 97704 |
| 29 | GTTTTAATACCATTTCA | GTtTTAataccattTCA | 29_3 | -20.2 | 97704 |
| 29 | GTTTTAATACCATTTCA | GttTTAataccatUTCA | 29_4 | NA | 97704 |
| 29 | GTTTTAATACCATTTCA | GTTTtaataccatTtCA | 29_5 | -18.4 | 97704 |
| 29 | GTTTTAATACCATTTCA | GTtTtaataccatTTCA | 29_6 | -18.6 | 97704 |
| 29 | GTTTTAATACCATTTCA | GTttTaataccatTTCA | 29_7 | -19.0 | 97704 |
| 29 | GTTTTAATACCATTTCA | GTTtTaataccatTTCA | 29_8 | -19.8 | 97704 |
| 29 | GTTTTAATACCATTTCA | GTtTTaataccattTCA | 29_9 | -19.0 | 97704 |
| 29 | GTTTTAATACCATTTCA | GTttTAataccattTCA | 29_10 | -19.4 | 97704 |
| 29 | GTTTTAATACCATTTCA | GttTTAataccattTcA | 29_11 | -15.9 | 97704 |
| 29 | GTTTTAATACCATTTCA | GTTTtAataccattTCA | 29_12 | -19.2 | 97704 |
| 29 | GTTTTAATACCATTTCA | GttTTAataccAttTCA | 29_13 | -18.7 | 97704 |
| 29 | GTTTTAATACCATTTCA | GttTTAataccattTCA | 29_14 | NA | 97704 |
| 29 | GTTTTAATACCATTTCA | GTTtTAataccatttCA | 29_15 | -19.2 | 97704 |
| 29 | GTTTTAATACCATTTCA | GTtTTaataccaTttCA | 29_16 | -18.5 | 97704 |
| 29 | GTTTTAATACCATTTCA | GTTtTaataccatTtCA | 29_17 | -18.3 | 97704 |
| 29 | GTTTTAATACCATTTCA | GTtTTaataccatTtCA | 29_18 | -18.3 | 97704 |
| 29 | GTTTTAATACCATTTCA | GTTTtAataccatTtCA | 29_19 | -18.5 | 97704 |
| 29 | GTTTTAATACCATTTCA | GTTtTAataccatTtCA | 29_20 | -19.5 | 97704 |
| 29 | GTTTTAATACCATTTCA | GTttTaataccaTTtCA | 29_21 | -18.5 | 97704 |
| 29 | GTTTTAATACCATTTCA | GTtttAataccatTTCA | 29_22 | -18.4 | 97704 |
| 29 | GTTTTAATACCATTTCA | GTtTtAataccatTTCA | 29_23 | -18.7 | 97704 |
| 29 | GTTTTAATACCATTTCA | GaTtAataccattTCA | 29_24 | -16.3 | 97704 |
| 29 | GTTTTAATACCATTTCA | GttTTAataccattTCA | 29_25 | NA | 97704 |
| 29 | GTTTTAATACCATTTCA | GttTTaataccattTCA | 29_26 | -17.5 | 97704 |
| 29 | GTTTTAATACCATTTCA | GttTTAAtaccattTCA | 29_27 | -19.1 | 97704 |
| 29 | GTTTTAATACCATTTCA | GttTTAatAccattTCA | 29_28 | NA | 97704 |
| 29 | GTTTTAATACCATTTCA | GttTTAataccAttTCA | 29_29 | NA | 97704 |
| 29 | GTTTTAATACCATTTCA | GttTTAataccattTCA | 29_30 | NA | 97704 |
| 29 | GTTTTAATACCATTTCA | GttTTAataccattTCa | 29_31 | -17.2 | 97704 |
| 29 | GTTTTAATACCATTTCA | GTtTTAataccattTCA | 29_32 | NA | 97704 |
| 29 | GTTTTAATACCATTTCA | GttTTAataccattTCA | 29_33 | -18.6 | 97704 |
| 29 | GTTTTAATACCATTTCA | GttTTAAtaccatUTCA | 29_34 | NA | 97704 |
| 29 | GTTTTAATACCATTTCA | GttTUAataccattTCA | 29_35 | NA | 97704 |
| 29 | GTTTTAATACCATTTCA | GtUTTAataccattTCA | 29_36 | NA | 97704 |
| 29 | GTTTTAATACCATTTCA | GtUTTAataccatUTCA | 29_37 | NA | 97704 |
| 29 | GTTTTAATACCATTTCA | GUtTTAataccattTCA | 29_38 | NA | 97704 |
| 29 | GTTTTAATACCATTTCA | GUtTTAataccatUTCA | 29_39 | NA | 97704 |
| 29 | GTTTTAATACCATTTCA | GUUTTAataccattTCA | 29_40 | NA | 97704 |
| 29 | GTTTTAATACCATTTCA | GUUTTAataccatUTCA | 29_41 | NA | 97704 |
| 29 | GTTTTAATACCATTTCA | GTttTAataccatttCA | 29_42 | -18.4 | 97704 |
| 29 | GTTTTAATACCATTTCA | GTtTTAataccatttCA | 29_43 | -19.2 | 97704 |
| 29 | GTTTTAATACCATTTCA | GTTTtaAtaccatttCA | 29_44 | -18.4 | 97704 |
| 29 | GTTTTAATACCATTTCA | GTTtTaAtaccatttCA | 29_45 | -18.3 | 97704 |
| 29 | GTTTTAATACCATTTCA | GTtTTaAtaccatttCA | 29_46 | -18.3 | 97704 |
| 29 | GTTTTAATACCATTTCA | GTTTtAAtaccatttCA | 29_47 | -18.7 | 97704 |
| 29 | GTTTTAATACCATTTCA | GTttTAAtaccatttCA | 29_48 | -18.9 | 97704 |
| 29 | GTTTTAATACCATTTCA | GTTtTAAtaccatttCA | 29_49 | -19.7 | 97704 |
| 29 | GTTTTAATACCATTTCA | GTtTTAAtaccatttCA | 29_50 | -19.7 | 97704 |
| 29 | GTTTTAATACCATTTCA | GTTttaaTaccatttCA | 29_51 | -18.2 | 97704 |
| 29 | GTTTTAATACCATTTCA | GTTTtaaTaccatttCA | 29_52 | -19.0 | 97704 |
| 29 | GTTTTAATACCATTTCA | GTttTaaTaccatttCA | 29_53 | -18.1 | 97704 |
| 29 | GTTTTAATACCATTTCA | GTTtTaaTaccatttCA | 29_54 | -18.9 | 97704 |
| 29 | GTTTTAATACCATTTCA | GTtTTaaTaccatttCA | 29_55 | -18.9 | 97704 |
| 29 | GTTTTAATACCATTTCA | GTttTAaTaccatttCA | 29_56 | -19.2 | 97704 |
| 29 | GTTTTAATACCATTTCA | GTTtTAaTaccatttCA | 29_57 | -20.0 | 97704 |
| 29 | GTTTTAATACCATTTCA | GTtTTAaTaccatttCA | 29_58 | -20.0 | 97704 |
| 29 | GTTTTAATACCATTTCA | GTTttaATaccatttCA | 29_59 | -19.3 | 97704 |
| 29 | GTTTTAATACCATTTCA | GTtTtaATaccatttCA | 29_60 | -18.8 | 97704 |
| 29 | GTTTTAATACCATTTCA | GTTTtaATaccatttCA | 29_61 | -20.1 | 97704 |
| 29 | GTTTTAATACCATTTCA | GTtttAATaccatttCA | 29_62 | -18.8 | 97704 |
| 29 | GTTTTAATACCATTTCA | GTTttAATaccatttCA | 29_63 | -19.6 | 97704 |
| 29 | GTTTTAATACCATTTCA | GTttTAATaccatttCA | 29_64 | -20.6 | 97704 |
| 29 | GTTTTAATACCATTTCA | GTTTtaataccaTttCA | 29_65 | -18.5 | 97704 |
| 29 | GTTTTAATACCATTTCA | GTTtTaataccaTttCA | 29_66 | -18.5 | 97704 |
| 29 | GTTTTAATACCATTTCA | GTTTtaataccATttCA | 29_67 | -19.4 | 97704 |
| 29 | GTTTTAATACCATTTCA | GTttTAataccatTtCA | 29_68 | -18.7 | 97704 |
| 29 | GTTTTAATACCATTTCA | GTTTtaataccAtTtCA | 29_69 | -18.5 | 97704 |
| 29 | GTTTTAATACCATTTCA | GTTttaataccaTTtCA | 29_70 | -18.5 | 97704 |
| 29 | GTTTTAATACCATTTCA | GTTTtaataccaTTtCA | 29_71 | -19.4 | 97704 |
| 29 | GTTTTAATACCATTTCA | GTTtTaataccaTTtCA | 29_72 | -19.3 | 97704 |
| 29 | GTTTTAATACCATTTCA | GTTTtaataccatTTCA | 29_73 | -19.9 | 97704 |
| 29 | GTTTTAATACCATTTCA | GTTttAataccatTTCA | 29_74 | -19.2 | 97704 |
| 29 | GTTTTAATACCATTTCA | GTTTtAataccatTTCA | 29_75 | -20.0 | 97704 |
| 29 | GTTTTAATACCATTTCA | GTTttaataccAtTTCA | 29_76 | -19.1 | 97704 |
| 29 | GTTTTAATACCATTTCA | GTTTtaataccAtTTCA | 29_77 | -20.0 | 97704 |
| 29 | GTTTTAATACCATTTCA | GTtTTaataccaTTtCA | 29_78 | -19.3 | 97704 |
| 29 | GTTTTAATACCATTTCA | GTtttaataccATTtCA | 29_79 | -18.6 | 97704 |
| 29 | GTTTTAATACCATTTCA | GTTttaataccATTtCA | 29_80 | -19.4 | 97704 |
| 29 | GTTTTAATACCATTTCA | GTTTtaataccATTtCA | 29_81 | -20.2 | 97704 |
| 29 | GTTTTAATACCATTTCA | GTTTtaataccattTCA | 29_82 | -19.1 | 97704 |
| 29 | GTTTTAATACCATTTCA | GTttTaataccattTCA | 29_83 | -18.2 | 97704 |
| 29 | GTTTTAATACCATTTCA | GTTtTaataccattTCA | 29_84 | -19.0 | 97704 |
| 29 | GTTTTAATACCATTTCA | GTTttaAtaccattTCA | 29_85 | -18.6 | 97704 |
| 29 | GTTTTAATACCATTTCA | GTtTtaAtaccattTCA | 29_86 | -18.1 | 97704 |
| 29 | GTTTTAATACCATTTCA | GTTTtaAtaccattTCA | 29_87 | -19.4 | 97704 |
| 29 | GTTTTAATACCATTTCA | GTttTaAtaccattTCA | 29_88 | -18.5 | 97704 |
| 29 | GTTTTAATACCATTTCA | GTTtTaAtaccattTCA | 29_89 | -19.3 | 97704 |
| 29 | GTTTTAATACCATTTCA | GTtTTaAtaccattTCA | 29_90 | -19.3 | 97704 |
| 29 | GTTTTAATACCATTTCA | GTtttAAtaccattTCA | 29_91 | -18.1 | 97704 |
| 29 | GTTTTAATACCATTTCA | GTTttAAtaccattTCA | 29_92 | -18.9 | 97704 |
| 29 | GTTTTAATACCATTTCA | GTtTtAAtaccattTCA | 29_93 | -18.4 | 97704 |
| 29 | GTTTTAATACCATTTCA | GTTTtAAtaccattTCA | 29_94 | -19.7 | 97704 |
| 29 | GTTTTAATACCATTTCA | GTttTAAtaccattTCA | 29_95 | -19.9 | 97704 |
| 29 | GTTTTAATACCATTTCA | GTTttaataccAttTCA | 29_96 | -18.3 | 97704 |
| 29 | GTTTTAATACCATTTCA | GTTttaataccaTtTCA | 29_97 | -18.7 | 97704 |
| 29 | GTTTTAATACCATTTCA | GTTTtaataccaTtTCA | 29_98 | -19.5 | 97704 |
| 29 | GTTTTAATACCATTTCA | GTTtTaataccaTtTCA | 29_99 | -19.4 | 97704 |
| 29 | GTTTTAATACCATTTCA | GTtTTaataccaTtTCA | 29_100 | -19.4 | 97704 |
| 29 | GTTTTAATACCATTTCA | GTtttaataccATtTCA | 29_101 | -18.8 | 97704 |
| 29 | GTTTTAATACCATTTCA | GTTttaataccATtTCA | 29_102 | -19.6 | 97704 |
| 29 | GTTTTAATACCATTTCA | GTtTtaataccATtTCA | 29_103 | -19.1 | 97704 |
| 29 | GTTTTAATACCATTTCA | GTTTtaataccATtTCA | 29_104 | -20.4 | 97704 |
| 29 | GTTTTAATACCATTTCA | GttTTAataccattTCA | 29_105 | NA | 97704 |
| 29 | GTTTTAATACCATTTCA | gttTTAataccattTCA | 29_106 | -18.4 | 97704 |
| 29 | GTTTTAATACCATTTCA | GtttTAataccattTCA | 29_107 | -17.8 | 97704 |
| 29 | GTTTTAATACCATTTCA | GttTTAAtaccattTCA | 29_108 | NA | 97704 |
| 29 | GTTTTAATACCATTTCA | GttTTAaTaccattTCA | 29_109 | -19.5 | 97704 |
| 29 | GTTTTAATACCATTTCA | GttTTAatAccattTCA | 29_110 | -18.8 | 97704 |
| 29 | GTTTTAATACCATTTCA | GttTTAataccaTtTCA | 29_111 | -19.1 | 97704 |
| 29 | GTTTTAATACCATTTCA | GttTTAataccatTTCA | 29_112 | -19.4 | 97704 |
| 29 | GTTTTAATACCATTTCA | GttTTAataccatttCA | 29_113 | -17.6 | 97704 |
| 29 | GTTTTAATACCATTTCA | GttTTAataccaUtTCA | 29_114 | NA | 97704 |
| 29 | GTTTTAATACCATTTCA | GttTTAAtaccaUtTCA | 29_115 | NA | 97704 |
| 29 | GTTTTAATACCATTTCA | GttTTAataccaUUTCA | 29_116 | NA | 97704 |
| 29 | GTTTTAATACCATTTCA | GttTTAaUaccattTCA | 29_117 | NA | 97704 |
| 29 | GTTTTAATACCATTTCA | GttTTAAUaccattTCA | 29_118 | NA | 97704 |
| 29 | GTTTTAATACCATTTCA | GttTTAaUaccaUtTCA | 29_119 | NA | 97704 |
| 29 | GTTTTAATACCATTTCA | GttTTAaUaccaUUTCA | 29_120 | NA | 97704 |
| 29 | GTTTTAATACCATTTCA | GttUTAataccattTCA | 29_121 | NA | 97704 |
| 29 | GTTTTAATACCATTTCA | GtUTTAAtaccattTCA | 29_122 | NA | 97704 |
| 29 | GTTTTAATACCATTTCA | GtUTTAAtaccatUTCA | 29_123 | NA | 97704 |
| 29 | GTTTTAATACCATTTCA | GtUTTAataccaUtTCA | 29_124 | NA | 97704 |
| 29 | GTTTTAATACCATTTCA | GtUTTAAtaccaUtTCA | 29_125 | NA | 97704 |
| 29 | GTTTTAATACCATTTCA | GtUTTAaUaccattTCA | 29_126 | NA | 97704 |
| 29 | GTTTTAATACCATTTCA | GtUTTAAUaccattTCA | 29_127 | NA | 97704 |
| 29 | GTTTTAATACCATTTCA | GUtTTAAtaccattTCA | 29_128 | NA | 97704 |
| 29 | GTTTTAATACCATTTCA | GUtTTAataccaUtTCA | 29_129 | NA | 97704 |
| 29 | GTTTTAATACCATTTCA | GUtTTAaUaccattTCA | 29_130 | NA | 97704 |
| 29 | GTTTTAATACCATTTCA | GUUTTAAtaccattTCA | 29_131 | NA | 97704 |
| 29 | GTTTTAATACCATTTCA | GUUTTAataccaUtTCA | 29_132 | NA | 97704 |
| 29 | GTTTTAATACCATTTCA | GUUTTAataccaUUTCA | 29_133 | NA | 97704 |
| 29 | GTTTTAATACCATTTCA | GUUTTAaUaccattTCA | 29_134 | NA | 97704 |
| 30 | AGTTTTAATACCATTTCA | AGtTttaataccatTtCA | 30_1 | -18.1 | 97704 |
| 30 | AGTTTTAATACCATTTCA | AGttTtaataccatTtCA | 30_2 | -18.1 | 97704 |
| 30 | AGTTTTAATACCATTTCA | AGtTttaataccattTCA | 30_3 | -18.8 | 97704 |
| 30 | AGTTTTAATACCATTTCA | AGtTTtaataccatTtCA | 30_4 | -18.9 | 97704 |
| 30 | AGTTTTAATACCATTTCA | AGtTtTaataccatTtCA | 30_5 | -18.9 | 97704 |
| 30 | AGTTTTAATACCATTTCA | AGttTtaataccattTCA | 30_6 | -18.8 | 97704 |
| 30 | AGTTTTAATACCATTTCA | AGtTttaataccaTTtCA | 30_7 | -19.1 | 97704 |
| 30 | AGTTTTAATACCATTTCA | AGtTttaataccAttTCA | 30_8 | -18.9 | 97704 |
| 30 | AGTTTTAATACCATTTCA | AGtTttaataccaTttCA | 30_9 | -18.3 | 97704 |
| 30 | AGTTTTAATACCATTTCA | AGtTtTaataccaTttCA | 30_10 | -19.0 | 97704 |
| 30 | AGTTTTAATACCATTTCA | AGtTTtaataccaTttCA | 30_11 | -19.1 | 97704 |
| 30 | AGTTTTAATACCATTTCA | AGttTtaataccaTTtCA | 30_12 | -19.1 | 97704 |
| 30 | AGTTTTAATACCATTTCA | AGtTttaataccATttCA | 30_13 | -19.1 | 97704 |
| 30 | AGTTTTAATACCATTTCA | AGtTTtaataccatttCA | 30_14 | -18.6 | 97704 |
| 30 | AGTTTTAATACCATTTCA | AGttttaataccATttCA | 30_15 | -18.8 | 97704 |
| 30 | AGTTTTAATACCATTTCA | AGttTTaataccatttCA | 30_16 | -19.1 | 97704 |
| 30 | AGTTTTAATACCATTTCA | AGttTtaataccaTttCA | 30_17 | -18.3 | 97704 |
| 30 | AGTTTTAATACCATTTCA | AGtttTaaTaccatttCA | 30_18 | -19.1 | 97704 |
| 30 | AGTTTTAATACCATTTCA | AGttttAATaccatttCA | 30_19 | -19.8 | 97704 |
| 30 | AGTTTTAATACCATTTCA | AGtttTaATaccatttCA | 30_20 | -20.3 | 97704 |
| 31 | CAGTTTTAATACCATTTC | CAgtTttaataccatTTC | 31_1 | -18.2 | 97705 |
| 31 | CAGTTTTAATACCATTTC | CAgTtTtaataccatTTC | 3i_2 | -18.9 | 97705 |
| 31 | CAGTTTTAATACCATTTC | CAgtTtTaataccattTC | 3i_3 | -18.1 | 97705 |
| 31 | CAGTTTTAATACCATTTC | CAgTttTaataccattTC | 3i_4 | -18.5 | 97705 |
| 31 | CAGTTTTAATACCATTTC | CAgTttTaataccaTtTC | 3i_5 | -18.9 | 97705 |
| 31 | CAGTTTTAATACCATTTC | CAgTTttaataccattTC | 3i_6 | -18.6 | 97705 |
| 31 | CAGTTTTAATACCATTTC | CAgtTtTaataccatTTC | 3i_7 | -19.0 | 97705 |
| 31 | CAGTTTTAATACCATTTC | CAgtTTtaataccatTTC | 3i_8 | -19.0 | 97705 |
| 31 | CAGTTTTAATACCATTTC | CAGtTttaataccattTC | 31_9 | -18.8 | 97705 |
| 31 | CAGTTTTAATACCATTTC | CAgTtTtaataccattTC | 31_10 | -18.1 | 97705 |
| 31 | CAGTTTTAATACCATTTC | CAgtTTtaataccattTC | 31_11 | -18.2 | 97705 |
| 31 | CAGTTTTAATACCATTTC | CAGttTtaataccattTC | 31_12 | -18.8 | 97705 |
| 31 | CAGTTTTAATACCATTTC | CAgTTttaataccaTtTC | 31_13 | -19.0 | 97705 |
| 31 | CAGTTTTAATACCATTTC | CAgTtttaataccatTTC | 31_14 | -18.6 | 97705 |
| 31 | CAGTTTTAATACCATTTC | CAgtTTtaataccaTtTC | 31_15 | -18.7 | 97705 |
| 31 | CAGTTTTAATACCATTTC | CAGtTttaataccAttTC | 31_16 | -18.9 | 97705 |
| 31 | CAGTTTTAATACCATTTC | CAgtTttaataccATtTC | 31_17 | -18.7 | 97705 |
| 31 | CAGTTTTAATACCATTTC | CAgttTTAAtaccattTC | 31_18 | -20.3 | 97705 |
| 32 | TCAGTTTTAATACCATT | TCAGttTtaataccaTT | 32_1 | -18.8 | 97707 |
| 32 | TCAGTTTTAATACCATT | TCaGTtTTaataccaTT | 32_2 | -19.1 | 97707 |
| 32 | TCAGTTTTAATACCATT | TCAGtttTaataccaTT | 32_3 | -19.2 | 97707 |
| 32 | TCAGTTTTAATACCATT | TCAGttTTaataccaTT | 32_4 | -20.0 | 97707 |
| 32 | TCAGTTTTAATACCATT | TCAgttTTaataccaTT | 32_5 | -18.6 | 97707 |
| 33 | CAGTTTTAATACCATTTCA | CAgtTttAataccatTtCA | 33_1 | -20.1 | 97712 |
| 33 | CAGTTTTAATACCATTTCA | CAgTtttaataccatTtCA | 33_2 | -20.4 | 97712 |
| 33 | CAGTTTTAATACCATTTCA | CAgtTttaataccAtTtCA | 33_3 | -20.1 | 97712 |
| 33 | CAGTTTTAATACCATTTCA | CAgtTttaataccatTtCA | 33_4 | -20.0 | 97712 |
| 33 | CAGTTTTAATACCATTTCA | CAgtTttAataccatttCA | 33_5 | -19.8 | 97712 |
| 33 | CAGTTTTAATACCATTTCA | CAgtTttaataccaTttCA | 33_6 | -20.2 | 97712 |
| 33 | CAGTTTTAATACCATTTCA | CAgtTttaAtaccatttCA | 33_7 | -20.0 | 97712 |
| 33 | CAGTTTTAATACCATTTCA | CAgtTttaataccAtttCA | 33_8 | -19.8 | 97712 |
| 33 | CAGTTTTAATACCATTTCA | CAgTtttAataccatttCA | 33_9 | -20.2 | 97712 |
| 33 | CAGTTTTAATACCATTTCA | CAgTtTtaataccatttCA | 33_10 | -20.4 | 97712 |
| 33 | CAGTTTTAATACCATTTCA | CAgTtttaataccAtttCA | 33_11 | -20.1 | 97712 |
| 33 | CAGTTTTAATACCATTTCA | CAgttTtaataccatttCA | 33_12 | -19.7 | 97712 |
| 33 | CAGTTTTAATACCATTTCA | CAgttTtaataccatTtCA | 33_13 | -20.0 | 97712 |
| 33 | CAGTTTTAATACCATTTCA | CAgTtTtaataccAtttCA | 33_14 | -20.4 | 97712 |
| 33 | CAGTTTTAATACCATTTCA | CAgttTtAataccatTtCA | 33_15 | -20.1 | 97712 |
| 33 | CAGTTTTAATACCATTTCA | CAgttttAataccatttCA | 33_16 | -19.5 | 97712 |
| 33 | CAGTTTTAATACCATTTCA | CAgttTtAAtaccatttCA | 33_17 | -20.4 | 97712 |
| 34 | TCAGTTTTAATACCATTT | TCaGTttTaataccatTT | 34_1 | -19.1 | 97714 |
| 34 | TCAGTTTTAATACCATTT | TCAgtTtTaataccatTT | 34_2 | -19.0 | 97714 |
| 34 | TCAGTTTTAATACCATTT | TCaGTtTtaataccatTT | 34_3 | -18.7 | 97714 |
| 34 | TCAGTTTTAATACCATTT | TCagTtTTaataccatTT | 34_4 | -18.2 | 97714 |
| 34 | TCAGTTTTAATACCATTT | TCaGttTTaataccatTT | 34_5 | -18.4 | 97714 |
| 34 | TCAGTTTTAATACCATTT | TCaGttTtaataccaTTT | 34_6 | -18.1 | 97714 |
| 34 | TCAGTTTTAATACCATTT | TCAgTtTTaataccatTT | 34_7 | -20.1 | 97714 |
| 34 | TCAGTTTTAATACCATTT | TCAGttTtaataccatTT | 34_8 | -19.6 | 97714 |
| 34 | TCAGTTTTAATACCATTT | TCagTttTAataccatTT | 34_9 | -18.5 | 97714 |
| 34 | TCAGTTTTAATACCATTT | TCaGtttTAataccatTT | 34_10 | -18.7 | 97714 |
| 34 | TCAGTTTTAATACCATTT | TCaGTtTTaataccatTT | 34_11 | -19.9 | 97714 |
| 34 | TCAGTTTTAATACCATTT | TCAGtttTaataccatTT | 34_12 | -20.1 | 97714 |
| 34 | TCAGTTTTAATACCATTT | TCaGtttTaataccaTTT | 34_13 | -18.5 | 97714 |
| 34 | TCAGTTTTAATACCATTT | TCAGtTtTaataccatTT | 34_14 | -20.4 | 97714 |
| 34 | TCAGTTTTAATACCATTT | TCaGttTTaataccaTTT | 34_15 | -19.3 | 97714 |
| 34 | TCAGTTTTAATACCATTT | TCaGTttTAataccatTT | 34_16 | -20.3 | 97714 |
| 34 | TCAGTTTTAATACCATTT | TCAgttTtaataccatTT | 34_17 | -18.2 | 97714 |
| 35 | ATCAGTTTTAATACCATT | ATCaGttTTaataccaTT | 35_1 | -19.6 | 97715 |
| 36 | CACATAATTTATTCCCTTT | CAcAtaatttattcccTTT | 36_1 | -20.0 | 103232 |
| 36 | CACATAATTTATTCCCTTT | CAcaTaatttattccctTT | 36_2 | -19.8 | 103232 |
| 36 | CACATAATTTATTCCCTTT | CAcAtaatttattccCtTT | 36_3 | -19.9 | 103232 |
| 36 | CACATAATTTATTCCCTTT | CAcAtaatttattcCctTT | 36_4 | -19.7 | 103232 |
| 36 | CACATAATTTATTCCCTTT | CAcataatttattCcctTT | 36_5 | -19.7 | 103232 |
| 37 | TCACATAATTTATTCCCTT | TCacAtaatttattcCcTT | 37_1 | -19.8 | 103233 |
| 37 | TCACATAATTTATTCCCTT | TCacataatttattcCcTT | 37_2 | -19.8 | 103233 |
| 37 | TCACATAATTTATTCCCTT | TCacAtaatttattCccTT | 37_3 | -19.9 | 103233 |
| 37 | TCACATAATTTATTCCCTT | TCacataatttattCccTT | 37_4 | -19.9 | 103233 |
| 37 | TCACATAATTTATTCCCTT | TCaCataatttattcccTT | 37_5 | -19.8 | 103233 |
| 38 | TTCACATAATTTATTCCCT | TTcacataatttattCcCT | 38_1 | -20.2 | 103234 |
| 39 | TTCACATAATTTATTCCC | TTcacataatttATtcCC | 39_1 | -19.5 | 103235 |
| 39 | TTCACATAATTTATTCCC | TTcacataatTtATtcCC | 39_2 | -19.8 | 103235 |
| 39 | TTCACATAATTTATTCCC | TTcAcataatttatTcCC | 39_3 | -18.7 | 103235 |
| 39 | TTCACATAATTTATTCCC | TTcACataatttatTcCC | 39_4 | -19.8 | 103235 |
| 39 | TTCACATAATTTATTCCC | TTcaCataatttattCCC | 39_5 | -21.2 | 103235 |
| 39 | TTCACATAATTTATTCCC | TTCAcataatttattcCC | 39_6 | -21.0 | 103235 |
| 39 | TTCACATAATTTATTCCC | TTCacataatttatTcCC | 39_7 | -19.8 | 103235 |
| 39 | TTCACATAATTTATTCCC | TTCAcataatttatTcCC | 39_8 | -21.4 | 103235 |
| 39 | TTCACATAATTTATTCCC | TtCAcataatttatTcCC | 39_9 | -19.9 | 103235 |
| 39 | TTCACATAATTTATTCCC | TtCAcataatttatTCCC | 39_10 | NA | 103235 |
| 39 | TTCACATAATTTATTCCC | TtCaCataatttatTCCC | 39_11 | NA | 103235 |
| 39 | TTCACATAATTTATTCCC | TtCACataatttatTcCC | 39_12 | NA | 103235 |
| 39 | TTCACATAATTTATTCCC | TtCacataatttatTcCC | 39_13 | -18.3 | 103235 |
| 39 | TTCACATAATTTATTCCC | TtCacataatttatTCCC | 39_14 | NA | 103235 |
| 39 | TTCACATAATTTATTCCC | TtCacataattUatTcCC | 39_15 | NA | 103235 |
| 39 | TTCACATAATTTATTCCC | TtCacaUaatttatTcCC | 39_16 | NA | 103235 |
| 39 | TTCACATAATTTATTCCC | TUCacataatttatTCCC | 39_17 | NA | 103235 |
| 39 | TTCACATAATTTATTCCC | TUCAcataatttatTcCC | 39_18 | NA | 103235 |
| 39 | TTCACATAATTTATTCCC | TUCAcataatttatTCCC | 39_19 | NA | 103235 |
| 39 | TTCACATAATTTATTCCC | UTCacataatttatTcCC | 39_20 | NA | 103235 |
| 39 | TTCACATAATTTATTCCC | TTcacataaTTtattcCC | 39_21 | -19.4 | 103235 |
| 39 | TTCACATAATTTATTCCC | TTcacataattTattcCC | 39_22 | -18.9 | 103235 |
| 39 | TTCACATAATTTATTCCC | TTcAcataattTattcCC | 39_23 | -19.1 | 103235 |
| 39 | TTCACATAATTTATTCCC | TTcacataaTtTattcCC | 39_24 | -19.4 | 103235 |
| 39 | TTCACATAATTTATTCCC | TTcacataatTTattcCC | 39_25 | -19.7 | 103235 |
| 39 | TTCACATAATTTATTCCC | TTcacataaTTTattcCC | 39_26 | -20.7 | 103235 |
| 39 | TTCACATAATTTATTCCC | TTcacataaTttAttcCC | 39_27 | -18.7 | 103235 |
| 39 | TTCACATAATTTATTCCC | TTcacataatTtAttcCC | 39_28 | -18.6 | 103235 |
| 39 | TTCACATAATTTATTCCC | TTcacataaTTtAttcCC | 39_29 | -19.5 | 103235 |
| 39 | TTCACATAATTTATTCCC | TTcacataattTAttcCC | 39_30 | -20.0 | 103235 |
| 39 | TTCACATAATTTATTCCC | TTcacataaTtTAttcCC | 39_31 | -20.5 | 103235 |
| 39 | TTCACATAATTTATTCCC | TTcacataatTTAttcCC | 39_32 | -20.9 | 103235 |
| 39 | TTCACATAATTTATTCCC | TTcAcataatttaTtcCC | 39_33 | -18.8 | 103235 |
| 39 | TTCACATAATTTATTCCC | TTcacataaTttaTtcCC | 39_34 | -19.1 | 103235 |
| 39 | TTCACATAATTTATTCCC | TTcacataatTtaTtcCC | 39_35 | -18.9 | 103235 |
| 39 | TTCACATAATTTATTCCC | TTcacataaTTtaTtcCC | 39_36 | -19.9 | 103235 |
| 39 | TTCACATAATTTATTCCC | TTcacataattTaTtcCC | 39_37 | -19.4 | 103235 |
| 39 | TTCACATAATTTATTCCC | TTcAcataattTaTtcCC | 39_38 | -19.5 | 103235 |
| 39 | TTCACATAATTTATTCCC | TTcacataaTtTaTtcCC | 39_39 | -19.8 | 103235 |
| 39 | TTCACATAATTTATTCCC | TTcacataatTTaTtcCC | 39_40 | -20.2 | 103235 |
| 39 | TTCACATAATTTATTCCC | TTcacataaTTTaTtcCC | 39_41 | -21.1 | 103235 |
| 39 | TTCACATAATTTATTCCC | TTcAcataatttATtcCC | 39_42 | -19.7 | 103235 |
| 39 | TTCACATAATTTATTCCC | TTcacataaTttATtcCC | 39_43 | -20.0 | 103235 |
| 39 | TTCACATAATTTATTCCC | TTcacataaTTtATtcCC | 39_44 | -20.8 | 103235 |
| 39 | TTCACATAATTTATTCCC | TTcacataattTATtcCC | 39_45 | -21.3 | 103235 |
| 39 | TTCACATAATTTATTCCC | TTcacataaTttatTcCC | 39_46 | -19.0 | 103235 |
| 39 | TTCACATAATTTATTCCC | TTcacataatTtatTcCC | 39_47 | -18.9 | 103235 |
| 39 | TTCACATAATTTATTCCC | TTcacataaTTtatTcCC | 39_48 | -19.8 | 103235 |
| 39 | TTCACATAATTTATTCCC | TTcacataattTatTcCC | 39_49 | -19.3 | 103235 |
| 39 | TTCACATAATTTATTCCC | TTcAcataattTatTcCC | 39_50 | -19.4 | 103235 |
| 39 | TTCACATAATTTATTCCC | TTcacataaTtTatTcCC | 39_51 | -19.7 | 103235 |
| 39 | TTCACATAATTTATTCCC | TTcacataatTTatTcCC | 39_52 | -20.1 | 103235 |
| 39 | TTCACATAATTTATTCCC | TTcacataaTTTatTcCC | 39_53 | -21.1 | 103235 |
| 39 | TTCACATAATTTATTCCC | TTcacataatttAtTcCC | 39_54 | -18.7 | 103235 |
| 39 | TTCACATAATTTATTCCC | TTcAcataatttAtTcCC | 39_55 | -18.8 | 103235 |
| 39 | TTCACATAATTTATTCCC | TTcacataaTttAtTcCC | 39_56 | -19.1 | 103235 |
| 39 | TTCACATAATTTATTCCC | TTcacataatTtAtTcCC | 39_57 | -19.0 | 103235 |
| 39 | TTCACATAATTTATTCCC | TTcacataaTTtAtTcCC | 39_58 | -19.9 | 103235 |
| 39 | TTCACATAATTTATTCCC | TTcacataattTAtTcCC | 39_59 | -20.4 | 103235 |
| 39 | TTCACATAATTTATTCCC | TTcacataatttaTTcCC | 39_60 | -19.5 | 103235 |
| 39 | TTCACATAATTTATTCCC | TTcAcataatttaTTcCC | 39_61 | -19.7 | 103235 |
| 39 | TTCACATAATTTATTCCC | TTcacataaTttaTTcCC | 39_62 | -20.0 | 103235 |
| 39 | TTCACATAATTTATTCCC | TTcacataatTtaTTcCC | 39_63 | -19.8 | 103235 |
| 39 | TTCACATAATTTATTCCC | TTcacataaTTtaTTcCC | 39_64 | -20.8 | 103235 |
| 39 | TTCACATAATTTATTCCC | TTcacataattTaTTcCC | 39_65 | -20.2 | 103235 |
| 39 | TTCACATAATTTATTCCC | TTcacataaTtTaTTcCC | 39_66 | -20.7 | 103235 |
| 39 | TTCACATAATTTATTCCC | TTcacataatTTaTTcCC | 39_67 | -21.1 | 103235 |
| 39 | TTCACATAATTTATTCCC | TTcacataatttATTcCC | 39_68 | -20.4 | 103235 |
| 39 | TTCACATAATTTATTCCC | TTcacataaTttATTcCC | 39_69 | -20.9 | 103235 |
| 39 | TTCACATAATTTATTCCC | TTcacataatTtATTcCC | 39_70 | -20.7 | 103235 |
| 39 | TTCACATAATTTATTCCC | TtCacataatttatTcCC | 39_71 | NA | 103235 |
| 39 | TTCACATAATTTATTCCC | TtcacataatttatTcCC | 39_72 | -17.7 | 103235 |
| 39 | TTCACATAATTTATTCCC | TTCacataatttatTcCC | 39_73 | NA | 103235 |
| 39 | TTCACATAATTTATTCCC | TTcacataatttattCCC | 39_74 | -20.6 | 103235 |
| 39 | TTCACATAATTTATTCCC | TTcAcataatttattCCC | 39_75 | -20.7 | 103235 |
| 39 | TTCACATAATTTATTCCC | TTcacataaTttattCCC | 39_76 | -21.0 | 103235 |
| 39 | TTCACATAATTTATTCCC | TTcacataatTtattCCC | 39_77 | -20.9 | 103235 |
| 39 | TTCACATAATTTATTCCC | TTcacataattTattCCC | 39_78 | -21.3 | 103235 |
| 39 | TTCACATAATTTATTCCC | TTcacataatttAttCCC | 39_79 | -20.7 | 103235 |
| 39 | TTCACATAATTTATTCCC | TTcacataaTttAttCCC | 39_80 | -21.1 | 103235 |
| 39 | TTCACATAATTTATTCCC | TTcacataatTtAttCCC | 39_81 | -21.0 | 103235 |
| 39 | TTCACATAATTTATTCCC | TTcacataatttaTtCCC | 39_82 | -21.0 | 103235 |
| 39 | TTCACATAATTTATTCCC | TTcAcataatttaTtCCC | 39_83 | -21.1 | 103235 |
| 39 | TTCACATAATTTATTCCC | TTcacataaTttaTtCCC | 39_84 | -21.4 | 103235 |
| 39 | TTCACATAATTTATTCCC | TTcacataatTtaTtCCC | 39_85 | -21.3 | 103235 |
| 39 | TTCACATAATTTATTCCC | TTcaCataatttattcCC | 39_86 | -18.9 | 103235 |
| 39 | TTCACATAATTTATTCCC | TTcaCataatttAttcCC | 39_87 | -19.0 | 103235 |
| 39 | TTCACATAATTTATTCCC | TTcACataatttAttcCC | 39_88 | -19.5 | 103235 |
| 39 | TTCACATAATTTATTCCC | TTcaCataatttaTtcCC | 39_89 | -19.3 | 103235 |
| 39 | TTCACATAATTTATTCCC | TTcACataatttaTtcCC | 39_90 | -19.9 | 103235 |
| 39 | TTCACATAATTTATTCCC | TTcaCataatttatTcCC | 39_91 | -19.2 | 103235 |
| 39 | TTCACATAATTTATTCCC | TTcaCataatttAtTcCC | 39_92 | -19.3 | 103235 |
| 39 | TTCACATAATTTATTCCC | TTcaCataatttaTTcCC | 39_93 | -20.2 | 103235 |
| 39 | TTCACATAATTTATTCCC | TTcACataatttaTTcCC | 39_94 | -20.7 | 103235 |
| 39 | TTCACATAATTTATTCCC | TtCacataatttatTcCC | 39_95 | NA | 103235 |
| 39 | TTCACATAATTTATTCCC | TtCacataatttatTccC | 39_96 | -16.1 | 103235 |
| 39 | TTCACATAATTTATTCCC | TtCacataatttatTcCC | 39_97 | NA | 103235 |
| 39 | TTCACATAATTTATTCCC | TtCacataatttatTcCc | 39_98 | -16.5 | 103235 |
| 39 | TTCACATAATTTATTCCC | TTCacataatttattcCC | 39_99 | -19.5 | 103235 |
| 39 | TTCACATAATTTATTCCC | TTCacataatTtattcCC | 39_100 | -19.8 | 103235 |
| 39 | TTCACATAATTTATTCCC | TTCacataattTattcCC | 39_101 | -20.2 | 103235 |
| 39 | TTCACATAATTTATTCCC | TTCacataatTTattcCC | 39_102 | -21.0 | 103235 |
| 39 | TTCACATAATTTATTCCC | TTCacataatttAttcCC | 39_103 | -19.6 | 103235 |
| 39 | TTCACATAATTTATTCCC | TTCacataatTtAttcCC | 39_104 | -19.9 | 103235 |
| 39 | TTCACATAATTTATTCCC | TTCacataattTAttcCC | 39_105 | -21.3 | 103235 |
| 39 | TTCACATAATTTATTCCC | TTCacataatttaTtcCC | 39_106 | -19.9 | 103235 |
| 39 | TTCACATAATTTATTCCC | TTCacataatTtaTtcCC | 39_107 | -20.2 | 103235 |
| 39 | TTCACATAATTTATTCCC | TTCacataattTaTtcCC | 39_108 | -20.6 | 103235 |
| 39 | TTCACATAATTTATTCCC | TTCacataatttATtcCC | 39_109 | -20.8 | 103235 |
| 39 | TTCACATAATTTATTCCC | TTCacataatTtatTcCC | 39_110 | -20.1 | 103235 |
| 39 | TTCACATAATTTATTCCC | TTCacataattTatTcCC | 39_111 | -20.6 | 103235 |
| 39 | TTCACATAATTTATTCCC | TTCacataatttAtTcCC | 39_112 | -19.9 | 103235 |
| 39 | TTCACATAATTTATTCCC | TTCacataatTtAtTcCC | 39_113 | -20.2 | 103235 |
| 39 | TTCACATAATTTATTCCC | TTCacataatttaTTcCC | 39_114 | -20.8 | 103235 |
| 39 | TTCACATAATTTATTCCC | ttCacataatttatTcCC | 39_115 | -18.2 | 103235 |
| 39 | TTCACATAATTTATTCCC | TtCAcataatttatTcCC | 39_116 | NA | 103235 |
| 39 | TTCACATAATTTATTCCC | TtCaCataatttatTcCC | 39_117 | NA | 103235 |
| 39 | TTCACATAATTTATTCCC | TtCacAtaatttatTcCC | 39_118 | NA | 103235 |
| 39 | TTCACATAATTTATTCCC | TtCacAtaatttatTcCC | 39_119 | -18.4 | 103235 |
| 39 | TTCACATAATTTATTCCC | TtCacaTaatttatTcCC | 39_120 | -19.2 | 103235 |
| 39 | TTCACATAATTTATTCCC | TtCacataattTatTcCC | 39_121 | -19.1 | 103235 |
| 39 | TTCACATAATTTATTCCC | TtCacataatttAtTcCC | 39_122 | NA | 103235 |
| 39 | TTCACATAATTTATTCCC | TtCacataatttAtTcCC | 39_123 | -18.4 | 103235 |
| 39 | TTCACATAATTTATTCCC | TtCacataatttaTTcCC | 39_124 | -19.3 | 103235 |
| 39 | TTCACATAATTTATTCCC | TtCacataatttattcCC | 39_125 | -18.0 | 103235 |
| 39 | TTCACATAATTTATTCCC | TtCacataatttatTCCC | 39_126 | NA | 103235 |
| 39 | TTCACATAATTTATTCCC | TtCacAtaatttatTCCC | 39_127 | NA | 103235 |
| 39 | TTCACATAATTTATTCCC | TtCacataatttAtTCCC | 39_128 | NA | 103235 |
| 39 | TTCACATAATTTATTCCC | TtCaCAtaatttatTcCC | 39_129 | NA | 103235 |
| 39 | TTCACATAATTTATTCCC | TtCAcataatttAtTcCC | 39_130 | NA | 103235 |
| 39 | TTCACATAATTTATTCCC | TtCacataatttatTCCC | 39_131 | NA | 103235 |
| 39 | TTCACATAATTTATTCCC | TtCacataatttatTCCC | 39_132 | -21.3 | 103235 |
| 39 | TTCACATAATTTATTCCC | TtCacataatttatUcCC | 39_133 | NA | 103235 |
| 39 | TTCACATAATTTATTCCC | TtCacataatttatUCCC | 39_134 | NA | 103235 |
| 39 | TTCACATAATTTATTCCC | TtCacataatttaUTcCC | 39_135 | NA | 103235 |
| 39 | TTCACATAATTTATTCCC | TtCacataatttaUTCCC | 39_136 | NA | 103235 |
| 39 | TTCACATAATTTATTCCC | TtCacataatttAUTcCC | 39_137 | NA | 103235 |
| 39 | TTCACATAATTTATTCCC | TtCaCataatttaUTcCC | 39_138 | NA | 103235 |
| 39 | TTCACATAATTTATTCCC | TtCacAtaatttaUTcCC | 39_139 | NA | 103235 |
| 39 | TTCACATAATTTATTCCC | TtCacataattUAtTcCC | 39_140 | NA | 103235 |
| 39 | TTCACATAATTTATTCCC | TtCAcataattUatTcCC | 39_141 | NA | 103235 |
| 39 | TTCACATAATTTATTCCC | TtCacataatUtatTcCC | 39_142 | NA | 103235 |
| 39 | TTCACATAATTTATTCCC | TTCaCataatttattcCC | 39_143 | -20.2 | 103235 |
| 39 | TTCACATAATTTATTCCC | TTCaCataatttaTtcCC | 39_144 | -20.6 | 103235 |
| 39 | TTCACATAATTTATTCCC | TTCaCataatttatTcCC | 39_145 | -20.5 | 103235 |
| 39 | TTCACATAATTTATTCCC | TtCaCataatttatTcCC | 39_146 | -19.0 | 103235 |
| 39 | TTCACATAATTTATTCCC | TUCacataatttatTcCC | 39_147 | NA | 103235 |
| 39 | TTCACATAATTTATTCCC | TUCaCataatttatTcCC | 39_148 | NA | 103235 |
| 39 | TTCACATAATTTATTCCC | TUCacAtaatttatTcCC | 39_149 | NA | 103235 |
| 39 | TTCACATAATTTATTCCC | TUCacataatttAtTcCC | 39_150 | NA | 103235 |
| 39 | TTCACATAATTTATTCCC | TUCacataatttaUTcCC | 39_151 | NA | 103235 |
| 39 | TTCACATAATTTATTCCC | TUCacataattUatTcCC | 39_152 | NA | 103235 |
| 40 | ACATAATTTATTCCCTTTTT | ACataatttattccCtttTT | 40_1 | -19.7 | 103238 |
| 40 | ACATAATTTATTCCCTTTTT | ACataatttattccCtTtTT | 40_2 | -20.0 | 103238 |
| 40 | ACATAATTTATTCCCTTTTT | ACataatttattcCctttTT | 40_3 | -19.5 | 103238 |
| 40 | ACATAATTTATTCCCTTTTT | ACataatttattcCctTtTT | 40_4 | -19.8 | 103238 |
| 40 | ACATAATTTATTCCCTTTTT | ACataatttattcCcTttTT | 40_5 | -20.0 | 103238 |
| 40 | ACATAATTTATTCCCTTTTT | ACataatttattCcctTtTT | 40_6 | -19.8 | 103238 |
| 41 | CATAATTTATTCCCTTTTT | CAtaatttattccctTTTT | 41_1 | -19.8 | 103238 |
| 41 | CATAATTTATTCCCTTTTT | CAtaATtTattccctTtTT | 41_2 | -20.8 | 103238 |
| 41 | CATAATTTATTCCCTTTTT | CAtaatttattccCttTTT | 41_3 | -19.9 | 103238 |
| 41 | CATAATTTATTCCCTTTTT | CAtaatttattccCtTtTT | 41_4 | -19.4 | 103238 |
| 41 | CATAATTTATTCCCTTTTT | CAtaatttattccCtttTT | 41_5 | -19.1 | 103238 |
| 41 | CATAATTTATTCCCTTTTT | CAtaatttatTccCtttTT | 41_6 | -19.5 | 103238 |
| 41 | CATAATTTATTCCCTTTTT | CAtaatttatTccCtTtTT | 41_7 | -19.8 | 103238 |
| 41 | CATAATTTATTCCCTTTTT | CAtaatttattcCcttTTT | 41_8 | -19.7 | 103238 |
| 41 | CATAATTTATTCCCTTTTT | CAtaatttattcCcTttTT | 41_9 | -19.4 | 103238 |
| 41 | CATAATTTATTCCCTTTTT | CAtaatttattcCctTtTT | 41_10 | -19.2 | 103238 |
| 41 | CATAATTTATTCCCTTTTT | CAtaatttatTcCctttTT | 41_11 | -19.2 | 103238 |
| 41 | CATAATTTATTCCCTTTTT | CAtaatttatTcCctTtTT | 41_12 | -19.5 | 103238 |
| 41 | CATAATTTATTCCCTTTTT | CAtaatttatTcCcTttTT | 41_13 | -19.8 | 103238 |
| 41 | CATAATTTATTCCCTTTTT | CAtaatttattCcctTtTT | 41_14 | -19.3 | 103238 |
| 41 | CATAATTTATTCCCTTTTT | CAtaatttattCcCtttTT | 41_15 | -19.9 | 103238 |
| 42 | ACATAATTTATTCCCTTTT | ACAtaatttattcccTtTT | 42_1 | -19.7 | 103239 |
| 42 | ACATAATTTATTCCCTTTT | ACAtaatttattccctTTT | 42_2 | -20.0 | 103239 |
| 42 | ACATAATTTATTCCCTTTT | ACataatTtattcccTTTT | 42_3 | -19.9 | 103239 |
| 42 | ACATAATTTATTCCCTTTT | ACataatttattccCtTTT | 42_4 | -19.5 | 103239 |
| 42 | ACATAATTTATTCCCTTTT | ACataatttattccCTtTT | 42_5 | -19.8 | 103239 |
| 42 | ACATAATTTATTCCCTTTT | ACaTaatttattccCttTT | 42_6 | -19.5 | 103239 |
| 42 | ACATAATTTATTCCCTTTT | ACAtaatttattccCttTT | 42_7 | -20.1 | 103239 |
| 42 | ACATAATTTATTCCCTTTT | ACAtaatttattcCcttTT | 42_8 | -19.9 | 103239 |
| 42 | ACATAATTTATTCCCTTTT | ACataatttattcCcTtTT | 42_9 | -19.0 | 103239 |
| 42 | ACATAATTTATTCCCTTTT | ACataatttattcCctTTT | 42_10 | -19.3 | 103239 |
| 42 | ACATAATTTATTCCCTTTT | ACaTaatttattcCcTtTT | 42_11 | -19.8 | 103239 |
| 42 | ACATAATTTATTCCCTTTT | ACaTaatttattcCcttTT | 42_12 | -19.3 | 103239 |
| 42 | ACATAATTTATTCCCTTTT | ACAtaatttattCccttTT | 42_13 | -20.0 | 103239 |
| 42 | ACATAATTTATTCCCTTTT | ACataatttattCccTtTT | 42_14 | -19.0 | 103239 |
| 42 | ACATAATTTATTCCCTTTT | ACaTaatttattCccTtTT | 42_15 | -19.9 | 103239 |
| 42 | ACATAATTTATTCCCTTTT | ACataatttattCcCttTT | 42_16 | -19.4 | 103239 |
| 43 | CACATAATTTATTCCCTTTT | CAcAtaatttattcccttTT | 43_1 | -20.0 | 103239 |
| 43 | CACATAATTTATTCCCTTTT | CAcataatTtattcccttTT | 43_2 | -20.3 | 103239 |
| 43 | CACATAATTTATTCCCTTTT | CAcAtaatTtattcccTtTT | 43_3 | -20.8 | 103239 |
| 44 | CATAATTTATTCCCTTTT | CATaatttattccctTTT | 44_1 | -19.7 | 103239 |
| 44 | CATAATTTATTCCCTTTT | CAtaatttattccCtTTT | 44_2 | -18.9 | 103239 |
| 44 | CATAATTTATTCCCTTTT | CAtaatttattccCTtTT | 44_3 | -19.3 | 103239 |
| 44 | CATAATTTATTCCCTTTT | CAtaatttatTccCttTT | 44_4 | -18.4 | 103239 |
| 44 | CATAATTTATTCCCTTTT | CATaatttattccCttTT | 44_5 | -19.8 | 103239 |
| 44 | CATAATTTATTCCCTTTT | CAtaATttattccCtTTT | 44_6 | -20.5 | 103239 |
| 44 | CATAATTTATTCCCTTTT | CAtaatttattcCctTTT | 44_7 | -18.7 | 103239 |
| 44 | CATAATTTATTCCCTTTT | CAtaatttattcCcTTTT | 44_8 | -19.7 | 103239 |
| 44 | CATAATTTATTCCCTTTT | CAtaatttatTcCcttTT | 44_9 | -18.2 | 103239 |
| 44 | CATAATTTATTCCCTTTT | CATaatttattcCcttTT | 44_10 | -19.5 | 103239 |
| 44 | CATAATTTATTCCCTTTT | CAtaatttatTcCcTtTT | 44_11 | -18.8 | 103239 |
| 44 | CATAATTTATTCCCTTTT | CAtaatttatTcCctTTT | 44_12 | -19.1 | 103239 |
| 44 | CATAATTTATTCCCTTTT | CAtaatttattCcctTTT | 44_13 | -18.8 | 103239 |
| 44 | CATAATTTATTCCCTTTT | CAtaatttattCccTTTT | 44_14 | -19.8 | 103239 |
| 44 | CATAATTTATTCCCTTTT | CAtaatttatTCccttTT | 44_15 | -18.9 | 103239 |
| 44 | CATAATTTATTCCCTTTT | CAtaatttattCcCttTT | 44_16 | -18.8 | 103239 |
| 44 | CATAATTTATTCCCTTTT | CAtaatttattCcCtTTT | 44_17 | -19.7 | 103239 |
| 44 | CATAATTTATTCCCTTTT | CAtaatttattCcCTtTT | 44_18 | -20.0 | 103239 |
| 45 | TCACATAATTTATTCCCTTT | TCacAtaatttattccctTT | 45_1 | -20.0 | 103240 |
| 46 | CACATAATTTATTCCCTT | CAcAtaatttattccCTT | 46_1 | -19.8 | 103241 |
| 46 | CACATAATTTATTCCCTT | CAcaTaatttattcCcTT | 46_2 | -19.7 | 103241 |
| 46 | CACATAATTTATTCCCTT | CAcaTaatttattCccTT | 46_3 | -19.8 | 103241 |
| 46 | CACATAATTTATTCCCTT | CACataatttattcCcTT | 46_4 | -19.9 | 103241 |
| 46 | CACATAATTTATTCCCTT | CACataatttattCccTT | 46_5 | -20.0 | 103241 |
| 47 | TCACATAATTTATTCCCT | TCaCataatttattccCT | 47_1 | -19.5 | 103242 |
| 47 | TCACATAATTTATTCCCT | TCacataatttaTtccCT | 47_2 | -19.3 | 103242 |
| 47 | TCACATAATTTATTCCCT | TCacAtaatttattCcCT | 47_3 | -19.7 | 103242 |
| 47 | TCACATAATTTATTCCCT | TCacataatttattCcCT | 47_4 | -19.6 | 103242 |
| 48 | CTCATACTGCTCTTTCTA | CTcAtactgcTctttcTA | 48_1 | -21.7 | 151831 |
| 48 | CTCATACTGCTCTTTCTA | CTCatactgctCtttcTA | 48_2 | -23.3 | 151831 |
| 48 | CTCATACTGCTCTTTCTA | CtCAtactgctCtttcTA | 48_3 | -22.9 | 151831 |
| 48 | CTCATACTGCTCTTTCTA | CtCatactgctCtttCTA | 48_4 | -23.1 | 151831 |
| 48 | CTCATACTGCTCTTTCTA | CtCatactgctctttcTA | 48_5 | -20.5 | 151831 |
| 48 | CTCATACTGCTCTTTCTA | CtCatactGctCtttcTA | 48_6 | -21.8 | 151831 |
| 48 | CTCATACTGCTCTTTCTA | CtCatactgctCttTcTA | 48_7 | -21.8 | 151831 |
| 48 | CTCATACTGCTCTTTCTA | CtCatactgctCtttctA | 48_8 | -19.5 | 151831 |
| 48 | CTCATACTGCTCTTTCTA | CtCatactgctCtttcTA | 48_9 | -21.5 | 151831 |
| 48 | CTCATACTGCTCTTTCTA | CtCAtactgctCttUcTA | 48_10 | NA | 151831 |
| 48 | CTCATACTGCTCTTTCTA | CtCatactgctCtttcTA | 48_11 | NA | 151831 |
| 48 | CTCATACTGCTCTTTCTA | ctCatactgctCtttcTA | 48_12 | -20.9 | 151831 |
| 48 | CTCATACTGCTCTTTCTA | CTcatactgcTctttcTA | 48_13 | -21.7 | 151831 |
| 48 | CTCATACTGCTCTTTCTA | CTcAtactgctcTttcTA | 48_14 | -21.6 | 151831 |
| 48 | CTCATACTGCTCTTTCTA | CTcAtactgctctTtcTA | 48_15 | -21.4 | 151831 |
| 48 | CTCATACTGCTCTTTCTA | CTcAtactgctcttTcTA | 48_16 | -21.5 | 151831 |
| 48 | CTCATACTGCTCTTTCTA | CtCatactgctCtttcTA | 48_17 | NA | 151831 |
| 48 | CTCATACTGCTCTTTCTA | CtcatactgctCtttcTA | 48_18 | -20.8 | 151831 |
| 48 | CTCATACTGCTCTTTCTA | CtCatactgctCtttcTA | 48_19 | NA | 151831 |
| 48 | CTCATACTGCTCTTTCTA | CtCAtactgctCtttcTA | 48_20 | NA | 151831 |
| 48 | CTCATACTGCTCTTTCTA | CtCaTactgctCtttcTA | 48_21 | -22.3 | 151831 |
| 48 | CTCATACTGCTCTTTCTA | CtCatActgctCtttcTA | 48_22 | NA | 151831 |
| 48 | CTCATACTGCTCTTTCTA | CtCatActgctCtttcTA | 48_23 | -21.6 | 151831 |
| 48 | CTCATACTGCTCTTTCTA | CtCatactGctCtttcTA | 48_24 | NA | 151831 |
| 48 | CTCATACTGCTCTTTCTA | CtCatactgCtCtttcTA | 48_25 | NA | 151831 |
| 48 | CTCATACTGCTCTTTCTA | CtCatactgcTCtttcTA | 48_26 | -22.7 | 151831 |
| 48 | CTCATACTGCTCTTTCTA | CtCatactgctCTttcTA | 48_27 | -22.6 | 151831 |
| 48 | CTCATACTGCTCTTTCTA | CtCatactgctCtTtcTA | 48_28 | -21.8 | 151831 |
| 48 | CTCATACTGCTCTTTCTA | CtCatactgctCtttCTA | 48_29 | NA | 151831 |
| 48 | CTCATACTGCTCTTTCTA | CtCatactgctCtttcTA | 48_30 | NA | 151831 |
| 48 | CTCATACTGCTCTTTCTA | CtCatactgctCtttcTa | 48_31 | -20.4 | 151831 |
| 48 | CTCATACTGCTCTTTCTA | CtCAtactgctCtttCTA | 48_32 | NA | 151831 |
| 48 | CTCATACTGCTCTTTCTA | CtCatactgctCtttCUA | 48_33 | NA | 151831 |
| 48 | CTCATACTGCTCTTTCTA | CtCatactgctCttUcTA | 48_34 | NA | 151831 |
| 48 | CTCATACTGCTCTTTCTA | CtCatactgctCtUtcTA | 48_35 | NA | 151831 |
| 48 | CTCATACTGCTCTTTCTA | CtCAtactgctCtUtcTA | 48_36 | NA | 151831 |
| 48 | CTCATACTGCTCTTTCTA | CtCatactgctCtUUCTA | 48_37 | NA | 151831 |
| 48 | CTCATACTGCTCTTTCTA | CtCatactgctCUttcTA | 48_38 | NA | 151831 |
| 48 | CTCATACTGCTCTTTCTA | CtCAtactgctCUttcTA | 48_39 | NA | 151831 |
| 48 | CTCATACTGCTCTTTCTA | CtCatactgctCUtUCTA | 48_40 | NA | 151831 |
| 48 | CTCATACTGCTCTTTCTA | CtCatactgctCUUtCTA | 48_41 | NA | 151831 |
| 48 | CTCATACTGCTCTTTCTA | CtCatactgctCUUUcTA | 48_42 | NA | 151831 |
| 48 | CTCATACTGCTCTTTCTA | CtCatactgcUCtttcTA | 48_43 | NA | 151831 |
| 48 | CTCATACTGCTCTTTCTA | CtCatactgCtCtttcTA | 48_44 | -22.6 | 151831 |
| 48 | CTCATACTGCTCTTTCTA | CtCaUactgctCtttcTA | 48_45 | NA | 151831 |
| 48 | CTCATACTGCTCTTTCTA | CtCaUactgctCtttCTA | 48_46 | NA | 151831 |
| 48 | CTCATACTGCTCTTTCTA | CtCaUactgctCttUcTA | 48_47 | NA | 151831 |
| 48 | CTCATACTGCTCTTTCTA | CtCaUactgctCtUtcTA | 48_48 | NA | 151831 |
| 48 | CTCATACTGCTCTTTCTA | CtCaUactgctCUttcTA | 48_49 | NA | 151831 |
| 48 | CTCATACTGCTCTTTCTA | CUCAtactgctCtttcTA | 48_50 | NA | 151831 |
| 48 | CTCATACTGCTCTTTCTA | CUCatactgctCtttCTA | 48_51 | NA | 151831 |
| 48 | CTCATACTGCTCTTTCTA | CUCatactgctCtUtCTA | 48_52 | NA | 151831 |
| 48 | CTCATACTGCTCTTTCTA | CUCatactgctCUttcTA | 48_53 | NA | 151831 |
| 48 | CTCATACTGCTCTTTCTA | CUCatactgctCUttCTA | 48_54 | NA | 151831 |
| 48 | CTCATACTGCTCTTTCTA | CUCatactgctCUtUcTA | 48_55 | NA | 151831 |
| 48 | CTCATACTGCTCTTTCTA | CUCatactgctCUUtcTA | 48_56 | NA | 151831 |
| 49 | TCATACTGCTCTTTCTA | TCatactgctcTttcTA | 49_1 | -19.4 | 151831 |
| 49 | TCATACTGCTCTTTCTA | TCAtactgctcTttcTA | 49_2 | -20.8 | 151831 |
| 50 | GATAGTATCAACCCAT | GATagtatcAaccCAT | 50_1 | -20.5 | 151949 |
| 50 | GATAGTATCAACCCAT | GAtagtatcAAccCAT | 50_2 | -19.4 | 151949 |
| 50 | GATAGTATCAACCCAT | GAtagtatcaAccCAT | 50_3 | -19.1 | 151949 |
| 50 | GATAGTATCAACCCAT | GATagtatcAAcCcAT | 50_4 | -19.8 | 151949 |
| 50 | GATAGTATCAACCCAT | GATagtatcAacCcAT | 50_5 | -19.2 | 151949 |
| 50 | GATAGTATCAACCCAT | GATagtatcAACccAT | 50_6 | -20.3 | 151949 |
| 50 | GATAGTATCAACCCAT | GATagtatcAaCccAT | 50_7 | -19.3 | 151949 |
| 50 | GATAGTATCAACCCAT | GAtagtatcAACcCAT | 50_8 | -20.6 | 151949 |
| 50 | GATAGTATCAACCCAT | GAtagtatcAaCcCAT | 50_9 | -19.6 | 151949 |
| 50 | GATAGTATCAACCCAT | GAtagtatcAACCcAT | 50_10 | -20.9 | 151949 |
| 50 | GATAGTATCAACCCAT | GAtagtatCAAcccAT | 50_11 | -19.5 | 151949 |
| 50 | GATAGTATCAACCCAT | GAtagtatCAaccCAT | 50_12 | -20.9 | 151949 |
| 50 | GATAGTATCAACCCAT | GAtagtatCaAccCAT | 50_13 | -19.8 | 151949 |
| 50 | GATAGTATCAACCCAT | GAtagtatCaaccCAT | 50_14 | -19.4 | 151949 |
| 50 | GATAGTATCAACCCAT | GAtagtatCAacCcAT | 50_15 | -19.6 | 151949 |
| 50 | GATAGTATCAACCCAT | GAtagtatCAACccAT | 50_16 | -20.7 | 151949 |
| 50 | GATAGTATCAACCCAT | GAtagtatCAaCccAT | 50_17 | -19.7 | 151949 |
| 50 | GATAGTATCAACCCAT | GAtagtatCaaCCcAT | 50_18 | -20.5 | 151949 |
| 51 | TGATAGTATCAACCCAT | TGatagtaTcAAccCAT | 51_1 | -20.7 | 151949 |
| 51 | TGATAGTATCAACCCAT | TGatagtaTcaAccCAT | 51_2 | -20.4 | 151949 |
| 51 | TGATAGTATCAACCCAT | TGatagtatcAAccCAT | 51_3 | -20.2 | 151949 |
| 51 | TGATAGTATCAACCCAT | TGatagtaTcaaccCAT | 51_4 | -20.0 | 151949 |
| 51 | TGATAGTATCAACCCAT | TGatagtaTcAaccCAT | 51_5 | -20.1 | 151949 |
| 51 | TGATAGTATCAACCCAT | TGatagtatcaAccCAT | 51_6 | -19.9 | 151949 |
| 51 | TGATAGTATCAACCCAT | TGaTAgtatcaacCcAT | 51_7 | -20.7 | 151949 |
| 51 | TGATAGTATCAACCCAT | TGAtagtatcaacCcAT | 51_8 | -19.9 | 151949 |
| 51 | TGATAGTATCAACCCAT | TGAtagtatcaACccAT | 51_9 | -20.8 | 151949 |
| 51 | TGATAGTATCAACCCAT | TGatagtatcAaCcCAT | 51_10 | -20.4 | 151949 |
| 51 | TGATAGTATCAACCCAT | TGatagtatcAaCCcAT | 51_11 | -20.6 | 151949 |
| 51 | TGATAGTATCAACCCAT | TGatagtaTCAacccAT | 51_12 | -20.8 | 151949 |
| 51 | TGATAGTATCAACCCAT | TGatagtatCAAcccAT | 51_13 | -20.3 | 151949 |
| 51 | TGATAGTATCAACCCAT | TGatagtatCaAccCAT | 51_14 | -20.6 | 151949 |
| 51 | TGATAGTATCAACCCAT | TGatagtatCaaccCAT | 51_15 | -20.2 | 151949 |
| 51 | TGATAGTATCAACCCAT | TGatagtatCAacCcAT | 51_16 | -20.3 | 151949 |
| 51 | TGATAGTATCAACCCAT | TGatagtaTCaacCcAT | 51_17 | -20.0 | 151949 |
| 51 | TGATAGTATCAACCCAT | TGatagtaTCaACccAT | 51_18 | -20.9 | 151949 |
| 51 | TGATAGTATCAACCCAT | TGatagtatCAaCccAT | 51_19 | -20.4 | 151949 |
| 51 | TGATAGTATCAACCCAT | TGatagtaTCaaCccAT | 51_20 | -20.1 | 151949 |
| 51 | TGATAGTATCAACCCAT | TGatagtatCaaCcCAT | 51_21 | -20.9 | 151949 |
| 52 | TTGATGCTTCTAAATCA | TTgATgcttctaaATCA | 52_1 | -20.5 | 151990 |
| 52 | TTGATGCTTCTAAATCA | TTgatgcttcTaaATCA | 52_2 | -19.2 | 151990 |
| 52 | TTGATGCTTCTAAATCA | TTGAtgcttctaaaTCA | 52_3 | -19.8 | 151990 |
| 52 | TTGATGCTTCTAAATCA | TTGatgcttctaaATCA | 52_4 | -19.3 | 151990 |
| 52 | TTGATGCTTCTAAATCA | TTgATgcttctaaaTCA | 52_5 | -19.3 | 151990 |
| 52 | TTGATGCTTCTAAATCA | TTGAtgcttctaaAtCA | 52_6 | -19.0 | 151990 |
| 52 | TTGATGCTTCTAAATCA | TTGaTgcttctaaaTCA | 52_7 | -19.1 | 151990 |
| 52 | TTGATGCTTCTAAATCA | TTgaTgcttctaaATCA | 52_8 | -19.1 | 151990 |
| 52 | TTGATGCTTCTAAATCA | TTGatgcttcTaaATCA | 52_9 | -20.2 | 151990 |
| 52 | TTGATGCTTCTAAATCA | TTGaTgcttctaaATCA | 52_10 | -20.2 | 151990 |
| 52 | TTGATGCTTCTAAATCA | TTGatgcttCtaaATCA | 52_11 | -20.3 | 151990 |
| 52 | TTGATGCTTCTAAATCA | TTgatgcttCTAaatCA | 52_12 | -19.7 | 151990 |
| 52 | TTGATGCTTCTAAATCA | TTGatgcttCTaaatCA | 52_13 | -19.6 | 151990 |
| 52 | TTGATGCTTCTAAATCA | TTGatgcttCtAaaTCA | 52_14 | -19.2 | 151990 |
| 52 | TTGATGCTTCTAAATCA | TTGatgcttCtaAaTCA | 52_15 | -19.5 | 151990 |
| 52 | TTGATGCTTCTAAATCA | TTgatgctTCtaAaTCA | 52_16 | -19.4 | 151990 |
| 52 | TTGATGCTTCTAAATCA | TTgatgcttCtaaATCA | 52_17 | -19.2 | 151990 |

wherein non underlined capital letters are beta-D-oxy LNA nucleosides, lowercase letters are DNA nucleosides, all LNA C are 5-methyl cytosine, all internucleoside linkages are phosphorothioate internucleoside linkages, and underlined capital letters are 2′-O-methyl RNA nucleosides.

Pharmaceutically Acceptable Salts

In a further aspect, the invention provides a pharmaceutically acceptable salt of the antisense oligonucleotide or a conjugate thereof, such as a pharmaceutically acceptable sodium salt, ammonium salt or potassium salt.

Method of Manufacture

In a further aspect, the invention provides methods for manufacturing the antisense oligonucleotides of the invention comprising reacting nucleotide units and thereby forming covalently linked contiguous nucleotide units comprised in the antisense oligonucleotide. Preferably, the method uses phophoramidite chemistry (see for example Caruthers et al, 1987, Methods in Enzymology vol. 154, pages 287-313). In a further embodiment, the method further comprises reacting the contiguous nucleotide sequence with a conjugating moiety (ligand) to covalently attach the conjugate moiety to the antisense oligonucleotide. In a further aspect, a method is provided for manufacturing the composition of the invention, comprising mixing the antisense oligonucleotide or conjugated antisense oligonucleotide of the invention with a pharmaceutically acceptable diluent, solvent, carrier, salt and/or adjuvant.

Pharmaceutical Composition

In a further aspect, the invention provides pharmaceutical compositions comprising any of the aforementioned antisense oligonucleotides and/or antisense oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments, the pharmaceutically acceptable diluent is sterile phosphate buffered saline or sterile sodium carbonate buffer.

In some embodiments, the antisense oligonucleotide of the invention is in the form of a solution in the pharmaceutically acceptable diluent, for example dissolved in PBS or sodium carbonate buffer. In some embodiments, the antisense oligonucleotide of the invention, or pharmaceutically acceptable salt thereof is in a solid form, such as a powder, such as a lyophilized powder. In some embodiments, the antisense oligonucleotide may be pre-formulated in the solution or in some embodiments, may be in the form of a dry powder (e.g. a lyophilized powder) which may be dissolved in the pharmaceutically acceptable diluent prior to administration.

Suitably, for example the antisense oligonucleotide may be dissolved in a concentration of 0.1-100 mg/ml, such as 1-10 mg/the pharmaceutically acceptable diluent.

In some embodiments, the oligonucleotide of the invention is formulated in a unit dose of between 0.5-100 mg, such as 1 mg-50 mg, or 2-25 mg.

In some embodiments, the antisense oligonucleotide is used in the pharmaceutically acceptable diluent at a concentration of 50-300 μM.

Antisense oligonucleotides or antisense oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

Pharmaceutical compositions, such as solutions, may be sterilized by conventional sterilization techniques, or may be sterile filtered. The resulting solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the antisense oligonucleotide or antisense oligonucleotide conjugate of the invention is a prodrug. In particular with respect to antisense oligonucleotide conjugates the conjugate moiety is cleaved off the antisense oligonucleotide once the prodrug is delivered to the site of action, e.g. the target cell.

Applications

The antisense oligonucleotides of the invention may be utilized as research reagents for, for example, diagnostics, therapeutics and prophylaxis.

In research, such antisense oligonucleotides may be used to specifically modulate the synthesis of Naᵥ1.7 or in some aspects Naᵥ1.8 protein in cells (e.g. in vitro cell cultures) and experimental animals thereby facilitating functional analysis of the target or an appraisal of its usefulness as a target for therapeutic intervention. Typically, the target modulation is achieved by degrading or inhibiting the mRNA producing the protein, thereby prevent protein formation or by degrading or inhibiting a modulator of the gene or mRNA producing the protein.

If employing the antisense oligonucleotide of the invention in research or diagnostics the target nucleic acid may be a cDNA or a synthetic nucleic acid derived from DNA or RNA.

The present invention provides an in vivo or in vitro method for modulating SCN9A expression in a target cell which is expressing SCN9A, said method comprising administering an antisense oligonucleotide of the invention in an effective amount to said cell.

In some embodiments, the target cell, is a mammalian cell in particular a human cell. The target cell may be an in vitro cell culture or an in vivo cell forming part of a tissue in a mammal. In preferred embodiments, the target cell is present in the peripheral nervous system, such as the dorsal root ganglion.

In diagnostics, the oligonucleotides may be used to detect and quantitate SCN9A expression in cell and tissues by northern blotting, in-situ hybridization or similar techniques.

Therapeutic Applications

The antisense oligonucleotides of the invention, or the antisense oligonucleotide conjugates, salts or pharmaceutical compositions of the invention, may be administered to an animal or a human for the prevention or treatment of pain, such as chronic pain, neuropathic pain, inflammatory pain, spontaneous pain, or nociceptive pain. The antisense oligonucleotides of the invention, or the conjugates, salts or pharmaceutical compositions of the invention may be for use as a local analgesic. The pain which may be treated with the antisense oligonucleotides of the invention, or the antisense oligonucleotide conjugates, salts or pharmaceutical compositions of the invention may be the pain signal in the peripheral nervous system. Indications associated with pain with a significant peripheral component include for example, diabetic neuropathies, cancer, cranial neuralgia, postherpetic neuralgia and post-surgical neuralgia.

Pain which may be prevented, treated or ameliorated using the antisense oligonucleotide, antisense oligonucleotide conjugate, pharmaceutical composition or salt of the invention may for example be selected from the group consisting of pain associated with inherited erythromelalgia (EIM), paroxysmal extreme pain disorder (PEPD), trigeminal neuralgia, neurophathic pain, chronic pain, but also general treatment of nociceptive (e.g. decompression of a nerve), neuropathic pain (e.g. diabetic neuropathy), visceral pain, or mixed pain.

The invention provides for the antisense oligonucleotide, antisense oligonucleotide conjugate, composition or salt of the invention for the use for the prevention or for the treatment of pain, such as chronic pain, neuropathic pain, inflammatory pain, spontaneous pain, or nociceptive pain.

The invention further relates to use of an antisense oligonucleotides, antisense oligonucleotide conjugate or a pharmaceutical composition of the invention for the manufacture of a medicament for the treatment or prevention of pain, such as chronic pain, neuropathic pain, inflammatory pain, spontaneous pain, or nociceptive pain.

The invention provides for the antisense oligonucleotide, antisense oligonucleotide conjugate, pharmaceutical composition or salt of the invention for the use as a local analgesic.

The invention provides for the use of the antisense oligonucleotide, antisense oligonucleotide conjugate, pharmaceutical composition or salt of the invention for manufacture of a local analgesic.

The invention provides for the antisense oligonucleotide, antisense oligonucleotide conjugate, pharmaceutical composition or salt of the invention for the use for the prevention or for the treatment of pain associated with inherited erythromelalgia (EIIVI), paroxysmal extreme pain disorder (PEPD), trigeminal neuralgia, neurophathic pain, chronic pain, but also general treatment of nociceptive (e.g. decompression of a nerve), neuropathic pain (e.g. diabetic neuropathy), visceral pain, or mixed pain.

The invention further relates to use of an antisense oligonucleotide, antisense oligonucleotide conjugate or a pharmaceutical composition of the invention for the manufacture of a medicament for the treatment or prevention of pain associated with inherited erythromelalgia (EIIVI), paroxysmal extreme pain disorder (PEPD), trigeminal neuralgia, neurophathic pain, chronic pain, but also general treatment of nociceptive (e.g. decompression of a nerve), neuropathic pain (e.g. diabetic neuropathy), visceral pain, or mixed pain.

Methods of Treatment

The invention provides methods for treating or preventing pain in a subject, such as a human, who is suffering from or is likely to suffer pain, comprising administering a therapeutically or prophylactically effective amount of an antisense oligonucleotide, an antisense oligonucleotide conjugate or a pharmaceutical composition of the invention to a subject who is suffering from or is susceptible to suffering from pain, such as chronic pain, neuropathic pain, inflammatory pain, spontaneous pain, or nociceptive pain.

By way of example, the method of treatment may be in subjects whose are suffering from an indication selected from the group consisting of diabetic neuropathies, cancer, cranial neuralgia, postherpetic neuralgia and post-surgical neuralgia.

The method of the invention may be for treating and relieving pain, such as pain associated with inherited erythromelalgia (EIM), paroxysmal extreme pain disorder (PEPD), trigeminal neuralgia, neurophathic pain, chronic pain, but also general treatment of nociceptive (e.g. decompression of a nerve), neuropathic pain (e.g. diabetic neuropathy), visceral pain, or mixed pain.

The methods of the invention are preferably employed for treatment or prophylaxis against pain which is mediated by Naᵥ1.7.

Administration

The antisense oligonucleotide, antisense oligonucleotide conjugate or pharmaceutical composition of the present invention may be administered via parenteral administration.

In some embodiments, the administration route is subcutaneous or intravenous.

In some embodiments, the administration route is selected from the group consisting of intravenous, subcutaneous, intra-muscular, intracerebral, epidural, intracerebroventricular intraocular, intrathecal administration, and transforaminal administration.

In some advantageous embodiments, the administration is via intrathecal administration, or epidural administration or transforaminal administration.

Advantageously, the antisense oligonucleotide, antisense oligonucleotide conjugate or pharmaceutical compositions of the present invention are administered intrathecally.

The invention also provides for the use of the antisense oligonucleotide of the invention, or antisense oligonucleotide conjugate thereof, such as pharmaceutical salts or compositions of the invention, for the manufacture of a medicament for the prevention or treatment of pain wherein the medicament is in a dosage form for intrathecal administration.

The invention also provides for the use of the antisense oligonucleotide or antisense oligonucleotide conjugate of the invention as described for the manufacture of a medicament for the manufacture of a medicament for the prevention or treatment of pain wherein the medicament is in a dosage form for intrathecal administration.

The invention also provides for the antisense oligonucleotide of the invention, or antisense oligonucleotide conjugate thereof, such as pharmaceutical salts or compositions of the invention, for use as a medicament for the prevention or treatment of pain wherein the medicament is in a dosage form for intrathecal administration.

The invention also provides for the antisense oligonucleotide or antisense oligonucleotide conjugate of the invention, for use as a medicament for the prevention or treatment of pain wherein the medicament is in a dosage form for intrathecal administration.

Combination Therapies

In some embodiments, the antisense oligonucleotide, antisense oligonucleotide conjugate or pharmaceutical composition of the invention is for use in a combination treatment with another therapeutic agent. The therapeutic agent can for example be the standard of care for the diseases or disorders described above. In some embodiments, the compound of the invention is used in combination with small molecule analgesics which may be administered concurrently or independently of the administration of the compound or compositions of the invention. An advantage of a combination therapy of the compounds of the invention with small molecule analgesics is that small molecule analgesics have a rapid onset of pain relieving activity, typically with a short duration of action (hours—days), whereas the compounds of the invention has a delayed onset of activity (typically a few days or even a week+), but with a long duration of action (weeks—months, e.g. 2+, 3+ or 4 months+).

### EXAMPLES

### Example 1: Testing In Vitro Efficacy of Antisense Oligonucleotides Targeting SCN9A in SK-N-AS Cell Line(s) at Single Concentration(s)

SK-N-AS cells have been maintained in a humidified incubator as recommended by the supplier. The vendor and recommended culture conditions are reported in Table 4.

TABLE 4

| | | | | Incub. time | Incub. time |
|---|---|---|---|---|---|
| | | | Seeding density | before oligo | with oligo |
| Cell Line | Vendor | Culture Condition | (cells/well) | (hrs) | (hrs) |
| SK-N-AS | European | 37° C., 5% CO₂, | 9300 cells | 24 | 72 |
| (ECACC - | Collection of | 95% relative humidity, | seeded in | | |
| 94092302) | Authenticated | in active evaporation | 95 μL in | | |
| | Cell Cultures | incubator | SK-N-AS culture | | |
| | (ECACC) | (Thermo C10) | medium | | |

For assays, cells were seeded in a 96-multi well plate in culture media and incubated as reported in Table 4 before addition of antisense oligonucleotides dissolved in a volume of 5 μL PBS. The final concentrations of the antisense oligonucleotides are given in Table 6 below.

The cells were harvested 72 hours after the addition of antisense oligonucleotides (see Table 4). RNA was extracted using the PureLink™ Pro 96 RNA Purification kit (Thermo Fisher Scientific) according to the manufacturer's instructions and eluted in 50 μL of water. The RNA was subsequently diluted 10 times with DNase/RNase free Water (Gibco) and heated to 90° C. for one minute.

For gene expressions analysis, One Step RT-qPCR was performed using gScript™ XLT One-Step RT-qPCR ToughMix®, Low ROX™ (Quantabio) in a duplex set up. The primer assays used for qPCR are collated in Table 5 for both target and endogenous control.

TABLE 5

| Endogen contr. | Endog. contr. | Endogen. contr. | Target | Target | Target |
|---|---|---|---|---|---|
| assay | vendor | fluorophore | assay | vendor | fluorophore |
| GUSB: | IDT | HEX-ZEN | SCN9A: | IDT | FAM-ZEN |
| Hs.PTv.27737538 | (Integrated | | Hs.PT.58.20989243 | | |
| | DNA | | | | |
| | Technologies) | | | | |

TABLE 6
The relative Human SCN9A mRNA expression level is
shown as percent of control (PBS-treated cells)

| | | NAV1.7 | | | NAV1.7 | | | NAV1.7 |
|---|---|---|---|---|---|---|---|---|
| | | qPCR SP | | | qPCR SP | | | qPCR SP |
| | | probe1 | | | probe1 | | | probe1 |
| CMP | Conc | SK-N-AS: | CMP | Conc | SK-N-AS: | CMP | Conc | SK-N-AS: |
| ID NO | (μM) | AP011352 | ID NO | (μM) | AP011352 | ID NO | (μM) | AP011352 2 |
| 28_1 | 0.1 | 94.1 | 33_17 | 0.1 | 67.2 | 39_96 | 0.031 | 93 |
| 28_1 | 0.4 | 55.2 | 33_17 | 0.4 | 23.6 | 39_96 | 0.1 | 88.3 |
| 29_1 | 0.031 | 81.7 | 33_2 | 0.031 | 87.9 | 39_96 | 0.4 | 58 |
| 29_1 | 0.1 | 48.9 | 33_2 | 0.1 | 56.1 | 39_97 | 0.031 | 99.8 |
| 29_1 | 0.4 | 17.6 | 33_2 | 0.4 | 20.4 | 39_97 | 0.1 | 72.2 |
| 29_10 | 0.031 | 90.2 | 33_3 | 0.031 | 85.1 | 39_97 | 0.4 | 45.1 |
| 29_10 | 0.1 | 48.8 | 33_3 | 0.1 | 59.2 | 39_98 | 0.031 | 100 |
| 29_10 | 0.4 | 16.9 | 33_3 | 0.4 | 20.9 | 39_98 | 0.1 | 82.7 |
| 29_100 | 0.031 | 88.4 | 33_4 | 0.031 | 87.3 | 39_98 | 0.4 | 48.2 |
| 29_100 | 0.1 | 65.6 | 33_4 | 0.1 | 64.5 | 39_99 | 0.031 | 93.6 |
| 29_100 | 0.4 | 29.4 | 33_4 | 0.4 | 21 | 39_99 | 0.1 | 73.6 |
| 29_101 | 0.031 | 90.1 | 33_5 | 0.031 | 89.1 | 39_99 | 0.4 | 32.6 |
| 29_101 | 0.1 | 61.2 | 33_5 | 0.1 | 67.6 | 40_1 | 0.031 | 101 |
| 29_101 | 0.4 | 27.7 | 33_5 | 0.4 | 21.7 | 40_1 | 0.1 | 94.9 |
| 29_102 | 0.031 | 86 | 33_6 | 0.031 | 90.8 | 40_1 | 0.4 | 90.3 |
| 29_102 | 0.1 | 62.6 | 33_6 | 0.1 | 73.7 | 40_2 | 0.031 | 101 |
| 29_102 | 0.4 | 25.8 | 33_6 | 0.4 | 25.3 | 40_2 | 0.1 | 83.2 |
| 29_103 | 0.031 | 94.3 | 33_7 | 0.031 | 90.1 | 40_2 | 0.4 | 58.1 |
| 29_103 | 0.1 | 76.8 | 33_7 | 0.1 | 82.3 | 40_3 | 0.031 | 103 |
| 29_103 | 0.4 | 43.5 | 33_7 | 0.4 | 38.3 | 40_3 | 0.1 | 102 |
| 29_104 | 0.031 | 88 | 33_8 | 0.031 | 93.1 | 40_3 | 0.4 | 81.9 |
| 29_104 | 0.1 | 77.5 | 33_8 | 0.1 | 69.5 | 40_4 | 0.031 | 92.6 |
| 29_104 | 0.4 | 54.5 | 33_8 | 0.4 | 29.2 | 40_4 | 0.1 | 88.1 |
| 29_105 | 0.031 | 90.3 | 33_9 | 0.031 | 85 | 40_4 | 0.4 | 62.1 |
| 29_105 | 0.1 | 72 | 33_9 | 0.1 | 58.2 | 40_5 | 0.031 | 99.2 |
| 29_105 | 0.4 | 32.3 | 33_9 | 0.4 | 17.2 | 40_5 | 0.1 | 86.7 |
| 29_106 | 0.031 | 97.9 | 34_1 | 0.031 | 88.4 | 40_5 | 0.4 | 71.6 |
| 29_106 | 0.1 | 80.3 | 34_1 | 0.1 | 63.4 | 40_6 | 0.031 | 97.5 |
| 29_106 | 0.4 | 58.4 | 34_1 | 0.4 | 28.9 | 40_6 | 0.1 | 91 |
| 29_107 | 0.031 | 90.1 | 34_10 | 0.031 | 94.5 | 40_6 | 0.4 | 68 |
| 29_107 | 0.1 | 80 | 34_10 | 0.1 | 65.7 | 41_1 | 0.031 | 98.7 |
| 29_107 | 0.4 | 30.8 | 34_10 | 0.4 | 29.4 | 41_1 | 0.1 | 100 |
| 29_108 | 0.031 | 94.4 | 34_11 | 0.031 | 95 | 41_1 | 0.4 | 69.5 |
| 29_108 | 0.1 | 73.5 | 34_11 | 0.1 | 73.4 | 41_10 | 0.031 | 101 |
| 29_108 | 0.4 | 34.7 | 34_11 | 0.4 | 37.2 | 41_10 | 0.1 | 89 |
| 29_109 | 0.031 | 87.6 | 34_12 | 0.031 | 90 | 41_10 | 0.4 | 66.3 |
| 29_109 | 0.1 | 60.2 | 34_12 | 0.1 | 69.9 | 41_11 | 0.031 | 93.7 |
| 29_109 | 0.4 | 27.5 | 34_12 | 0.4 | 29 | 41_11 | 0.1 | 79 |
| 29_11 | 0.031 | 85.1 | 34_13 | 0.031 | 90.9 | 41_11 | 0.4 | 59.3 |
| 29_11 | 0.1 | 56.6 | 34_13 | 0.1 | 70.8 | 41_12 | 0.031 | 99.8 |
| 29_11 | 0.4 | 21.4 | 34_13 | 0.4 | 29.8 | 41_12 | 0.1 | 98.4 |
| 29_110 | 0.031 | 91.3 | 34_14 | 0.031 | 98.6 | 41_12 | 0.4 | 82.6 |
| 29_110 | 0.1 | 65.9 | 34_14 | 0.1 | 74.8 | 41_13 | 0.031 | 101 |
| 29_110 | 0.4 | 43.5 | 34_14 | 0.4 | 52 | 41_13 | 0.1 | 79.4 |
| 29_111 | 0.031 | 102 | 34_15 | 0.031 | 97.4 | 41_13 | 0.4 | 62.6 |
| 29_111 | 0.1 | 81.7 | 34_15 | 0.1 | 76.7 | 41_14 | 0.031 | 96.9 |
| 29_111 | 0.4 | 51 | 34_15 | 0.4 | 34.5 | 41_14 | 0.1 | 87.8 |
| 29_112 | 0.031 | 90.2 | 34_16 | 0.031 | 91.6 | 41_14 | 0.4 | 66.9 |
| 29_112 | 0.1 | 61.1 | 34_16 | 0.1 | 75.6 | 41_15 | 0.031 | 104 |
| 29_112 | 0.4 | 23.3 | 34_16 | 0.4 | 41.4 | 41_15 | 0.1 | 86.1 |
| 29_113 | 0.031 | 82.6 | 34_17 | 0.031 | 103 | 41_15 | 0.4 | 62.6 |
| 29_113 | 0.1 | 74 | 34_17 | 0.1 | 74.8 | 41_2 | 0.031 | 91.6 |
| 29_113 | 0.4 | 17.6 | 34_17 | 0.4 | 38.7 | 41_2 | 0.1 | 75.1 |
| 29_114 | 0.031 | 84.7 | 34_2 | 0.031 | 83.2 | 41_2 | 0.4 | 38.9 |
| 29_114 | 0.1 | 58.7 | 34_2 | 0.1 | 65.2 | 41_3 | 0.031 | 102 |
| 29_114 | 0.4 | 22.5 | 34_2 | 0.4 | 25.4 | 41_3 | 0.1 | 96.5 |
| 29_115 | 0.031 | 91.9 | 34_3 | 0.031 | 90.3 | 41_3 | 0.4 | 70.7 |
| 29_115 | 0.1 | 74.8 | 34_3 | 0.1 | 65.7 | 41_4 | 0.031 | 98.1 |
| 29_115 | 0.4 | 32.1 | 34_3 | 0.4 | 38.7 | 41_4 | 0.1 | 84.6 |
| 29_116 | 0.031 | 87.9 | 34_4 | 0.031 | 86.5 | 41_4 | 0.4 | 62.9 |
| 29_116 | 0.1 | 57.1 | 34_4 | 0.1 | 57.3 | 41_5 | 0.031 | 99.3 |
| 29_116 | 0.4 | 25.8 | 34_4 | 0.4 | 18 | 41_5 | 0.1 | 96.4 |
| 29_117 | 0.031 | 91.5 | 34_5 | 0.031 | 85.2 | 41_5 | 0.4 | 97.2 |
| 29_117 | 0.1 | 65.3 | 34_5 | 0.1 | 63.5 | 41_6 | 0.031 | 103 |
| 29_117 | 0.4 | 31.9 | 34_5 | 0.4 | 25.4 | 41_6 | 0.1 | 83.9 |
| 29_118 | 0.031 | 91.1 | 34_6 | 0.031 | 93.6 | 41_6 | 0.4 | 73.7 |
| 29_118 | 0.1 | 71 | 34_6 | 0.1 | 66.1 | 41_7 | 0.031 | 100 |
| 29_118 | 0.4 | 39.6 | 34_6 | 0.4 | 40.1 | 41_7 | 0.1 | 92.7 |
| 29_119 | 0.031 | 89.1 | 34_7 | 0.031 | 102 | 41_7 | 0.4 | 62 |
| 29_119 | 0.1 | 74.3 | 34_7 | 0.1 | 91.5 | 41_8 | 0.031 | 94.3 |
| 29_119 | 0.4 | 43.7 | 34_7 | 0.4 | 64.3 | 41_8 | 0.1 | 96.5 |
| 29_12 | 0.031 | 75 | 34_8 | 0.031 | 97.1 | 41_8 | 0.4 | 67.9 |
| 29_12 | 0.1 | 45.2 | 34_8 | 0.1 | 65.5 | 41_9 | 0.031 | 96.9 |
| 29_12 | 0.4 | 17.2 | 34_8 | 0.4 | 34.7 | 41_9 | 0.1 | 80.4 |
| 29_120 | 0.031 | 86 | 34_9 | 0.031 | 91.5 | 41_9 | 0.4 | 47.3 |
| 29_120 | 0.1 | 69.8 | 34_9 | 0.1 | 66.3 | 42_1 | 0.031 | 99.3 |
| 29_120 | 0.4 | 36.4 | 34_9 | 0.4 | 27.8 | 42_1 | 0.1 | 93.4 |
| 29_121 | 0.031 | 89.9 | 35_1 | 0.031 | 90.5 | 42_1 | 0.4 | 78 |
| 29_121 | 0.1 | 66.3 | 35_1 | 0.1 | 72.5 | 42_10 | 0.031 | 104 |
| 29_121 | 0.4 | 26.7 | 35_1 | 0.4 | 44.3 | 42_10 | 0.1 | 94.7 |
| 29_122 | 0.031 | 90.7 | 36_1 | 0.031 | 94.5 | 42_10 | 0.4 | 74.3 |
| 29_122 | 0.1 | 78.5 | 36_1 | 0.1 | 87.6 | 42_11 | 0.031 | 94.4 |
| 29_122 | 0.4 | 49.1 | 36_1 | 0.4 | 55.6 | 42_11 | 0.1 | 74.9 |
| 29_123 | 0.031 | 92.7 | 36_2 | 0.031 | 94.9 | 42_11 | 0.4 | 39.5 |
| 29_123 | 0.1 | 85 | 36_2 | 0.1 | 89.3 | 42_12 | 0.031 | 103 |
| 29_123 | 0.4 | 56.1 | 36_2 | 0.4 | 62.4 | 42_12 | 0.1 | 79.9 |
| 29_124 | 0.031 | 91.7 | 36_3 | 0.031 | 96.5 | 42_12 | 0.4 | 46.9 |
| 29_124 | 0.1 | 75.4 | 36_3 | 0.1 | 84.8 | 42_13 | 0.031 | 110 |
| 29_124 | 0.4 | 51.2 | 36_3 | 0.4 | 53.1 | 42_13 | 0.1 | 87.7 |
| 29_125 | 0.031 | 99 | 36_4 | 0.031 | 93.7 | 42_13 | 0.4 | 55 |
| 29_125 | 0.1 | 91.7 | 36_4 | 0.1 | 78.9 | 42_14 | 0.031 | 99.8 |
| 29_125 | 0.4 | 79.3 | 36_4 | 0.4 | 53.3 | 42_14 | 0.1 | 91.8 |
| 29_126 | 0.031 | 95.5 | 36_5 | 0.031 | 92.8 | 42_14 | 0.4 | 65.6 |
| 29_126 | 0.1 | 94.6 | 36_5 | 0.1 | 84.5 | 42_15 | 0.031 | 95.9 |
| 29_126 | 0.4 | 90.4 | 36_5 | 0.4 | 76.3 | 42_15 | 0.1 | 78.9 |
| 29_127 | 0.031 | 108 | 37_1 | 0.031 | 100 | 42_15 | 0.4 | 48.3 |
| 29_127 | 0.1 | 102 | 37_1 | 0.1 | 71.2 | 42_16 | 0.031 | 99.1 |
| 29_127 | 0.4 | 58.9 | 37_1 | 0.4 | 33.4 | 42_16 | 0.1 | 85 |
| 29_128 | 0.031 | 93.3 | 37_2 | 0.031 | 99.2 | 42_16 | 0.4 | 66.1 |
| 29_128 | 0.1 | 80.6 | 37_2 | 0.1 | 87 | 42_2 | 0.031 | 99.2 |
| 29_128 | 0.4 | 52.9 | 37_2 | 0.4 | 48.7 | 42_2 | 0.1 | 92.4 |
| 29_129 | 0.031 | 95.2 | 37_3 | 0.031 | 85.1 | 42_2 | 0.4 | 78.6 |
| 29_129 | 0.1 | 78.8 | 37_3 | 0.1 | 63.6 | 42_3 | 0.031 | 90.3 |
| 29_129 | 0.4 | 51.7 | 37_3 | 0.4 | 32.7 | 42_3 | 0.1 | 70.6 |
| 29_13 | 0.031 | 81.4 | 37_4 | 0.031 | 93.6 | 42_3 | 0.4 | 36.9 |
| 29_13 | 0.1 | 55.5 | 37_4 | 0.1 | 71.4 | 42_4 | 0.031 | 99.8 |
| 29_13 | 0.4 | 29.4 | 37_4 | 0.4 | 44 | 42_4 | 0.1 | 83.3 |
| 29_130 | 0.031 | 94.5 | 37_5 | 0.031 | 91.8 | 42_4 | 0.4 | 60.8 |
| 29_130 | 0.1 | 93.8 | 37_5 | 0.1 | 64.4 | 42_5 | 0.031 | 100 |
| 29_130 | 0.4 | 84.2 | 37_5 | 0.4 | 30.9 | 42_5 | 0.1 | 83.4 |
| 29_131 | 0.031 | 90.2 | 38_1 | 0.031 | 99.4 | 42_5 | 0.4 | 61.1 |
| 29_131 | 0.1 | 74.2 | 38_1 | 0.1 | 69.5 | 42_6 | 0.031 | 92.6 |
| 29_131 | 0.4 | 48.3 | 38_1 | 0.4 | 45.6 | 42_6 | 0.1 | 77.4 |
| 29_132 | 0.031 | 94.7 | 39_1 | 0.031 | 94.2 | 42_6 | 0.4 | 42.7 |
| 29_132 | 0.1 | 84.3 | 39_1 | 0.1 | 60.6 | 42_7 | 0.031 | 102 |
| 29_132 | 0.4 | 59.8 | 39_1 | 0.4 | 34.1 | 42_7 | 0.1 | 94.7 |
| 29_133 | 0.031 | 102 | 39_10 | 0.031 | 85.2 | 42_7 | 0.4 | 80.1 |
| 29_133 | 0.1 | 79.9 | 39_10 | 0.1 | 59.1 | 42_8 | 0.031 | 103 |
| 29_133 | 0.4 | 57.3 | 39_10 | 0.4 | 25.8 | 42_8 | 0.1 | 92.2 |
| 29_134 | 0.031 | 93 | 39_100 | 0.031 | 88.8 | 42_8 | 0.4 | 72.4 |
| 29_134 | 0.1 | 90.1 | 39_100 | 0.1 | 62.2 | 42_9 | 0.031 | 98.5 |
| 29_134 | 0.4 | 83.6 | 39_100 | 0.4 | 34.6 | 42_9 | 0.1 | 83.6 |
| 29_14 | 0.031 | 82.9 | 39_101 | 0.031 | 90.4 | 42_9 | 0.4 | 60.4 |
| 29_14 | 0.1 | 47.3 | 39_101 | 0.1 | 71.7 | 43_1 | 0.031 | 91.9 |
| 29_14 | 0.4 | 14.3 | 39_101 | 0.4 | 44.2 | 43_1 | 0.1 | 88.3 |
| 29_15 | 0.031 | 79.3 | 39_102 | 0.031 | 92 | 43_1 | 0.4 | 63.9 |
| 29_15 | 0.1 | 52.7 | 39_102 | 0.1 | 68.1 | 43_2 | 0.031 | 99.2 |
| 29_15 | 0.4 | 18.6 | 39_102 | 0.4 | 42.5 | 43_2 | 0.1 | 85.8 |
| 29_16 | 0.031 | 78 | 39_103 | 0.031 | 94.4 | 43_2 | 0.4 | 60.6 |
| 29_16 | 0.1 | 52 | 39_103 | 0.1 | 76.5 | 43_3 | 0.031 | 95.8 |
| 29_16 | 0.4 | 22.2 | 39_103 | 0.4 | 45.5 | 43_3 | 0.1 | 65.3 |
| 29_17 | 0.031 | 77.7 | 39_104 | 0.031 | 90.1 | 43_3 | 0.4 | 35.3 |
| 29_17 | 0.1 | 46.9 | 39_104 | 0.1 | 77 | 44_1 | 0.031 | 94.6 |
| 29_17 | 0.4 | 16.1 | 39_104 | 0.4 | 51.5 | 44_1 | 0.1 | 93.3 |
| 29_18 | 0.031 | 78.3 | 39_105 | 0.031 | 92.5 | 44_1 | 0.4 | 60.4 |
| 29_18 | 0.1 | 44.7 | 39_105 | 0.1 | 76 | 44_10 | 0.031 | 92.2 |
| 29_18 | 0.4 | 14.1 | 39_105 | 0.4 | 54.7 | 44_10 | 0.1 | 80.4 |
| 29_19 | 0.031 | 89.6 | 39_106 | 0.031 | 102 | 44_10 | 0.4 | 53.5 |
| 29_19 | 0.1 | 57.1 | 39_106 | 0.1 | 80.1 | 44_11 | 0.031 | 98 |
| 29_19 | 0.4 | 25.8 | 39_106 | 0.4 | 48 | 44_11 | 0.1 | 82.6 |
| 29_2 | 0.031 | 87.4 | 39_107 | 0.031 | 90.7 | 44_11 | 0.4 | 72.8 |
| 29_2 | 0.1 | 49 | 39_107 | 0.1 | 80.4 | 44_12 | 0.031 | 99 |
| 29_2 | 0.4 | 15.9 | 39_107 | 0.4 | 56.1 | 44_12 | 0.1 | 87.2 |
| 29_20 | 0.031 | 84.9 | 39_108 | 0.031 | 100 | 44_12 | 0.4 | 67.3 |
| 29_20 | 0.1 | 58.2 | 39_108 | 0.1 | 75 | 44_13 | 0.031 | 88.6 |
| 29_20 | 0.4 | 23.1 | 39_108 | 0.4 | 53.4 | 44_13 | 0.1 | 85.1 |
| 29_21 | 0.031 | 86.7 | 39_109 | 0.031 | 101 | 44_13 | 0.4 | 61.6 |
| 29_21 | 0.1 | 57.4 | 39_109 | 0.1 | 95.1 | 44_14 | 0.031 | 99.3 |
| 29_21 | 0.4 | 25.5 | 39_109 | 0.4 | 100 | 44_14 | 0.1 | 85 |
| 29_22 | 0.031 | 89.6 | 39-11 | 0.031 | 87.4 | 44_14 | 0.4 | 54.2 |
| 29_22 | 0.1 | 50.7 | 39-11 | 0.1 | 61.7 | 44_15 | 0.031 | 95.3 |
| 29_22 | 0.4 | 19.8 | 39-11 | 0.4 | 30.4 | 44_15 | 0.1 | 89.4 |
| 29_23 | 0.031 | 85.3 | 39-110 | 0.031 | 83.8 | 44_15 | 0.4 | 67.5 |
| 29_23 | 0.1 | 53 | 39-110 | 0.1 | 79.3 | 44_16 | 0.031 | 94.7 |
| 29_23 | 0.4 | 20.8 | 39-110 | 0.4 | 48.3 | 44_16 | 0.1 | 93.7 |
| 29_24 | 0.031 | 92.5 | 39-111 | 0.031 | 91.9 | 44_16 | 0.4 | 59.4 |
| 29_24 | 0.1 | 56.8 | 39-111 | 0.1 | 79.8 | 44_17 | 0.031 | 102 |
| 29_24 | 0.4 | 20.5 | 39-111 | 0.4 | 50.4 | 44_17 | 0.1 | 92.6 |
| 29_25 | 0.031 | 78.2 | 39_112 | 0.031 | 95.6 | 44_17 | 0.4 | 67.6 |
| 29_25 | 0.1 | 41.3 | 39_112 | 0.1 | 78.1 | 44_18 | 0.031 | 97.9 |
| 29_25 | 0.4 | 10.6 | 39_112 | 0.4 | 48.4 | 44_18 | 0.1 | 84.7 |
| 29_26 | 0.031 | 84.2 | 39_113 | 0.031 | 90.7 | 44_18 | 0.4 | 59.7 |
| 29_26 | 0.1 | 53.2 | 39_113 | 0.1 | 82.1 | 44_2 | 0.031 | 103 |
| 29_26 | 0.4 | 14.1 | 39_113 | 0.4 | 59.4 | 44_2 | 0.1 | 93.6 |
| 29_27 | 0.031 | 90.6 | 39_114 | 0.031 | 92.5 | 44_2 | 0.4 | 67.4 |
| 29_27 | 0.1 | 56.1 | 39_114 | 0.1 | 72.3 | 44_3 | 0.031 | 101 |
| 29_27 | 0.4 | 24.1 | 39_114 | 0.4 | 53.9 | 44_3 | 0.1 | 90.5 |
| 29_28 | 0.031 | 84.9 | 39_115 | 0.031 | 98.7 | 44_3 | 0.4 | 66.9 |
| 29_28 | 0.1 | 49.5 | 39_115 | 0.1 | 90.4 | 44_4 | 0.031 | 104 |
| 29_28 | 0.4 | 19.4 | 39_115 | 0.4 | 75.4 | 44_4 | 0.1 | 91.6 |
| 29_29 | 0.031 | 84 | 39_116 | 0.031 | 88.5 | 44_4 | 0.4 | 68.4 |
| 29_29 | 0.1 | 60.5 | 39_116 | 0.1 | 64.5 | 44_5 | 0.031 | 98.1 |
| 29_29 | 0.4 | 23.4 | 39_116 | 0.4 | 29.3 | 44_5 | 0.1 | 85.7 |
| 29_3 | 0.031 | 82.3 | 39_117 | 0.031 | 87.5 | 44_5 | 0.4 | 59.1 |
| 29_3 | 0.1 | 45.9 | 39_117 | 0.1 | 71.2 | 44_6 | 0.031 | 88.7 |
| 29_3 | 0.4 | 16.5 | 39_117 | 0.4 | 33.1 | 44_6 | 0.1 | 80.7 |
| 29_30 | 0.031 | 85.1 | 39_118 | 0.031 | 100 | 44_6 | 0.4 | 37.4 |
| 29_30 | 0.1 | 38.4 | 39_118 | 0.1 | 84.4 | 44_7 | 0.031 | 102 |
| 29_30 | 0.4 | 17.7 | 39_118 | 0.4 | 39.8 | 44_7 | 0.1 | 85.7 |
| 29_31 | 0.031 | 83.5 | 39_119 | 0.031 | 81.6 | 44_7 | 0.4 | 65.7 |
| 29_31 | 0.1 | 46 | 39_119 | 0.1 | 66 | 44_8 | 0.031 | 95.4 |
| 29_31 | 0.4 | 13.9 | 39_119 | 0.4 | 29.9 | 44_8 | 0.1 | 82.1 |
| 29_32 | 0.031 | 82.6 | 39_12 | 0.031 | 92.7 | 44_8 | 0.4 | 46.1 |
| 29_32 | 0.1 | 43.4 | 39_12 | 0.1 | 60.9 | 44_9 | 0.031 | 106 |
| 29_32 | 0.4 | 19.5 | 39_12 | 0.4 | 32.4 | 44_9 | 0.1 | 89.5 |
| 29_33 | 0.031 | 74.8 | 39_120 | 0.031 | 82.4 | 44_9 | 0.4 | 62.3 |
| 29_33 | 0.1 | 39.3 | 39_120 | 0.1 | 66.4 | 45_1 | 0.031 | 94 |
| 29_33 | 0.4 | 10.4 | 39_120 | 0.4 | 28.7 | 45_1 | 0.1 | 73.6 |
| 29_34 | 0.031 | 89.6 | 39_121 | 0.031 | 91.1 | 45_1 | 0.4 | 45.7 |
| 29_34 | 0.1 | 54.9 | 39_121 | 0.1 | 68.8 | 46_1 | 0.031 | 99.4 |
| 29_34 | 0.4 | 21.2 | 39_121 | 0.4 | 43 | 46_1 | 0.1 | 72 |
| 29_35 | 0.031 | 81.6 | 39_122 | 0.031 | 86.2 | 46_1 | 0.4 | 48.7 |
| 29_35 | 0.1 | 52 | 39_122 | 0.1 | 70.7 | 46_2 | 0.031 | 92.9 |
| 29_35 | 0.4 | 13.5 | 39_122 | 0.4 | 45.3 | 46_2 | 0.1 | 71.6 |
| 29_36 | 0.031 | 84.3 | 39_123 | 0.031 | 91.3 | 46_2 | 0.4 | 40.1 |
| 29_36 | 0.1 | 52.5 | 39_123 | 0.1 | 77.3 | 46_3 | 0.031 | 100 |
| 29_36 | 0.4 | 20.7 | 39_123 | 0.4 | 43 | 46_3 | 0.1 | 78.7 |
| 29_37 | 0.031 | 87.1 | 39_124 | 0.031 | 97.2 | 46_3 | 0.4 | 49.1 |
| 29_37 | 0.1 | 57.2 | 39_124 | 0.1 | 74.5 | 46_4 | 0.031 | 95.4 |
| 29_37 | 0.4 | 24.1 | 39_124 | 0.4 | 51.1 | 46_4 | 0.1 | 69.3 |
| 29_38 | 0.031 | 84.1 | 39_125 | 0.031 | 97.7 | 46_4 | 0.4 | 45.5 |
| 29_38 | 0.1 | 50.6 | 39_125 | 0.1 | 93.1 | 46_5 | 0.031 | 95.3 |
| 29_38 | 0.4 | 17.6 | 39_125 | 0.4 | 62.6 | 46_5 | 0.1 | 81.5 |
| 29_39 | 0.031 | 89.4 | 39_126 | 0.031 | 84.4 | 46_5 | 0.4 | 52.1 |
| 29_39 | 0.1 | 53.8 | 39_126 | 0.1 | 65.2 | 47_1 | 0.031 | 80 |
| 29_39 | 0.4 | 22.1 | 39_126 | 0.4 | 31.1 | 47_1 | 0.1 | 60.2 |
| 29_4 | 0.031 | 80.2 | 39_127 | 0.031 | 89.9 | 47_1 | 0.4 | 29.2 |
| 29_4 | 0.1 | 43 | 39_127 | 0.1 | 69 | 47_2 | 0.031 | 96.2 |
| 29_4 | 0.4 | 12.2 | 39_127 | 0.4 | 29.8 | 47_2 | 0.1 | 84.1 |
| 29_40 | 0.031 | 77.3 | 39_128 | 0.031 | 88.8 | 47_2 | 0.4 | 61.6 |
| 29_40 | 0.1 | 50 | 39_128 | 0.1 | 70.2 | 47_3 | 0.031 | 103 |
| 29_40 | 0.4 | 14.8 | 39_128 | 0.4 | 37.5 | 47_3 | 0.1 | 65.7 |
| 29_41 | 0.031 | 90.8 | 39_129 | 0.031 | 95.3 | 47_3 | 0.4 | 31.2 |
| 29_41 | 0.1 | 55.2 | 39_129 | 0.1 | 72.4 | 47_4 | 0.031 | 89.2 |
| 29_41 | 0.4 | 22.7 | 39_129 | 0.4 | 45.1 | 47_4 | 0.1 | 74.7 |
| 29_42 | 0.031 | 91.1 | 39_13 | 0.031 | 91.6 | 47_4 | 0.4 | 42.3 |
| 29_42 | 0.1 | 63.5 | 39_13 | 0.1 | 67.4 | 48_1 | 0.031 | 92.8 |
| 29_42 | 0.4 | 24.2 | 39_13 | 0.4 | 35.2 | 48_1 | 0.1 | 72.1 |
| 29_43 | 0.031 | 86.4 | 39_130 | 0.031 | 93.6 | 48_1 | 0.4 | 42 |
| 29_43 | 0.1 | 60.5 | 39_130 | 0.1 | 86.2 | 48_10 | 0.031 | 98.2 |
| 29_43 | 0.4 | 19.8 | 39_130 | 0.4 | 57.5 | 48_10 | 0.1 | 66.2 |
| 29_44 | 0.031 | 93.8 | 39_131 | 0.031 | 89.9 | 48_10 | 0.4 | 44.8 |
| 29_44 | 0.1 | 88.4 | 39_131 | 0.1 | 70.1 | 48-11 | 0.031 | 94.5 |
| 29_44 | 0.4 | 46.9 | 39_131 | 0.4 | 34.7 | 48-11 | 0.1 | 79.2 |
| 29_45 | 0.031 | 91.6 | 39_132 | 0.031 | 90.3 | 48-11 | 0.4 | 54.6 |
| 29_45 | 0.1 | 77.1 | 39_132 | 0.1 | 72.6 | 48_12 | 0.031 | 97.5 |
| 29_45 | 0.4 | 36.7 | 39_132 | 0.4 | 34.8 | 48_12 | 0.1 | 92.9 |
| 29_46 | 0.031 | 83.8 | 39_133 | 0.031 | 94.8 | 48_12 | 0.4 | 73.8 |
| 29_46 | 0.1 | 65.4 | 39_133 | 0.1 | 74 | 48_13 | 0.031 | 96.5 |
| 29_46 | 0.4 | 33.6 | 39_133 | 0.4 | 48.3 | 48_13 | 0.1 | 85.9 |
| 29_47 | 0.031 | 86.6 | 39_134 | 0.031 | 94 | 48_13 | 0.4 | 58.7 |
| 29_47 | 0.1 | 69.7 | 39_134 | 0.1 | 64.7 | 48_14 | 0.031 | 95 |
| 29_47 | 0.4 | 41.9 | 39_134 | 0.4 | 30.2 | 48_14 | 0.1 | 71.5 |
| 29_48 | 0.031 | 105 | 39_135 | 0.031 | 89 | 48_14 | 0.4 | 48.7 |
| 29_48 | 0.1 | 75.2 | 39_135 | 0.1 | 69.2 | 48_15 | 0.031 | 95.8 |
| 29_48 | 0.4 | 42.3 | 39_135 | 0.4 | 39.2 | 48_15 | 0.1 | 78.5 |
| 29_49 | 0.031 | 96.6 | 39_136 | 0.031 | 96.9 | 48_15 | 0.4 | 40.9 |
| 29_49 | 0.1 | 67.5 | 39_136 | 0.1 | 70.7 | 48_16 | 0.031 | 99.5 |
| 29_49 | 0.4 | 45.2 | 39_136 | 0.4 | 33 | 48_16 | 0.1 | 100 |
| 29_5 | 0.031 | 89.4 | 39_137 | 0.031 | 97 | 48_16 | 0.4 | 101 |
| 29_5 | 0.1 | 55.6 | 39_137 | 0.1 | 77.6 | 48_17 | 0.031 | 98.8 |
| 29_5 | 0.4 | 17.7 | 39_137 | 0.4 | 44.7 | 48_17 | 0.1 | 91.3 |
| 29_50 | 0.031 | 97 | 39_138 | 0.031 | 91.1 | 48_17 | 0.4 | 71.7 |
| 29_50 | 0.1 | 72.1 | 39_138 | 0.1 | 70.1 | 48_18 | 0.031 | 99.7 |
| 29_50 | 0.4 | 44.8 | 39_138 | 0.4 | 44.1 | 48_18 | 0.1 | 87.9 |
| 29_51 | 0.031 | 89.6 | 39_139 | 0.031 | 93.4 | 48_18 | 0.4 | 81.5 |
| 29_51 | 0.1 | 73.7 | 39_139 | 0.1 | 75.4 | 48_19 | 0.031 | 93.8 |
| 29_51 | 0.4 | 43.3 | 39_139 | 0.4 | 44.2 | 48_19 | 0.1 | 75 |
| 29_52 | 0.031 | 89.8 | 39_14 | 0.031 | 93 | 48_19 | 0.4 | 41 |
| 29_52 | 0.1 | 80.3 | 39_14 | 0.1 | 61.5 | 48_2 | 0.031 | 84.6 |
| 29_52 | 0.4 | 53.5 | 39_14 | 0.4 | 28.8 | 48_2 | 0.1 | 59.7 |
| 29_53 | 0.031 | 95.9 | 39_140 | 0.031 | 97.1 | 48_2 | 0.4 | 31.4 |
| 29_53 | 0.1 | 85.3 | 39_140 | 0.1 | 75.4 | 48_20 | 0.031 | 93.2 |
| 29_53 | 0.4 | 49.2 | 39_140 | 0.4 | 49.8 | 48_20 | 0.1 | 70.7 |
| 29_54 | 0.031 | 94.2 | 39_141 | 0.031 | 87.9 | 48_20 | 0.4 | 46.4 |
| 29_54 | 0.1 | 81.4 | 39_141 | 0.1 | 66.2 | 48_21 | 0.031 | 95.9 |
| 29_54 | 0.4 | 55.4 | 39_141 | 0.4 | 32.7 | 48_21 | 0.1 | 75.4 |
| 29_55 | 0.031 | 102 | 39_142 | 0.031 | 89.1 | 48_21 | 0.4 | 51.6 |
| 29_55 | 0.1 | 85.4 | 39_142 | 0.1 | 73.1 | 48_22 | 0.031 | 96.1 |
| 29_55 | 0.4 | 57.1 | 39_142 | 0.4 | 44.8 | 48_22 | 0.1 | 69.7 |
| 29_56 | 0.031 | 89.9 | 39_143 | 0.031 | 91 | 48_22 | 0.4 | 47.9 |
| 29_56 | 0.1 | 76.7 | 39_143 | 0.1 | 79.3 | 48_23 | 0.031 | 104 |
| 29_56 | 0.4 | 41.6 | 39_143 | 0.4 | 42.6 | 48_23 | 0.1 | 80.5 |
| 29_57 | 0.031 | 89.3 | 39_144 | 0.031 | 95.3 | 48_23 | 0.4 | 56.3 |
| 29_57 | 0.1 | 86 | 39_144 | 0.1 | 78.7 | 48_24 | 0.031 | 89.5 |
| 29_57 | 0.4 | 72.6 | 39_144 | 0.4 | 62.1 | 48_24 | 0.1 | 74.8 |
| 29_58 | 0.031 | 96.6 | 39_145 | 0.031 | 97.1 | 48_24 | 0.4 | 44.4 |
| 29_58 | 0.1 | 94.1 | 39_145 | 0.1 | 81 | 48_25 | 0.031 | 99.3 |
| 29_58 | 0.4 | 77.2 | 39_145 | 0.4 | 60.8 | 48_25 | 0.1 | 75.8 |
| 29_59 | 0.031 | 95.5 | 39_146 | 0.031 | 91.2 | 48_25 | 0.4 | 54.3 |
| 29_59 | 0.1 | 93.1 | 39_146 | 0.1 | 62.1 | 48_26 | 0.031 | 87.8 |
| 29_59 | 0.4 | 52.7 | 39_146 | 0.4 | 31.9 | 48_26 | 0.1 | 78.6 |
| 29_6 | 0.031 | 80.8 | 39_147 | 0.031 | 91.6 | 48_26 | 0.4 | 38.9 |
| 29_6 | 0.1 | 45 | 39_147 | 0.1 | 66.1 | 48_27 | 0.031 | 92.2 |
| 29_6 | 0.4 | 12 | 39_147 | 0.4 | 29.6 | 48_27 | 0.1 | 67.4 |
| 29_60 | 0.031 | 101 | 39_148 | 0.031 | 89.5 | 48_27 | 0.4 | 38.5 |
| 29_60 | 0.1 | 76.1 | 39_148 | 0.1 | 69.7 | 48_28 | 0.031 | 94.4 |
| 29_60 | 0.4 | 57.4 | 39_148 | 0.4 | 36.9 | 48_28 | 0.1 | 66.1 |
| 29_61 | 0.031 | 95.6 | 39_149 | 0.031 | 101 | 48_28 | 0.4 | 43.6 |
| 29_61 | 0.1 | 85.6 | 39_149 | 0.1 | 72.5 | 48_29 | 0.031 | 92.9 |
| 29_61 | 0.4 | 64.2 | 39_149 | 0.4 | 37.6 | 48_29 | 0.1 | 70.1 |
| 29_62 | 0.031 | 98.9 | 39_15 | 0.031 | 80.7 | 48_29 | 0.4 | 43.7 |
| 29_62 | 0.1 | 77.5 | 39_15 | 0.1 | 65.2 | 48_3 | 0.031 | 90.2 |
| 29_62 | 0.4 | 46.5 | 39_15 | 0.4 | 30.4 | 48_3 | 0.1 | 67.4 |
| 29_63 | 0.031 | 94.6 | 39_150 | 0.031 | 101 | 48_3 | 0.4 | 38.3 |
| 29_63 | 0.1 | 81.6 | 39_150 | 0.1 | 75.5 | 48_30 | 0.031 | 97.4 |
| 29_63 | 0.4 | 58.8 | 39_150 | 0.4 | 45.4 | 48_30 | 0.1 | 74.6 |
| 29_64 | 0.031 | 96.3 | 39_151 | 0.031 | 87.7 | 48_30 | 0.4 | 42.3 |
| 29_64 | 0.1 | 68 | 39_151 | 0.1 | 71.5 | 48_31 | 0.031 | 94.4 |
| 29_64 | 0.4 | 40.9 | 39_151 | 0.4 | 35.9 | 48_31 | 0.1 | 74.4 |
| 29_65 | 0.031 | 85.4 | 39_152 | 0.031 | 94 | 48_31 | 0.4 | 43.7 |
| 29_65 | 0.1 | 62.2 | 39_152 | 0.1 | 60.9 | 48_32 | 0.031 | 93.7 |
| 29_65 | 0.4 | 23.1 | 39_152 | 0.4 | 38.4 | 48_32 | 0.1 | 70.3 |
| 29_66 | 0.031 | 89.2 | 39_16 | 0.031 | 87.4 | 48_32 | 0.4 | 47.8 |
| 29_66 | 0.1 | 83.2 | 39_16 | 0.1 | 61.5 | 48_33 | 0.031 | 94.1 |
| 29_66 | 0.4 | 45.9 | 39_16 | 0.4 | 31.5 | 48_33 | 0.1 | 76.9 |
| 29_67 | 0.031 | 90 | 39_17 | 0.031 | 88.4 | 48_33 | 0.4 | 50.5 |
| 29_67 | 0.1 | 75.3 | 39_17 | 0.1 | 60.1 | 48_34 | 0.031 | 100 |
| 29_67 | 0.4 | 36.7 | 39_17 | 0.4 | 26.4 | 48_34 | 0.1 | 105 |
| 29_68 | 0.031 | 87.4 | 39_18 | 0.031 | 88.6 | 48_34 | 0.4 | 103 |
| 29_68 | 0.1 | 58.8 | 39_18 | 0.1 | 54.5 | 48_35 | 0.031 | 91.3 |
| 29_68 | 0.4 | 24 | 39_18 | 0.4 | 25.9 | 48_35 | 0.1 | 73.7 |
| 29_69 | 0.031 | 91.5 | 39_19 | 0.031 | 87.9 | 48_35 | 0.4 | 45.6 |
| 29_69 | 0.1 | 74.4 | 39_19 | 0.1 | 54.5 | 48_36 | 0.031 | 96.3 |
| 29_69 | 0.4 | 39.5 | 39_19 | 0.4 | 24.1 | 48_36 | 0.1 | 73.5 |
| 29_7 | 0.031 | 83.1 | 39_2 | 0.031 | 86 | 48_36 | 0.4 | 54.7 |
| 29_7 | 0.1 | 51.8 | 39_2 | 0.1 | 58.9 | 48_37 | 0.031 | 95.8 |
| 29_7 | 0.4 | 14.8 | 39_2 | 0.4 | 35 | 48_37 | 0.1 | 76.5 |
| 29_70 | 0.031 | 102 | 39_20 | 0.031 | 86.3 | 48_37 | 0.4 | 51.9 |
| 29_70 | 0.1 | 62.1 | 39_20 | 0.1 | 57.6 | 48_38 | 0.031 | 81.9 |
| 29_70 | 0.4 | 27.7 | 39_20 | 0.4 | 28.8 | 48_38 | 0.1 | 69.5 |
| 29_71 | 0.031 | 90.7 | 39_21 | 0.031 | 102 | 48_38 | 0.4 | 37.7 |
| 29_71 | 0.1 | 62.2 | 39_21 | 0.1 | 78.8 | 48_39 | 0.031 | 92.3 |
| 29_71 | 0.4 | 31.2 | 39_21 | 0.4 | 59.6 | 48_39 | 0.1 | 72.5 |
| 29_72 | 0.031 | 94.6 | 39_22 | 0.031 | 95.8 | 48_39 | 0.4 | 49.7 |
| 29_72 | 0.1 | 62.2 | 39_22 | 0.1 | 88.8 | 48_4 | 0.031 | 84.4 |
| 29_72 | 0.4 | 31.9 | 39_22 | 0.4 | 58 | 48_4 | 0.1 | 65.7 |
| 29_73 | 0.031 | 84.6 | 39_23 | 0.031 | 102 | 48_4 | 0.4 | 38.8 |
| 29_73 | 0.1 | 62.2 | 39_23 | 0.1 | 78.4 | 48_40 | 0.031 | 91.7 |
| 29_73 | 0.4 | 28.4 | 39_23 | 0.4 | 54.7 | 48_40 | 0.1 | 76.7 |
| 29_74 | 0.031 | 83.4 | 39_24 | 0.031 | 102 | 48_40 | 0.4 | 51.7 |
| 29_74 | 0.1 | 60.3 | 39_24 | 0.1 | 82.4 | 48_41 | 0.031 | 90.5 |
| 29_74 | 0.4 | 21.3 | 39_24 | 0.4 | 59.8 | 48_41 | 0.1 | 76 |
| 29_75 | 0.031 | 82.6 | 39_25 | 0.031 | 99.1 | 48_41 | 0.4 | 50.9 |
| 29_75 | 0.1 | 63.2 | 39_25 | 0.1 | 84.4 | 48_42 | 0.031 | 88.5 |
| 29_75 | 0.4 | 36 | 39_25 | 0.4 | 54.8 | 48_42 | 0.1 | 70.8 |
| 29_76 | 0.031 | 93.9 | 39_26 | 0.031 | 95.8 | 48_42 | 0.4 | 46.9 |
| 29_76 | 0.1 | 68.5 | 39_26 | 0.1 | 79.7 | 48_43 | 0.031 | 95.5 |
| 29_76 | 0.4 | 34.4 | 39_26 | 0.4 | 59.6 | 48_43 | 0.1 | 75 |
| 29_77 | 0.031 | 103 | 39_27 | 0.031 | 93.2 | 48_43 | 0.4 | 44.8 |
| 29_77 | 0.1 | 84.5 | 39_27 | 0.1 | 91.1 | 48_44 | 0.031 | 93.9 |
| 29_77 | 0.4 | 61.1 | 39_27 | 0.4 | 78.4 | 48_44 | 0.1 | 78.1 |
| 29_78 | 0.031 | 95.3 | 39_28 | 0.031 | 104 | 48_44 | 0.4 | 59.4 |
| 29_78 | 0.1 | 63.7 | 39_28 | 0.1 | 90.9 | 48_45 | 0.031 | 99.2 |
| 29_78 | 0.4 | 31.6 | 39_28 | 0.4 | 65.9 | 48_45 | 0.1 | 82.6 |
| 29_79 | 0.031 | 92.8 | 39_29 | 0.031 | 98.6 | 48_45 | 0.4 | 53.7 |
| 29_79 | 0.1 | 71.6 | 39_29 | 0.1 | 87.9 | 48_46 | 0.031 | 93.5 |
| 29_79 | 0.4 | 31.7 | 39_29 | 0.4 | 73.7 | 48_46 | 0.1 | 84.2 |
| 29_8 | 0.031 | 82.3 | 39_3 | 0.031 | 96.6 | 48_46 | 0.4 | 64.7 |
| 29_8 | 0.1 | 52.3 | 39_3 | 0.1 | 61.5 | 48_47 | 0.031 | 93.5 |
| 29_8 | 0.4 | 19.5 | 39_3 | 0.4 | 27.8 | 48_47 | 0.1 | 83.6 |
| 29_80 | 0.031 | 95.7 | 39_30 | 0.031 | 104 | 48_47 | 0.4 | 68.1 |
| 29_80 | 0.1 | 71.9 | 39_30 | 0.1 | 97.1 | 48_48 | 0.031 | 96.6 |
| 29_80 | 0.4 | 30.4 | 39_30 | 0.4 | 76.1 | 48_48 | 0.1 | 90.4 |
| 29_81 | 0.031 | 96.9 | 39_31 | 0.031 | 95.7 | 48_48 | 0.4 | 78.1 |
| 29_81 | 0.1 | 88.7 | 39_31 | 0.1 | 93.2 | 48_49 | 0.031 | 96.9 |
| 29_81 | 0.4 | 51 | 39_31 | 0.4 | 75.7 | 48_49 | 0.1 | 88.6 |
| 29_82 | 0.031 | 83.7 | 39_32 | 0.031 | 95.1 | 48_49 | 0.4 | 66 |
| 29_82 | 0.1 | 59 | 39_32 | 0.1 | 91.9 | 48_5 | 0.031 | 93.5 |
| 29_82 | 0.4 | 16.2 | 39_32 | 0.4 | 68.3 | 48_5 | 0.1 | 72.6 |
| 29_83 | 0.031 | 89.9 | 39_33 | 0.031 | 91.5 | 48_5 | 0.4 | 41 |
| 29_83 | 0.1 | 62.2 | 39_33 | 0.1 | 86 | 48_50 | 0.031 | 89 |
| 29_83 | 0.4 | 21.7 | 39_33 | 0.4 | 49.2 | 48_50 | 0.1 | 79.7 |
| 29_84 | 0.031 | 83.2 | 39_34 | 0.031 | 94.4 | 48_50 | 0.4 | 39.1 |
| 29_84 | 0.1 | 48.3 | 39_34 | 0.1 | 83.4 | 48_51 | 0.031 | 87.5 |
| 29_84 | 0.4 | 11.3 | 39_34 | 0.4 | 69.5 | 48_51 | 0.1 | 69.8 |
| 29_85 | 0.031 | 98 | 39_35 | 0.031 | 91.9 | 48_51 | 0.4 | 36.8 |
| 29_85 | 0.1 | 76.2 | 39_35 | 0.1 | 87.4 | 48_52 | 0.031 | 91.8 |
| 29_85 | 0.4 | 45.8 | 39_35 | 0.4 | 65.8 | 48_52 | 0.1 | 78.5 |
| 29_86 | 0.031 | 100 | 39_36 | 0.031 | 95.6 | 48_52 | 0.4 | 52.7 |
| 29_86 | 0.1 | 84.2 | 39_36 | 0.1 | 92.1 | 48_53 | 0.031 | 94.5 |
| 29_86 | 0.4 | 58.4 | 39_36 | 0.4 | 75.8 | 48_53 | 0.1 | 69.6 |
| 29_87 | 0.031 | 95.3 | 39_37 | 0.031 | 91.1 | 48_53 | 0.4 | 43.6 |
| 29_87 | 0.1 | 88.1 | 39_37 | 0.1 | 89.7 | 48_54 | 0.031 | 89 |
| 29_87 | 0.4 | 54.5 | 39_37 | 0.4 | 55.8 | 48_54 | 0.1 | 69.1 |
| 29_88 | 0.031 | 93.7 | 39_38 | 0.031 | 101 | 48_54 | 0.4 | 49.3 |
| 29_88 | 0.1 | 74.6 | 39_38 | 0.1 | 83.4 | 48_55 | 0.031 | 93.1 |
| 29_88 | 0.4 | 36.7 | 39_38 | 0.4 | 76.1 | 48_55 | 0.1 | 72.6 |
| 29_89 | 0.031 | 88 | 39_39 | 0.031 | 98.2 | 48_55 | 0.4 | 51.6 |
| 29_89 | 0.1 | 71.9 | 39_39 | 0.1 | 95 | 48_56 | 0.031 | 90.1 |
| 29_89 | 0.4 | 41.6 | 39_39 | 0.4 | 67.7 | 48_56 | 0.1 | 81.2 |
| 29_9 | 0.031 | 82.7 | 39_4 | 0.031 | 84 | 48_56 | 0.4 | 46.2 |
| 29_9 | 0.1 | 41.2 | 39_4 | 0.1 | 63.2 | 48_6 | 0.031 | 79.2 |
| 29_9 | 0.4 | 11.8 | 39_4 | 0.4 | 30.4 | 48_6 | 0.1 | 69.3 |
| 29_90 | 0.031 | 82.2 | 39_40 | 0.031 | 93.6 | 48_6 | 0.4 | 42.1 |
| 29_90 | 0.1 | 72.3 | 39_40 | 0.1 | 80.9 | 48_7 | 0.031 | 92.6 |
| 29_90 | 0.4 | 44.9 | 39_40 | 0.4 | 61.6 | 48_7 | 0.1 | 67.9 |
| 29_91 | 0.031 | 96.3 | 39_41 | 0.031 | 90.3 | 48_7 | 0.4 | 48.7 |
| 29_91 | 0.1 | 84.2 | 39_41 | 0.1 | 71.4 | 48_8 | 0.031 | 85.6 |
| 29_91 | 0.4 | 40.9 | 39_41 | 0.4 | 46.8 | 48_8 | 0.1 | 66.5 |
| 29_92 | 0.031 | 97.2 | 39_42 | 0.031 | 88.6 | 48_8 | 0.4 | 38.4 |
| 29_92 | 0.1 | 79.1 | 39_42 | 0.1 | 62.6 | 48_9 | 0.031 | 91.6 |
| 29_92 | 0.4 | 48 | 39_42 | 0.4 | 43.5 | 48_9 | 0.1 | 70.6 |
| 29_93 | 0.031 | 96 | 39_43 | 0.031 | 89.1 | 48_9 | 0.4 | 40 |
| 29_93 | 0.1 | 87.5 | 39_43 | 0.1 | 79.3 | 49_1 | 0.031 | 100 |
| 29_93 | 0.4 | 60.8 | 39_43 | 0.4 | 45.6 | 49_1 | 0.1 | 85.7 |
| 29_94 | 0.031 | 99 | 39_44 | 0.031 | 88 | 49_1 | 0.4 | 64.9 |
| 29_94 | 0.1 | 86.3 | 39_44 | 0.1 | 69.3 | 49_2 | 0.031 | 98.1 |
| 29_94 | 0.4 | 63.5 | 39_44 | 0.4 | 45.5 | 49_2 | 0.1 | 82.4 |
| 29_95 | 0.031 | 91.7 | 39_45 | 0.031 | 92 | 49_2 | 0.4 | 60.8 |
| 29_95 | 0.1 | 71.6 | 39_45 | 0.1 | 63.9 | 50_1 | 0.031 | 104 |
| 29_95 | 0.4 | 38.3 | 39_45 | 0.4 | 43 | 50_1 | 0.1 | 91.6 |
| 29_96 | 0.031 | 93.7 | 39_46 | 0.031 | 95.7 | 50_1 | 0.4 | 66.8 |
| 29_96 | 0.1 | 59 | 39_46 | 0.1 | 83.4 | 50_10 | 0.031 | 98.6 |
| 29_96 | 0.4 | 24.7 | 39_46 | 0.4 | 49.5 | 50_10 | 0.1 | 75.3 |
| 29_97 | 0.031 | 90.8 | 39_47 | 0.031 | 95.6 | 50_10 | 0.4 | 46.8 |
| 29_97 | 0.1 | 62.5 | 39_47 | 0.1 | 88.4 | 50_11 | 0.031 | 101 |
| 29_97 | 0.4 | 26.7 | 39_47 | 0.4 | 61.3 | 50_11 | 0.1 | 95.7 |
| 29_98 | 0.031 | 94.4 | 39_48 | 0.031 | 106 | 50_11 | 0.4 | 99.1 |
| 29_98 | 0.1 | 66.2 | 39_48 | 0.1 | 97.7 | 50_12 | 0.031 | 98.1 |
| 29_98 | 0.4 | 30.8 | 39_48 | 0.4 | 80.6 | 50_12 | 0.1 | 89.8 |
| 29_99 | 0.031 | 78.4 | 39_49 | 0.031 | 92.1 | 50_12 | 0.4 | 75.4 |
| 29_99 | 0.1 | 62 | 39_49 | 0.1 | 80.1 | 50_13 | 0.031 | 105 |
| 29_99 | 0.4 | 26.9 | 39_49 | 0.4 | 52.9 | 50_13 | 0.1 | 86.9 |
| 30_1 | 0.031 | 94.8 | 39_5 | 0.031 | 89 | 50_13 | 0.4 | 80.9 |
| 30_1 | 0.1 | 92.8 | 39_5 | 0.1 | 62.2 | 50_14 | 0.031 | 102 |
| 30_1 | 0.4 | 78.8 | 39_5 | 0.4 | 37.5 | 50_14 | 0.1 | 92.7 |
| 30_10 | 0.031 | 86 | 39_50 | 0.031 | 97.9 | 50_14 | 0.4 | 73.5 |
| 30_10 | 0.1 | 72.8 | 39_50 | 0.1 | 78.8 | 50_15 | 0.031 | 102 |
| 30_10 | 0.4 | 33.9 | 39_50 | 0.4 | 67.7 | 50_15 | 0.1 | 100 |
| 30_11 | 0.031 | 91.7 | 39_51 | 0.031 | 98.5 | 50_15 | 0.4 | 91.4 |
| 30_11 | 0.1 | 74.7 | 39_51 | 0.1 | 97.4 | 50_16 | 0.031 | 92.3 |
| 30_11 | 0.4 | 30.5 | 39_51 | 0.4 | 62.7 | 50_16 | 0.1 | 91.5 |
| 30_12 | 0.031 | 101 | 39_52 | 0.031 | 89.3 | 50_16 | 0.4 | 95.3 |
| 30_12 | 0.1 | 81.8 | 39_52 | 0.1 | 88.7 | 50_17 | 0.031 | 99.2 |
| 30_12 | 0.4 | 47.5 | 39_52 | 0.4 | 62.3 | 50_17 | 0.1 | 95.4 |
| 30_13 | 0.031 | 96.8 | 39_53 | 0.031 | 100 | 50_17 | 0.4 | 96.4 |
| 30_13 | 0.1 | 80.5 | 39_53 | 0.1 | 84.9 | 50_18 | 0.031 | 99.2 |
| 30_13 | 0.4 | 38.2 | 39_53 | 0.4 | 69.3 | 50_18 | 0.1 | 88.6 |
| 30_14 | 0.031 | 96.1 | 39_54 | 0.031 | 102 | 50_18 | 0.4 | 76.8 |
| 30_14 | 0.1 | 78.2 | 39_54 | 0.1 | 81 | 50_2 | 0.031 | 99.2 |
| 30_14 | 0.4 | 39 | 39_54 | 0.4 | 53.9 | 50_2 | 0.1 | 93.3 |
| 30_15 | 0.031 | 99.1 | 39_55 | 0.031 | 94.4 | 50_2 | 0.4 | 87.1 |
| 30_15 | 0.1 | 83.3 | 39_55 | 0.1 | 83.6 | 50_3 | 0.031 | 94 |
| 30_15 | 0.4 | 58.7 | 39_55 | 0.4 | 60.8 | 50_3 | 0.1 | 85.7 |
| 30_16 | 0.031 | 95.6 | 39_56 | 0.031 | 92.8 | 50_3 | 0.4 | 68.2 |
| 30_16 | 0.1 | 69.9 | 39_56 | 0.1 | 81.6 | 50_4 | 0.031 | 88.4 |
| 30_16 | 0.4 | 27.3 | 39_56 | 0.4 | 67.2 | 50_4 | 0.1 | 86.9 |
| 30_17 | 0.031 | 97.9 | 39_57 | 0.031 | 95.1 | 50_4 | 0.4 | 65.1 |
| 30_17 | 0.1 | 86.9 | 39_57 | 0.1 | 85.9 | 50_5 | 0.031 | 98.8 |
| 30_17 | 0.4 | 58.6 | 39_57 | 0.4 | 57.4 | 50_5 | 0.1 | 85.1 |
| 30_18 | 0.031 | 92 | 39_58 | 0.031 | 99.5 | 50_5 | 0.4 | 54.7 |
| 30_18 | 0.1 | 71.4 | 39_58 | 0.1 | 96 | 50_6 | 0.031 | 101 |
| 30_18 | 0.4 | 30.7 | 39_58 | 0.4 | 71 | 50_6 | 0.1 | 88.3 |
| 30_19 | 0.031 | 92.5 | 39_59 | 0.031 | 89.7 | 50_6 | 0.4 | 65.1 |
| 30_19 | 0.1 | 71 | 39_59 | 0.1 | 86.8 | 50_7 | 0.031 | 102 |
| 30_19 | 0.4 | 36.9 | 39_59 | 0.4 | 65.2 | 50_7 | 0.1 | 96.7 |
| 30_2 | 0.031 | 102 | 39_6 | 0.031 | 89.2 | 50_7 | 0.4 | 78.3 |
| 30_2 | 0.1 | 100 | 39_6 | 0.1 | 56 | 50_8 | 0.031 | 106 |
| 30_2 | 0.4 | 95.2 | 39_6 | 0.4 | 29.1 | 50_8 | 0.1 | 101 |
| 30_20 | 0.031 | 98.2 | 39_60 | 0.031 | 104 | 50_8 | 0.4 | 88.1 |
| 30_20 | 0.1 | 68.6 | 39_60 | 0.1 | 95.8 | 50_9 | 0.031 | 99.9 |
| 30_20 | 0.4 | 25.4 | 39_60 | 0.4 | 64.4 | 50_9 | 0.1 | 90.8 |
| 30_3 | 0.031 | 104 | 39_61 | 0.031 | 92.5 | 50_9 | 0.4 | 89.1 |
| 30_3 | 0.1 | 104 | 39_61 | 0.1 | 77.4 | 51_1 | 0.031 | 105 |
| 30_3 | 0.4 | 92.8 | 39_61 | 0.4 | 54.5 | 51_1 | 0.1 | 98 |
| 30_4 | 0.031 | 111 | 39_62 | 0.031 | 96.9 | 51_1 | 0.4 | 108 |
| 30_4 | 0.1 | 103 | 39_62 | 0.1 | 89.5 | 51_10 | 0.031 | 95.8 |
| 30_4 | 0.4 | 96.6 | 39_62 | 0.4 | 70.7 | 51_10 | 0.1 | 94.1 |
| 30_5 | 0.031 | 98.8 | 39_63 | 0.031 | 97.5 | 51_10 | 0.4 | 90.3 |
| 30_5 | 0.1 | 98.4 | 39_63 | 0.1 | 85.2 | 51_11 | 0.031 | 102 |
| 30_5 | 0.4 | 94.2 | 39_63 | 0.4 | 61.9 | 51_11 | 0.1 | 95.3 |
| 30_6 | 0.031 | 106 | 39_64 | 0.031 | 94.8 | 51_11 | 0.4 | 89.3 |
| 30_6 | 0.1 | 104 | 39_64 | 0.1 | 93.2 | 51_12 | 0.031 | 98.2 |
| 30_6 | 0.4 | 104 | 39_64 | 0.4 | 77.9 | 51_12 | 0.1 | 100 |
| 30_7 | 0.031 | 102 | 39_65 | 0.031 | 92.7 | 51_12 | 0.4 | 94.9 |
| 30_7 | 0.1 | 101 | 39_65 | 0.1 | 78.9 | 51_13 | 0.031 | 107 |
| 30_7 | 0.4 | 101 | 39_65 | 0.4 | 53.3 | 51_13 | 0.1 | 107 |
| 30_8 | 0.031 | 89 | 39_66 | 0.031 | 93.2 | 51_13 | 0.4 | 113 |
| 30_8 | 0.1 | 64.4 | 39_66 | 0.1 | 91.7 | 51_14 | 0.031 | 95.8 |
| 30_8 | 0.4 | 31.5 | 39_66 | 0.4 | 71.7 | 51_14 | 0.1 | 88.7 |
| 30_9 | 0.031 | 90.1 | 39_67 | 0.031 | 95.6 | 51_14 | 0.4 | 80.7 |
| 30_9 | 0.1 | 81.3 | 39_67 | 0.1 | 90.5 | 51_15 | 0.031 | 101 |
| 30_9 | 0.4 | 41.9 | 39_67 | 0.4 | 75.2 | 51_15 | 0.1 | 91.9 |
| 31_1 | 0.031 | 87.5 | 39_68 | 0.031 | 88.3 | 51_15 | 0.4 | 88.6 |
| 31_1 | 0.1 | 56.9 | 39_68 | 0.1 | 93.2 | 51_16 | 0.031 | 101 |
| 31_1 | 0.4 | 20.8 | 39_68 | 0.4 | 58.8 | 51_16 | 0.1 | 104 |
| 31_10 | 0.031 | 102 | 39_69 | 0.031 | 104 | 51_16 | 0.4 | 83.3 |
| 31_10 | 0.1 | 64.2 | 39_69 | 0.1 | 105 | 51_17 | 0.031 | 96.7 |
| 31_10 | 0.4 | 28.5 | 39_69 | 0.4 | 83.6 | 51_17 | 0.1 | 101 |
| 31_11 | 0.031 | 84.9 | 39_7 | 0.031 | 82.4 | 51_17 | 0.4 | 97.9 |
| 31_11 | 0.1 | 63.1 | 39_7 | 0.1 | 54.4 | 51_18 | 0.031 | 103 |
| 31_11 | 0.4 | 23.9 | 39_7 | 0.4 | 22.9 | 51_18 | 0.1 | 97.8 |
| 31_12 | 0.031 | 93.6 | 39_70 | 0.031 | 98.3 | 51_18 | 0.4 | 89.1 |
| 31_12 | 0.1 | 70.7 | 39_70 | 0.1 | 90.1 | 51_19 | 0.031 | 99.7 |
| 31_12 | 0.4 | 36.3 | 39_70 | 0.4 | 71.8 | 51_19 | 0.1 | 104 |
| 31_13 | 0.031 | 88.2 | 39_71 | 0.031 | 104 | 51_19 | 0.4 | 87.6 |
| 31_13 | 0.1 | 56 | 39_71 | 0.1 | 97.6 | 51_2 | 0.031 | 98.6 |
| 31_13 | 0.4 | 24 | 39_71 | 0.4 | 84.9 | 51_2 | 0.1 | 101 |
| 31_14 | 0.031 | 93.6 | 39_72 | 0.031 | 96.9 | 51_2 | 0.4 | 92.2 |
| 31_14 | 0.1 | 58.4 | 39_72 | 0.1 | 105 | 51_20 | 0.031 | 101 |
| 31_14 | 0.4 | 24.8 | 39_72 | 0.4 | 83.8 | 51_20 | 0.1 | 103 |
| 31_15 | 0.031 | 90.7 | 39_73 | 0.031 | 85.7 | 51_20 | 0.4 | 103 |
| 31_15 | 0.1 | 62.4 | 39_73 | 0.1 | 74.9 | 51_21 | 0.031 | 102 |
| 31_15 | 0.4 | 31.4 | 39_73 | 0.4 | 38.3 | 51_21 | 0.1 | 92.7 |
| 31_16 | 0.031 | 93.9 | 39_74 | 0.031 | 93.3 | 51_21 | 0.4 | 79.8 |
| 31_16 | 0.1 | 61.4 | 39_74 | 0.1 | 82.1 | 51_3 | 0.031 | 99 |
| 31_16 | 0.4 | 31.2 | 39_74 | 0.4 | 57.6 | 51_3 | 0.1 | 99.7 |
| 31_17 | 0.031 | 84.7 | 39_75 | 0.031 | 95.8 | 51_3 | 0.4 | 83.6 |
| 31_17 | 0.1 | 62.7 | 39_75 | 0.1 | 63.8 | 51_4 | 0.031 | 111 |
| 31_17 | 0.4 | 30 | 39_75 | 0.4 | 29 | 51_4 | 0.1 | 92.3 |
| 31_18 | 0.031 | 82.2 | 39_76 | 0.031 | 97.2 | 51_4 | 0.4 | 86.7 |
| 31_18 | 0.1 | 65.1 | 39_76 | 0.1 | 78.5 | 51_5 | 0.031 | 102 |
| 31_18 | 0.4 | 26.2 | 39_76 | 0.4 | 51.8 | 51_5 | 0.1 | 95.4 |
| 31_2 | 0.031 | 90.3 | 39_77 | 0.031 | 95.1 | 51_5 | 0.4 | 104 |
| 31_2 | 0.1 | 53.5 | 39_77 | 0.1 | 84.1 | 51_6 | 0.031 | 95.9 |
| 31_2 | 0.4 | 20 | 39_77 | 0.4 | 61.9 | 51_6 | 0.1 | 89.2 |
| 31_3 | 0.031 | 87.1 | 39_78 | 0.031 | 97.1 | 51_6 | 0.4 | 74.1 |
| 31_3 | 0.1 | 44.3 | 39_78 | 0.1 | 77.5 | 51_7 | 0.031 | 94.1 |
| 31_3 | 0.4 | 12.3 | 39_78 | 0.4 | 47.9 | 51_7 | 0.1 | 76.4 |
| 31_4 | 0.031 | 89.1 | 39_79 | 0.031 | 94.7 | 51_7 | 0.4 | 47.6 |
| 31_4 | 0.1 | 54.3 | 39_79 | 0.1 | 78.5 | 51_8 | 0.031 | 99.5 |
| 31_4 | 0.4 | 17.7 | 39_79 | 0.4 | 45.1 | 51_8 | 0.1 | 83.9 |
| 31_5 | 0.031 | 86.2 | 39_8 | 0.031 | 89 | 51_8 | 0.4 | 60.5 |
| 31_5 | 0.1 | 52.5 | 39_8 | 0.1 | 60.6 | 51_9 | 0.031 | 104 |
| 31_5 | 0.4 | 31.6 | 39_8 | 0.4 | 30.8 | 51_9 | 0.1 | 84.7 |
| 31_6 | 0.031 | 82.6 | 39_80 | 0.031 | 102 | 51_9 | 0.4 | 70 |
| 31_6 | 0.1 | 59.3 | 39_80 | 0.1 | 92.5 | 52_1 | 0.031 | 105 |
| 31_6 | 0.4 | 19.1 | 39_80 | 0.4 | 64.3 | 52_1 | 0.1 | 94.6 |
| 31_7 | 0.031 | 89.5 | 39_81 | 0.031 | 101 | 52_1 | 0.4 | 110 |
| 31_7 | 0.1 | 55.1 | 39_81 | 0.1 | 83.4 | 52_10 | 0.031 | 98.4 |
| 31_7 | 0.4 | 21.8 | 39_81 | 0.4 | 62.3 | 52_10 | 0.1 | 97.5 |
| 31_8 | 0.031 | 80.8 | 39_82 | 0.031 | 103 | 52_10 | 0.4 | 101 |
| 31_8 | 0.1 | 61.8 | 39_82 | 0.1 | 77.5 | 52-11 | 0.031 | 103 |
| 31_8 | 0.4 | 25.2 | 39_82 | 0.4 | 57.9 | 52-11 | 0.1 | 98.3 |
| 31_9 | 0.031 | 90.9 | 39_83 | 0.031 | 101 | 52-11 | 0.4 | 99.4 |
| 31_9 | 0.1 | 68.7 | 39_83 | 0.1 | 84.7 | 52_12 | 0.031 | 98.2 |
| 31_9 | 0.4 | 28.7 | 39_83 | 0.4 | 54 | 52_12 | 0.1 | 95 |
| 32_1 | 0.031 | 90.8 | 39_84 | 0.031 | 98.6 | 52_12 | 0.4 | 75.3 |
| 32_1 | 0.1 | 80.3 | 39_84 | 0.1 | 92.7 | 52_13 | 0.031 | 101 |
| 32_1 | 0.4 | 41.4 | 39_84 | 0.4 | 68 | 52_13 | 0.1 | 101 |
| 32_2 | 0.031 | 110 | 39_85 | 0.031 | 92.2 | 52_13 | 0.4 | 92.3 |
| 32_2 | 0.1 | 100 | 39_85 | 0.1 | 87.7 | 52_14 | 0.031 | 91.2 |
| 32_2 | 0.4 | 68.9 | 39_85 | 0.4 | 59.4 | 52_14 | 0.1 | 94 |
| 32_3 | 0.031 | 94.9 | 39_86 | 0.031 | 97.7 | 52_14 | 0.4 | 107 |
| 32_3 | 0.1 | 73.6 | 39_86 | 0.1 | 74.7 | 52_15 | 0.031 | 101 |
| 32_3 | 0.4 | 32.8 | 39_86 | 0.4 | 36.8 | 52_15 | 0.1 | 99.8 |
| 32_4 | 0.031 | 89.3 | 39_87 | 0.031 | 99 | 52_15 | 0.4 | 108 |
| 32_4 | 0.1 | 83.9 | 39_87 | 0.1 | 93.5 | 52_16 | 0.031 | 101 |
| 32_4 | 0.4 | 55.2 | 39_87 | 0.4 | 71.8 | 52_16 | 0.1 | 97 |
| 32_5 | 0.031 | 97.4 | 39_88 | 0.031 | 94.4 | 52_16 | 0.4 | 85.3 |
| 32_5 | 0.1 | 72.6 | 39_88 | 0.1 | 86.4 | 52_17 | 0.031 | 99.8 |
| 32_5 | 0.4 | 32.1 | 39_88 | 0.4 | 48.9 | 52_17 | 0.1 | 95.2 |
| 33_1 | 0.031 | 76.2 | 39_89 | 0.031 | 99.6 | 52_17 | 0.4 | 88.4 |
| 33_1 | 0.1 | 53.2 | 39_89 | 0.1 | 80.6 | 52_2 | 0.031 | 95.8 |
| 33_1 | 0.4 | 17.9 | 39_89 | 0.4 | 55.2 | 52_2 | 0.1 | 94 |
| 33_10 | 0.031 | 94.6 | 39_9 | 0.031 | 81.7 | 52_2 | 0.4 | 88.4 |
| 33_10 | 0.1 | 60 | 39_9 | 0.1 | 58.6 | 52_3 | 0.031 | 100 |
| 33_10 | 0.4 | 25 | 39_9 | 0.4 | 23.3 | 52_3 | 0.1 | 100 |
| 33_11 | 0.031 | 82 | 39_90 | 0.031 | 98.2 | 52_3 | 0.4 | 85.7 |
| 33_11 | 0.1 | 68.6 | 39_90 | 0.1 | 74.3 | 52_4 | 0.031 | 104 |
| 33_11 | 0.4 | 30.1 | 39_90 | 0.4 | 52.3 | 52_4 | 0.1 | 98.3 |
| 33_12 | 0.031 | 101 | 39_91 | 0.031 | 87 | 52_4 | 0.4 | 93.9 |
| 33_12 | 0.1 | 88.9 | 39_91 | 0.1 | 65.1 | 52_5 | 0.031 | 101 |
| 33_12 | 0.4 | 61.2 | 39_91 | 0.4 | 34.4 | 52_5 | 0.1 | 104 |
| 33_13 | 0.031 | 95.6 | 39_92 | 0.031 | 98 | 52_5 | 0.4 | 107 |
| 33_13 | 0.1 | 88.8 | 39_92 | 0.1 | 85.8 | 52_6 | 0.031 | 111 |
| 33_13 | 0.4 | 35 | 39_92 | 0.4 | 58.4 | 52_6 | 0.1 | 94.9 |
| 33_14 | 0.031 | 92.4 | 39_93 | 0.031 | 99.7 | 52_6 | 0.4 | 103 |
| 33_14 | 0.1 | 65.7 | 39_93 | 0.1 | 79 | 52_7 | 0.031 | 102 |
| 33_14 | 0.4 | 32.6 | 39_93 | 0.4 | 51.8 | 52_7 | 0.1 | 106 |
| 33_15 | 0.031 | 94.1 | 39_94 | 0.031 | 99.4 | 52_7 | 0.4 | 108 |
| 33_15 | 0.1 | 61.2 | 39_94 | 0.1 | 81.3 | 52_8 | 0.031 | 97.7 |
| 33_15 | 0.4 | 26.5 | 39_94 | 0.4 | 52.5 | 52_8 | 0.1 | 92.6 |
| 33_16 | 0.031 | 89.7 | 39_95 | 0.031 | 107 | 52_8 | 0.4 | 84.5 |
| 33_16 | 0.1 | 67.1 | 39_95 | 0.1 | 86.9 | 52_9 | 0.031 | 100 |
| 33_16 | 0.4 | 30.4 | 39_95 | 0.4 | 59.8 | 52_9 | 0.1 | 93.2 |
| | | | | | | 52_9 | 0.4 | 92.9 |

### Example 2: Caspase Analysis on Selected Compounds from Example 1

Mouse 3T3 cells were cultured in DMEM, and HepG2 cells were cultured in MEM. All media was supplemented with 10% (v/v) fetal bovine serum. Cells were cultured at 37° C. and 5% CO₂. One day before transfection, cells were plated in 100 μL growth medium without antibiotics in a 96-well plate at a density that resulted in 60%-70% cell confluency at the time of transfection. Lipofectamine® 2000 (Invitrogen) was used for transfections in 96-well plates. Antisense oligonucleotides were diluted to the required concentration to a total volume of 25 μL in Opti-MEM™ (Invitrogen) and mixed with 25 μL transfectioncomplex (0.25 μL Lipofectamine® 2000 and 24.75 μL Opti-MEM™). After 20 min incubation, 50 μL antibiotic-free medium was added to the solution and mixed. After removing the medium from the wells, 100 μL antisense oligonucleotide:transfection agent solution was added to the cells and incubated for 24 hrs (LNA-ASO transfections). All transfections were performed in triplicates. Caspase-3/7 activity was determined 24 hrs after oligonucleotide transfection using the Caspase-Glo® 3/7 Assay (Promega) according to the manufacturer's instruction on a VICTOR3™ plate reader (Perkin Elmer). Caspase analysis results are shown in Table 7.

### Example 3: Testing In Vitro Potency of Antisense Oligonucleotides Targeting SCN9A in SK-N-AS Cell Line in a Concentration-Response Assay

SK-N-AS cells were maintained in a humidified incubator as recommended by the supplier. The vendor and recommended culture conditions are reported in Table 4 of Example 1.

For assays, cells were seeded in a 96-multi well plate in culture media and incubated as reported in Table 4 before addition of antisense oligonucleotides dissolved in a volume of 5 μL PBS. For the concentration response experiment, oligonucleotides in Table 9 were diluted in 10-steps 3.16-fold (½ log) dilutions to final concentration in cell growth media spanning from 31.6 μM to of 0.001 μM. This allowed testing of 8 compound pr. 96-well plates leaving 16 wells with PBS controls.

The cells were harvested 72 hours after the addition of antisense oligonucleotides (see Table 4). RNA was extracted using the PureLink™ Pro 96 RNA Purification kit (Thermo Fisher Scientific) according to the manufacturer's instructions and eluted in 50 μL of water. The RNA was subsequently diluted 10 times with DNase/RNase free Water (Gibco) and heated to 90° C. for one minute.

For gene expressions analysis, One Step RT-qPCR was performed using gScript™ XLT One-Step RT-qPCR ToughMix®, Low ROX™ (Quantabio) in a duplex set up. The primer assays used for qPCR are collated in Table 8 for both target and endogenous control.

TABLE 8

| Endogen contr. | Endog. contr. | Endogen. contr. | Target | Target | Target |
|---|---|---|---|---|---|
| assay | vendor | fluorophore | assay | vendor | fluorophore |
| GUSB: | IDT | HEX-ZEN | SCN9A: | IDT | FAM-ZEN |
| Hs.PTv.27737538 | | | Hs.PT.5840099024 | | |

Quantities of SCN9A mRNA were calculated based on standard curves included on each qPCR plate. Input RNA for standard curves was RNA from PBS-treated wells on the same cell plate (as described above). Quantity was normalized to the calculated quantity for the endogenous control gene assay run in the same well (GUSB). Relative Target Quantity=QUANTITY_target gene (SCN9A)/QUANTITY_endogenous control gene (GUSB). The RNA knockdown was calculated for each well by division with the median of all PBS-treated wells on the same plate. Normalized Target Quantity=(Relative Target Quantity/[mean] Relative Target Quantity]_pbs_wells)*100.

To generate the EC50 values in Table 9, curves were fitted from the Normalized Target Quantity of SCN9A, using a four Parameter Sigmoidal Dose-Response Model.

TABLE 9
EC50 of compounds tested in concentration response experiments.

| | NAV1.7 qPCR probe2 SK-N-AS |
|---|---|
| CMP ID NO | GMean EC50 (μM) |
| 29_33 | 0.02858 |
| 29_25 | 0.04035 |
| 39_9 | 0.04910 |
| 39_19 | 0.04947 |
| 31_4 | 0.05136 |
| 39_18 | 0.05207 |
| 534_1 | 0.05489 |
| 33_1 | 0.06123 |
| 39_10 | 0.06259 |
| 29_15 | 0.06407 |
| 29_22 | 0.06462 |
| 39_7 | 0.06614 |
| 29_10 | 0.06925 |
| 39_17 | 0.06998 |
| 31_1 | 0.07083 |
| 33_2 | 0.07392 |
| 29_26 | 0.07477 |
| 39_20 | 0.07490 |
| 31_3 | 0.07884 |
| 29_34 | 0.07905 |
| 29_11 | 0.08061 |
| 29_35 | 0.08138 |
| 31_5 | 0.08303 |
| 533_1 | 0.08414 |
| 29_29 | 0.08432 |
| 33_3 | 0.08799 |
| 39_13 | 0.09649 |
| 29_24 | 0.09668 |
| 39_6 | 0.09912 |
| 39_2 | 0.10601 |
| 532_1 | 0.10894 |
| 47_1 | 0.10983 |
| 39_1 | 0.11236 |
| 48_9 | 0.11620 |
| 48_10 | 0.13282 |
| 48_8 | 0.15498 |
| 517_8 | 0.18771 |
| 521_4 | 0.20459 |

### Example 4: Testing Compound Selectivity and Potential Off-Target Profile

Selected compounds (31_1, 39_9, and 29_25) were tested for their propensity to affect other targets than the intended SCN9A target.

The following materials were used:
Human iCell GlutaNeurons, StemCell Technology GNC-301-030-001, Lot #101442 (R1034)
RhLaminin-521, Biolamina #LN521, 100 μg/mL
Brainphys Neuronal medium, StemCell Technology #05790
iCell Neural Supplement B #M1029
iCell Nervous System Supplement #M1031
N2 Supplement 100×, Thermo Fisher Scientific #17502-048
24-well culture plate, Costar #3524
Laminin from Engelbreth-Holm-Swarm murine sarcoma basement membrane, Sigma #L2020, 1 mg/mL
Treatment compounds: 31_1, 39_9 and 29_25, 5 mM stocks in PBS, +4° C.
10×HBSS with Ca2+/Mg2+, Gibco #14065-049
RLT plus Lysis buffer, Qiagen #1053393+1% b-mEtOH, Sigma #M7522

Human iPSC-derived cortical glutamatergic neurons (hGNs) were prepared by thawing frozen cell suspensions of human iCell GlutaNeurons according to the manufacturer's protocol (StemCell Technology). Freshly thawed cells were re-suspended in growth media (96 mL BrainPhys Neuronal medium; 2 mL iCell Neural Supplement B; 1 mL iCell Nervous System Supplement; 1 mL N2 Supplement, 100×) and seeded in 24-well plates to a seeding density of 375,000 cells/well. The 24-well plates were freshly coated with laminin by adding 400 μL/well of 1×HBSS with 10 μg/mL Laminin-521 to each well and incubating for 4 hours at 37° C. Cell culturing conditions are summarized in Table 10. For the first week of culturing, 50% percent of media was changed every day. After the first week until the start of compound addition (day 14), 50% of media was changed every second day.

TABLE 10

| | | | | Incub. time | Incub. time |
|---|---|---|---|---|---|
| | | | Seeding density | before oligo | with oligo |
| Cell Line | Vendor | Culture Condition | (cells/well) | (days) | (hrs) |
| Human iCell | StemCell | 37° C., 5% CO₂, | 375.000 cells/well, | 14 | 72 |
| GlutaNeurons | Technology | 95% relative humidity, | 24-well plate. | | |
| | GNC-301-030-001 | in active evaporation | Freshly coated | | |
| | | incubator. | with laminin. | | |

After 14 days of cell culture, the test compounds (31_1, 39_9 and 29_25) were added directly to the cell growth media to their final concentrations; 3 μM and 30 μM. These concentrations roughly correspond to 50- and 500-fold of their EC50 values for SCN9a in SK-N-AS cells. After 72 h of incubation, the media was removed and the cells were lysed in 600 μL/well 1% b-mEtoH/RLT buffer and thereafter stored at −80° C. until total RNA isolation.

RNA Sequencing Analysis:

Total RNA was isolated from the iPSC-derived glutamatergic neurons using RNeasy Mini Kit (Qiagen) and further processed into sequencing libraries using the TruSeq Stranded mRNA kit (Illumina) according to manufacturer's instructions. Libraries were sequenced (2×50 bp) on a NovaSeq instrument (IIlumina). To estimate gene expression levels, paired-end RNASeq reads were mapped onto the human genome (hg19) by use of the short read aligner GSNAP. Mapped reads for all RefSeq transcript variants of a gene were combined into a single value, read counts per gene, by applying SAMtools version 1.5 and customized in-house tools. Subsequently, read counts were normalized by sequencing library size and gene length according to Mortazavi et al. (Nat Methods 2008 July; 5(7):621-8), denoted as rpkms (number of mapped reads per kilobase transcript per million sequenced reads). A negative binomial regression model was derived to correct for potential confounding factors with the inclusion of covariates. The contrasts of interest for differential gene expression analysis were: each LNA at two different concentrations (3 μM and 30 μM) against the vehicle. Each condition had 4 replicates. The implementation was conducted in R using the DESeq2 package (Love M I, et al., Genome Biology 2014; 15:550 et seq.).

Two analyses were carried out. The first analysis looked at all genes that showed a change in expression as compared to the control with an adjusted (adj.) p-value/FDR threshold of 0.05. The second analysis focused on off-target candidate genes, which were defined as those that were down-regulated (defined as logFC<0 and adjusted p-value<0.05) and (i) were either among the top-1% of predicted off-target genes based on the binding affinity predictions or (ii) had 1 mismatch with the corresponding un-spliced transcript.

Table 11 summarizes the overall results from both analyses, whereas Tables 12 and 13 show more details on the results from the first and second analysis, respectively. The data showed that compounds 31_1 and 39_9 were very selective for SCN9A knock-down. At 3 uM, compound 31_1 had zero candidate off-target genes.

TABLE 11
Total number of genes either down- or upregulated after incubation
with either 3 μM or 30 μM of compound.

| | Candidate off-target | | Global transcriptomic | |
|---|---|---|---|---|
| CMP ID | genes* | | impact assessment† | |
| NO | 3 uM | 30 uM | 3 uM | 30 uM |
| 31_1 | 0 | 11 | Down: 5 Up: 1 | Down: 106 Up: 70 |
| 39_9 | 1 | 8 | Down: 3 Up: 0 | Down: 23 Up: 5 |
| 29_25 | 36 | 80 | Down: 268 Up: | Down: 1719 Up: |
| | | | 64 | 1278 |

*Genes that show reduced expression vs. control condition with adjusted p-value < 0.05 and (i) are among the top 1% predicted off-target genes based on the binding affinity predictions or (ii) can bind to the corresponding unspliced transcript with 1 mismatch.
†Genes that show a difference in expression level vs. control at adj. p-value < 0.05.

TABLE 12
Dysregulated genes at 3 uM of the indicated compounds.

| | |
|---|---|
| Downregulated | Upregulated |

| CMP ID | Gene | % | Gene | % |
|---|---|---|---|---|
| NO | symbol | KD | symbol | Increase |
| 31_1 | (SCN9A) | −99 | GRIN2C | 84 |
| | UPF2 | 31 | | |
| | IPO5 | 22 | | |
| | COPA | 15 | | |
| | NETO2 | 16 | | |
| 39_9 | (SCN9A) | −96 | 0 | — |
| | MORC4 | 92 | | |
| | NEGR1 | 22 | | |
| 29_25 | 268 | — | 64 | — |

TABLE 13

| Off-target candidate genes* | |
|---|---|
| 3 uM | 30 uM |

| CMP ID | Gene | % | Gene | % |
|---|---|---|---|---|
| NO | symbol | KD | symbol | KD |
| 31_1 | (SCN9A) | −99 | (SCN9A) | −100 |
| | | | TBC1D23 | 55 |
| | | | CEP83 | 51 |
| | | | MDGA2 | 36 |
| | | | ZFHX4 | 33 |
| | | | PAK5 | 31 |
| | | | LYPLAL1 | 29 |
| | | | MAGE | 28 |
| | | | TRIM66 | 26 |
| | | | LPP | 23 |
| | | | TMEM183A | 19 |
| | | | LSAMP | 19 |
| 39_9 | (SCN9A) | −96 | (SCN9A) | −98 |
| | MORC4 | 92 | MORC4 | 98 |
| | NEGR1 | 22 | NEGR1 | 68 |
| | | | FARS2 | 56 |
| | | | ADAMTS17 | 74 |
| | | | EXOC4 | 29 |
| | | | MUC19 | 59 |
| | | | SCN3A | 17 |
| | | | SYT1 | 17 |
| 29_25 | (SCN9A) | −98 | (SCN9A) | −99 |
| | DPP6 | 93 | NT5DC3 | 97 |
| | CSMD1 | 90 | MACROD2 | 95 |
| | UPF2 | 87 | CSMD1 | 94 |
| | PRKRIP1 | 78 | CTNNA2 | 93 |
| | KCTD20 | 75 | FAM189A1 | 93 |
| | CAT | 70 | MDGA2 | 93 |
| | NT5DC3 | 69 | ATG10 | 93 |
| | NF2 | 68 | UPF2 | 92 |
| | MACROD2 | 64 | ADAMTS3 | 88 |
| | MDGA2 | 58 | FLRT2 | 84 |
| | FAM189A1 | 53 | GALNT18 | 84 |
| | ATG10 | 49 | CNTNAP5 | 82 |
| | CLCN3 | 49 | MAGE | 81 |
| | CTNNA2 | 45 | CNKSR3 | 81 |
| | PTPRD | 44 | PRKRIP1 | 79 |
| | FLRT2 | 41 | SLF2 | 72 |
| | CNTNAP5 | 40 | CCSER1 | 72 |
| | TENM3 | 39 | PRKN | 72 |
| | NKAIN2 | 39 | POLB | 71 |
| | WAC | 38 | NTAN1 | 70 |
| | MAGE | 36 | PCDHGB1 | 70 |
| | RPTOR | 36 | IL1RAPL1 | 67 |
| | SLF2 | 34 | LTBP1 | 67 |
| | DPH5 | 31 | PTPRD | 66 |
| | KIAA1958 | 29 | KIAA1958 | 65 |
| | EPCAM-DT | 28 | SPINK5 | 65 |
| | SPINK5 | 27 | TENM4 | 62 |
| | POLB | 26 | TENM3 | 61 |
| | ICA1 | 26 | LINC01876 | 56 |
| | STK38L | 25 | EPCAM-DT | 55 |
| | STAG1 | 23 | TTLL5 | 54 |
| | ZNF280D | 23 | ERN1 | 54 |
| | EDIL3 | 21 | ERC2 | 52 |
| | CCSER1 | 21 | ZNF280D | 52 |
| | ASCC3 | 18 | STAG1 | 50 |
| | DCLK2 | 17 | KCTD16 | 50 |
| | | | NCKAP5 | 50 |
| | | | PTPRZ1 | 49 |
| | | | SVIL | 47 |
| | | | MARCHF8 | 46 |
| | | | PCDHGA3 | 44 |
| | | | BZW2 | 41 |
| | | | EDIL3 | 40 |
| | | | GRB14 | 39 |
| | | | DCLK2 | 36 |
| | | | DCC | 33 |
| | | | XDH | 33 |
| | | | PAK5 | 31 |
| | | | STK38L | 31 |
| | | | PER3 | 30 |
| | | | NCOA2 | 29 |
| | | | GALNT13 | 29 |
| | | | KLHL2 | 27 |
| | | | CADPS | 26 |
| | | | WDR17 | 26 |
| | | | VRK2 | 26 |
| | | | DMD | 23 |
| | | | MICU1 | 20 |
| | | | CDC42 | 19 |
| | | | NRCAM | 18 |
| | | | ASCC3 | 17 |
| | | | ANKRD24 | 16 |
| | | | CDH4 | 14 |
| | | | IL11RA | 14 |
| | | | DPP6 | 96 |
| | | | KCTD20 | 95 |
| | | | CAT | 94 |
| | | | NF2 | 93 |
| | | | FHIT | 93 |
| | | | NKAIN2 | 89 |
| | | | RPTOR | 85 |
| | | | CLCN3 | 70 |
| | | | ICA1 | 70 |
| | | | WAC | 67 |
| | | | PIP4K2A | 48 |
| | | | DPH5 | 45 |
| | | | RARB | 45 |
| | | | WIPF3 | 33 |
| | | | PDE4B | 27 |
| | | | MYO19 | 20 |

*Defined as down-regulated genes (defined as logFC < 0 and adjusted p-value < 0.05) that (i) were among the top-1% of predicted off-target genes based on the binding affinity predictions or (ii) could bind to the corresponding un-spliced transcript with 1 mismatch.

### Example 5: In Vivo Testing of Compound 31_1

The goals of this study were to assess tolerability and to optimize delivery of compound 31_1 to dorsal root ganglia (DRGs) in cynomolgus monkey (Macaca fascicularis), comparing high flushing vs low flushing volumes (post-intrathecal dose injection), as well as to obtain pharmacokinetic (PK) and pharmacodynamic (PD) readouts in DRGs at several time points. Dose levels were to be kept “adaptive” (i.e. dosing staggered and doses adjusted based on emerging findings). The route of administration was chosen because it was the anticipated human therapeutic route and the route that could provide the best delivery of the compound to DRGs.

Approach

The study animals were assigned to six groups respectively denoted Groups 1 to 6, with three study animals in each group), where possible based on existing social groups and stratified body weights.

The animals were dosed by intrathecal bolus injection of 1.0 mL solution of compound 31_1 followed by artificial cerebrospinal fluid (aCSF) flush of either 0.5 mL/kg body weight (Groups 1 and 2) or 0.1 mL/kg (Groups 3 to 6). For details, see Table 14. Prior to administration, at least 0.5 mL CSF (up to the approximate dose volume, as feasible) was collected and used for CSF analysis.

Group 1 and 5: Terminal sacrifice on Day 43 of the dosing phase. Group 2, 3 and 4: Terminal sacrifice on Day 15 of the dosing phase. Group 6: Terminal sacrifice on Day 64 of the dosing phase.

Tissue Collection

At sacrifice, two pairs of DRGs were collected from each side (left, right) at each level of the spinal region (lumbar, thoracic, cervical), the exact weight of all DRG samples were recorded. From the spinal cord, two samples (max 50 mg, the exact weight is recorded) were dissected from lumbar, thoracic and cervical areas. From the brain, four samples (max 50 mg, the exact weights were recorded) were dissected from each of the following brain regions: frontal cortex, occipital cortex, cerebellum, hippocampus. All samples were placed in into appropriately labelled 2.0 mL Precellys homogenization tubes, snap frozen in liquid nitrogen and stored at −70° C. or below until further analyzed.

Samples were homogenized for bioanalytical analysis. All tissues were received frozen in Precellys tubes, and 800 μl ice-cold Cell Disruption Buffer (PARIS Kit, Catalog #AM1921, Ambion by Life Technologies) were added to each tube. The samples were homogenized on a Precellys homogenizer (program depending on tissue type). The homogenate was split in aliquots for e.g. RNA isolation and exposure analysis by hELISA.

Exposure Analysis by hELISA

Materials and Methods

The reagents and materials are shown in Table 15. The homogenates were brought to room temperature (RT) and vortexed before adding to dilution plates. As a reference standard for the hELISA, Compound 31_1 was spiked into a homogenate pool from un-dosed samples. The spike-in concentrations were prepared so that they were close to the antisense oligonucleotide (ASO) content of the samples (usually within −10 fold).

The samples were diluted in 5×SSCT buffer. Dilution factors ranged from 5-fold (low concentration plasma) to 10,000-fold dilutions for CSF early time points. Tissue samples were diluted at least 10-fold. Appropriate standards matching sample matrix and dilution factor were run on every plate. Samples and standards were added to a dilution plate in the desired setup, and dilution series were made. 300 μL sample/standard plus capture-detection solution was added to the first wells and 150 μL capture-detection solution to the remaining wells. A two-fold dilution series of standards and samples was made (6 steps) by transferring 150 μL liquid sequentially. The diluted samples were incubated on the dilution plate for 30 minutes at RT.

TABLE 15
Reagents used for hELISA analysis of compound 31_1.
Reagents table

Dilution plate: Polypropylene 96-well plate with round bottom.
Roche StreptaWell High Bind, 96-well plate clear: Cat. No. 11989685001.
5 x SSCT buffer, pH 7.0: 750 mM NaCl, and 75 mM sodium citrate,
containing 0.05% (v/v) Tween-20.
2 x SSCT buffer, pH 7.0: 300 mM NaCl, and 30 mM sodium citrate,
containing 0.05% (v/v) Tween-20.
Substrate (AP): Blue Phos Substrate, KPL product code 50-88-00.
Anti-Digoxigenin-AP, Fab fragments: Roche Applied Science, Cat. No.
11 093 274 910.
PBST, pH 7.2: Phosphate buffered saline, containing 0.05% (v/v)
Tween-20.
Compound ID NO# 31_1
5′-Biotinylated capture probe (Table 16)
3′-Digoxigenin-conjugated detection probe (Table 16)
Capture-detection solution: Solution of capture probe 35 nM and
detection probe 35 nM in 5xSSCT buffer (Table 16).

TABLE 16
Probes used for hELISA quantification of Compound 31_1.

| Type | Modification | Base Sequence | Sugar modification |
|---|---|---|---|
| Capture probe | 5′-Biotin-conjugated | 5′-GAAATGGT-3′ | Fully LNA-modified |
| Detection probe | 3′-Digoxigenin-conjugated | 5′-TAAAAETG-3′ | Fully LNA-modified |

Next, 100 μL of liquid was transferred from the dilution plate to a streptavidin plate. The plate was incubated for 1 hour at RT with gentle agitation (plate shaker). The wells were aspirated and washed three times with 300 μL of 2×SSCT buffer. To each well, 100 μL anti-DIG-AP diluted 1:4000 in PBST (made on the same day) were added and incubated for 1 hour at RT under gentle agitation.

The wells were then aspirated and washed three times with 300 μL of 2×SSCT buffer. Finally, 100 μL of freshly prepared substrate (AP) solution were added to each well.

The intensity of the color reaction was measured spectrophotometrically at 615 nm after 30 minutes of incubation with gentle agitation. Raw data were exported from the readers (Gen5 2.0 software) to excel format and further analyzed in excel. Standard curves were generated using GraphPad Prism 6 software and a logistic 4PL regression model. Data points were reported as the mean value of the technical replicates.

Results

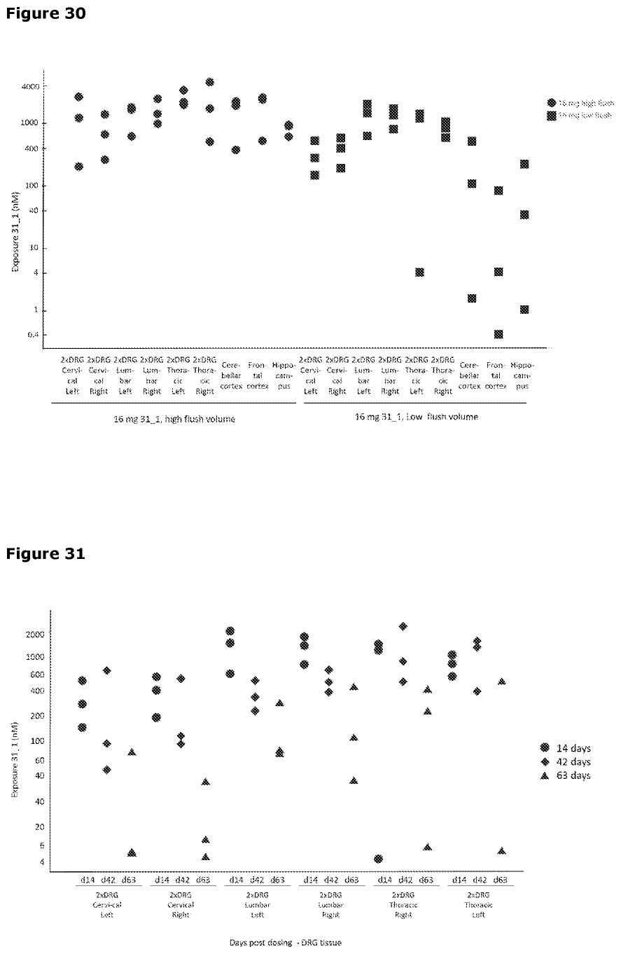

The data showed that both high and low flushing volumes generated high exposure of Compound 31_1 in cynomolgus DRGs in all of the lumbar, cervical and thoracic regions (FIG. 30). Moreover, the exposure was not significantly different from right and left side DRGs. The low flushing volume resulted in much lower exposure in all analyzed brain regions (frontal cortex, cerebellum, and hippocampus) whereas high flushing volume resulted in high exposure in all brain regions (FIG. 30).

The highest measured exposure was achieved at the 14 days post-dosing time point, where Cmax exceeded 1000 nM in lumbar DRGs (FIG. 31).

Expression Analysis by RNA Sequencing

RNA was isolated from the tissue homogenate using the PARIS Kit (Catalog #AM1921, Ambion by Life Technologies) according to the manufacturer's protocol.

Sequencing is performed using a ribosomal depletion protocol and 20 million paired-end (PE) reads (2×101 bp) are obtained. Data analysis is performed after quality assessment, including removal of short reads (reads <50 nucleotides) and a quality below Q30. PE reads are mapped to cynomolgus monkey genome (reference sequence Macaca_fascicularis_5.0 (macFas5), downloadable from UCSC genome browser) and the gene expression analysis is performed using the software CLC Genomic Workbench version 20.

A set of genes whose expression correlate with the expression of SCN9A in saline treated animals has been found in previous studies. This set of genes is denoted “HK genes”, and the Pearson correlation between the expression of each HK gene and that of SCN9A is greater than 0.95. The geometric mean of the HK genes in saline treated animals is calculated and denoted by GM_{HK}. In each sample the GM_{RK} is used to normalize the expression of SCN9A (X) by the following formula (Formula I), where Xₛₐₗᵢₙₑ is the expression of SCN9A in saline, treated animals:

$$Normalised SCN9A expression ( \% saline ) = \frac{\frac{X}{{GM}_{HK}}}{mean ( \frac{{X}_{saline}}{{GM}_{HK}} )}$$

In view of the high measured exposure in lumbar DRGs and potency of the compound, efficient inhibition of the target can be expected.

TABLE 7
Caspase Analysis Results

| | Caspase_{—} | | | |
|---|---|---|---|---|
| | Activation_{—} | Caspase_{—} | | Caspase_{—} |
| | Assay_BALB3T3_{—} | Activation_{—} | Caspase_{—} | Activation_{—} |
| | Clone_A31: | Assay_BALB3T3_{—} | Activation_{—} | Assay_BALB3T3_{—} |
| | AP013716; | Clone_A31: | Assay_BALB3T3_{—} | Clone_A31: |
| | CASPASE_{—} | AP013716; | Clone_A31: | AP013716; |
| CMP | ACTIVATION_SCORE; | PCT_POS_CTRL; | AP013716; | Mean; |
| ID | CONCENTRATION | CONCENTRATION | Mean; | CASPASE_{—} |
| NO | (μM) | (μM) | PCT_POS_CTRL | ACTIVATION_SCORE |
| 29_33 | 0.1 | 0.1 | 18.9 | 0 |
| 48_9 | 0.1 | 0.1 | 8.9 | 0 |
| 39_13 | 0.1 | 0.1 | −2.8 | 0 |
| 39_14 | 0.1 | 0.1 | −4.5 | 0 |
| 29_11 | 0.1 | 0.1 | 20.8 | 0.5 |
| 29_14 | 0.1 | 0.1 | 14.7 | 0 |
| 48_5 | 0.1 | 0.1 | 1.8 | 0 |
| 39_20 | 0.1 | 0.1 | −10.0 | 0 |
| 39_19 | 0.1 | 0.1 | −13.0 | 0 |
| 39_18 | 0.1 | 0.1 | −13.0 | 0 |
| 39_17 | 0.1 | 0.1 | −7.0 | 0 |
| 39_15 | 0.1 | 0.1 | −11.2 | 0 |
| 39_16 | 0.1 | 0.1 | −12.5 | 0 |
| 29_41 | 0.1 | 0.1 | 2.5 | 0 |
| 29_34 | 0.1 | 0.1 | 7.9 | 0 |
| 29_37 | 0.1 | 0.1 | 4.5 | 0 |
| 29_39 | 0.1 | 0.1 | 5.7 | 0 |
| 29_40 | 0.1 | 0.1 | 0.3 | 0 |
| 29_4 | 0.1 | 0.1 | 45.8 | 2 |
| 29_35 | 0.1 | 0.1 | 10.1 | 0 |
| 29_36 | 0.1 | 0.1 | 5.9 | 0 |
| 29_38 | 0.1 | 0.1 | 6.0 | 0 |
| 48_10 | 0.1 | 0.1 | 17.8 | 0 |
| 39_12 | 0.1 | 0.1 | −4.9 | 0 |
| 39_11 | 0.1 | 0.1 | −4.9 | 0 |
| 39_10 | 0.1 | 0.1 | −7.7 | 0 |
| 39_9 | 0.1 | 0.1 | −11.1 | 0 |
| 29_32 | 0.1 | 0.1 | 4.5 | 0 |
| 29_31 | 0.1 | 0.1 | 18.7 | 0 |
| 29_30 | 0.1 | 0.1 | 12.9 | 0 |
| 29_13 | 0.1 | 0.1 | 23.7 | 0.5 |
| 29_29 | 0.1 | 0.1 | 16.0 | 0 |
| 29_28 | 0.1 | 0.1 | 7.1 | 0 |
| 29_27 | 0.1 | 0.1 | 12.1 | 0 |
| 29_26 | 0.1 | 0.1 | 4.7 | 0 |
| 29_25 | 0.1 | 0.1 | 3.8 | 0 |
| 29_24 | 0.1 | 0.1 | 1.6 | 0 |
| 48_8 | 0.1 | 0.1 | 4.9 | 0 |
| 48_4 | 0.1 | 0.1 | 21.1 | 0.5 |
| 48_7 | 0.1 | 0.1 | 13.6 | 0 |
| 48_6 | 0.1 | 0.1 | 8.6 | 0 |
| 48_3 | 0.1 | 0.1 | 30.0 | 1 |
| 48_2 | 0.1 | 0.1 | 32.8 | 1 |
| 47_1 | 0.1 | 0.1 | −12.2 | 0 |
| 33_3 | 0.1 | 0.1 | 17.2 | 0 |
| 33_2 | 0.1 | 0.1 | 23.4 | 0.5 |
| 33_1 | 0.1 | 0.1 | 22.1 | 0.5 |
| 31_5 | 0.1 | 0.1 | 4.6 | 0 |
| 31_4 | 0.1 | 0.1 | 5.7 | 0 |
| 31_3 | 0.1 | 0.1 | 16.8 | 0 |
| 31_2 | 0.1 | 0.1 | 12.2 | 0 |
| 31_1 | 0.1 | 0.1 | 14.6 | 0 |
| 48_1 | 0.1 | 0.1 | 31.1 | 1 |
| 39_5 | 0.1 | 0.1 | −9.2 | 0 |
| 39_4 | 0.1 | 0.1 | −5.1 | 0 |
| 39_8 | 0.1 | 0.1 | −6.5 | 0 |
| 39_3 | 0.1 | 0.1 | −13.3 | 0 |
| 39_7 | 0.1 | 0.1 | −5.2 | 0 |
| 39_2 | 0.1 | 0.1 | 4.4 | 0 |
| 39_1 | 0.1 | 0.1 | −6.8 | 0 |
| 39_6 | 0.1 | 0.1 | −6.6 | 0 |
| 29_23 | 0.1 | 0.1 | 8.6 | 0 |
| 29_22 | 0.1 | 0.1 | 18.7 | 0 |
| 29_2 | 0.1 | 0.1 | 49.0 | 2 |
| 29_8 | 0.1 | 0.1 | 32.8 | 1 |
| 29_7 | 0.1 | 0.1 | 33.9 | 1 |
| 29_6 | 0.1 | 0.1 | 37.6 | 1 |
| 29_1 | 0.1 | 0.1 | 76.4 | 3 |
| 29_3 | 0.1 | 0.1 | 42.9 | 2 |
| 29_10 | 0.1 | 0.1 | 36.7 | 1 |
| 29_12 | 0.1 | 0.1 | 24.3 | 0.5 |
| 29_9 | 0.1 | 0.1 | 33.5 | 1 |
| 29_21 | 0.1 | 0.1 | 17.0 | 0 |
| 29_20 | 0.1 | 0.1 | 16.0 | 0 |
| 29_19 | 0.1 | 0.1 | 7.9 | 0 |
| 29_18 | 0.1 | 0.1 | 12.5 | 0 |
| 29_17 | 0.1 | 0.1 | 17.3 | 0 |
| 29_5 | 0.1 | 0.1 | 30.9 | 1 |
| 29_16 | 0.1 | 0.1 | 18.7 | 0 |
| 29_15 | 0.1 | 0.1 | 16.9 | 0 |

| | Caspase_{—} | | | |
|---|---|---|---|---|
| | Activation_{—} | | Caspase_{—} | Caspase_{—} |
| | Assay_Hep G2: | Caspase_{—} | Activation_{—} | Activation_{—} |
| | AP012864; | Activation_{—} | Assay_Hep G2: | Assay_Hep G2: |
| | CASPASE, | Assay_Hep G2: | AP012864; | AP012864; |
| CMP | ACTIVATION_SCORE; | AP012864; | PCT_POS_CTRL; | Mean; |
| ID | CONCENTRATION | Mean; | CONCENTRATION | CASPASE_{—} |
| NO | (μM) | PCT_POS_CTRL | (μM) | ACTIVATION_SCORE |
| 29_33 | 0.1 | 45.6 | 0.1 | 2 |
| 48_9 | 0.1 | 11.3 | 0.1 | 0.25 |
| 39_13 | 0.1 | −2.2 | 0.1 | 0 |
| 39_14 | 0.1 | −3.5 | 0.1 | 0 |
| 29_11 | 0.1 | 36.2 | 0.1 | 1 |
| 29_14 | 0.1 | 40.4 | 0.1 | 2 |
| 48_5 | 0.1 | 6.3 | 0.1 | 0 |
| 39_20 | 0.1 | −4.0 | 0.1 | 0 |
| 39_19 | 0.1 | −5.2 | 0.1 | 0 |
| 39_18 | 0.1 | −5.6 | 0.1 | 0 |
| 39_17 | 0.1 | −3.9 | 0.1 | 0 |
| 39_15 | 0.1 | −4.9 | 0.1 | 0 |
| 39_16 | 0.1 | −5.3 | 0.1 | 0 |
| 29_41 | 0.1 | 56.4 | 0.1 | 2 |
| 29_34 | 0.1 | 22.8 | 0.1 | 0.5 |
| 29_37 | 0.1 | 46.1 | 0.1 | 2 |
| 29_39 | 0.1 | 51.2 | 0.1 | 2 |
| 29_40 | 0.1 | 57.8 | 0.1 | 2 |
| 29_4 | 0.1 | 81.3 | 0.1 | 3 |
| 29_35 | 0.1 | 38.9 | 0.1 | 1 |
| 29_36 | 0.1 | 51.3 | 0.1 | 2 |
| 29_38 | 0.1 | 50.0 | 0.1 | 2 |
| 48_10 | 0.1 | 2.7 | 0.1 | 0 |
| 39_12 | 0.1 | −4.3 | 0.1 | 0 |
| 39_11 | 0.1 | −3.7 | 0.1 | 0 |
| 39_10 | 0.1 | −4.1 | 0.1 | 0 |
| 39_9 | 0.1 | −5.1 | 0.1 | 0 |
| 29_32 | 0.1 | 53.6 | 0.1 | 2 |
| 29_31 | 0.1 | 45.5 | 0.1 | 2 |
| 29_30 | 0.1 | 47.3 | 0.1 | 2 |
| 29_13 | 0.1 | 82.8 | 0.1 | 3 |
| 29_29 | 0.1 | 33.5 | 0.1 | 1 |
| 29_28 | 0.1 | 54.6 | 0.1 | 2 |
| 29_27 | 0.1 | 59.6 | 0.1 | 2 |
| 29_26 | 0.1 | 3.7 | 0.1 | 0 |
| 29_25 | 0.1 | 17.6 | 0.1 | 0 |
| 29_24 | 0.1 | 4.8 | 0.1 | 0 |
| 48_8 | 0.1 | 9.6 | 0.1 | 0 |
| 48_4 | 0.1 | 20.7 | 0.1 | 0.5 |
| 48_7 | 0.1 | 37.3 | 0.1 | 1 |
| 48_6 | 0.1 | 53.3 | 0.1 | 2 |
| 48_3 | 0.1 | 13.2 | 0.1 | 0 |
| 48_2 | 0.1 | 53.9 | 0.1 | 2 |
| 47_1 | 0.1 | −4.4 | 0.1 | 0 |
| 33_3 | 0.1 | 21.7 | 0.1 | 0.5 |
| 33_2 | 0.1 | 38.8 | 0.1 | 1 |
| 33_1 | 0.1 | 39.0 | 0.1 | 1 |
| 31_5 | 0.1 | 6.7 | 0.1 | 0 |
| 31_4 | 0.1 | 28.0 | 0.1 | 1 |
| 31_3 | 0.1 | 18.0 | 0.1 | 0 |
| 31_2 | 0.1 | 51.2 | 0.1 | 2 |
| 31_1 | 0.1 | 21.4 | 0.1 | 0.5 |
| 48_1 | 0.1 | 19.3 | 0.1 | 0 |
| 39_5 | 0.1 | −4.7 | 0.1 | 0 |
| 39_4 | 0.1 | −3.4 | 0.1 | 0 |
| 39_8 | 0.1 | −3.2 | 0.1 | 0 |
| 39_3 | 0.1 | −5.2 | 0.1 | 0 |
| 39_7 | 0.1 | −1.8 | 0.1 | 0 |
| 39_2 | 0.1 | 2.3 | 0.1 | 0 |
| 39_1 | 0.1 | −3.6 | 0.1 | 0 |
| 39_6 | 0.1 | −3.4 | 0.1 | 0 |
| 29_23 | 0.1 | 61.8 | 0.1 | 3 |
| 29_22 | 0.1 | 38.6 | 0.1 | 1 |
| 29_2 | 0.1 | 128.6 | 0.1 | 3 |
| 29_8 | 0.1 | 128.8 | 0.1 | 3 |
| 29_7 | 0.1 | 49.3 | 0.1 | 2 |
| 29_6 | 0.1 | 79.1 | 0.1 | 3 |
| 29_1 | 0.1 | 107.9 | 0.1 | 3 |
| 29_3 | 0.1 | 87.0 | 0.1 | 3 |
| 29_10 | 0.1 | 39.4 | 0.1 | 1 |
| 29_12 | 0.1 | 101.8 | 0.1 | 3 |
| 29_9 | 0.1 | 82.7 | 0.1 | 3 |
| 29_21 | 0.1 | 43.3 | 0.1 | 2 |
| 29_20 | 0.1 | 59.2 | 0.1 | 2 |
| 29_19 | 0.1 | 90.1 | 0.1 | 3 |
| 29_18 | 0.1 | 74.8 | 0.1 | 3 |
| 29_17 | 0.1 | 63.1 | 0.1 | 3 |
| 29_5 | 0.1 | 82.5 | 0.1 | 3 |
| 29_16 | 0.1 | 55.4 | 0.1 | 2 |
| 29_15 | 0.1 | 18.2 | 0.1 | 0 |

TABLE 14
Animal groups and doses.

| | | | Dose | Dose | | Animals/ | Necropsy After | | |
|---|---|---|---|---|---|---|---|---|---|
| Group | Treatment | Group | Level | Volume | Flush | Group | 2 | 6 | 9 |
| No. | code* | Description | (mg) | (mL) | Volume | Females | Weeks | Weeks | Weeks |
| 1 | G1 | Control | 0 | 1 mL | 0.5 mL/kg | 3 | — | 3 | |
| 4 | G2 | High dose + high | 16 mg | 1 mL | 0.5 mL/kg | 3 | 3 | — | |
| | | volume flush | | | | | | | |
| 3 | G3 | High dose + low | 16 mg | 1 mL | 0.1 mL/kg | 3 | 3 | — | |
| | | volume flush | | | | | | | |
| 2 | G4 | Low dose + low | 8 mg | 1 mL | 0.1 ml/kg | 3 | 3 | — | |
| | | volume flush | | | | | | | |
| 5 | G5 | High dose + low | 16 mg | 1 mL | 0.1 ml/kg | 3 | — | 3 | |
| | | volume flush | | | | | | | |
| 6 | G6 | High dose + low | 16 mg | 1 mL | 0.1 ml/kg | 3 | — | — | 3 |
| | | volume flush | | | | | | | |

1. An antisense oligonucleotide of 10 to 30 nucleotides in length comprising a contiguous nucleotide sequence of 10 to 30 nucleotides in length, which wherein the contiguous nucleotide sequence is fully complementary to a region of a human SCN9A pre-mRNA, and wherein the region is nucleotide positions 97704-97732, 103232-103259, 151831-151847, or 151949-152006[H] of SEQ ID NO: 1.

2. (canceled)

3. The antisense oligonucleotide of claim 1, wherein the contiguous nucleotide sequence is 100% identical to a sequence selected from the group consisting of SEQ ID NOs: 28-52; or at least 14 contiguous nucleotides thereof.

4-5. (canceled)

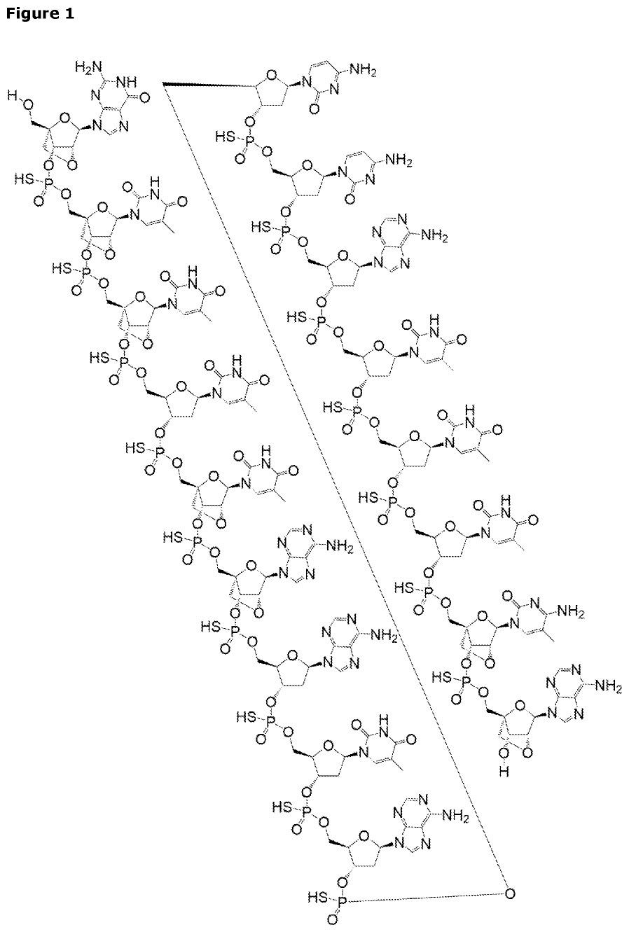

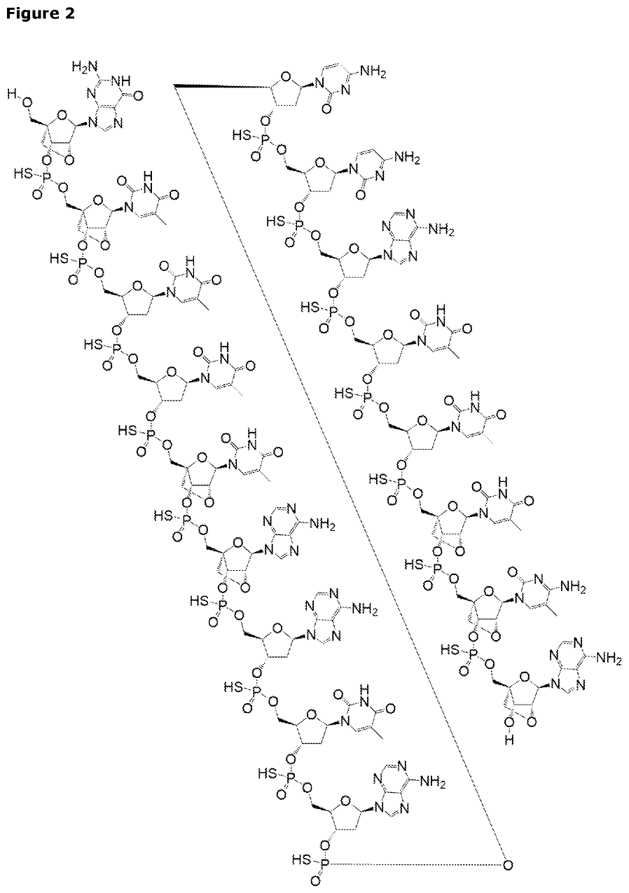

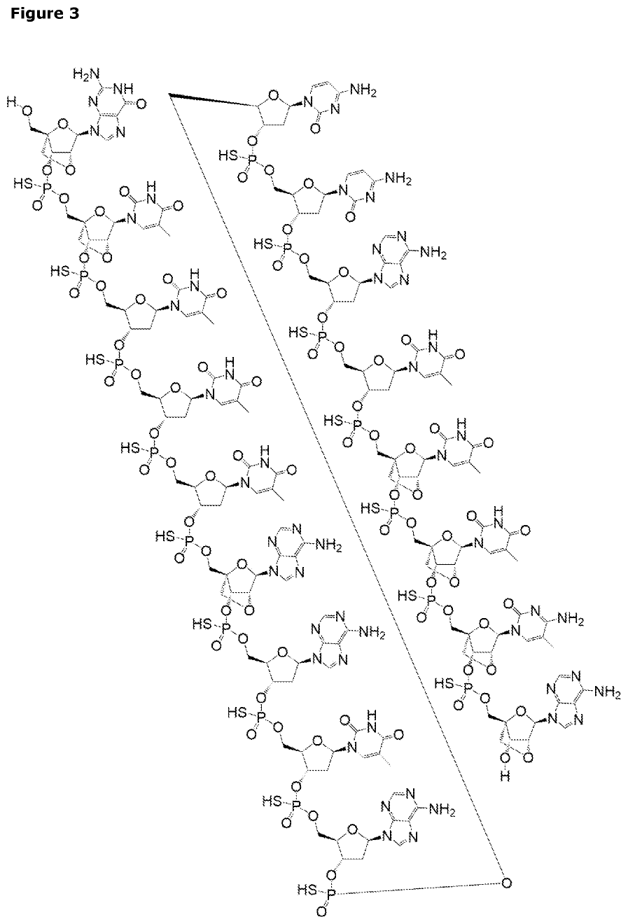

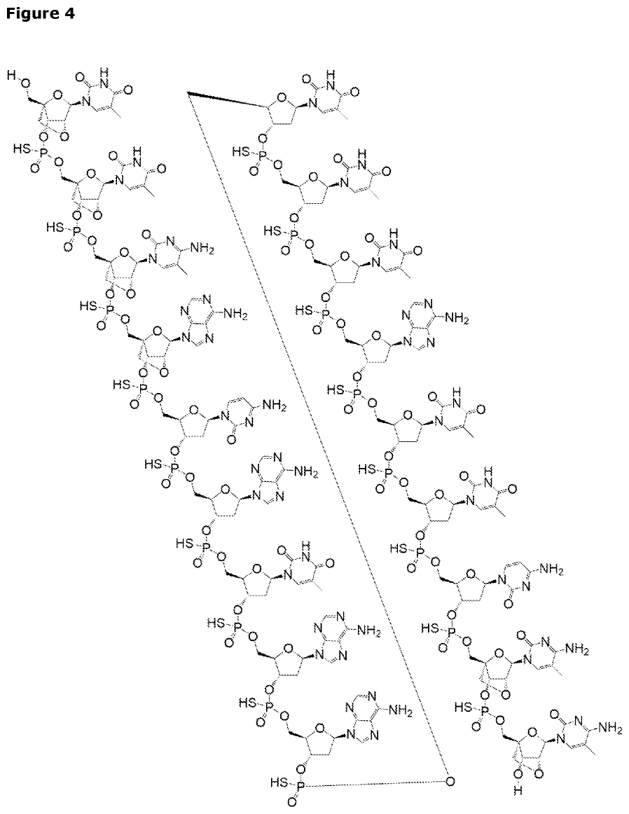

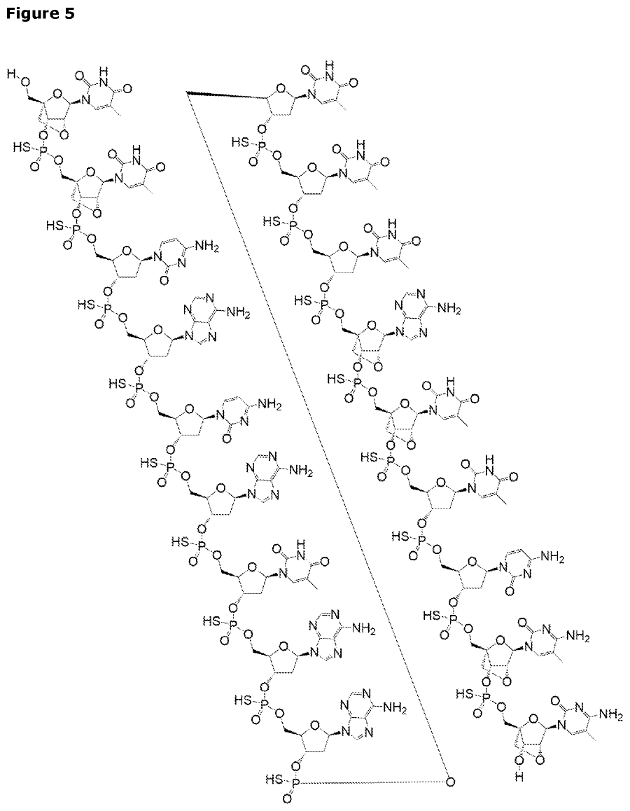

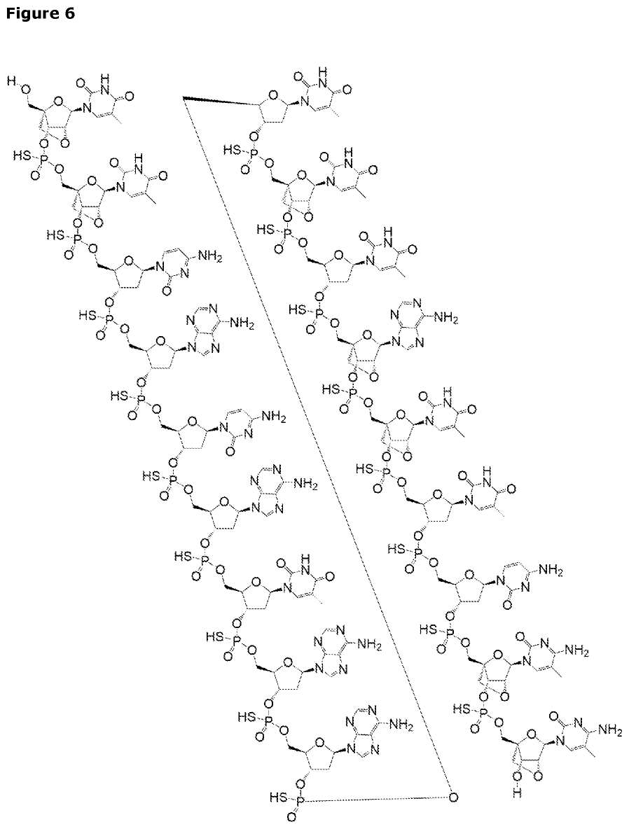

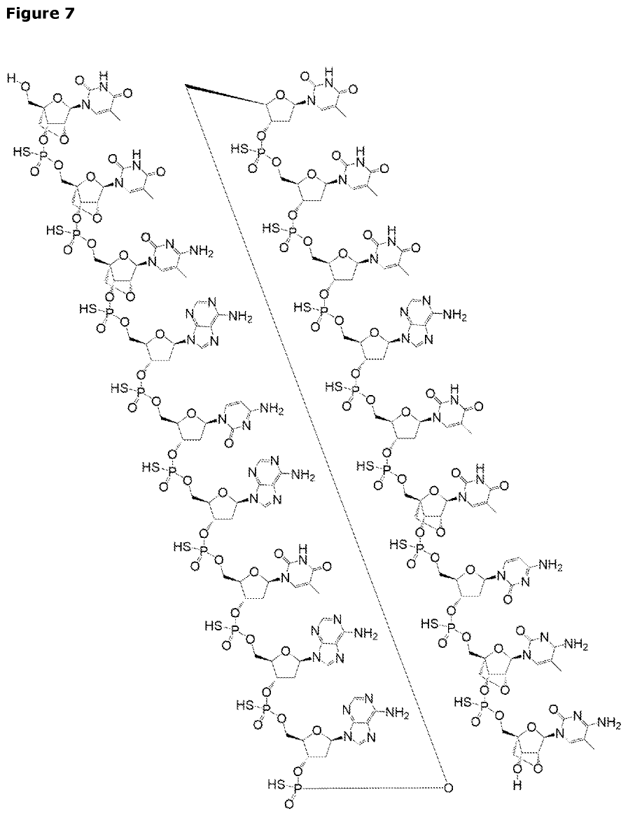

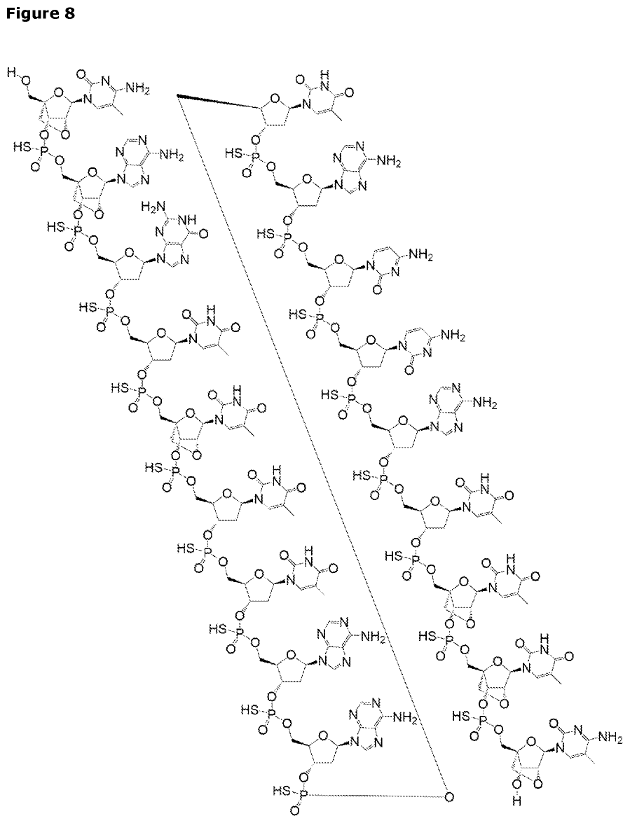

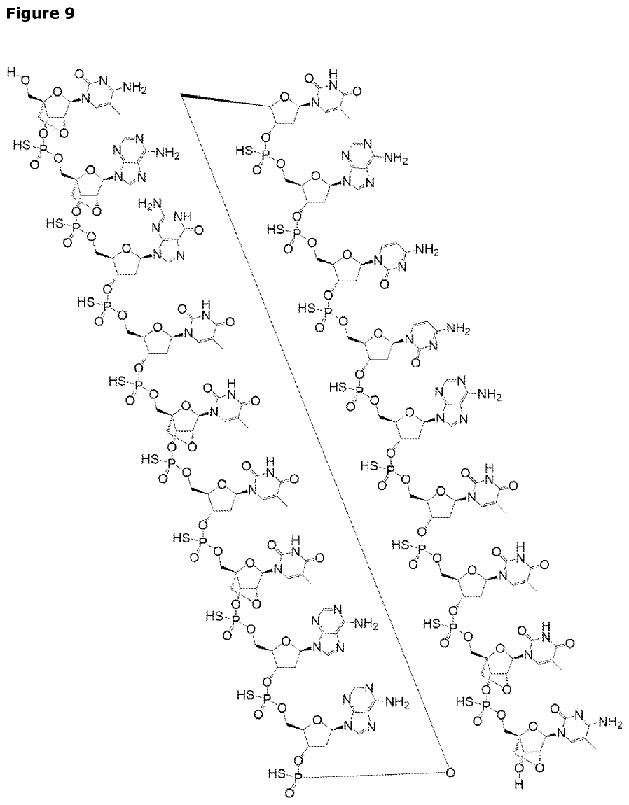

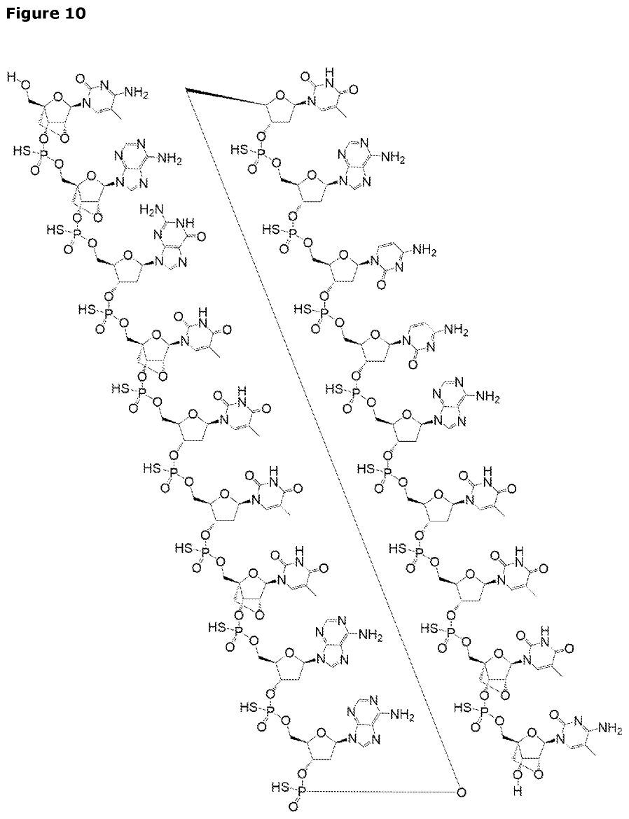

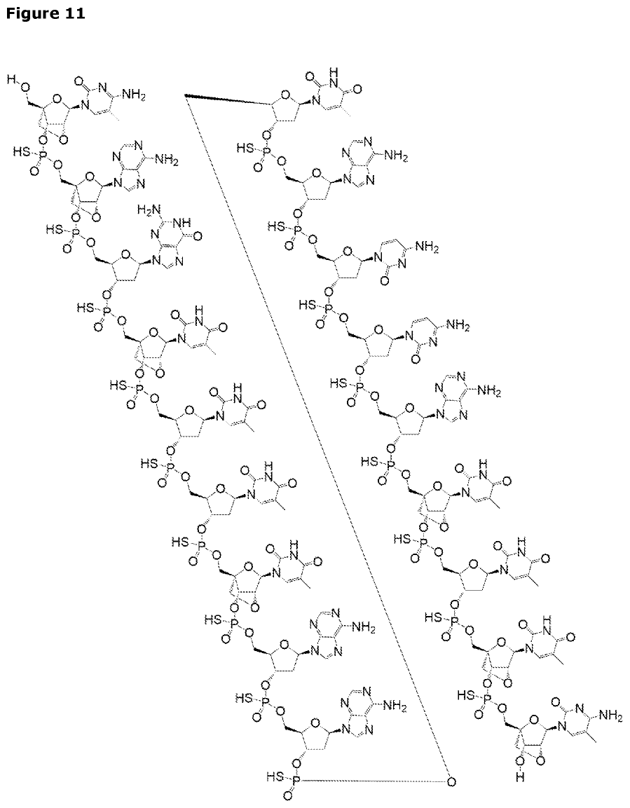

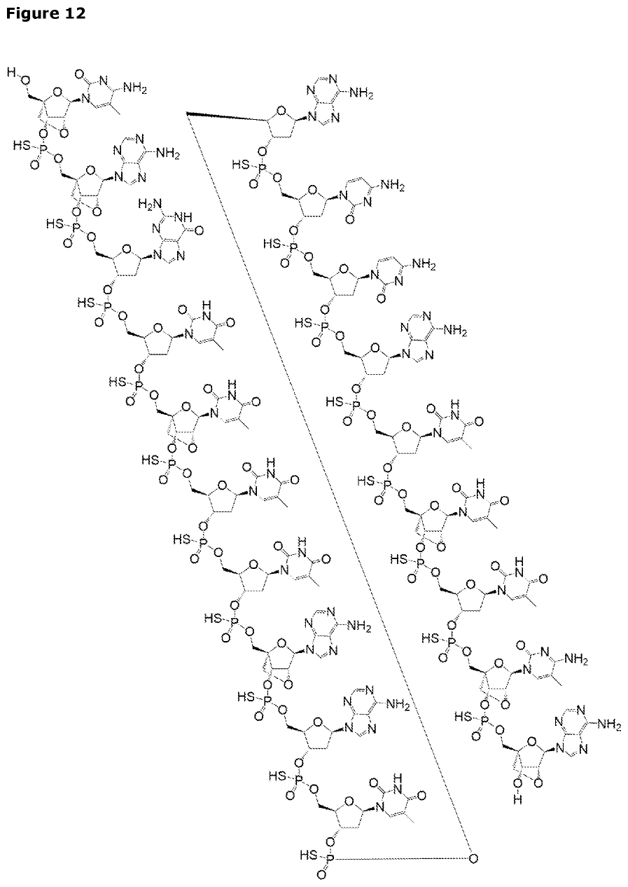

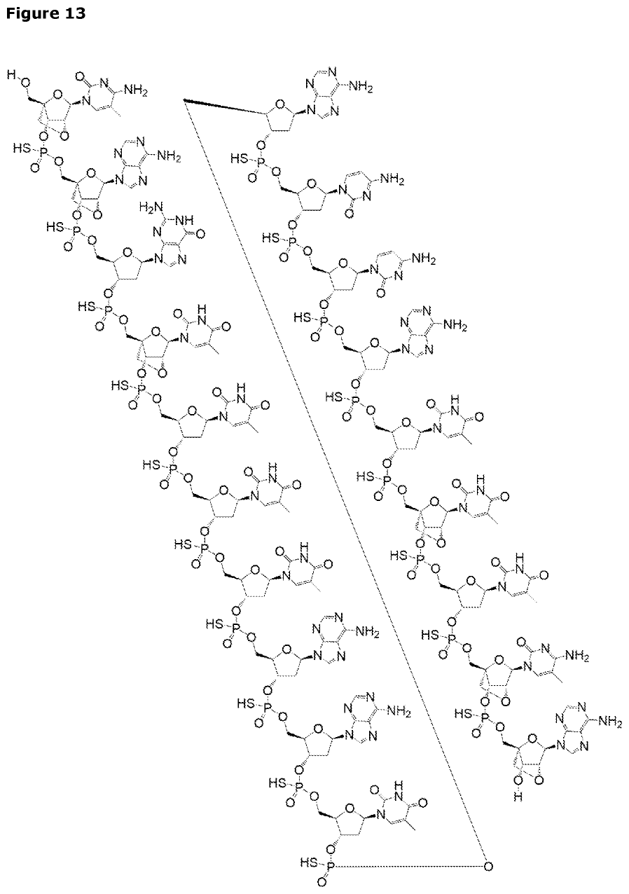

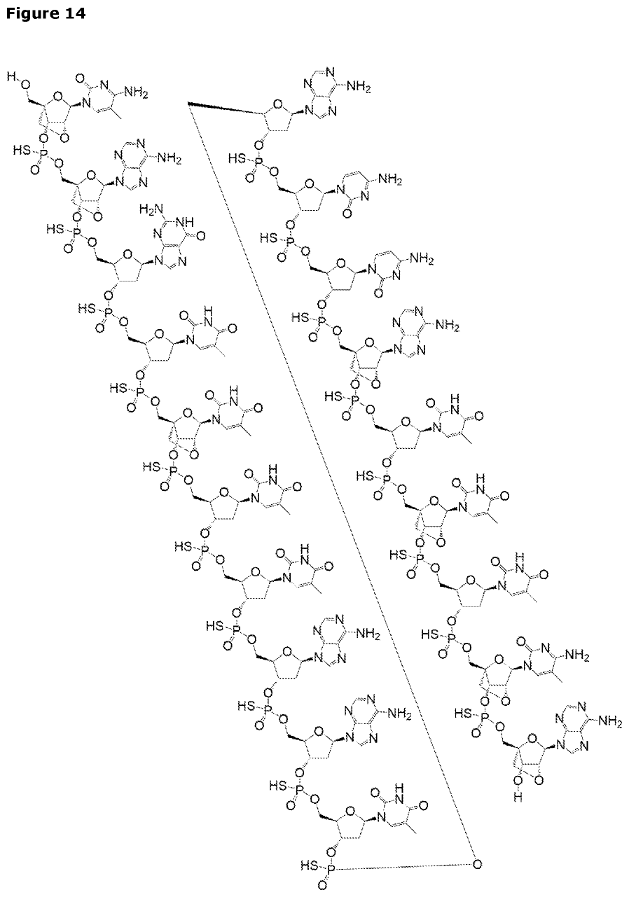

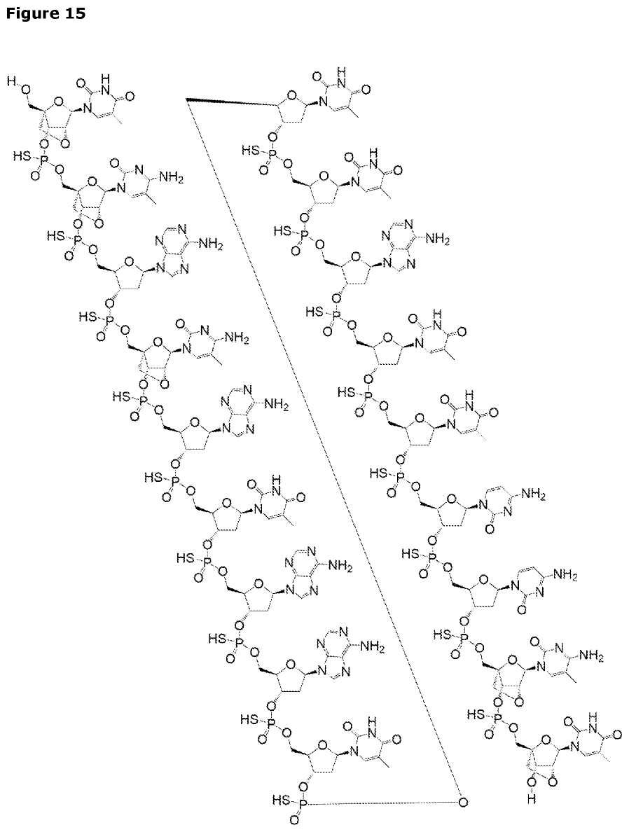

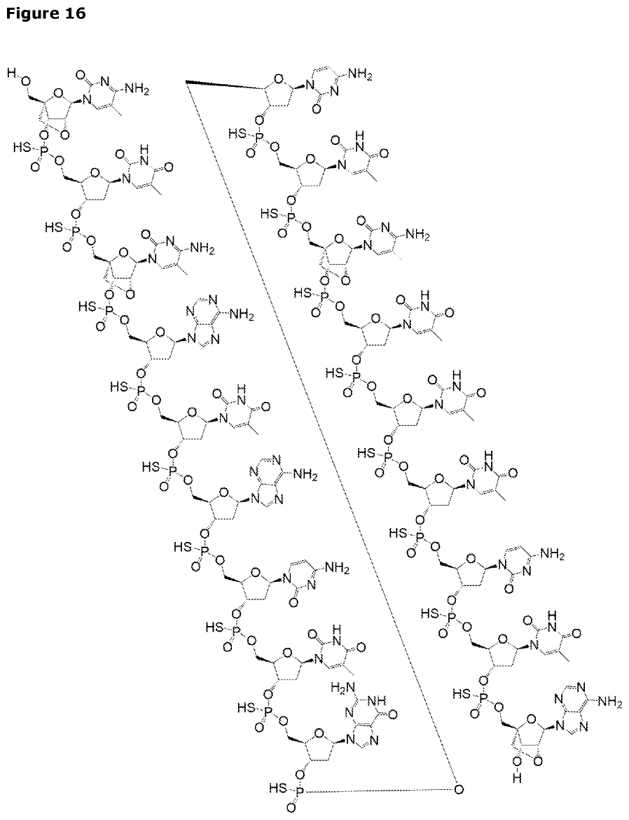

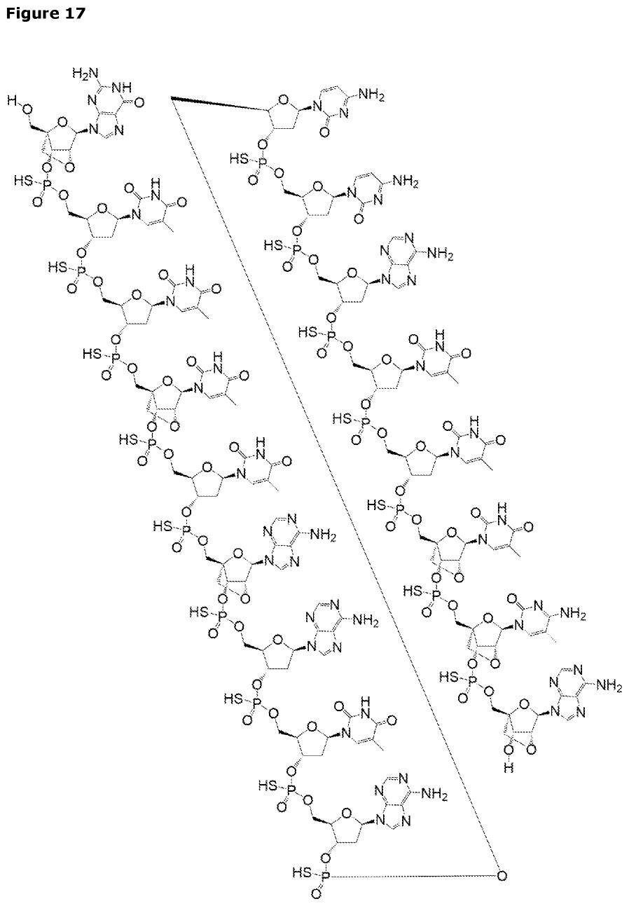

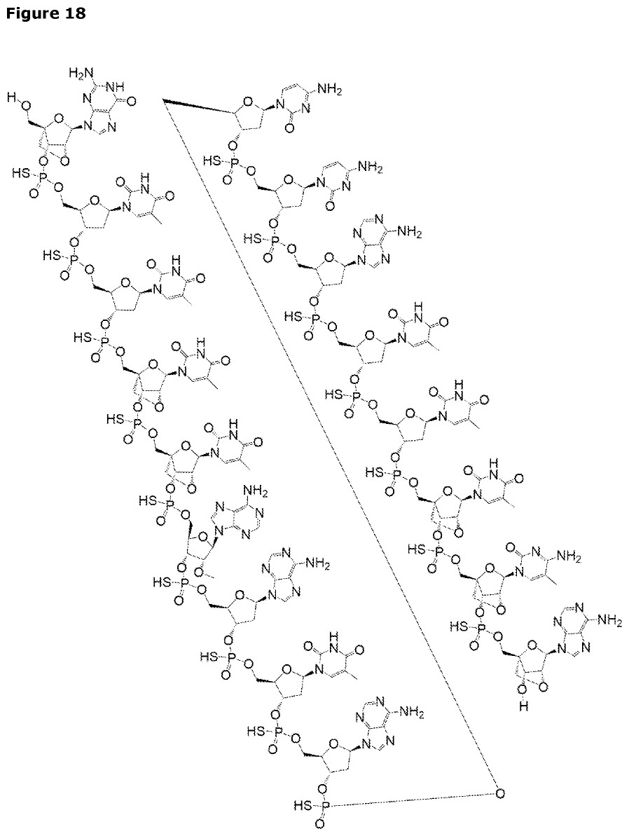

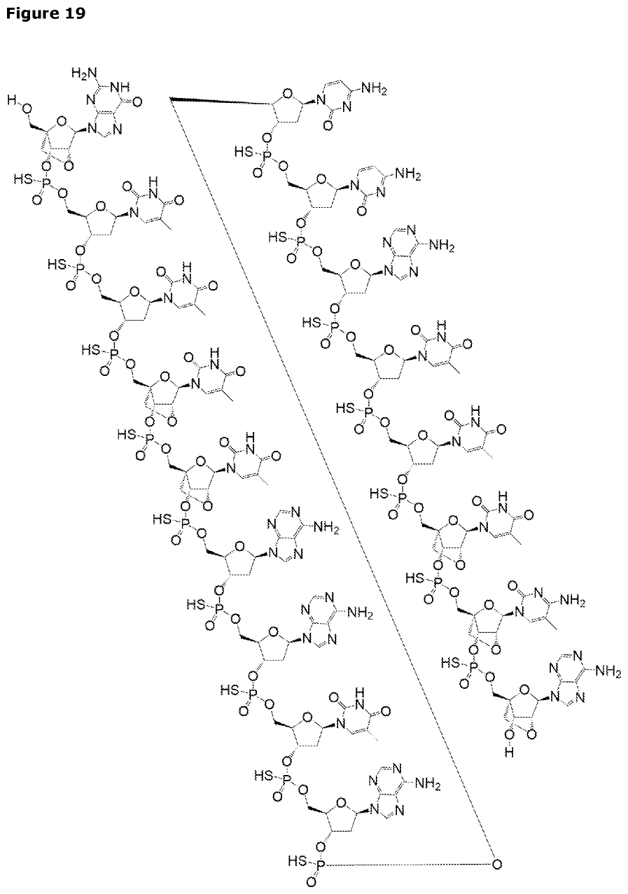

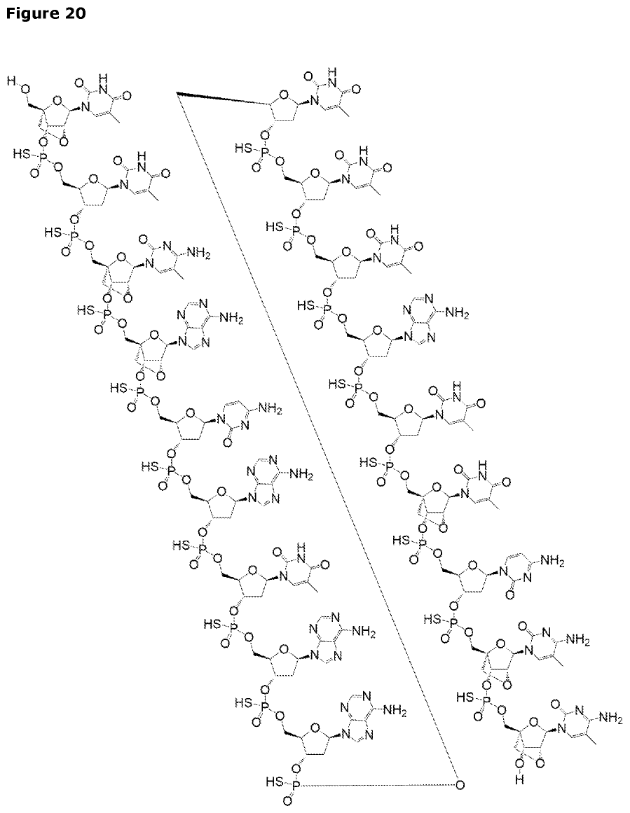

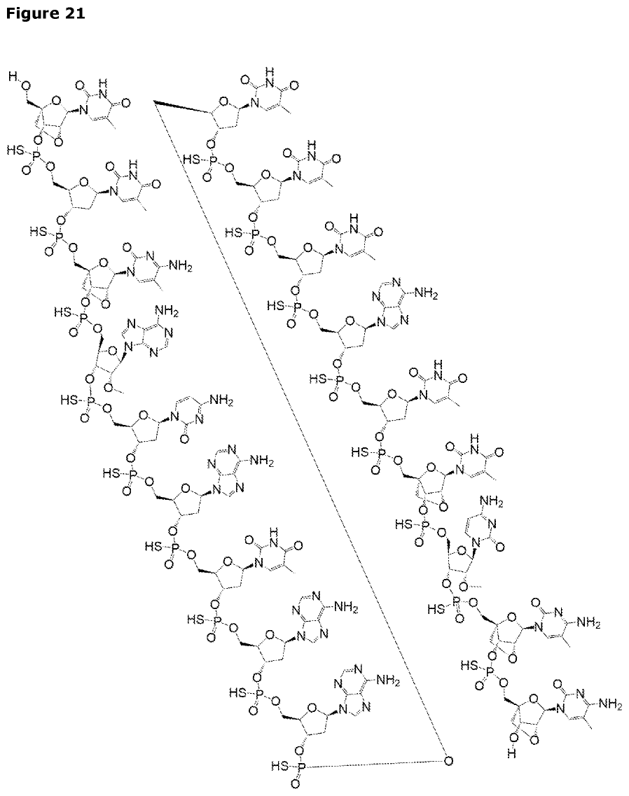

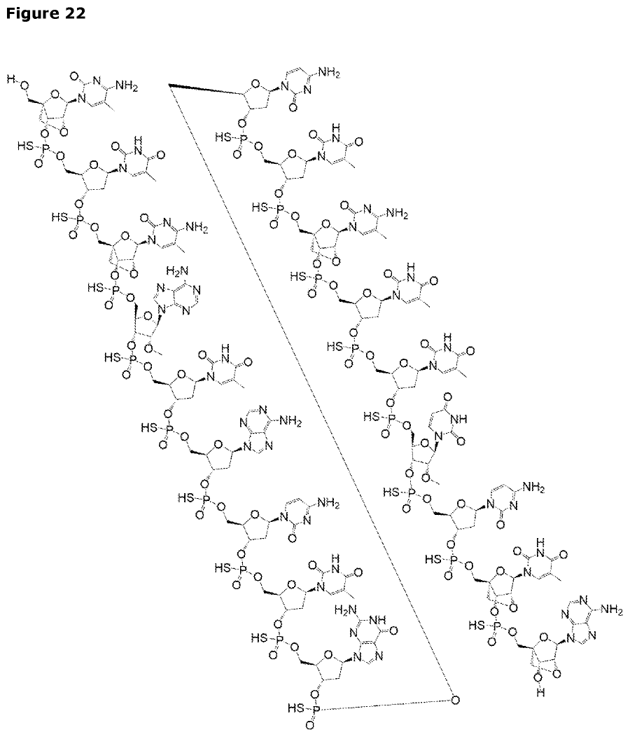

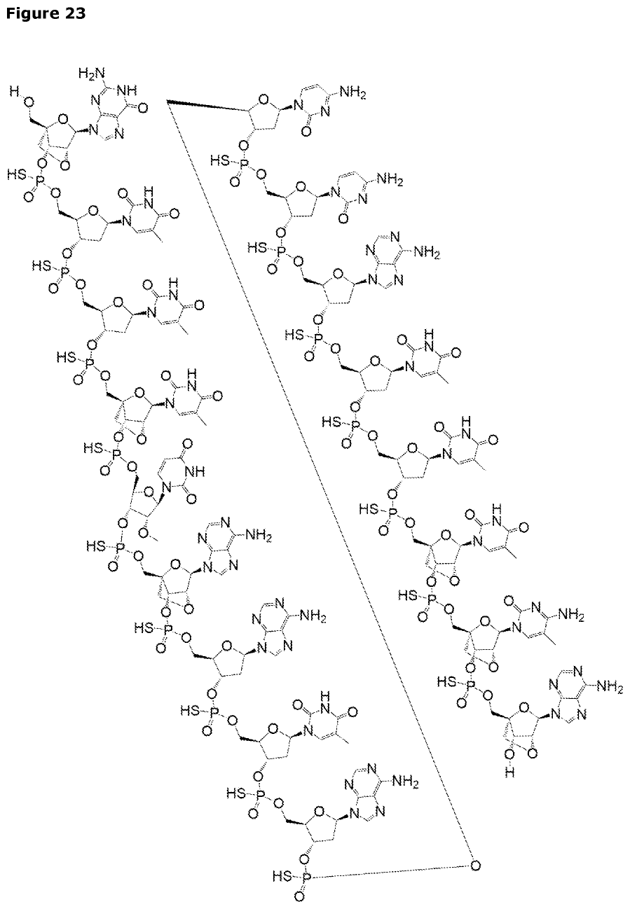

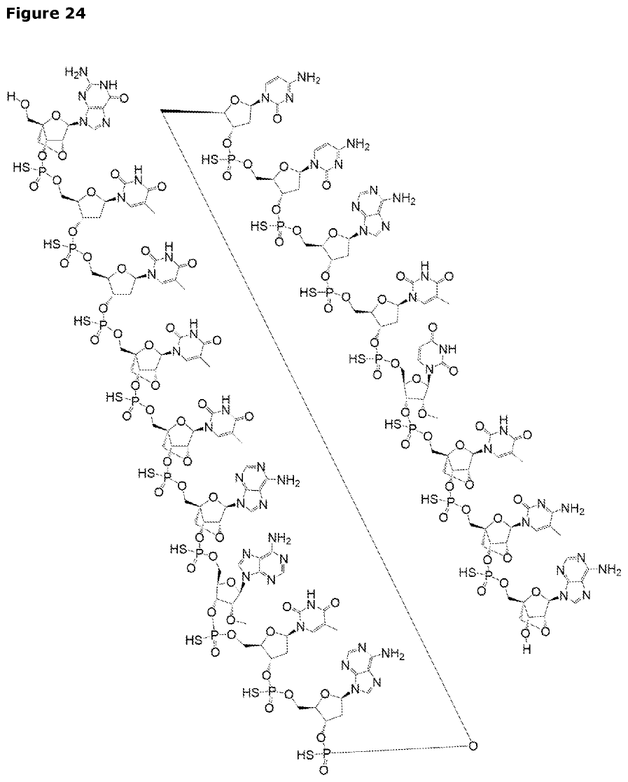

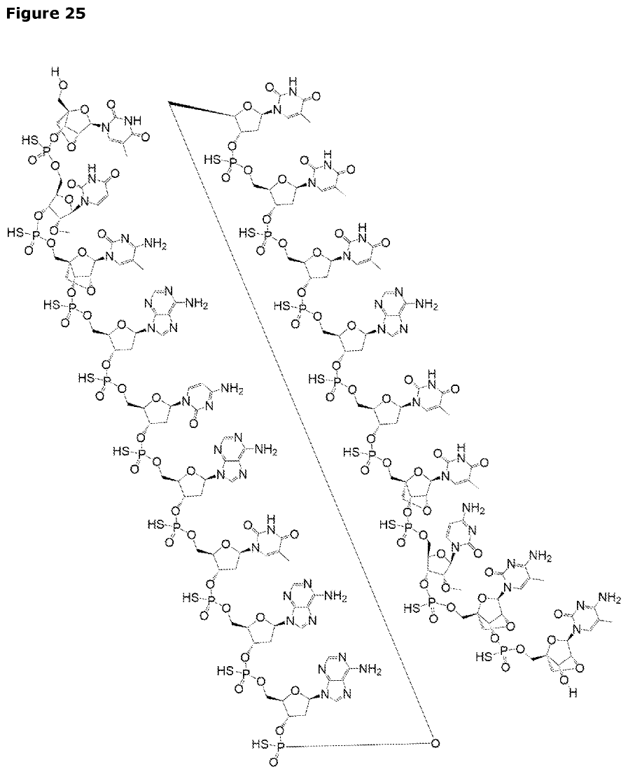

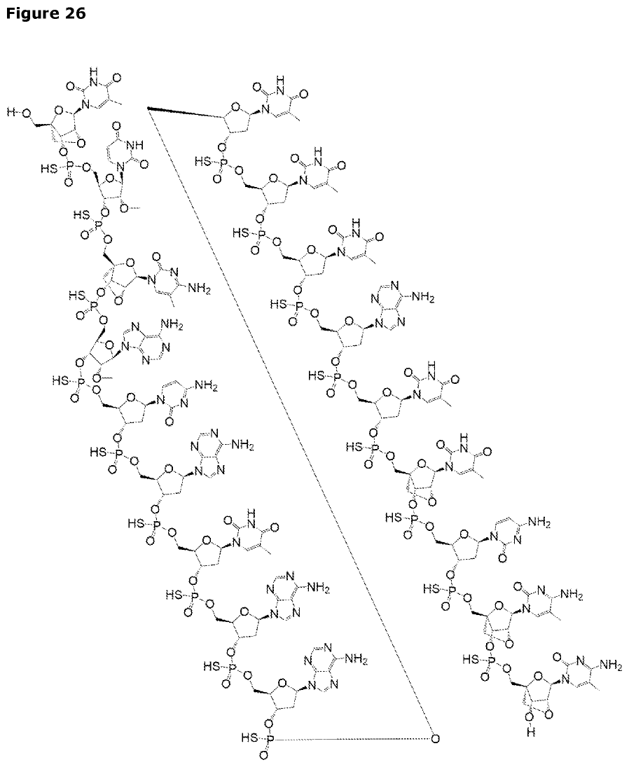

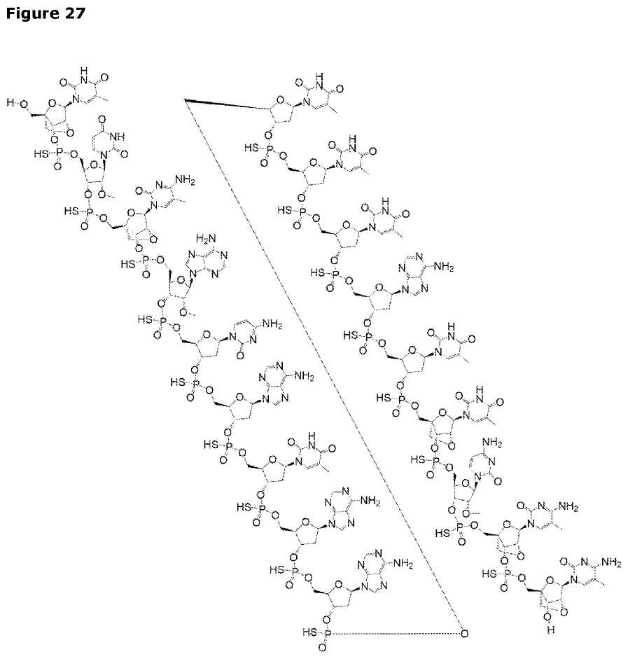

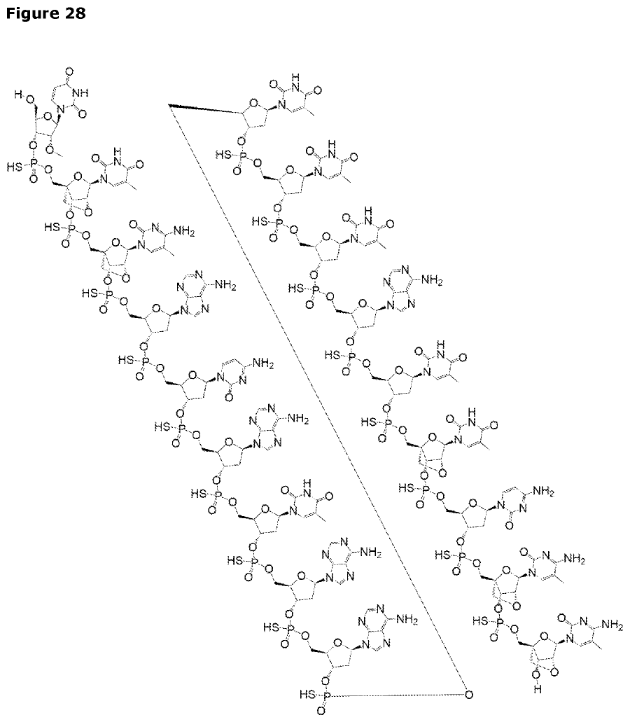

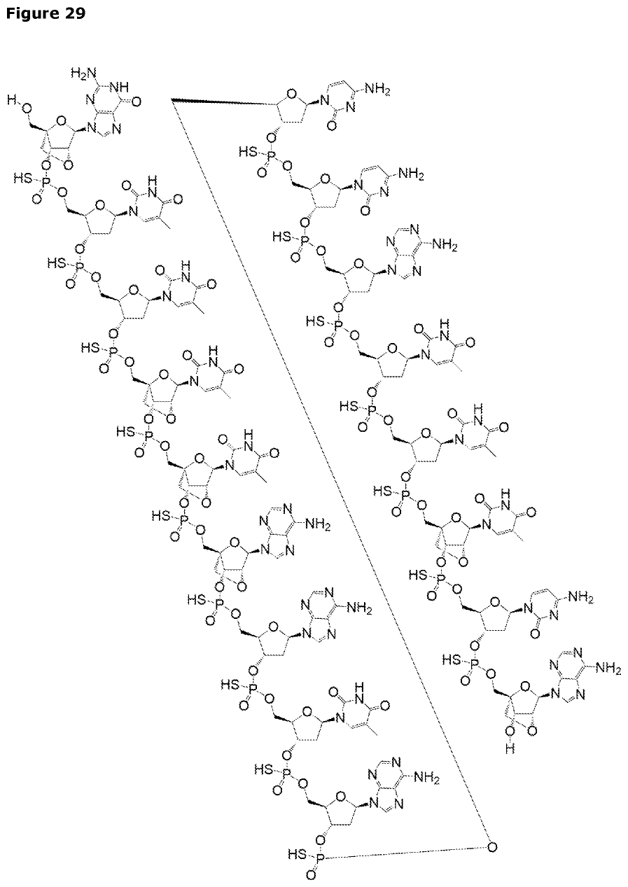

6. An antisense oligonucleotide as shown in any one of FIGS. 1-29, or a pharmaceutically acceptable salt thereof.

7-12. (canceled)

13. An antisense oligonucleotide of formula CAgtTttaataccatTTC (CMP ID NO: 31_1) or a pharmaceutically acceptable salt thereof, wherein capital letters are beta-D-oxy LNA nucleosides, lowercase letters are DNA nucleosides, all LNA C nucleobases are 5-methyl cytosine, and all internucleoside linkages are phosphorothioate internucleoside linkages.

14-24. (canceled)

25. An antisense oligonucleotide of formula TtCAcataatttatTcCC (CMP ID NO: 39_9) or a pharmaceutically acceptable salt thereof wherein capital letters are beta-D-oxy LNA nucleosides, lowercase letters are DNA nucleosides, all LNA C nucleobases are 5-methyl cytosine, and all internucleoside linkages are phosphorothioate internucleoside linkages.

26-34. (canceled)

35. A conjugate comprising the antisense oligonucleotide of claim 13, and at least one conjugate moiety covalently attached to the antisense oligonucleotide.

36-37. (canceled)

38. A pharmaceutical composition comprising the antisense oligonucleotide of claim 13 and a pharmaceutically acceptable diluent, solvent, carrier, salt, and/or adjuvant.

39. A method for inhibiting SCN9A expression in a target cell which is expressing SCN9A the method comprising administering the antisense oligonucleotide of claim 13 in an effective amount to the cell.

40. The method of claim 39, wherein the method is an in vivo method or an in vitro method.

41. A method for treating or preventing pain in a human subject who is suffering from or is likely to suffer from pain, the method comprising administering a therapeutically or prophylactically effective amount of the antisense oligonucleotide of claim 13 to the subject, thereby preventing or alleviating the pain.

42. The method of claim 41, wherein the pain is:
(a) chronic pain, neuropathic pain, inflammatory pain, spontaneous pain;
(b) nociceptive pain;
(c) pain caused by or associated with a disorder selected from the group consisting of diabetic neuropathies, cancer, cranial neuralgia, postherpetic neuralgia, and post-surgical neuralgia;
(d) pain caused by or associated with inherited erythromelalgia (EIM) or paroxysmal extreme pain disorder (PEPD) or trigeminal neuralgia;
(e) chronic pain, nociceptive pain, neuropathic pain, visceral pain, or mixed pain; or
(f) lower back pain or inflammatory arthritis.

43-46. (canceled)


SEQUENCE LISTING (52 sequences)

SEQ ID NO: 1 - DNA, 180817 bp, hsapiens
tgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgctagcggcaggcgtcccctgagcaacaggagcccagagaaaaagaagcagccctgagagagcgccggggaaggagaggcccgcgccctctcctggagccagattctgcaggtgcactgggtggggatgatcggcgggctaggttgcaagtaagtgccttttcttttgctgcttctgtggggaggggaggagaagccctcggtctttcgcccgtgctcgcctcaaccgccacacccacctgtgtctattttcacatctcggttctggaaccctgtattacgccacctggaaagaagagaggggagaagcttgaccgggtggttccagcaatgggaggaagaagggctggaagcgtagttatttcgatagctgggcagctcctgtgcggttttgactctgtgggtttgctgccaggttctgaaactgtggaaaggaagggtagcaatgcctggtggtttactgtcttttatttctgtttacgctagcagagttgttagatggacttcaattcaaggggcagctcacattttttacgagcaaaaagcatgtgctgtatttcccatctgcaaagctgagcgtgcagaaccagcttagcagatacaacctgtggggacagaaaagaaccccgcaccccttaagagcacacaaacacaaataattgacaggacagctctgcccctatttctcagcgcagattatttatttattttttcgaataagcactttatagtgaaattcagaaaagatgcagaatccttttaactttttatcgaattcaacttgggaaactcatttacttttaggaaaaaatggatattttcgtgccttatattcaagacaaaaatcagaacaagaaactagaaacattaatgccagtttttgtgattatagtaaacatttcactcattattttagttttttttttttaagttggagcgttcatagattatatctactctgatgttaagagcaatttacatgagaaacttaggggggtttttaagaagctagtgtcagtaagataaaagccaaatcaaagtaagctttcaaggaagtgatagcttgtcagttactgaataggaagtcagtgttgggaagttcagttcttttcagaaacatatttattattcttttctatttcttcttccctgtagacaagataaagattttctttctctttctttccttgttatttaaatatattttataatttttgctttaacatcaatataaacctattttatataaactgcaaaccaaatccaattattaaactgttcattttattgcaaattcttcagattttttccactatttaaaaaagtttataattttttcttataattatggtgagattttttaaaaattattgattagtttgaaagctagtcttatctgatccctgtgtcgcaaattttcatgattgttagactagcttatcaacaacaaaactctagacagaatatggtatttgatgtaaagaagaatgtgccctctatttaggcaactgatagtttggcttaagacaagttttttaaaattttgtagaggtttaaaaggaggtttccttaagtaagccactgtgcagttgtgggactcagtgatgtgtttttacagtgactggatatatgatatgccttgattcctatggtgcagggctaggctcacagagtacctgatgcttctacagaagaaagatgagtcaggtctggctctctgccaaaagcatcttgggcctcctttcccctagggcctgcatataatcatgggagcccttctgtggtaatgcttctatgtttttactacctgttgctgtgctgagtttcagaagcaaaatagataaacaaataaataaattcagaagagcaatcagtagggttctatttatttaatataatcaacccactttctctgtggcaaggaaaaaagtagaatttttcattttacatgaataataaaatctattttttatccaatttctattcctattagttatgcaaaagagtgtgaagtgctagaaggatttatatggtacttttctaagacaagtcatgatcttgtaaaagagcttaaagtttattgatgtaaatggaacattctagatgtcataatgataataatttatgactttccctaaaactctgtctcagattctttcctcccatcttcattatctgaataaatgccttcaccatccaagtattgaaatcacctttgattgctctcttccttattactccatttcagccctttccagcctgtaagtagatctatcaatcttatctcccaaacttatttctatttcatcatacgtctcagatgcctctcattcaccctcttttctgtatctacacggccaccaaattagttcaggtcatcatcataatttacttggaatcctgccacagcctccatatcaatctctccacttctgtaattttctcccctgcaattcattctctatacaaagcaataccatcctgtctcccctaacaaacattggctcacaactgcatttaaaaaaaaaaaaaaagaggaagaagaagaggagaagaagaaataaacatggcaacacagagaccaattagcaacaacagtaaatgcctttcctcagcatatggtttactctctgttctgtcccttctctccaacctcaccagaacctattctcttctcattaaggtgctcctgcccaggtcgtggctaaaacacatgaaacaatagcactctttaaggccttttcaaatgctgttcccaatgcctgaactctatgcgtgcttactccttcttaaactttaaatctcaactcgaggtttttttgttttggaaagatcatttttgatcatcttgttaaaagtaggttgtcccgtgttggtcttgatgttggaaccttgtttctttcatactgcttgttacagcatgcaggtattttgtgaatttgcataccaaatactccaagggaacccagactatgtcttcattgttgacttagcttccagaatagtgcctggcatcaagtagtagggggttcgtagatatttatataaataataaaatacaaaaatatggattcttccagaataaagcatattaaccctggaaacatgaattttgtgattctattttttatttgtcaagcatttagtatttttacaatatcatggtatgatattgaggaggtaaaattaaaatatcctgagatgtattctctcttttatcagtgtaggtgataatgtacttaaaggtaagtaatttgtaatgcattcttagaaaggcagtgaccacaaatgtattaaaatatattttaagaaaaaattggattgcataaactaatatggttataagtgactccaaatctaaaatgataaccttgttaggtttaaatctgtatcatctaacagaaatttgagggccagtcaatagtcactgactatgaatttaaaatattcttttaaaataaacatagatgcttatgtgagctcagttgatagaaatgtagtcagatgaggcggaggccataagttaggtttccacacggtctaaattgatgtttgccccaggtacaaagggcacattatccaaatatttgaagcattctctaaggaaggttcagagaggcaatgtgggcttatccatgaagacccatcactgttattagagtggaagtaaggactgcaaacttcatgctcataaatgattgtgtcattttcatactagaattttaccacacacacactcacactcacacacacacacacacacactttgtatggtgatttgcctcccccctttatttttgcagactatgcttaaagatattaaacaaacaactagttttgctattaacatttggttgaaaatttagctttcaattaggtgacaaactgattaatgatttttttatgtagcaaatggccagccacagatttttggtaaataatttggttaccacattacatcttgaaggactaaattatttcactaattgtgagcagggatttttaaaaatttgctctgaagaggacatattgtggatgttgtattggagggcatctgtgtactaattgaagatggtccttcatattacacatcacaaattagctaatttattctttacagaagtattatgacatcttcctttagaactgtgtagagttagattaccacttgagaggcaaggtcatattaaaatagatattataaaattgtataaaaatattttatatgaggatttatgagagcatccagagaatgtgggtagaatacttttgtggaatatgagataacaattttggtgtcctgccattctaaaaaatgaaatttcttcattgaaggtatcacttaccctttcctctgccttgctggaaggaaatggctttaggcaaaagaaaaagccatcagctcttggcagctaaaggtggaatgaagtttggaggaatgaaacccttctgggtggtgaggaaaaatgccaggccatgataaagtggggaaggaagaggggaatgccttgaacagtgccagtaagcataatctgccacagacaagtttgaattgacatatgtagaactgaattattctatatctttttagttctatagagaaaaccgttctttccacgtctctttttaaaataacaacttactgtaagcaaaaaatatgttgttgttgtttctttaacacaggagatgaactatctttagattttttaaagtgatataacatggagccaaaaatgccatatattactgttgaaacctaacgttactttaactgtaatttccttgctattcattttatgatttcctcttgttatgtcaagtgttagagagactgctattactgatcataaatttctttaaaatgtctccttggattttttacaaagctttctgaatcatttatcaaaatacaataaagctctaattgcacctaggattgttcacttggaaatttagaaagagtgtgtacatttacaattaagttagcatcctgccctgcaatgaatgccctttttctttttttttttccttcttctctcctacatctcatcttccaaatgtttgatatgtacgccagtaaggcaaattttactccacttttaggtcaaactattagtacaaataattggaaaattgagcctctaaagaaagataaataagattagaagcatgcattaaaatataagtattaaaaacattcccataagatttttaataaaaattttatgtgctatatgcacacctagactccctgtattatctgatttttaaagattcttttttttttttattttttatttttttgagacggagtctcgctctgtcgcccaggctggagtgcagtggcgcgatcttggctcactgcaagctccgcgtcctgggttcacaccattctcctgcctcagcctccgagtagctgggactacaggcgcctaccaccgtgcccggctaatgtttttgtatttttagtagagacggagtttcaccatgttagccaggatggtctcaatctcctgacctagtgatccgcccgcctcgacctcccaaagtgctgggattacaagcatgagccaccgcgctcagcccattatctaatgatgatgatgatgattattattattattattattattattattattattattattattttaactgggggtaatttcactggatgtggtatctgtcaacttttactagttgctcctacactatatgtttttaaatccaatgtaattattttaaaaatatgtcatcttcttgagagtctatactttagaatttgaaaaaagaagtaaatgtagatattcaaatccatgcagatgagtaaaacagggaaaaatgagaaaaatgtttcatgataataaaatattatgtcaggcacagctggtcacccctgcaatcccatcactttgggaggccgaggctggaggattgcttgatccaggagttctagatcagcctgggcaataaaaggaaaatctgtttctacaaaaaaattaaaatttatatttcatatattgatttaagctctactaaatgactattctttcaagtctaatagacagtgtatcttttagggctcagagtaaagataatttattgaggacattttgaataggatgaattaaatgctttattggtaagtgatgcctttatctgagtgattccttctaggtaaatattatgggaccaatctttgtaaatgatctttgcatggaacaaaatattataaagttgaagtgatgaatttgtttccctcttaacaaaatatgtattcttgttaccccatttacaaacttggctcatggtaattcaggtaggaaaaagggaagtggcctactcagggatcaactgggaataaggttcttataatcccatgagatcagctgacagacttttaggaatgaacacttgtagaacagctaggcagagaagtgcctttcaaaatttatattacatctctactatatttcagactgtaaaatatttttataaagtgttaaaagcacagataaataaatttgagttcaaattagtatagtgaaaggaagtttatttttcccaaatatttcctttactcagtatgtggtcttagtcaaatattaatttcttaaatattgctttcctgtaaattagtagtatttacttctaaggactatattatatgttatatatagagtatattatatattaggagatatatatgtatatacatcgtaatttgttttttccaaaacagccactttagtgtcttacacatgataagtagatatgagtagatgttcaataatggtatctaggcatttaataacttagaatattataatcaaaatgcattcttttttttttttttgagaccgagtttcactcttgttgcccaggctggagtgcaatggtacgatcttggctcactgcaacttccgcctcccaggttcaagcaattctcctgcctcagcctcccgagtagccgggattacaggcatgagccacctcgcctggctaattttgtatttttagtagagataggatttcaccaggttgggcaggctggtcttgaactcctgagttcaggtgatctgcccgcctaggccccccaaagtgctgggattataggcgtgagccaccatgcccagcctctttttttttttttttttaattaagtttcaaggtacatttgcaggatgtgcaggttttttacataggtaaaggtgtgccggtttcactctgttgcccaggctggagtgcagtggtacgatgttggctcgctgcaacctctgcctcccagattcaagtgattctcctgcctcagcctcccaagtagctgggattacaggcatgtaccaccatgcccggctaattttgcagttttagtagagatggggtttcaccatgttggccagactagtctcgaattcctgacctcaggtgatctgcccacctcggccttctaaagtgttgggattacaggtgtaagccactgtgcaaggccagcaattatgctgttacaaggaaaaatacatacggaaaagtttacatcttgctaacattactgataatattaatgtaacagtcttatgtatggacgtctccttaatgctaactgagcagtttgttaagggattttcatatgctccgtttaaatcctcatgaccaccatttgaggtgagaacactaaggctcagaaattacctaacctttccaaagactcacaatggttactaaacttgaataacatacacatttcttttataggaaagaaatcttatatattccaagaatacgtttgcacataccccacacagatatacctggtggttacgctaccaagcattctatcaaatatgtttaacccttaatggagctaaaataaaattacatttgcaatcaaacatactaaatcatgtaattacagctaatagaaaacaacaaaaaaagaaagatggatgactgtttcaattttcttcttggacaccaattcctgaggggtatattgtgttaagtcaaaatagagattttggtggaagtttgaagacattatgaaaagagtacctatgcatttaaagtgtttttgaagttgttttataggttgaatgacttcactttcctggtctaggagtattattgcactactgtgacttgactttgctatgattcgcatattttgttttaacattaaaaaactgaaatgggaaaaaggccttccccaaggtagaaaagatttgaaaaaagaggaatggtgtaattaaaagggaaatatgtataaaatgtctagggtgggccaggcgcggtggcttacgcccgtaatccagcactttggaaggctgaggcaggctgatcacttgaggtcaggagttggagaccagcctggtcaacatagtgaaaccccatctctactaaaaaaaatacaaaaactatccaggcatggtggccagcacctgtagtcccagcttctcgggaggctgaggcaggagaattgcctgaacctgggagatggaggttgcagtgagctgagatcgagccactgcactcctgcctgggcaacagagcgagactcagtctcaagaaaaaaaattaaaaaaatgtctaggatgaagtttattctgaaatggagaagaaacagggaatgaagttacacacgttgagatcctggagctagcagatggagaagtatgaaaagcaaagtgatcttgtgcaggataagatgatggtgttcattggaagtatttatatatcttggaaaataaaaatttagatattctatattggaggagtacaaatatttagagcatcacaaattgatacacacaaatgaaacctaaatcacagtacttattgtcatgaagactcaattatctaagtcagttaaattgtctaacatcagtaatgcaagaagttgacttttaagggttttaaaacagttttggaaaactaatttattttacattatgtttataataatttttgcatgcaatttttttaaatatctattatccacttagatactgtactttgggatataaaaaccatgggaaatttggcccttgagttctatgatacgtctcaatctattttagtgatttgtggattggtagcttatcctctagactaagtggttaaggttttatcagcactgcaatgagataatcatctcacacctgttaagatggctgttataaaaaagtgaaaggtgttgttgagaatgtgaagaaaatgaaagcattgtccactgttggtgggaatgtataatagtgtagctgctatggaaaataatatggaagttcctcaaaaaattaaaaatagaactaccatatgatccagcaattgaaatcagaatctcaaagagatatatgcacttctgtgtttactgcagcattattcacaatagcaaaaatatgaaaacaacccaaatgatcatcaacagataaaagagtaaagaaaatgtggtctgtatataaaatagaatattatctagccttaaaaaaagaaggaaatcttactatttgtgacagcatgcatggacctagaagagacatgctaagtgaaataagtaaagggacacgaaaacttttgacagtgacagatatgcttaacaggtagactggcagataagaaaaaacaaagttaaagctgaaatttccaactgatttatcttagcctagggattaatcattagatccggtctagttggcctccaaaatgtgtaggtcaggagttggcatccaagttggtattggtgccattgaggtttgtagggagggaagacctggcagagatcccaagggcagtggtaagcggctgctgagtccctccaggccagttccaagaactcacaacctcactcctctcagcatttatactcttcctggaatttaaaagcaattccttctttccttccctctttcttttttccctctcttccttcttcataaagttttattgtagacatcagatatagcaatgaacaacacatggagatgtgacgccgattttaagagataatgaagagataaatattcgccactatccatcattgtaccataagatatgctgtgaaaaacaataaaacaggacagagaattgagagaaggaggtattatttaaagtggtctgaaaaattttatacagggtatttgagtagagacatgaagtaaatgaaaaactaagggagctagctaaacaactgaagaatcattctagtcacaaaaaacagcaattgcaaaagccctaaggcatatttgatgtatttaaggagaagcaagaaaaccagcatggttgaaatacagacagcaagattgaaagcagaagatgagggtttttcttgattttgtagggattactaacctgcgtcgagggctgcgtgataaaccaatgctaaaattaccagcatgggtacaactttggaaagggatgagtgtgctcttttttaactggatctctttctggacatgggtaacataaaaccagaaaaaaatgtttgagaagtttctaatttctatacaaaaataatgtttttgtctttttttgtgatttagatgtaaatgtatatgtacatgcatgtatatgtatttatgtactgatttactatttatgtatatgtaaatgtatttatatttaaatgtcctatattctcttcctgtctgtaatggattggattcttggaacctcttactatttggggcagggggcagggctaataagacaaagattacaattgagagtaactttaaatgtttgcattttggaaacttaatggtgggagtacagtaatattttgtatatttcgatcaattttgatgtttattatatgattatttctcatgaaatagtggtactgcatagatataaatgactactgcctagcaaaacagataccaaggagacaccgcatgttgtcagggttgaccggttgctaactgctgtgattttgttctcttactgactctttctgccttacggatctcaaaacacgctagtgaatttaaaaatttatattttggtaaaatatatataacaaaatttaccattttaaccatttcaaatgtacagtttagaggtattaagcacattcatgttgtacacgatcaccaccatccatcttcagaacattttcatcattccaaacaaaaactcggtaactattaaacaatgactccacactcctctcttcccctatcacccagtaaccactattctactttctctatgaatgtgactattctaggtacctcatgtaagtggaaccctatagcatttgtcttttcatgtctggcttatttcacttagcataatgttttcaaggtgttcatccgggtcgtagcatatatcagaattttattcatttttaaggctgtgtagtattttattgtgcatttatacacattttgtttatctgttcatctgttgatagacatttgggttgttttcactttttggctactgtgattagtgctgctatgaacattggtgtacagataactacatgcgtcccttctattaattgtttaggatgtataccctaaagtagaattgctggatcatgtgatatttctgtttaattttttgaggaaccaccacagtgttttccacagcagctgcactattttatattcctatcagcaatgcacaagggttacaatttctccacacccttgtcaatgtttcttttccttttttttttttaaacaataaccatccgaccaagtgtgaagtgatattttattgtggttttgatttgcatttcccaaatgatcggtgatgttgagcatcttcccatgtactgattgactttctttggaaaaatgtcttttcaaatcctttgttcatttttaatctgcttgtttattttttgttgaggagtaggaattttttacatattgccaatgttaatcccttatcagatatatgatttgcaattattttctcccatcatatggattgccttttcactttctagtagtgttctttgatgcacaacagtttcaattttgacaaaatccaattcagctctttttttcttttgttgaccgtgcttttggttccatgtccatgtgccaaatctaatgtctcgaagatttttccctatgatattttcttagaaatttacagttctaactctgacgtttaggtctgtgattcattggtgattttttaaaaattcctatataattcgtagactctaagagagtttccagctttcccatacccattgtccccagagatctggtttttagtctgaatgtgccttactctgaacatgtgccttccatttcaagaactcccatttcattttgttttgttctgttttttcttttataccagtattaaaacttttgagttgttgtataaatatactagcaatttaggagaattcctgatgggaaagaatatccttgcctagctagttaatgaatttagggacccttcattttctaatcctagatatgattaaatcaaacaaggattcattgtgctgtagttgtaatccacacagaccattttacaaggctttgcacagagaaggtgcttaatacttgagtaaacaaattgaattaatgagtaagaaagaaaagctatcttgttttctcaacggtcaagtagatcaatgatttctactatttagaatcttagatttattttaataaagcaagttcaaaaatctcctctctctgttaatttttagtaaagtcacttatttttcactcagttttgttaaagtgttttcagaagagtttgttaccgaaaactgaagttttaaggttagtgtttataccacagatatttcttgtttttagacaatccttgtggaggttaagtcccctaggcaataccttttcccactggggagttacaagcaaatgaaaaatggtgactatttatggaaactccaggtcactaagtaaattttgcattttcatgaaaatatgacatatggacatttaaaaaaatgaacaggtcaattcatcagttagaataataaaaagaagtctgtaaataaaagttaagcacctttgtcattgaattgttgtgaaccgtttttgcctatgcacttgctttatctcagaatccaagcctcctggtcaaagttatattcctaataaatttaaaaataataatttaataattttaattccttctggaatataaatttgatcaggagtcttggattctgagataaagcaaatgtttaggcaaaacacttccttctggaatataaatttgaggaaggataggaattattttcttcctattcatttgtgatcctgtgattgttatctaatttggaatattcttcatgactccaatcctatttatatctgtgaggttctgaaaattctgccccaaaatggcagaaagtaagaaatactatcacacatttaattcacccttaaaaaaatagccataccctttatctttgaatatatttttaacaacaacaacagaaagagaatgcattttaaacaatatcacatgagtttcatcatattcccagattatggtgagggttcctagataccttgccataatcacagcctttagttccaagagagagcagagaaataagaaacacaggaagtcatatctatgaacatgttagtttgcaaaaggtttgttagtaaactcatgttatttataccatagtactttaccatctccagatgaatttccactaaataaactgttcctttagactcctcaaaccagattacccccttcctttgctaatatagattctcaaacaatgtcttcatcaaaaatcttatgagtagatgtcactagttttgacaatgtccacaaatgttccaaagaatactttggcatgcatgcttcttaactcacttattttgatattgaattaaagggcttttaacaattcctgccccatcaggaatatttcatcagcagctgttactgtgcttggattttgccttcttgaatgaatacctagtcttttctcactttagcattaagttctggagcatccgctgctaatgaaaaatcctctgactcatgctagagcattttcatttttttaaaaaaacaatgttccttgtatcttgctggctattgcctaagctcattattttaaacttttgcataattctgctcagtcctagagggtcatcattactgtcaatgattaaacttttatgcacttgccacatttattattttcttactttcgatcattttattatttttctttggttcccatgatattccaatgtttttgttattaaatctttcaacatcctcggtgtgttcagtgtatccacatttatcaaacctgatggatttaagaatttatataaccttgaaagcaaggcccacatcctgtagacactgaataaactaatgaactttgtattggcaggactaaaatggctgtgtgtggcatatattgccacctgttggtagaaggcaatgtagtcttggttttgctataagcatacattttagtctttaaaatattctgagttgaaagctccctaagaagaaaaggttttgtatatgcaatacaaaagcttttctattataaagtaagcaaatatattctcgttcctaagaacattagctttaggtatacaaaatcaacttagaagaaagttgctaaaatcactaataataacagttacaatgtattgtgcactgaaagtgtaacaggaaatgtgctaagttattaaaattcagtgtctaattttttccactaaaataaccttaaaggattatcattttcagctccggtttgctatataagaactgagctttagagaagttaattaaattccccaaggacataagctagtaagcagtagagatgggatgtgaacccatgcctctctcactctggagccatgaatttaacaactgggttatgctgtcttactattttactgcagatctatattttaaattaaagccataaacatgtttggcaagataaaacataggtaacaaatatactccacataagattttcgtaaggttttgtgtttattggccaactcatggcttattatttaaaaatttcagtctgtatagggaagaaggataataatcttgctgttatttatttagcctaggcactgtggtaggcactttaccaaaaccatttctattctacatgataactttataagacagatatatttctctctttcatttatggggaatttgcataatgctgcagagctaattatatggaataaacaggaaattcaaacctaagtctgcctggtgccaaagttattactatgcactttctcctaaatcatgttgatgttttgagggaagtggatacatgtataataatattggaaatacattttctctctaattatttaaataagggtatagaacctgttctgcagccataagtctgtgataattttgtgctctcaataaagtcttaactaacagagagatgggatttaacgtgaactaagaagtattttctgtcttcctttatacatgctgtacagataagactttatgaattttttattaaaacgttcagatatgccagcaaatgttgacactaatcttaagttttcccagggtaatacacttcttaagattataaactttaatttctttttttttttttcttttttttttttttttgagacagagtttcgctcttgttgcccaggctggagtgcaatggcccgatctcagctcactacaatccacctcccgggttcaagcagttctcctgcctcagcctcctgagtagctgggattacaggcatgcgccaccatgaccagctaattttgtatttttagtagagacggggtttctccatgttggtcaggctggtcttgaattcctgacctcaggtgatccgcctgcctcggcttcccaaagtgctgggattataggcgtgagccaccacgcccggcaaactttcatttcttataaacttgaggaatactatattttaaaaggctatacataagactaacttctatcttaataataaaataataaatataatataaatgattattacagggtattatgaaatacccaaatcacaatattttaataatcatagaatttagaaatagtccctatcagttgtctgaatataaaattatcgcatatacagcttaatttgttttgcataccagttgttcaactactataaaatgccataaaattgaatgattttaggacatgaaatattgaactactcttgttgaagttattaaactgatttgccatctaccaaattaagcatttctaagaaataataattattttaagaatttatttacatgattaacataatgttacttatgaaggatgcaatggtaagatttcagcaatcaggcaagtatttaatgaaaagtatttggtttcatatatattgtatttgatttcatatatattgatacacttaacattagaggttctaaaatgagtagccagcctgcctgtttttcttgtgatattatacttggcatactttgtttttataatctaaagaagaggtattaaaaaaagagtcagagtgcttaccattggcagttccttttaaccttttaatttataaagacattgcaatcccaatgaaaataaaaatagaaattttcccatttggaattagataatctggatctagttgggaaattgtacacacataaaagagtaatatctaatagcaccaaccctaatacataatgttattttatgcaaatagggtaattaaaatagtatggtgacctacatagatagttcattggaagaaaatacgatatccagaaatagacatagttacatatgagaattataaaggtagcatcttaaataggttgaaagaaatattatttaatagttatcattgggacacctgggtagtcatgtggataaaaataaattcagattactgctttgccacctaacccaagatacattttagtggaatcaaatatttaaaataacatacaaaccacaaaactgctagaagaaaacatgtgaaggttttgttttcttttttcttttcacctgagatggataatatatcaatgtcattacaaggtttgagggaggaatatctcacatatgcatgtgaaaacccagtcatcacacttatgaagtacttgaagtagaaattattttctaagataatataaaacacaggagccactggagaaaggattggtgaattcaatttaattaaaatgtagatgcttttaaaagataaactaccataaaccaaaagaaacaaaaacagcaaacttacaaaaatatttgtattgtataccacagaaaaaaagctaattttattattttgttaagagttcctaaattaataaaacactgatagtctattaaaaaagtggacaagactacatacagacagagcacagaaaaaaaagcacaaatggcatctgaggatatgaaaagatccctgacctcactcacaaaagaaaatgcaaactaaaatgataagaattaagaagttttcggccgggtgtggtggctcatgtttgtgatcccagcactttgggaagctgagatgggtggatcacatgaggtcaggagttggagaccagcctggccaacatggtgaaaacctgtctctactaaaggtacaaaaattagccgggtgtggtggtgggtacctctaatccgagctactcaggaggctgaggcaggagaactgcttgaacccaggaggcagatgttgcagtgagctgagatcgcaccaaggcactccagcctgggcaatgagagagcgactccatctcaaaataaataaataaataaataaataaataaataaataaataaataaataaaagttttattccccacttattaggctcacaaagatgaaaaaaaatttatgaaatactttattgaaggtatgtgcaaacaatccctgacacattgtttggggagagcatggattgataggcctttatgaaggagttaccaatataccaataacctccttggtagtttcaaaaggtggcatctttccctgtttggtattggagacccaatacaagaaatgggaaagtgagcatcaagcaatgcaagctttattcgatggccaaagaatggagaagtgcaagtatggcgcacaagctaacttcttgactagtgaggagtgaggaaattgaaatagaaagtatatctaatgaagaagttggtcattaagagtgaggggaggaatgttcatgtcttttccagaaataggtggtgaacttcttggaaccagagtgccgccttcctttttgtccttttatggcttcttctggtcattgtcatggggattatcaactgtcatggcactggtgggggtgtcatttagcatgaaaatgagattataatgatgcctgaggtcttcttgaagtcctttagtcaactatcttggttctaagcaatctcagctgctctggttacaaatggaacattttacctgtttcttaaagatcagcagagttagggtaggatagaaattcagctatggccctgtaggcgttacactaggtaacaaaagagaattcggcaatattttattatggcaaattaaaaacgcacatacaattatgactcagcaatttcactctgaagcatttgtcttacaaataaaataccgtactgtgaaactattcaagagaatgtcctcacttataagtgagagctaaatgatgagaacacatggacacataggtgggaacaacacacactggggcccattggagggtggagggtggcaggagggaggggatcaggaaaaataactaataagtacttggcttaagaccttggtgatgaaataagatgtagaacaaaaccctgtaacacaagtttccctgtgtaacaaacctgtgcatgtacccctgaacttaaaataaaagttacaaaaagagcccacaaaagagtaggttaaaaaaagaaaaaaactaatgagaagataattattgtagctttgggaaacaacctaaatgttctacagtgtgggaatgactaaataatttgggtacagctgtacaataggactttatgaagctgctataaaaagaagtagctttatacatgctgatacagaaagatttccaagtctgatttttaagtgacgaaggaaggtatgaaacaatgtttatatagggtattatttgcatgaaaagaaaaatgaatagatgtatacatgctgataaaaccacatggatggatacacaaggggttggtactgtggttgtccacaggggaggaatttggatggctgggaaacattgaagagtgagagaattatttttcgttatacatacttttgagttttgtggctaaatttattcttcaaaaataaatataagaatgctatttatggggaaaaaatgggagtcataaacctgcaatttggacctaacttttcaacttaaaaatgttctgttcttggagttactaactgctctgatgcttaggatgataatcaaataatagttcctctaaaatatgtctggaatgaacttgaatgaatgaatgaatgaatgttttttgcaaaccattatgtgtgtaaaaatgtagagtattaatataaggtttatttagaatagagtcatctgcattaaagaaacttcgttatacaaaaatctaaataaaaaaatgttgtacagtagctctgaggaggtggatgacccaaacactgagaaaagaaaagaggcagaggctgagcaggatagttcagtttgaggtaaagaagcagcctcacccagcctcagacaagactccagggactgtgtgaaaaacctcattttggccaggcgcagtggctcacgcctgtaatcccagcattttgggaggccaaggcgggcagatcacaaggtcaagagatcaagaccatcctggccaacatggtgaaaccctgtcgctactaaaaatacaaaaattagctgggcgtggtggcaggcacctatagtcccagctactcaggaggctgaggcaggagaatcacttgaatccaggaggcagaggttatagtgagctgagatcatgccactgcactccagactgagaatctgtcaaaaaaaaaaaaaaatctcatttctaggcctatgtttcccagaaatatgtcatgaaaataaaactgccctcccaatgggaaaacatatatatgcacaaaaaggtaaatatttttaatgttaaagttactgcataattcctttaaagagtaattatatatttctattcatttttgaaaaccagttttagtaaagacaaagttaaaagatgacaaactggcaaaagaaaaaaaaaagaaatttttgtgaaaaggtatcaatatccttagcgtgtgcacacacacccacacacacacacaatcccttggcaaacaagaaaataatgaatatttcaatagaaaaaagatcaagagactaaagacactgaccaaagaagaactagaaatgtttcacgaaattcacattttagataaaaacgttttaaatatcaaattatgaagtttttttttaaatgactatacttaacattgtgggaaaatatatgcttacatgttgctaaaaatgatatacatttttatagcctttctggaaacattttgtcagtatgtatatatatataaaagaagacaggttgggtgtggtggctcatgcctgttatcccagtgctgccaagaggcagaggttgcagtgatggagtgagccgagatcatatcactgcactccagcctgggtgacagagagagactccattaaaaaaaaaaattagccagacatagtggtgcacacctgtagtcctagctacttgggaggctgaggtgggaagaacacttgagcccatgaggtttggaggttgcagtcagctatgattgcaccacagcctgggtaacagtaggagacattgtctctttaaaaaaaaaaatcagtaattttcttggaataggttaatttcatttatttttttctatgcttttcagttttctaaattttctcccaatgaaaatttgctatatttatattcaaccatattaataaccccaaattagtcacatgtgcaactttctgcaaataaatgtgtcttggatttaatggaaatgaaatcttttcagtatagcttctccctgttatttgcgtacaagcttcaaactgcataaactattttaatggcaaaaattaagttagggttaatcaaccatctaggattgtatctgtgcctaggtttctaaaacaattaaatacttgatcgctttgttggatgttcaaagatttggcaatttgtccttaaatatatgtttcaaataaaatatctacaattttaaggtgcttaagtactggtgaacatgtcaaaatgtgcctaatgtatttttaagttctgctgtgaacttaaaaactttgggatttgtgtaatcccaaattagagaattttaaactttagttggtgtccaccatattgtcccccttttcatccatctgaccaaacttttaaccttcctgtttatggctggggaaaaaaaaagctactttaattaaacctaagcagatagtgttgtttttaaattgtatttcgatgtcactaaaatggggaaacaggttgtaatagcagaatccttatattttttgcacatatcagcctccataaaaggatggaaattgctttgtttttcaatgttttaatttttttaatgaggagaaaaagagcaattgagagtgccataaattaaagatagcgtttctgatgtaaaatcaatgatagttatggattattgtaaatgtttgaagatgctcttggaacactgaaaaatcttagaaaattttgttaaataataattaaaattgtcttaaattagtgtgacatctatgttaaaggcaatataattagatagcgtgcttcttcaagtgcttgggttaggagaagtatttcaccatcttcattcatcaagcatttaacaattttatatgatgtgccagatatcatgttataccttggtgataatacatgcctaagacaaatttcctgtctatgaagtttggaatctagttgaaggagacaggcgtgatgaaagaaataaagcactgtagaagtaggataaaaatccgcatttaggggactgctggcagctcaaagcagactggagatatgggagaaagagaagctgatgaagggttgaatttatatttgagggaaggaaggaggcgaagtcagatcttttcatacaagaggtattgagccaaatattacaaagcaagtaatcaggtgatcaggatagttgaaactgttcagataaagggaacattatgaggatgcagaaattatctagctgacctttggggaaattaaacgtagaataatgtagctggaatgtagactgcaaatagggaaacagaagaggctgtcagcatcaggttagtaaaaccttctatgacagaccaaagacatgacaggttattctgtacatagtggggagctgtagattataatttttttttagtcatgggaagattatcaaatttcctattgcagacaaagggtctcttgttcattgaatgaatattacataatttattcaaacttgcttggtgttaggcaactaaatgttttaaggcatttttttgtagcacagctcttttttctcttttttttttttttggaaacagggactcactatttttgtatttttgaagggaaggggtttcatcatgttggtcaggctggacttgaactcctgacctcaagtgatctgcccacttcggcctcccaaagtgctgggattacagacattagccaccacgcccagaaagcacagatcttttttcaaaagaaatttgtgatgttttaataagaaagaaattaaaaaaaaaacagatacaatggaaagatatcgatcctggaacttttaaatcttggtttagagaaaacagtttattatttgaacaacttcaaacactcaagttaaaaagcactaacaaggcacatctcacttgcacaaaaatgataccaattatcaattgttggtggcaacagaatggagcagcacagaaatctggagacccgttttcagttgaatgctcatagttctaaaataggctctgatttggaattccttgtcttacttttcagtttgattaacgtggaagtctcaatatcttcttatgctgtgtgtaggaaaagtatcttgatcagtatatagaagaatatcccacaatgttactgcaactaagaaaaaattaacccattggagaaaacatttttaatcagttcattaagagtctaatgaaggtgcatatacgaaaagcaagatgtactttaattaatgattgatgttttgtgattgaagcttgctttcctaaataatatttctatacttcagtacagaatgtaaggtccttgctaatgccttatttggtttcttagtttcagtcttactgcagtctgcttttattgtaggcttattacatttatcaccatcattaatttaccatggtgttttatctttttaacttgttatttagatcacattgtataatgctttatttctggtcttgtagcaaaggaatgcatacaacaagcatgtaaaactttattgaatagaacagtctttaaaggggaatcatccataagtcagataaatccatgagtagtatagatttatctactgtaaattttctgtcaggctgttaaaagtagggccgtgtaaatcttactgtcattcacattataataggcacacttaccaatataccaactttatagttttttctccccattttacgggtttagggcttattttttttgagatgtcaagtactattttaacattgtacgtgtttcttaagaaacgccatagaatgaagacaatagggaagtctatttgtgatatattctagctcacttacattttttactgtatcttacaacagtaaacatatcacactatacttgaatttaaattttttcaagtatcattgcattattttgcacataaacatattcttagatggtatcaggttgatgatgatgagcttcatggtaattatttaaaactccataatgagatttgtttccaatctaatcaagaagggtaagaatttggggaaaaaaaatctgttctatagtaattaaagttagccatatgggcacttggctctaaagacttggatgtagtgaatctacccaacccccaagactaatgacttggatggtcttactgtattctcttgtccatagcaaagactgatgaatccagccagggaattctccaccagctctgcttctctgaaaccacaaagtcaggaaatgtaatcagagtgctcagctgaaccagagcaagagctttcctaatggctaactgtgactttagaaatcacatccattagataaagtgattaaatatacatcagtcttttggcaagagagccatgaagatattgcaatagaaccttaccacatgacccacatcttttgcttgaaaaagaaacatgttttcacctttgacacatatcatccctaaattcctcagctcagttgcaggaagcaattttttcatgataagttgtatgcagttcataattttatataaaccataaagtaattgtgtggttgataaactttataaggaaaatgtttctgactttctcatttcaccaccctgatgtgtgggtttttgttttgtattattcttctttttaggcttttcatgcttcaagacttcaagatattttgttaaattcttcccttggaagcctttgtgtcgccaaatcagatttttttaaaacttagatatgaatgtcttaagtgtctacttaggaaatatgtgtgtatgtctgtgtgtgcgtctgtgcttatatatagagagcaggcaaattactcttagcactctgaaaaagctttttttgttaaaataatgttttaaaaatattataaaaaaagtcaccgtcaaaatagcattcgctttatatttgtagttgaaataattcctgtttcttactcttagttataaccatcttttgattaataatatactgcccttcacattttaatacacatgcaactaagccgctgatgtaatacatatacttaaaatgttctttttgagctttgaataaccttttgccttttccctggtgatcaccagtgggcaagatgattgacagagaatgtgtttacaaacatggaatcattgatgactaagttaaaagtgacctcataaaatatagttctaattcttatttacagacaaaattctaagagctcaattcaatgtgtctaggataatagtgggcatagttaaataaataataggaagtgattatcatttacaaaatttcattagtgaattactaaagtatacccctatgcatactaattcctcttccccaaatcaaaacttctcatattcttaaatttctgaattcctaaaatcagcagagatgaagggatggaccagcaactataattttgttgtgttttgtgtttctgaggcacatatagggatacctagtttcatgaaaccaagtggtggggacattacacacaaagagaaagctgttaaaggcagaatgcccagacaagaatgaaaatgaaattcctggaggggcatccccatagtttttctgataatctctctccttacataaaaagcttgaattactggtaaattatccatgcaggaataatagaatcatagaatttttgtagtataaggaattttattgacatcatagttgaaagcagtcattttgtaaatgtttttggtgcccagagatactttaattatggcagaggaagttatagacaagtccacgaaccaggagtagaaaagttttcaaatgagaaatattgcagaccaagcatgttaattttattttattttttattttttattttttattttttttgtgagagggagtttcactcttgttgcccaggctggagtgcaatggcgcaatcttggctcgccacaacctccgcctcccaggttcaagcaattcttctgcctcagcctcctgagtagctgggattacaggcatgccccaccacgcctggctaattttgtatttttagtagagacagggtttctccatgttggtcagactggtctcgaactcccgacttcaggtgatccacctgcctcggccttccaaagtgctgggattacaggcgtgagccaccatgcccggtctgaggtattgttatatacacggcaactctagccttataatgcattgcccttatagtctacttagggtttttaggaatattgtgacctaatttaaaagtttgtagagagattctaattttcagaattaacagtactttcattttctctgaccattgaaatactcctaaattgcaaaatgaagtggaagtacacagtagattagtaaaacatatggttcaatgcataaattgaaataaataaaatgagactatttagaaaatgataacaaatacaacagtaaaaagaactcattacataactttgtgacttttttctttttctttcattggttccttacattttgaactctttctcactttttctgtttccttctttttaaagtaaggaatagagaaagagtgtatacaactgaataagtatttattgattaccttctatgtaccccattgggctaaatatgggagtagaaagatgaataagatggtcacagcttgggggagggtgattatgtcaaatcatgatgagaggagttttttgtttgtttgtttgtttgtttgtttgttttgacggagttttgctcttgttgcccatcctgaagtgcaatggcgtgatctcggctcaccgcaatctccgctcctgggttcaagcgattctcctgcctcagccttccgaggagccgggattataagcacgcgccaccacgcccggctaattttgtattttttttagtagagatggagtttctccatgttggtcaggctggtcttgaacttccgacctcaagtgatctgcccacctctgcctcccaaagtgttgggattacagacgtgagccactgcacccggccactactttggaggtttaagaaaagtagttgtaggtttaaaactcttggactggctcctgagggaagaattaaaatgccctattggagcatagccacctatctaggatcttgagtcctagataggtgggtgggaatgagtatcaggaaattgggctgggaccacttaggacaggcccttgtgggttaatcccataagctattgagaagccactgatgattttttaacagaagagagatgtgatcaatttttaattaccactctgacagtactatatattctgtggataagacaaaatgagagtggaaacctagagacttggagaccattgtgatagtacagataaaagataaagagaaaaagcaaagggaaagggtttagggttgatttagaaagtataatcaatagcccttgttgattagatgttactctattctgagttcccagggagtaaatggtactttttattactagcactggctactgactggcaatcatttaaatgagcgggttaaaataagagtatatgaaaaattagagtttgtggaacttgaggggcatatctaggctggaaaatagctacttgtcattttttttttttaatgttggaagactttctattcttagtttgtaggaaaaatgtacatgataatcaacaaaccgttacacatgtgctaaggtggtatattaactaattttcattggctcatgagttacatgctacctggcatcctcttgcctcttcctctctggagccacagattgttagcagagaagctggaagaacctaccatcttgccataggaaaaaggtttggtccttctggaacatgcctgtgaaaagttgccaatttcctatgtaggtcctggacttatctggaattcaaatatgactagacaccatgtattttatctggcactccgatcctatgtgtttttttttaaatatttatcctcctttgtctcttcatgaccttattatacaagttctagatcagagtgtaacagcctctttaaattctgtggcatgaactttaaatgcctgtgaagaatggggctgggttacgattggctaccagtgaattccaggtaatttagacacttcctggatttgttttccgtgtcatcatttattgattacttcatataaagtcttaatgtgtttattactcaggaaaaatgatggaatagtagcagcaatgccacaggtgtgttgtgaacattaattagttaattacaaagcatttagttgagaggcattctaaatagcacagaactcaaggctttcctccccaaccaggggtccacttaaatagctctttactaagtcaggagaaacaaacacatcttgtacatgaggtacatcaaccactaggcatgttgatctcccagggtgattttctgcatgaaaacctatcccctgggtgtcaagagctttctaagagcaatgattcatgaaccctctggatttaggacaatgctatgaaattattttcatctgtattctgaaataattacttgatgtcctattaaagtattttatattttttgccagaagttgtaggtgtaattggaaacaacagaatactcaaaaaaataaaagatcccaaaggagaataagaagaaagagagagaggaatgggctataaagacggcaagagtgatggaccagtgcctaaagattttagacagactctcaaatttactcttcttatgttctctctaaacaccattcttattagttcagcatcactgtaataactaaaaatattttaaaaagtggacaaggcagatgtcagcaaattgatgtggagaaatgggacagacacatggatgactcactcccagtcccaattccctattttattttttcatttgaaatatccatctcttcagagttctgtctagtgtgaggttcaaacaatttaaatgcttttctcaaaactattgtgcaaatgtctgtttttctgtatttttgtgtctttctgatgttcctctcctggcttttcacctactatcacaaattacatactttattatgtacttttatatatcatatacttttcaatgagcttgattcccacctgaacccacgctttagagagaacaatgataggtttctctgcacctgctccatatttagtaagtttccaattccttttggtgtcaaacaatagcaggtatctctgcagtcccttaggtcactaatgtgcttggttttgttgtcagggtagcagctgaaccatatctttcacaaacaggagatacatattttttcccagcttgagcagagcatctcacatttattaggagaaatgcccctagaggcccatctgtgtgtctttgacggctcaagtattatgtactttaagaagtttaatacattttctactggacagtattgagttgttttaaaatattaaaatcttactttgctgagataaataaaaggtattcaggcttttccaagacaagtatgagaaatgccaagttacaacaggttcactaatgagggagaagaaaagtttcatgataatactatgatctttgctattgcatttggaatttagttagacctaaataatgagaagacttttgtgatgatctattcaatttttatggcctatggtctttttgagaaattgattaaggcaggggaaaggggatagtggtagggcatcactgactcttgttttcttgcttgtttactcttttcacgaagtggtaatacaaattacctcccacagcagcacaggttactcaaaagcccagaggagcaggagaatgagacattccttattcatatttcagtaaggtattcttctccatctttgttgtttcaaggcagatgacaaaatttagtagccattttagtgcagaatactcccaaattcagggaggaagctagattcagttgtttttcagtaatattggaaagtgtaatgtgtaatgcccatgtttaaacagttgctttcttcctaacatgataaacttctatcgttagaagatattctcttgggatgaaaataaaaagatatagcataggccccgtttattttatttggggaggaggtagtagtagcggaaaatataaccagcgataccatcttcattactaatgatgacaacttacaacatcaggagctcaggaagaaggaaagcaataaataccttcatcttcaacaggattttctggcaattatatctgtttttgtttgtatgtttgtttgttttgtcctctattaatttgaaatgcctagattgtttttctggaacttagttttatatcaatcaataaaccccttttccctacttttttttgttactttttttcaagagcatactgacctcaacgtggtgaaagttttcaaaagaggcggatataagcttaaaacctgcctctgcccactcctgatctatctcaaatgaacatctgtaactctccatgaattaaaatgaggataataacatgttctaattgttatagaaaaaattacatttgtagcacagggtagctgctttatgcatatttttttctaagttatgcacaccacaattcagtactaattttgtcatgatgtcctgaagaaatcctaaaaatagagattctgtgaaagaacattgagtaccacataatcaagatatatatgtcttgattcattgattgattcattcattcagggcattatatagcagtcatttttttgctacccttccacatggatgatagttaaagactctactgaagccactagatacccatcttatacacaggctgtaatattttaactcctggagtaattggggtatattcacaataaaatcagctcaaatctaatttatttctttcatgaggtctgaaatgtagtcaaagcttccatttagttcctcgagaaaaagcttctggcaaagataacaaaggacatgattcaaatgctttctgtcttacactgctgcttcacatcaggggcagaggattttatgtttcatggccttctcctcccacatattttgggttttctcatctaatcatacagctatgatgtcagtgatggaaatttccacctcttagtttggtatcttgattgttttttctttccctgtttgcacaaaggattgtaggtggctttccagaaaagagacatgaaaaggatcactaaaaggagcttcaaaacaaattagattatttgttgtggggagaaaaatcatagcagcaggaaatttcattgtgccttttacacaagatctaacatgctgatgaattgctagtataatgatgatttatagagctgagtttcttttgaccaagcccagtactcttgcaaggttaaacaattctaatggcggatgacttgaaggactattttctaagcgaatgtacaaattacaagtccttagtttcgtaagatgctgtttcatcaaaggcttttctttgtggtttctcatatttcatattcatgttgactcccaggtgatagttaatggtatcttttttagtagtgctattatcagcttcaaatgaaataatagataataaaaagccttattattaatgtcatattttgatatatttttaactttataaggctttttagtattattaatgtcatattttgatatacttttaaccttatacttcatttattctaagtaatgtgatgtgatcaataatttttcaattaagtacttttctttttgtctgtgctagtttagtttagttgattggaatatgatgttactgtaactaaagcaaagtttggattcctgtaaaaagacttagcatttctccatagaaaggcatatgtacttttagataattctccatgatcctaatctgttatctcacaagtaaagaattgacacttagcattttgagatttaatatgtattgagtggttcttgtgctttgcccctctactcactttacattataaagcatctacataaaatgatttgtacattatctattgtaatgcacacaataatataatgaagatggggataattgctatctccactttttcttttcttttcttttcttttctttttttgaaacacaatctcactctgtctcccaggctgagtgcagtggcacagtcacagttcactgcaacctccatccccaggggctcaagcaaacatcctgtctcagcctcttgagtagctgagatccactcccctcggcctcccaaagtgctgggattataggcatgagccatcacacctggccttccactttttcaatgaggaaaccgtggtttagagaagtaaagacatgttatcacatgcaacaagaaaaaaacagaactgcaattcaaatcaggttctttctcatagttcttaataccatcctatagtcctcaaaccaaaggttccctcttgcttgactttgagatgatgtatagaatcactcttgaaataaaataaatattttatttaatttttttctttttcccagaaaagtaatctagtattttgtatcaatcaaaaacttctattttgaaaatacaagcattcctagttctataaaatatcaacatatgtaaagtctcctaaaagctttgggtttttatatgtgaagcctttacttctgttttctttttgaaataaaacattgattagaattctaaataggttatcacaaatctataatttttttcctataaaacaagcaaagtatatgacaaaaaaatcggtgctcttttttcacctcaccaagaaaattagtgtaattatttcatatgaataagactttttacctgcactacattatttagttaattttacaggctctagaaagatgttatgcaagagaagacatttcaattaatctgatgttgaaagaatgctagttttaaaactcaatgcactctatgtgctaaaaacgtttttgcagcatgaaggtagggatggacagggacatgtttgcctgagttctccagggaagctttggtggtggatgtatgcaaagggcataatctgtggtttccaaagttgaatatgcacacctaagaacaatgctggaagagctttgtcttcattcttatctttaaaagtttcaattttattcaagatcaaggtaatgatagatttaaaaaataatgataaaataaaaaatagtttcaattttgtgtatatttttaaatgcatatgttttacactgcatgtgtagattgtttataatttagcaagcatataaattcatttattgggagatcttttattattattggtattgctattattgagctggtaagggtgatgatcaaaaataattggagactctattctaaacgtgaggtagaaaattttctctgactctaatggtttaatctttaaaaaacagtttaagccacactataattaaagccataatgtttgtaacacttaattataaggatggaaaacttggtcctatagatattatcaccttaaggaaaggcaggaaagctaactgcagtagttgaccattggcacattgaaggggtcatgactggcctgcagtgtcaaaaagtgaccaaacagggacatctccatatggcattcattgactagattggttctattactgattgaagtcatttgtgttttttgtacaaactttaaatagcccaaagattctaaacgattttaaagtaaatgcaatatattattagtattatatagtctatacatacgtgctgatgtgtttgtgcgcttttctctatttatagggcatcataatttctcaaatataaggtataatataatcaggacaagacttgggaacaacatagtatacataataatcacatagttattattttgacaaacatttatgccacacatgcagaaacacaagtctgcaaccctccctatgtattgacattattctgcaagtatgcttacaaaacaatataagagagatcagaatatcctagtattcttaaggttaaaaccgaaacctttcttgtatttcctgaatgaaatgacagattaatgttattgatgaaatgaatatggcttggcttccttgagacacaggctgttagtaaaaaagcaattaagaaaataataaagaaatgaaacagcaattcctcttcctttttcctttcttctgtactgtgtcatagcaaaagtaatataatagcaactaattataggaaatctcccagactttgtaattggatctttcattttttcccacagcaactgtgaactaagaatttattcacagagcagctgtagtttgtttccagtaactcttagcagtgtctctttgtggagtcttaaagagacccatgtacatatttaggcatattggctaaactagtgccttttaaaacatctgcaggtctattgttgccaattctttcattgtcttctatcaaattaacttttcttagacggtataaagtgggagaagccactgaactcaaagctcaaagcctgacttctggtcatagcttcaatgtttccttaaacatgacttggtaacaagcaccaaactttgtaaatctcaatgtcacctttggtaagaaaaatgaaaggaatctttgcagtttatattttagagcattgctatatgtgaaagagctgtgaaaattataaaccagtgtaactcaccatgtcattatatagatatgtctaagttattcttgaatgtgaaatttccatttgcttgacaaatgtcactctattacatacatgatagttaaaagcatcttattttccatttgttccccagcccagttttcaacttttcttctaaaaaggtaaggtcaattaagagatatttccaaaggaaatagttctttagaaaatactttagtatatcaatgcacataataatacaaagaaatgcagttataaaaataaaatatagaataaatacacagctaccatattcttggcatatatttgtacatgaagaaataaacttcaaagccaggcaggacggctcatgcctgcaatcccagcactttgggaggctgaggtgggtggatcacttgagcccaggagttcaagaccagccggggcaacatagtaagaccccatttctataaaaaatagacaaaacttagccgggtgttgtggcgcctgcctgcagtcctagctacttgagaggctgaggtgggagggtcacttgaatcccagaggtggaggctgcagtgagctgtcaatgcactactgcactacagcctgggtgacacagaaatacactatcaaaaaagaaagaagagaaaggaaagaaagaaagaaaaagaaagaaagaaagaaagaaagaaagaaagaaagaaagaaagaaagaaagaaagaaagaaagggaaagaaaagaaaggagagagggaaggaaggaaaagaaagaaagaaagaaaaaaagaaagagaaagaaagaaaacgttgtgatatgattagaggactaagtcttaacgattagaagcctgatttttaaaaaattcaaaaaccaagaatcagtagtagtagttgagaaagtaaaactgagtgaaaagtagttttgcaatcaatgtggaaacactatttggtaaaatgcacttgtacttgaccttcaagtaatagatatgtcaatgttaaaatggcatgttttgaaagaaaacccatgtcaaatagtcgatataaattatagattatatttctcagataattataaattataaaatttctgagtacattcttcccaagatgtgttatttgaggcttttgtcttcactcattttcattaaggagactggtttcccatttgttttctcttttctttgttttcaagctgtaatactgtagagtaaaccctagccaggtgcttgtcctgccttacttcatcccctctttctaacagactacccgtgtaacttcctcaccagtgaaataggagacatagttcctgaggttaaatcatttactcaggttcatacaagagtggtaaagccaacatttggacttataatttcctacaatatgctacctctctagatagtagaattaaatttcaaagagaaatgtcaggagaaattccatattctacactaaggttctcatctattttattttccaccatattattacatagaagttatttaatttaaaagtctgattttaaggtaacctttatactattaactctcctccttgaagtgctacatttacaagtaattaacaaatactgtttgagcaagcacttgttcaggtgccagggactccagtttttgttaatttaaatatagatgcttgatatcatttattggggtgaaagagaataaaacaccttctcacccaccttcctaaattcattatagttaacagagggagtctataaaaatactaggacatagggactcattacattctaacaaaattctaagaacatgcatgtctttccccaggcatttagcttcccctccctcctcctattttttcatatcttaaagtaagatctaaattaaaaatttagaaattcccaaattatgttgctgttgataaaaggccactacatgtgaaatcagccttttctgtgatacattatatatcagagagtcagctattacttgtttgtgcctttaagaaagttaagaccaacttacctgtcattttaaatttgatttccgaagagcagtgtgccagaaatttggcttaaacagaccaacaaagcgtggcttctaggtgttcctgatgggcaggcttttttcaattaacttcctggggattgttttatttaattacaaggacagtttaccagataaactactcactgattgaatttctaaactcaagtctgctgaataaagccaaatgagtcatcgataaaaattcagatgccaagacctttgcaacaggtacatactctaagccatgtatttctgcattttaaagttttaaaacatccttcctctaatgttatcttttttttctatactggcctggggagtaacagatcttagttcaaatcctggctctgctacacacagctgtgttaccttgagcaaagtatctaattccatagataacgctttagtttccttagttgtgaattgaaaaattcagtgaacccaagttgagacgcattctacaaaataactgatttgtattctgcccaattgtcaggtcacgaaagaaaagaaaacaattgaagttctgttgcagataaaaagagactaatgaggtataacaactaaatgcaataaaaaatgatgtaatttaagtaagatttgcaattcagatagtaatatcatactggtgttaattttcttgattactatggttatataattgttaacattataggaagtggggcagggggtacaagggaatatttagtactatttttgctcctttcctataattagctcacaataaaaaatttaaaaaacaaataaaagttatggagagaggagaagccaacaaatagcataagtatagaactgtttttgtacacatgggcgttagacagatagtaatagtaatatcttgtataattttaatataaagagcaccgaaagagaaaaaaaaataagccctaaatcaatgccgagggtgtcatctttctttggaacccccttaatgcagtgactcctgtaagtgggcatgtccctttcatacagagttgcagttgttacaattgataagggttcatgtgagacaagcaattttgaagcagggcaactcccacaactgctcaatgtggacctcatctccttgtatttgcatagatatgctttgctgcatggattgctgcaaaattgctgaaaagactcccatacaaggagaatgcctggcactactcctccgtttgaaagctctggtttctctcttgtatcctgggaagagtgctgccaagtagtccatagacctcttcttatttccttaacttgctgtgtacattttgaagcatttatgtgtcagtttatttaactccccctgcctaataatgataataacaataatagaggtatgtattgagttcttattggttcaagtatgattctagagtgtttatatgcattaactcatttaaatattcaaaatccttttcatactcattttaccgatgagtaaactgagatacacagttaagtaacttaactaaggtgacctagcttttcagtgttggagtctggattaaaaagaaggtagcctggcttgcacttttcagcctttagaaatatacatacgaccatatccatctgctgaattccaactcaactttccttctttcctataccagctcaaaccaatttctaaccccttgtgtttccaaggtacttttaaaatacttccattcaagctgttttgccatgttttgtactatgatcatttatgtacacgtctttcttaaaaaccattagcctcttgggactaggaatattcctgggcaatgtcaaaatactctttatatgctggtgtctccaaagttactatttccaacccagcctcccattttaagttccagagtaacatggttagctacttattggacctgtctactttgtgtctcccaggcatctcaaacacaggatgtccaaaattggatttatcatttttcttccctgttctttaagcctcatcctcctagtttcctctcataagaaatcactggagtattaacttggcaggccaagtcagatacctgaaagtaatcttggatgctcatctcacttctggaatcccgagttcaactgccatttccagcctttcattatctttcaaatgtaaccacatacctatacctcaatatgtttccatacccccaccaatcatatcagttttattcacttggtaaccgatctttctgctgtaaatttaccctctagactatgtgaaaagtgagttttacaaaatatggacttgatcattttcctgcttaatctcttcaaaatcatacatctaccttcctctggaccccctttctctccactttctttgttaaggaaatagtcccttaaaaataatgaaaataatcagtcaatattttccaatgagttagtgtcaatgcccagggtctacaactcagcacaggtatcctttattaagtgtactataaaaggagtaccaaagcttagagcttaatcactggatgacactggaatgattacaactgaaaacagttagtgattagccttggtggatatattggaagcctagaatacctctcagaagatatagaactaaaaacccagccagcctctcaggccgtttacagtgtaaacaaagcaattagagccaaaggagcaagtaagttctaaggaccctggcattgttaaatggaaaaagcctccaggtgaaattgtggtctaagcataggtttctcaacctcagcttaatcgattttgggggcattcctataggaactttagcagcctctctgtcctctacccaccaggtgtccatagcaagcacccttcttcctccatgttcccatgagggaggagagtccgatatctatacagtatatgaaactaggaaatgtatgtaatgattttgtcctgcaccctttattggctttgtcaagacagcctaaatttcctgtatcttagcttttttcaataaattacaaaaatttgacagttggagccatttttctgcgacagtaattggtgattaaatcgcttaaatgccaacattagccaaaatgtttacctctacaaatgagaatctttacaatccctatcctctttctagcaggtttggctaagggaacaccaaaagcacaggcagaaaattacaattacaattgtccctcagtatcagtgagggattggttctaggacctcatgagatactgaaatctggagatgctcaagttctttacataaaatggtgtaacatttgcacataatctattcacatcctaccgtatactttaaatcatctctagattacttataatacctaatccaatgtaattgttgttattctgcattcattaggaaataatgacaagaaaaaaaactctgtacatgctcagactgtgcttatttttcatattcaagaaaggaaaagaatggtctttaagcccaagtttcaagttgtgaacaggtaatcatcattatttatcattaactcagaatcagtacacaggctctgaaaatcagctaaatctgtgaaattctcattccagtggccatgtttctgaaaataaaccagtcaataactgcctcactccagtaactatgcttctgaaagttagcaagtgaaggccagtccaagcttttgaaagtctgctaatgataaactcccagtcccgaacatcaccctgtcagtgcctgcccctatggtgctagagaacagccaatcagtaagtgtcccactctagtgaccatgtttccaaaatttagaaggtcaacggcagtcttactccagtgtccacactaacacagctggaagtccttgagtctctaaacactctgcacatttccccaaaccttatataatgtcagctgtggctttgttccaggaacttatctctcacaaatacagttctctcttgctggcaaacagaacgtttagatttttggcatccaatatttagttcaatttactgtaagttcacaagtttgcaatcctttcattggctcaactcttgtttatgtttagttatgagtttgtacctgtgagtgactgacagtgacaaagctgtgagattatcaaattcaatcttttcaattcctgggtcaattactaatatccattttttatttgtgtagaagtaaactcaaaaatgtcaaaatgcaagtaagttttgtgataaattttatgaacaatggatgtgcattaaacaaagaaaaactccttttgaagctctttgtgcagcatacaattgtttagctaatattgaaaatggtgaaaacttggatgtaagccagcaaatactaacagctaagcaaatagatgtatgatttcctcaacttgtaccaaagaaagtaataaaatcacatcttattgaaagaaattcagatgaagaagataaaataattgcagcacaagttgtcttccactttgtatatcatacaaggttttaagctcacatgacttaatttgaattgtgctacaagtattttttaatgcaaaattgtaagtaaattttctagtgctataggacaaaatctactgccgtaataaaaattagaataactccatttactgttacagagattaccacagctcaaatgttttacttttttatggcatagccatgaatgcaagtaatcacaatttgggggaaaaattcctttactcgtataataattttctcataaaaagacttgcttacaaggctattgtaacacgccttccagatgagtcttcagaaacaacaagaaattgttgcagagaccctatgattgagttagtagtcataaaaattatacttattttgtggaggtaatacaaatataaattggtggatatttgcataaaggtgtgatatttattgaaagttaaagcaaagtataaatgggactatgaacagtgttgcctcttctgccatattctacatgacgctgctcaaacagcatctgatatcagtaaagtaattgccagtaaagaaattgtcaagaaattattctatttcagcatttattctgttcagactctactaatgaagaatttttgtaattttgttggcattcgttattttcagttctttcacattttaaaattcactggctctctttaatagatgcagttgagagaattcttagtttatttaaagcaccgaaatcatactttaattctgaagaaaaggtctcacaaattcttgctgacttttaaaataacccattgagtaaagactacttatttcttgctcatagcattccatatcttttagcaataggattatgcaatttgaaagaagagtaaacattgttaattgatgccctccattgcttacaaagagttgttgaatgtattaaagataaaatggatgaaaagtttatcccttgtatgccaaggctgttttaagaggaacaacacaacttagggacagggaaaaagtccgtattgaagtggataattatttttacatttgtcatgagtatattttgaaatgtattttatcccttgaagaatttgatcctttaatggaacaggagaaattatatgcactcaaagaagggaataaaaattcatgacaatgttctgttttgtcagtggaaaatatttttaaaagttgtagatcaaaatatgcaatctaatcctgaaaaataaaaaaaaacaagttatgtcatgaaatatggtattgtgttctttagcaaaagagtaagtcccagagttaactttaagtcaatacatgttcagaagtccagcttataaggctaacactgaacatgttcaataagctggacttataagctggacttcacgatgttctcattaatgaatgttgaatggagaaaagagaaaatttggatgtgaccactatggaagacatgttacattgtagggggaacataggctttaactataaggaattttataagtcaactctgcaaaacaaggagctgttaatgaaagccaatcatcgaggaaattttcagaaaagaaatctctaatttacatactgatcatttattttagataaacaattagtaaattttaagattaagactgttctgtaaaaacatttccatttatggtcagaatatgtatacacaatgataaataattcaaatttaactgactgtgtattctttttgctaactctggtaactccacctggaggtagggaagaattatcctgttgaggaccactggtttagaccaagataaaatattatagcattcttacaattattatttaataactttattatactatttatatttcttaaaataaatatattcttggatttgccatattttcttgaggacaaaaatatttttgaaatataataaaatataattttaaaagaaaatttaattttaaaatattttagagtattgaaagtttgttttattgtagtaagttagcatcatcaggttgcttatggaaggcttatggtcctgcaagattaaataatggcttggttttgttatatttacatctgtgatttcatttttaaaaactaagaataatagtgtagaagatataaaagcagtgtgtatatttaaagtgaattttaaggacggacttagtccaaaataaaagttttggactgtggatattgcagaaatattattaataaagataatgagcaaaatcatcatatctattttatagtataagaagtaatatgataggaacaatattgaaatggattctgcatttacagactgcattatgtttcttttaaatcactacttctgaggtcaagtgattaattttcaagtgattcagaagaaatgctttaatcacttagcaaaactgtcatagccaaaaggagcttgttttggtcataacctagttcccacaaagtgagccaaaagtaatttctcatattaagcaaagatattgattacaagcaagagaaacaaacactggctgtcccaagccaaatggagatttattggaagttgttgggtactcatgcattcattggaaggctttgggaaactagagcagctgtataggaaaatgagctgacaaaagggaaatctagaggctgtttggcagacccagctggctagactactggtcactgctgccacacaggattctactatagctgtacatagacccagcacaagggaatttcaataaaatcttcctttatttggcttcattcaccaagttcaaagtaaagggcaacagaatccatttgaataagcttaggagatgatggcctcaccacttaaaataacaggagtggaatggaaatatagtcactttagtcactttagctttccacggcaggtgtcaaggctctgaattccatcaccactacacacaatgggaaatacctgttcattctagataagaaggaagattggatattggatagaaaaaaaagtagacatataattaggtcatatcatatgacattctttgtcatttttgactttttatattaaagtagtgatggaaagttagtggaatatgaaacaaaagttacaaggatgagcaataaaataaacagaaattatattaacattgagcaatgaactataacaaagttttgtcataccagaagcaaaagtaaaaagtattatttctcaaagaagaagacactcatctcttatagcactattaaataaattcttggattgtgtaataatccagtttataaagaagagtgaatcatatgaatatgttgaggaaaaatattaggatgtcttgctcatttgccataaaatggccaaacatgttgagataacaaactcttcatacatcactacattgaggatattgataacagagtggttgggtgttgtcgatttagagtatataagattagcttctgaataaggacactgtacaggctctgatatcttacaactcatatgtgaaagaaacttgatggaggtgttcccaaatttgacagtaatactgaaaattttcatgccattaccaaaaacaagttgtaaaactcagagaaatttgctcaactatcagtaatgaacatacacacactatactcagttgtggtaaaagaaagactaaatttttctatcttctccattacaaaattgatacaaagtaaagggaagatcaaagagaatgaagccaaaaatgaagggaagtcctaaagaaatgtgttaaggagtaaattaataaaattttggaatttatggtattggttaattatttttaatacagagacaacttatttaatttttgttctcacttttaataaacattcacttttatattaatctgtcttttgtgcctaatttgcccttgtatttttgcattttttctcaacctcctcccccagttgtataagccacagatccataaaacctggatctgtttataggccacagtgggttagatcaggggtttccatcccctgggccagtgacagatactggtccatggcctgttaggaaccaggctgcacagcaggaggtgagcagcagcccagtgaacattaccacctgagttcttcctcctgttagatcagccttggcattagactctcagagaagtgcaagctttattgtgaactgcacatacgagggatctaagttgcacactccttatgagactctaactaatgcctgatcatctgaagtggaatagtttcgtcccaaaacctacctccttccctgctgccctgaaaaaattgtcttccatgaaaccagtccttggttccaagaaggttggggactgctggattgccaggtgttatcactaagagagcaaataggtaaaatgtctggtaatatacactggaatattatgcagtagttagaagtgcagattagatgtatttacaacaacatgaatgctaagtactttaaatcatagtgcttagtaaataaaaacaaacaatgagaaaatgtatgtaaattagagattcatgtgtacaatattcaattttcaagaaaacagaaagatgttcataaacacattataatggttgctaagcatgggaatgaaaaacaggtagagtggcagagaatgggatatgggggcaatggagaaaaaatattaataaaaggaggctttgcatggccaatgataacaattggccacaaactgggaatttaacctggaacttgaggttaaaaaaatctgtagaatgcaggaactttggttgatattatccacaatacaagtaagttatgatttaacctttagaaggaaaataagctttgtgcatcctttaaaatttttaattagaatagtttgaagactagacaagtacaggaatagaaaatctagagagtttatgaagctatatatttcaaattccatcgaaggacaaagccaaagatgattttttttcttgtttggaaaagttgagagtaagtaaggacaatcagacttgatgacaaagccaaaaatgaaggagacagctcctggatcatagcaaactgttaacacaagagggaggaattgtaggagatacctgtcatagtacagaccagcaaatttctactcatcttccttccctgaccaaaatctttcttcctaagtccaagttaggtgccttttctgtgtgttctcattgctctcatatagtaggtgtgttgtaattgtttacctgctggttttccccaacagactgtaagccctagaggacaaggatcaaggccacctagttagtattgtgtcccaggcacctggcaccttaaaaatatttgttgctcttacctccaaacaacaacaacaacccccccccaacaataacaaacaacaaaaatgggtaacaactagagatggtagatatgttaatttcctttactatggtaaccttttttttactatctacatgtatccaatagcatcatgttgtataccttaaatatacacaataaagtgtattttaaaatatctgttgaagagtaagtaaaatgaaatgaacctattaaaggaatatcagtataatatttcacacatgtgaccattattttcataacttgctgcgggggtaaaccttaaaatcaagactaccttcattttgttttggtaatttgaaaagtaaggaaccgataatatttaggcatttttttactttaatcttttgtagtttaataaagaaaaatttccaaactacattctgttaattgtgctagcaagttattatgatggcattttaaaaaattaatgcaaataataatgatgcagataatcaatttaattgatggctggtcacttaagatcgctttctaattttcagttattaaaaggtctttcaaaataaggctacttttgtaaaaaaataatttttatactatattgtacattaagggaattcatggatggtgttttctacacaacagagaaaaaattttatgtaaacctgagagcaattactttaccaattaaatcactcaaatgttgtagactaaaataattgaaagtcaaaataagattaaagaaaatcagttttcatatacaaataatggtaagggattcaagaaaataaaaattggacttaatgcattttaacctatatagttttgaaaaccataaaacataaaccttattgctttgtgtagtcatttttcacctttgtagagtccatttttccttacatgtaaacaggcgcctattctaatattcttcatttcttgctgtgtttccattctttttgtctaagatcttttccttctgcctaaagtacttcctttagtgtatattttagtacaatgattaattctctcaacttgagttttcagaaaaagaagtttttaaaaacattttacgttcatctttttaacttttcattaagtataacatacttaaataaaaatgcatatatcatgttctcgcttataagtgagacacatagaggggaaaaacacacactggaacctttcaaatacagtagagggtgggaggaggcagaagatcaggaaaaataaccaatgagtactaggtctaatacctgggtgatgaaataagctgtaaaacaaacccccatgacacaagtttacctatgtaacaaacctggacttgtactcctgaacttaaaataaaagttaaaaaaaaaatgcatatatcctaaaaatatagtttgatgaattcttcacaaacgaaacaaaccatgtaaccaatactgggatgaaaacacagaattccttcactctagaataatattttcactctctctaatgttccagagtgtcagggactacaaaggctacaaaaccaaatgacaggtctgctttgttccaagcaatgttacaggctaaagatacactgcttgtaggatttcatactcttaaaagttcccaggtacataaaattctactaattagttaaatgaagctgtgtattcatggacaatagttaaattgtgacacagatgagtgtacctagcaccttgttagagaaagctccttagtatcaatgagatgtgaaggacatctaagagttatcagggctagaggtctggggagagttatcctggctcatgctagtcttttagtgaaatggcctggacaaattcatagtgaagatgggcagtcaattgataagattatattatatttgactgtttatttctaataagattataaaaagattataaagttaaaagcaaattgaggtgttgtagtgaggaaagaggattgtatagattcataatggctggctcaattccagtgtttttactctacttaagtgtatagatttggttaatttttaaaatttcaggtaacaaataatatggtcatcattgaaatccctaaatataataaattacaaccattgtttttactgaatctagactaggggaagagtaagataaaacaagataagtagaatgttggcaatttaaaaaattgtgccaggctcagtggttgatgcctgtaaccccagcactttgggaggccaaggagggcagatcacttgagcccaggagttcaagacgagcctgggcaacatggtgagaccttgtctctacaaaaaatgctttaaaaaattagcccagcatggtggcacgcacctgtggtcccagctacttgggaaggtgggaggtcgcttgagtgcacgaggatgatgctccagtgagccgtgatcacagtattgcactgcaacctgggcaacagagtgagtgagaccttgtgtttataagaaaatttatacctttcaaaaatttaaactcatactttctgcccatattacagctgcctccttctgtggtataaacgcaatattaaatattgtgtttatttttatcaaaataagacagaaattctcaagacagaaaatgaaaagttttgtcaaggggtttcacagacctttcattgaattttaattgcaatgctaaacaatatttgaagataacccatagtggtcccagtcttcagcctgaagtcaagaacctttagttatctgaatgttctcaagctccaagactccctaggatgttatttaagcaaagtgcaggatttccaaacactagtgattaatatatgacatctgcttactacagcactgttgtgcagccacaaaatctttcagaaaagttcacaataactatggcttactttgataaagaatagtcagtaggatggttagtctaatatgctctcataaaaaaagaaaagtattatgaggtggtgccaggaccttgggtcctatttggctatttctccaaggatctctcaatggtaagatcatcatatatctaagcttgcctagaacagtccaaggtcattcctgctgtcttggtattttctgtgttgtgtagcatttattcttgacacaaaatcctggcttggaacataaatgctgtagacatcctgccaaggattgaatcaaatttataagtgtaaccctgacagctagggtagagctgtctatttccttcctccaaagatgttatcaggtatttagattagtaaaatgagaaatactgagatacccttataattcaaggagcacataggaatggatatagacttggtttaagtattatttttcccctgtattctccagattcaatcgtttcgcacctttgactttcttatctaaactgtagagctattcaatgactaaattgtacttcttattgtctttgaatgtttcagctttatcttgttattgacatatagaatttttattatcttgatatttcacttattcaatcggaggccaggtgtaggaaggactccaatattttcttggtgccaagacatagaacagagttttcagatccatctggaaatttttctgttttcatgggagaaggcctataagagttcatactggaaggaaacattgcctctctcatctctcctgatgacatatttctcttttatcatgggaagtcttatgttatgagtagataagttatgaaggtgatgttatcgaacataggagagtaatttaaaatattattgacaaaaggggcttttgtagttataaaccagggttaagcgtatccttgtcagccagagtggagctgtatttttgtcttgctctgatgatgttatcaggtgttcagagctcagtaaaatgagaatctcagagtcaaatacatttcttttatctctatgttacctcacagttcctaaagtaacacagaaatcaaaagtcagccttttgtaccttaacggcatcattaagatggatatgtaataaagcttcaatttaacaacttaaaagatatttagtatgcttaccatggttgagggagttaaattgtacttattctaaaagaaaatacacacagattccctcataagtgtatttttttagcctattataataagacataatgactctcaagtataattaggactcagacattgtgactccagtttagttctatgtttataagtcataagctcattcctgacccaggagagtgcatttatagaaaattatcctcccaggcatgattactttaagaaaactgatcagcatttctaatggatctcctattgatcaaagcaatgctattttgtactgtgtaaaatatgtatttttaatgtattaattgtaattcattttaattaaaagtggttcaaatttatgtctattatcacttaggtatattatagataatatagataatggcaatgcatttacctgataaattcaaattcactgagctattattacctttatttaatattagaataattctacatctgtgatttccttttgcttaaacttttgtcttggaatagatttatggaaaaatggtgcagatagtaaggacagtttcaatataccccacatccagtttcccctgttattaacagtttatattagtatggcgcatttgtcacaattaacaaaaccatgtgatacatcattattattaacaaatgcccttaccctattcagagttccttagtttttcactaatttcctttttctctttcaagatctcacccaggctaccacattacatttaaccatcataccatcatatcaccttaagcatctctaggctgtcgcaatttctcaggctttccttgtttttgatgatgttgatactttgagtgagtataggttaggtattttgtagaatgttcctgaattggtatttgtctgatatttttctcatggttagattaaggctataagttttgggaaggaagatcacatatgtaaaaggccattctcatcacatagtatcaatggtacacattgtcaatgagttatcgttgttgatgttaactatgatttaaataaactttttaggttctcatgttctgattctatggtgccttgagaatgctttaaaaattaatggtacttcagtcaagattttacctgcaggaaaaaatcactatgtgtttctagagaagtgacattttcaaagcagtatttcattttacaacagttagcatcaataaaaatgtattttttatttatctttacatttgtccactgtcttaaatatttatactgactcaatctcattcagattttggcagatcttccaaatgattaattggaccaattttaaataaataaatttatttatttatttgagatggagtcccgctctctggttcaggctggagtgcagtggcgcgatctcggctcactgcaacctctgcctgctgggttcaagtaattctcctgcctcagcctcctgagtagctgagattgcaggcgcccacaccacacctggctaattgttgtaattttatcgaaataagattaagttatttacaggtaagaggctcagtgatggaatgcaaaacagcctgaaattattatagagttgtgagcatgtagctattttcctgctccttgaaataagaaaatatagagttgtgagcgtgtagctattttcctgctccttgaaataagaaaatgtcatatcatagtccctctaatcaagattaaagaaacaaacaagtaaacactacttgctttctacaagagatgtcaaaagttatcaaaggaaacatactgtttaaaggaattatctgggagatatttttaagagaataacttattctcttaaataaatatagctttcatatataagataaacattaattattacaaaagcacaacatattacaaaattatatggacataatatgcacatactctaactgagccacaatagaattggtgccctgacactaataaatcatttgggaactgcagtttggagtatttagtacctgttatgtggccaagaaggtaatagaggccactgagaccttttccagtctaatataatgggcacaactcacattttttccttttagttattaggtaaaaattagtgttggtgtttacttcagtgattttttagaggatttacaatttttactgtgaacacttagggaaacccacttcccacctcatcctatagattttccctatgtgaagggttttcatagtaccttggggatgaaaggaagtcccccaaataattttcctgatgtctgcttccaatatttgacttcaagaataccgttttccaaatagtatgttcagagccataccaggcacacagagtctctaaaactctggagagactgtaatatttctttgcttgaaggaagggatcattcttgtattctgttcagagattacattaatgatatttaaatgtttctcagaagataaaccaagtgttggaatttatttgcttttgaccctgttcagcaatattcaatgaataaaaaagaaaaaagtcttaatgatagttcttgagccacatgactttgtctaattaaaaaatattttgcaagtcttcatataaagttcaaaacaacaaaggtgaaatgatgggaattttcttaagaaaggaaattctgcagtgacccaatcctttgagcaatcaacagtcattctagggatactcttgtattgtttaaaattacattttacaaaataaataatgtcctaaatgcctcaacatttttagaatcagaaaagttgcttaactgggatgtccaagatcatttagctagtaagtagtgaatccagtattcaaaacccaggctttctagctcctgaacttgtcctagactcactactctaaactaaatctctgtttaatattacatgctcatatgaaatgtttcatgtttaaaaggccaatacattcccataaatggaaatatcctcatttaatataattaatatattttaattatggaagcatgataaaattatccaaagcaaggaattaaaatagaaggatgagacaagttaataatcttatgagctccatgaatacgtgttcattatgtgcaaaactcttcctgttaagcactttatatacagttaaccatcacaatctaaaccactcttcggagcatgattattaactcaatttttcaaactaggaatcttaggctcagagaggttatgtcatctccacagggtcaacagctctaaaatgtggagcacaaattgtaatgggaaaggccaagatgattattgacctgtgagccactgaaaccagtgtgaattgttctgtatggaatagaagtacacttcatgatatatattgtaactgttgctgaaggattttattcattttacattggatcaaatggaaatcgagctgtatattaaagattttacaaaagctccaaattaagaagaaactatagaaactcaaataaaatgaaattttttttaaaaaagtgttttctaagtagtgtttcataaagtaatacaaatttttagttattaggctgataaaataaatctatttcttttagtatctttgtcctttctttttgaatgttgttacacattcctgaaagttggaagatcagcgttttaaaataaatgctaggaaagacctaatgtaactcttgattattttacagcatcaagagttcttccttcttcttgcttattgcttgacaacagattgactgaggatgctttagagctgacaggtttggaggatgagggatgagtgagtatgagcaaaactctgaggaggagcactgcatgcctcaaaagaacttggagagaataagagttaccaaaaaaaaaaaaaaaaaaagattttgccaggctataattggaatgaagtgatataagtgacatgaatgatgcttaaaatgttgagaattaaaaagtaaatattaaaacattgaaaaacttttttttaccctttttggtttatcatcttccttctctccttcaacttccatttttttcattcagtttttcccctcattccaactcttctttctttaattgaacataatagagactcggagttcaaaatatgtgttgtttctattgaattgatagattttttttttttttttgagacagggtctctgtctgtcatccaagctggaccgcagtggtgtgatcatggctcactgcagactcaacctccccaggctcaggtgatcctcccaccttagctactctcaagcagctgagactacaggtgcacagctccatgcctggctaatttttgtacttttcctagagacagggtgtcactgtgttgcccatgctgttcttgaactcctgccctcaagtgattcacccgccttggcctccgaaaatgttggaattacaggcacgagccactgtgcctggtctattgaatggaatttatgtttatcctttccaattttaaaatagttaactttaaagttgtgtattttacattcattgtcttcaaaccaaacaaaatccaaaaacttttctactttttgaattgaattttttctctccttttgaataataaaacctggtcattgatgtatgtgtactcatctgatatttttgatttaacaaaatcaaatggagggttcacgttagtattttatgctttttaaaagtagacatgcttatgctcttccctaaaacttttatttcagcattacttagataagccctggcatctgtatttttttaggttacacagatgatttttttgtataccctggtcaatagccacagtggagtatacctgcattctgatatatacactcttgacaaccatagagacaaatgatacttagctgcatttttaacattacctaacataagtgaaagggattttaaaacagtagtattttggaaaacagcagataataaaacattaattttgtgttgtgggttattggtattaagtatcatgtaatgcccacatctcaaagctcactatggaagaattggtgtcagataataggaagattgatattgttgaaaaccatgcattcagcaagccagccattggtagcacccaatgaagtgttcagtattcatctaagcagagaaagtagaagacacaatctttactgtaactttgagtgtttgcagtgtgtcagatggtcccttgaagaagaaatattaataaaaatgaattgcaaattgtctccaatttacctgaaagagatcaaaataatgtatctaaatatatggtgtttgaaaggtgtagagtcagtccatatttttcaagcccagtgaaggtaacagaacagtttctacttaagcagctgtttgttggtttctttggctcaagagagaggtataatatcttcttttattttacagctttttaatcataataataactattataaaattaacaaatgcacatggtaactaattcaaacaatttttgctttatccctttaaagccattatctagggaataatcacttcccgcttttttaaaaaaaagtttttctgatattttcctttttatctctaaataatatgcttatgtctcatttttatttcatcaacttatgtaatgttttctaactttctaatttgaaaatgaggaatttattttatttatgtaaacttttctctttctgccccattctcaaattccaagcagttatgttttcatttctaagtaattctatgggttggatttatacctttaaatggaatatacctgaacctctatttcttgttacttaaactttagccaggatgcactaactgccttcaatgtaagataaggggtcagctctccaacttttccctcaccactaatccccctctacctctcaatttttgtaagctattccattacttttacactgataatatttaacacatatatgtgtatatcttgtctttgtgtaaataaagttctataattgtacttacattttatatatgctttggtaattggcacatttgtgagatgattaactgatacaatttaacaatattataattatgtatgtatttttcattttaaaacaagtaacatggtttgatctagaggaggaatttaaacctctcattcaaactctgctacttgaagaacacatcaaactcaagtggatcctctctgcttcaattctaccagttgttcaagttcatgcctcattttgaattgattcctatttggacaatatggctatttacacgttctaaagaaacaaattccctttgcttttattcacactatttgtgaaacatcttgtattctatgtgttttagaagtacttttttttttttgctctaccttaatccaaagtaacttaaaaaaaaaagataaaaaggagttaaattttgaattttcttatacctagaaatatattttgtttgccatcacacttgataataggttgtctgataatagaatttgattcaaaatattttctctcatcacactgaaagcattgattgtgttctagtgtcaattatggctaaggaaattttaatggcaatctaattttcattctttttagttagttatcttatttttggaagattataaaatcttgtttctattgttaatgctctaaactttcacaagaatggaacttttaaattgatcttcttttgtacttttagcttgatgactcttggtgaattttctgccattactttgtttattatttcctatctactatttgctgctctcttgagactaatgtcgaacaacatggattattctgtatcttctaagttttattttgtacttttttaatttttaaaattctcctaaaatatatagaaaaatctaagatatagaactccatgcatttttacatatacatattttcccatgtaaccatcacatagatcaaggtatagaacgtttctagcacctgagaatgctcactggtgccccattctggtaaagtgccccttttgggtaaagtaaaaggtaactacaattatgacttctccttttcagcaagcattctactgaaattttttgtttcacaaagcatgacctttacccagaattgatgttgttgtttttggtagcctattttttttttttaataaaagatactctgtctcctcaaatttctctaggaatactaactttaacttctcaatatcccacaatgtttgcttttttttttctaaagttaaaagtttggtctgttcatcttagtctttcaattttatgtttggtagatcttcattaccaatttattcttgataatctattaatatttatggaagaaaggccaggttggtgttggccaggtttgcatctgtctttatacaggtctgtttcccagacaaatcctctcttgaaagagagggatgactgaaaggtctgtgtatatacatgtatggtgtactgacaggcaggctacattttaggatgtatggatgaggagtagataagggagtcacctttccctcgactccaccctggttctagctaaaatggacatggataccctaggagtcctaccttaattaatgcagatattatccttttggtgatagcttggagataaaccactctgctacatatttaacatagagagggaagtgggacaatgttttctgcaacattattgaataaataatccatggattaatcaacttgttttctgatctacatcacatatttgccatagaaatctgtgacttctctgctagaaagaaattctaacattctcagatgagtgttctagactgtagcttctgctttgatctcctcactagggtaatcttatgatttatgataactttttttgtcttcctctggtttttagtgatattatctaacatttcgccactgtacatctttgtttttactgcaatggaaatttgaaggccagagagtgtgataaacacatgggataagaccattttcatgaactgggagctgacattgctttgactggaaagatgtgtgaagaaaaatagatgctagatagacaaatacatgatatgatatttggacagacatagatccatagatggataagcatagataaaacatatctatatatatgtgtgtgtctgcatacatagaaaataagaaagagttcagaataaatatttccatattgattttaactataggaaatcttagatacaagaataacacattcattttttaattttagtagttgcctaagtattctctaatatgagattaatggtgagtgggtaagatagtaatgagaaaacaaaagtactgcctttatatgcaacattgggatttcattctaaagaagatacaatcatagtaatgttaataaaagtggaaatgtagcaatatatttttcaaaattgtaaattaaacatgacaagaataaatattgattgatttcattgactatataaaattaggtcaaataattttaaatgatttttttcaaagtatattttctttcttctggatagaaaaacatgacattttcgttgtacaaatttgaaaagtaaagtataaagacataaaaataatccataatctcttaactagggctaaactttgttaaatgcattgtgtatttaaattaaatatcattttaaaaattattattaatttacatctatctgtgtgtataacctggattccacttattatgcattatatttttataattcatttaattcattaaatagtctttgaacgctctctttcaaagctgctcattatttcattatacattcattacttcatttagtgtaacccttactgttaggattttggattgattataatttttcaatattataaaaatattgtagtaaatatatttatacatgttagaggacacctgagattactgtctttgaataaattcctagaaggaaaattatttggcaatataaacgaattcacgtgtttaaggcttttgatagatattacaaaattaccctcaaaaagtttgtacactttatttatttatttgagacggagtctcgctctattgcccaggctggagtgcagtggcacgatcttggctcactgcaagctccgcctcctgggttcacgccattctctgcctcagcctcccgagcagctgggactataggcgcctgccaacatgcctggctaatttttttgtacttttagtagagacagggtttcacagtgttcgccaggttggtctcgatctcctgacctcatgatctgcctgcctgggcctcccaaagttctgggattataggcgtgagccaccgcacccagacagtacacattatttattactttcatccagacaggcaacactgatttcccctataacctctctaatatcactattacttttgttaaaatctttgctaatgattaataggccaaatttggttccccatgtttattttaatatttgtttctttgattactagtgaggtttgacatggttacaagtgtttattaaccatttgtatttttagataattatgttttttactaaattttatgagatatattaatatctttattcattaaaggaaaagcacttcatctgtcatttgtattgtactttttttattttgtggatgccttccacttttaaaaaccttcattgttatgagattaaatctagcattctttatctttttcgattttgcctttggtgtaatgctttacaagtcctctctactctcacattaaataagtattcattttcacttttattagaatgtttgagctttatttttccatttaaatatttactccaccagaaatctcaaatcttaaactaaatgcctgactaatgaaaactcctgtggcctgtttatccagaagactaagggaagaggaaagaagttgtagtcacacataggtcctgccccatcattgcatcttctttgtgtgtgttggtgaagtgtgaggctccatttttacttatgttgccctctgtaaccctgtcagcctatcatcttgccaccccactggcctggtacaataacttccaagcaaactcagatgccaactaaagctgctcttcttcaaagctataaaattagtaaaatgaaaggaagtttcacctctccaaactagaggatgatgatgaaatctgatcaaggtctcttcaacttccatttcacagatcatgatgttaagcaggtgcttagtgtcttcctttaatttagcttgatcagggaaatattgcccatgcatggtaccactagcattgaccaccctttgcctctgcttttttcatatactttagtcattctggagctgtgttgagcatgtgcatgggggttgggggagacagagacagaggagaataggaatattggaagaatcaaactggtaatggacatctgagttcacaacatctttgtacctagatttctgtcaaataattcttgaaagagatggtaccctccagttgaatgtagaatctacaaaaacaccagttattcaagccaacttttcttcaagagccacagtatgtctacactgaaagtctagtgaggcaagttatcccgtcttctcacctttatgatttccaaaagtgttgaggaatctccaaagcacaagtggcaactgctttgatttgattcattgatttgtttcctttttactaaatctcttcctattctgaggacaattttcacatattttaggattcaaaattcttaatgcatattcatagtagttttattaagatataatttacacaccataaaaaatcaccattttaaagtgtatcactcagtggtttttagtatgttcacaaaggtatgcaacaatcatcattatgtagttttagaatatttttatcaccccaaaagaagccccatactctttaaccattaaccacataaccatcaataatcacttcctattacacccagcctctgacaaaaaatactctacattttgtttctggggatttgcctattctggacatttcataagatggaatcatatcgtatgtggccttttggctctgtgttctttcacggggttttcatcacatccatgttgtaacatgtacctatacttcatttgtttttattgctaaatactaattcagtgtttgaatatcccacattcctttatccatttattcattggtgagtaatgaggttgtgtttactttttggctagtatgaataatgctgctgaacattcatgtaaacgttcttgtgtggacatatgttttaaattcccttgagtctgtaccaagtagtggaattgccgagtcataacttactctttacctttcagaatcatcctacttttgaatgtgtatatgaaatatcccttatttttcactacattctttaaaaacctttatcctaactttggtttccatcaaaatacaggcatacttcattttatggggctttactttattgcacttcaagatactgcattctttacaagttgaaggtttgtggcaaccctgcattaagcaagtctgtcagcgtaattttttcaacagcatgtgctcacttcatgcatctgtgtcacactttggcaattctcacaatatttcaactttgtaattgccattatttaattattactgtatgtgttatggtgatctatgatcagtgatctttgttgttactgtggtaattgttttggggtgctatgaaccacatccacataagacagcaaacaatcattaaatgtgacgtgttctgtttcagcatccctatctctctccctttcctcaggcttccctgttccctgacacacaacgatgttaaaattaggccaattagtaacccattcccacccctaatgttcaagtaaaggaagaattgcgcatttctcactttaaatcagaagctagaaatgattaagcttagtgaggaaggcatgttgaaagccaagataggccaaaatctaggcctcttgcactaagtacttagccaagttgtgaatgcaaaggaaacattcttgaaggaaatcaaatgtgctactccagtgaacacatgaatgataagaaaacaaaacagccttattgcaaatatagagaaagttttagtgatctagatagaagatcaaccagccacaacatttccttaagccaaagcctaatccacagcaaggccataactccaattgtatgaaggctgagaggtgaagaagctggaaaaaaaaggtttgaagctggcagacggtaccccatgagattgaaggaaagaagtcactgccataacataaaaatgcaaggtgaaacagcaagtgccaatggagaagatgcagcaagttattcagaagatctagctaagatcattgataagagtggttacattaaacaacagatttgcaatgaaggtctaacagccttctactagaagaatatattatagctagaaaggagaagtcaatgtctggcttcaaagcttcaacgaacaggctgactctcttgttaggggataatgcagctggtgactttaaattgaagcaaatgctaatttatcgttcagaaaatcttagaattatattatttaattattcagaatattaataattatttactattcagaaaattacataataattatattatttactattcagaaaattacataataattatattatttactattcagaaaattaaataattatattctttactattcagaaaattaaataataactatattatttaattattcagaaaattaaataataattatattatttaatcattcagaaaatctcttaagaattatactaaatctactctgcctgtgctctataaatagaacaacaaagcctggatgacagcacgtctgtttacagcatggttcactaaatattttaggcccagtgttgatacttatagctcaggaaaaaaaaaaaagatttctttcaaaatatcactgcctattgacaatgtgcctggtcactcaagagctctaacggagaggtacaagggcattaatgttattttcatgcctgctaacgctatccattctgtagcctatgggccaaagagtaatttcaacttttaagtcttattttttaataaatacattttataagactatagctgccatagatagtgattcctctgatgggtctgggaaaagtaaattgaaaatcttctggaaaggattcaccattctagatgttattagaacattaatgatttgtgggaggagggcaatatacaaacattaataggaaattggaatgagttgattccaactctcatggatgactatgagaggtttaagacttcagtgatggaaagaactgcagatgtggtggaaatagcaagagaattagaattaagagatggagcctaaaattgtgactgaattgctgccatctcgcggtaacttgaatggatgaggagttgcttctgattgataagcaaagaaagtggtttcgtgagatggaatcttctcctggtcaagatgttatgaacattgttgaagtgacagcagaggatttagaatattacataagtttagttggtaaaacagcatcaggctggagagtggtgctaccaaaggcattatcataaagacttttaaaagaggaaagttgagttgaattgcaccaaaggtaaatatagacgaaaagaaggggaatttaggaaaatttactcagaaaattgtcattttttactttaaagctatgaagcgtaaacttcaaatttctgagtataaagatggttagaaggcacgaaagataagtcccgcccattgcctgacacatagtaatcccttaacaagctgataattctagaacatgttaccttttgtagttgaaactaaaaatctgtttatgttgttattattagtttttaatgggcctttcttggcaggcaaatagttaagtctttatttctttgtttccatccaggcctcttatgtgaggagctgaagaggaattaaaatatacaggatgaaaagatggcaatgttgcctcccccaggacctcagagctttgtccatttcacaaaacagtctcttgccctcattgaacaacgcattgctgaaagaaaatcaaaggaacccaaagaagaaaagaaagatgatgatgaagaagccccaaagccaagcagtgacttggaagctggcaaacagctgcccttcatctatggggacattcctcccggcatggtgtcagagcccctggaggacttggacccctactatgcagacaaaaaggtgagtttattttgacttcagtggtcagtttctgttggcttccttctgtataaaaattattttaaattgagattagtgaaatttagtgaataaccatatcatgaccagaatgttaaattaaatatatatatatatatatatatatatatatatatatatatatatataggatattcagaatctgtactttttcaaatttttcattgaaattggctctggcattggcatactacttttaatttttttttttaattatactttaagttttagggtacatgtgcacattgtgcaggttagttacatatgtatacatgtgccatgctggtgcgctgcacccactaactggtcatctagcattaggtatatctcccaatgctatccctcccccctccccccaccccaccacagtccctagagtgtgatattccccttcctgtgtccatgtgatttcattgttcaattcccacctatgagtgagaatatgcggtgtttggttttttattcttgtgatagtttactgagaatgatgatttccaattgcatccatgtccctacaaaggacgtgaactcatcattttttatggctgcatagtattccatggtgtatatgagccacattttcttaatccagtctatcattgttggacatttgggttggttccaagtgtttgctattgtgaataatgccacaataaacatatgtgtgcatgtgtctttatagcagcatgatttatagtcctttgggtatatacccagtaatgggatggctgggtcaaatggtatttctagttctagatccctgaggaatcgccacactgacttccacaatggttgaactagtttaccaccaacagtgtaaaagtgttcctatttctccacatcctctccagcacctgttgtttcctgactttttaatgattgccattctaactggtgtgagatggtatctcattgtggttttgatttgcatttctctgatggccagtgatgatgagcattttttcatgtgttttttggctgcataaatgtcttcttttgagaagtgtctgttcatgtcctttgcccactttttgatggggttgtttgtttttttcttgtaaatttgtttgagttcattgtagattctggatattagccttttgtcagatgagtaggttgcgaaaattgtctcccattttgtaggttgcatgttcactctgatggtagtttattttgctgtgcagaagctctttagtttaattagatcccatttgtcaattttgtcttttgttgccattgcttttggtgttttagacatgaagtccttgcccatgcctatgtcctgaatggtaatgcctgggttttcttctagggtttttatggttttaggtctaatgtttaagtctttaatccatcttgaattgatttttgtataaggtgtaaggaagggatccagtttcagctttctacatatggctagccagttttcccagcaccatttgttaaatagggaatcctttccccattgcttgtttttctcaggtttgtcaaagatcagatagttgtagatatgtggcgttatttctgagggctctgttctgttccattgatctatatctctgttttggtaccagtaccatgctgtttgggttactgtagccttgtagtgtagtttgaagtcaggtagcgtgatgcctccagctttgttcttttggcttaggattgacttggcgatgcgggctcttttttggttccatatgaactttaaagtagttttttccagttctgtgaagaaagtcattggtagcttgatggggatggcattgaatctgtaaattaccttgggcagtatggccattttcacgatattgattcttcctacacatgagcatggaatgttcttccatttgtttgtatcctcttttatttccttgagcagtggtttgtagttctcctcgaagaggtccttcacatcccttgtaagttggattcctaggtattttattctctttgaagcaattgtgaaatttagtataatgtttagtataacatagtatacattgtttagtataattgtttagcataacatttttggacatgacacagtgatctgccaaattttataatcatacctattttttcctgctaataattaactctcatgtgtatgcagacccaaaaatcacttaaaagtttttaatgagaatgaaaattgctaatttcaattttaaaataccaacaattctttttattaaaatttcaagtataaggtaacttgatcttgccaatactatgtagtatgttgtgatttttcatgttagctcagatgacctccggtttataaagtctccctgataattttgacagtcatgtgaccctgttccctcttcactctcattcttccccctttatatttttctgtatgatatctgtcacatcttgccttatattagttactttcttcttttcatttttctaggaagttccttgaatacaaaactcatgccttatttttgttcaaactccttttcattacctgttatgatatagtttctgactatacatgtattagtcttttttttttttttttttttgagacagagtctccctctgttgcccaggctggagtgccgtggcgcgattttggctcactgcaagctccgcctcccgggttcacgccattctcccacctcagcctccagggtagctgggactacaggtacccgccaccacacctggctaattttgtttttgtatttttagtagagacggggattcaccgtgttagccaggatggtctcgatctcctgacgttgtgttctgcccaccttggcctcccaaagtgctgggaacacaggcgtgagccaccacgcccggccgtattagtccatttttacactgctataaagaactacctgagactgggtaatttataaagaaaagagatttaatttactcactgttctgaatggcaggggaggcctcagaaaacttacaatcatggtcgaaggcaaaggggaagcaaggcacatcttaacatggtgcaggagagggagaaagaggagggaaatgccgcacttttaaacccatcagatctcatgagaactcactcactcactatcatgagaacagcactggggaaaccgctcccatgatccaatcacttaccaccaggtccctcccctgagacgtggggattacaattcaaaatgagatttgggcggggacacagagccaaaccatgtcaatacaattgcagaaagaatggatgaataattttttaaaaatacagtctactgagatttaagacaaaaaggattgtacttgaggactaggtattaattttaaaataattttccatgtaaaagtgaattttacattaaattgtatttagatagaactgatacagtgaaagggagcaggaaaataaaaggacacctgaaaacaatagactctgatttgttttcttgagggaacgctgccgcatacaaataaaaagcctgactcacagacttacctcctgaatacggtgagaaatgcttattgccagaaacccactgataaccctatataaaatttagaaagtcagtgtctttcagctaacacaagtcatcttcctgccaaccggagaaaaataaggctgggatttgaactttgtctttctgatttctaagccaatgctttaggttgctttaatattttgttgttactgctggttgtttttatttcttaactgaatactaaagctatggaaaaggacacttatggaatggctacaataacatttcaagaatgtttgcttctattcatatcgttttttaaattaccgtggttagtaagtgaggaatcctcttaatttaagtcccttttaaaagctaatattagtattataactaaaataaagaaattgaactcagtgattggaaggattcttccaactctgacgttccttcaacttaggaaaatattttttgaaataactcatattttggaacatgtgctgctaaccataacaattgccagcaagtattaaaattctcaaggttttatgataaaaatattagagatagctagtttctatttggctttcttaaaatgcagtgtaatggaagggaaagaagttgtactttaaaataacttaaccacagttattgttactggtgaaagaaaatgaaaaatatgaaatacagtattttgaaagctctaagcaatacatgctaacaaatagctcaatttttaaagttactatgaagaagtggacttggagttcattggccaaggtgacattgatagatgcgttgatgacattggaaccacattgctggagtctgatggcagtgtttcttcaatataacactgccatgtagaaaggtaaactgctgatattgatgtgaaaaattgatattttggattctcaatttcatcctttctttttcctcctgcagactttcatagtattgaacaaagggaaaacaatcttccgtttcaatgccacacctgctttatatatgctttctcctttcagtcctctaagaagaatatctattaagattttagtacactcatatccttttaaaaatgattacatccagtggcactttatggtgtaatttttgctattttattcaaatatagatgttataccttttgcaccttgaaagactctggttttagtttattaatttcagtattattaatataatttgtcatgggtctcagagaagactgggagagtggcaatattaagattttaggatatgttacttttatttattactctcttgttcaaagagacaaaatagtctacaagctcatttagaatttttttcttaggaaaagttgtaagcatttcaacaagtgtcatcctcaaatatttcaaattcccactgtcgtctcttttgttccttgattctaagctaccttattcagcatgctcatcatgtgcactattctgacaaactgcatatttatgaccatgaataacccaccggactggaccaaaaatgtcgagtaagtgggtataagtacattttaatatagttttggtattatcatttcatcctttccttttcctgccaggaagttactgcatttatgaagatggggaatctttaccttcccatgttatatgcttgcttttcttttgttctttacctcctctatgatattaacctgctagattagaaaaattagatgctacagtgttttctcaaagaatgaacttgtcagatgctccatcctgttcttagacatgttacacatattttattcttgggtgtatgttttataattatatgggtgtatttttaaatgattgggtgaatttttgatagaagcaatgtaggaaatttttaggcagatctgagacctatattcttagttatttcaagtacctctgctttctgggagtgtttctgatattgacattttaatattttctggatagacctattgtagtttaatagggtgtgtccataacccaacagccatcctccaatatatgtcttccagtggttaattagagaaaataagttataaagatttacatggtggttgtattcttttcacatctagtatcccaatggaatcttgtgtttaggtacacttttactggaatatatacttttgaatcacttgtaaaaatccttgcaagaggcttctgtgtaggagaattcacttttcttcgtgacccgtggaactggctggattttgtcgtcattgtttttgcgtaagtactttcagctttttgaaacggcaaatttatgaaaatctcaggcagcacagtgttccaataacagcaaacctctggggtctgatttttccagcatggaatgtctgtatttaaaggaaaatggaagctatatgaaagctagtcattgataggcaaatactaaatttttcaaaaagactatctcccactcagaggtatgtgacatcattgagattgatcatcttcctccatgctcagcacttggatggatagtctatgcagtgccaattaggtttacactttttccaagttcttgtttaaaaatgaataaaacctataagaagcacacaaaaaactcaggcctttaaatacaagtacagtgctctttatattaccctgtattattcaattattttatatttttgtattaaaatataaaagtatatatttgtaaaagagctgtcactgcttactaagcctaagattaatgcattgtatacacattatatatacactcagtttattttcaaggaaaatattacattacagtataattttaatttattcataattgttactcttttcccaaatgtatttatcagaaaaatggatttttacaatctcatatctagatattctattttccgttaatgactttgaattatcaaagtaatccaggttcactggagccacctagacaatacagaagggtcttaagattcctaatatccgattccttcatccctgcctaacctcttttccatcaggctccctccttttgaggtcacatatgatggggcagttccatctgctacctgctgctaagaaaacattgtgcaaagaagactgcctcttctcataggtctcctttttttcctattggtacaattcaaatactacaaaattcacaattttaaagtatacaattcagtgtttttcagtatattgacaaacttgcgcaacatcaccactacctaattagagaagtttaacaaccccaaatagaaactctgtactcattagcagtcacttcaatttccttctctccctggccccttggaaaccactcatctatgttttatctctatggatttgtctatttggatttttttatgaatggaaatatcaaacagtatgcagccttttgtgtctgacacttagcatactattttcaatgttttataaaaaattaaaagtctaaattaccataatgttaagggtccaacaattgaaacatttagttgtttacgcatttaagtgtatgaatccaatttaataatatataatatctgtattttgtttgcatcaaatgagtcaaatcaatgctaattaaattaagttagttatgatagcaaaaaagacttgcagaactatacatttaaaatagaagccccaaacgtagaaaataccttgactaaaggctcaggtaagtatcatagactctatctaaattctgaataattctgatttaattctacaggtatttaacagaatttgtaaacctaggcaatgtttcagctcttcgaactttcagagtattgagagctttgaaaactatttctgtaatcccaggtaagaagtaattggtgtgaagcattaggccactcataactccaactatttgggagttgttctttgttcgtttgtgtgtgtgtcgtcactgtgtgtataaactcccctattacagatatgtgacagagtttgtggacctgggcaatgtctcagcattgagaacattcagagttctccgagcattgaaaacaatttcagtcattccaggtgagagtggggttaaacaccagggctgactttgatcttttgaaagaaagacataaaaaaaacctttctttatgcaattttaattaaatttgatttcttctgttttccacaatcattatcaaaagtcaacctttgagtttaatcaattgcatgggtctttaggatgaggatgactacttggctcacatctcaacttgaaatcttccctcctgtctgagggtttgtttttccatttgcttatacaggaaggaaaatttcaatccattttaattgattttaaaatacttaggaaagagatcataaatatcagcatggggatattgcatgacattgttttttattttttctcagcaaaccatttctttattgaaaattattacaaggtgtcactatatgctgtaatttgttttggtgccatttatagagaagtggtttgtttgacaaacattagaacttacaaaaattatttttaaagtggtttgaatagccaaattgttcattttgacaacctaggtattctttgagaatatgttcctatttttaaccatttgctgtaatatataaaaacaaagtatatataaaatagcaaacccagaaggctttttttgaattaggttacttaaggtcattgatttgatataatgcatgactttctaggaaagcttgtgttttttgtttcgattcagaggctttatgtcattacaaacacttttttctcccattttcaggcctgaagacaattgtaggggctttgatccagtcagtgaagaagctttctgatgtcatgatcctgactgtgttctgtctgagtgtgtttgcactaattggactacagctgttcatgggaaacctgaagcataaatgttttcgaaattcacttgaaaataatgaaacattagaaagcataatgaataccctagagagtgaagaagactttagaagtaagaatgtccttgcatttgttattaggttgaaataatgctaaaaacattgctctcttttaataatgaaatgttgctttcattttaatcatttcaaagctattcaaatatatagaaagctgctacagtgtgaatttgggtcatgttatatatgattttcataaatcatcctggttctaagctatctagcagtactggcaaaaggaacacaggatagttccataattcctaatttctatatcataaaaaagaattattttctctagagaaataaatgtagccttgaattaataacattaaccattacatttaacaatataatagattatatattttatataattttattatatagtacatacttatatttattatatataatgtgacatatttattatgatagatttttatacttaatagattgtactcataactataacatcttaaagttctattaatcatatataatgataatattaattatcaaatataaagatagtatctgtatcttgatagtattcctaaagatttgtacaaagcaaaatcatttgctcagctttatcaacatccactttgtaaccaatgacgagtttcatagctgtttttaatggcttccttcaatataggctgatggcaagatatcaaagtttagttcagtagaattatttctgtaggtaccacatgagatcatctgtatgcactctcagaagtctgcttaattcagaagtatcaattagagtttcttacataaagcatgaattgaatgttctctaaacaattatcccctagcatggtgtctctcactggaacggttgatgaatattggatgaagatatgatcaagattgtactttctgtctgacacctggaatgtatctatctgatggtgcccacgtgaatacataaatcttaatagataatttgacccaggtgtggtatggaagtcacgaaacctttcaaatatatgtgctgttgccaaaaggaaatgagggaggaaaggtcaggattctccttagataataaaaagtaatatacatcattttcttattttaccaaatgtgtcctttctcactcactgtgataaataaaatcaaataacacctagcccacatggtaaaagactataaataaaatgacagaaaaaaatagaagccttaacaagaaaactgaaagttttgaagtaaaatgtttagcatatatatttttactaccaccatttgatgaggaatttagcatattttccccttctttttatatttaccctcttttccagtttctgattgttacaaacatgatttgaaacttatttccaatggatggagaattttatttcttaatttatatgctgtaaaattaatatatctgatgagtaacttacatttaatattgtagattattttaccacacttatttaaacttaagttttctcactgggatctgtccttttattctgagtcttgctcctatttctgagtttatactgtgaatggttttcttaacctttgttaaaagaaaaattttcaacaaattaaatttagcagcttatttcagcaatgaaatagtatttattgctgaaataagataataataataatgatttaggcagcactcagaaccaagagaagttcggagagctcaactcagcaacatgagcaggagtatttatagacagaaaaagggagtgacgcacaaaaacagcttgattgcttatagcttgacatttgccttatgtggacattgtctgataaatcagcaggctgtggttggctgaagctcagctgctgtgattggctgagacagctacttgttacaagaatatactaatagattagaatgcaatttgtttacatatgaagttagattgcaattctctatgtacacagacagcctttaatcaaatttaatttaattcatcgctttctggaattgcttcatgtatttttttttttttttgctaaaaaataatacaaaaaaagaagaaaaaaaaagaaaccccgtctctactaaaaatacaaaaaattagccgggcatggtggtgggcgcctgtaattccagctactcaggaggctgaggcaggagaatcgcttgaactcgggaggcagaggttgcggtgagccgagatcgtgccattgcactccagcctgggcaacaagagcgaaactgtctcaaaaataaataaattaataaattaataataataatagagcccttgagttatgtgaattatctttatattatatttaaacatgaatgcatgacaattagctaataatgaatcttataccacaatcctgtcttccaaatctgtacattattctttattttcttctgaaatgtagttttagtgaagaggtgtttaaatcaagtctgaattttgcttctgtttcctatgtcatagaagccatttttgactgcataattgattcaaactttcatttattggtgcgtttattacatggtttccctgagctatagcaataaatgatagcctcagcccttgctttcacagagcttatcatctagtgggaatgaaaaaaaatgtttaagggggaacagacaaataaattaaaaatatctcaagttataataaatgccatgcaaaggaagtgagtctcaggtgctgagatatcaactagatgagaagacatctctgaggtgatggcatttaagctgagatctaaaagatagtaagaagccataaaatagtttaagtgtgggtgtgagtctatgtgtggggggtgctgcttcaattaggagaaatagcatgggcaaaggtcctgaataaagggagggtatgggtttaaagaatggaaaaacagcagcgtggtttggtaagagaaaggaagaatggcaacagagaaagtagcagaaaaaataggggtcatatcatgcaggacatttcagcccatgggaaggagtttgtattttattttaaatgcagtcatttttattgctgtctgtcttgtgcacaactgattgagcaagggtagatggtagatgggaataagaaaggtaagttgaaggttgcttctctcatctaggcaagagaaaataaaggtttcatctagagtgatggcagagaaatgaagagaaaaaagatgagctcaacatacaaagtctgtagagcttgctgatggattaaagaagacaggaagcattgaggatgacttctcagtttctggctgcaataactggatgggtggtgatggttaatgaggaaatggtggacacatttgtagaggaaggtcactattagctagctaaaattaaaatgagatgcctttgagatgtccaggtggatatataatatagagaattggagatttaagtatagaaatcagagggcctgctgagtttggagataaattccagagcaatcagcaaaaagaaaggacacacacacctctacctctgaacatccctgatatttttaagtagatagagaatgggaaacctggtatttaaagcaatctggtaccaagatctacaactctgacatttagaattcaggtagaaaagaggacccagaaaagaacacagagaaagagccgccaataaagtaagaagcaaagatataaataatgaagccaagagaagaaaacatattgtagaaacagatgtgcttgtgtagaatcatgctgcaagatcaaggagtaaggatgatgaagaaagaaatatgcattaggttaagtaacagggaagtcatcagagaatttcttgagaaaatttgggtaagtggtagggttcaaggtaagaataaggtgcatacaggggtgaaaagaagaggtcagaaagaaggtaatatgtgtagacaattctcccaagaaatttggctataaagggaagcagaataatggggcaaatggctggcatttaaaaaaatatatgggtttttgtatggatgttttgctagtgataatgaatgagtagttatatatgaatcaattcaggagagagagggagaagtcaagtcctcgtgaaagtgaaagagtaaaggggagcgacagctccaccgaaacaggaggaaagatagaagagaagactgcagagttatcaaaagatgattgagttcaaacccaatggtttttatttcctcaaagtataagatgggatcattgcctgagagtgcaaatgaggaagagaaaattctacagatgatttgatgagaatgcattatatatatggtcatatcagggcatgagaaatttagtgtattttgagtgcaaatttgatgttggagattacttacttaaaatataaccattcacttggttgtgagagtttctccaccaacattcagccatttggtagccagcatggaaaagacagatgccagattatgtgtagactgtggttctgccaggttaggcactgatgataatgaatgcccatggatttaagctgtacaaagagtgacaagaacttggttgatagatcatgagaaagtgctaaggcaaatgaattggcaatatttaaaagatcaaggaattataatggtaaaggcaatctacacagagaacctgagcataatctaaaagcattttctggtgcctatagcaaagtattttccgaaatgcctttccaaattcacaatcaaataaattaacccactgtctcacctcctgctgaaaaacatacaatttgtagatttcctttggtgttgctgtctactttgatgctcatcaaacacttttttatgctgctacagatgaaagtggggttcatgtgcacagtgtgtagctaaatgtgcagtattatggagaaatatactgtggttatattgggctaaaacaggatattctcttcctcctgtttttctaatatttgacatttaatagagaaagttttgaaaaaaccgcaatattggccttcactactaacaggaattttcctatctccttcatctacagctgttttcttgtgagaactacatttgccagagtgtagagtatcatgtgcaggccctctgcccagtcctgatcccctgcactagttttctcagagttgcattatttgcccttcgttggggatatactcttcacttccataggcccaagccatacacaattgtcataagttgtctttctgagcaagggcagaattaccatttttttttctattttgctacattaatcttgaaaattaatttaaaaacttcacccagaatcttcattagatttaatatcaattctgtatttttgttatatcaattttcattttccccatacgtattttagtgataagttggcatttatttacccccacccacaacctttgccgttatgctgcattcccttttaaaaataggtttattgaggaataatttatatgccacaaaattcctttttttaaactgtataactcattttttaaactatactcagagttgtgcaacgatcgccactatctaatttaaaatattttcatctttcccaccaagaaacccagggcccgttagtagtcattccccatttcccttttctcccagcccctggcaaccactaatctactttctgtccctagggatttacccactctgtagatttaatatacatggaagcatataatatatggctttttgtatctggcttcttacacctagcataatgttttcaaaatttatccatggtatagtatgtatctatgcttcatttctttttattgctaaatgatatttcattgtatgtatatgctatattttgtttatacattcatcagttgatggacatttgggttatttccgtcttttggttataatgcataatgttgctgtaaacattcatgtacaagtttttgtggaaatacattttcaattttcagttctcagtatatacctagtagtgggattactgagtcatggttctttttttttttttttttttttttttttttttgagatggagtctcactctgtcgcctaggctggaatgcagtgtcgctatctcggctcactgtaagctccgcctcccgggttcacaccattctcctgcctcagcctgccaagtagctgggactacaggtgcccgccaccacgcccagctatttttttgtatttttagtagagacggggtttcattgtgttagccaggatggtctcgatctcctgacctcgtgatctgcccgcctcgacctcccaaagtgctggcattacaggcgtgagccactgcgcccggccgtgattcattctttatctttcagaatcatcccacttttgaatatgcacaagcaatatttcatttttctttgcattcaattttttcccatttttaaagatgttaaccttaaatcttcttgaagccaaccatcttaataatttaatgtctagttgtttagaaattattgtaaagttttctacaaacctagagtgagctacctgcattgctttacctttcatcattcttatactgtgttaaatacagtagtatagcagtgtgctaatagacagctccagagcccattcatataattcagctcctcagtggttgataaatgtgaaccagcattagtttaaaggataattctaaagtaatccatccttattaaaatattttggatgtttatgtatgcaaaattgcatcttatcctctcccatactcactctaccaggcccatccttcccgaggttcaggagtgttttcaggaaagcttttcatgcacattttcacctacatttgcacctcaaactgcaaggctttattgttttatctgtgtcttctaagtaagataaaatgatgatgtgtaattcaatcaatagttttatctcaaattgtataatagtttaactaattataataactcctgtttttgagcagctagtacagaatgtgtggatcacagtatcaagtggtttacatgcaatatcctatttatactatttaacaactttataaagtaggtattcttatacgtattttacagatgcagcagctaaggcttaatggcttaaagataattcacttacttaagaagggatagaagcaacattcaaatccaagaatatctgatttcatagtctttcttccatgattactccaaaagtctcccaataatcagattttttaatttgacctgagtaaaataggaagtcaggttcttatagtagaatagatatttaagagagatgacaaaaggttttttagtcatttaaacatttactatacaaggatagatctgcccccagtcttaggaacaagccttaacagcaaatgacagaagagttgatgagaacttttactactaaaaattagcacaaagaataccatgaaaggtgaaacatggaaaatctgcattgattacattgtcatctcctcaacacctagaattagtaggtcctaagtaagtatggactggtgaatgaaaagtaagataattttagaaaacaaattttactcacttatgttactgccttactgagtctgcctcttccggagtattgtaccacataaggatgttttagtcaatagcggaccatatatataatggtgatctcataagattataatactgtatttttttactgtaccttttctgtctttagatacacactttccattgtataacagttgcctacaatattgagtacagtagcccactctacaggtttgcagcccaggagcaataggctataccacatagcctaggtgtatggtagcctataccatctaggtttgtataagtgcaccctaggatgtttgcacaaggacgaaacgtcctaatgatgcatttctcagaatgtatcacagtcattaagtgacacatgaccgtacctttaatcatacttaaaatccaaactcatttctatgatatgcaaattccgacatgatccggcccctgcctacctctgccatcacaatcctccctctcaccacactgtagccacactggccatcttgctggcttttccacacaccaacttcatctctgccttagggctttagttctttctcttcccccagcccaaatatctcttcaccagtattttcatggctgtcttctttgtcacacttaggtcttagtttaaatatcatctccttagaaagaccgtccctgactgcagcctaccacgttgccttaatttattttctttagatcatttatcactatctgaaaatatcttgtatatttatttatttacttaaatttttatccaaccacaacctccaccctcacctaaacataaaggaggtctcataaaactaatgattttatttatcgtgtttattgtcatgtttattgcctagcctctaagagattatcaataaatatttgagggagaatatatgagaaaatagtaaataaatggatgcatattgcctgggaccaggcctgaatttgtagataaatgtctatatcaattaatttacctcctttatcacaatcacagattaaagtctgtgatgttataactgttcaaattcttcttcaacagaatatttttattacttggaaggatccaaagatgctctcctttgtggtttcagcacagattcagggtatgtaatatttgttttctttttagtctaaaggctgaaagagaaggaaaagaatgttcagtcagtttgcaaattagtctttgattctatattgctttttatatatttcatggtattatttgactcattctgtcccacagcaaatccagtctttaaaatgttcacagttaatgcatactcttgaaagcaagactttgagacagtgctcgaattaacacaagacagaagtcatataaatcttaagaaggtagaaaatgttacttctagaaaactccaccaaaagttggttggataatttcaggtcttctttctggaaaacgaagagtcacaaagtctatttttctttctactcccaagttccactcattctccaattaaatgtgactcataggtgactgaatgtcatgttttggccactctaagcaaagggattccttggacttgggtacacatattttaagaatattgtatatataagaatattgtatatattcaactgtccatcttgctgcttctctcttacaccaacttcattttgtagcatcagctgcataagctcttgcttttccccctatgcagacatctattcatcaggctctgtgtacactggcaacataggaataaactggataaggttgctgttcccagaagaatctcagaatattgtatttatttaatggcatcaggaaaaaccagtccaccaattttagaaagaaaaaagtcttaaagctgcacatttttatgaacttagacaattactatattttgactattattgactctaatctattccaagaattatacagacagaaagagctttggtaacagcctacatatcaatatttactgaaccaagaactatcacaaaacgtctgttgaaaatttccatgttttaatgatgatgatgctaatggcagctatgatttattaaacactttctatgtgcaggatgtaagctaagtaatttgtacacattatcttatttaaacttattacaagtcttattttcttgaggtagatgttattatcctattttcacagacagacttatgccttgaagaatttggtaaagtcactaagtagtcagtagccaaatcatgtattcttaatccttattcaatattgtccccctatagaagaaaccttgagtttgtaacttttcagtaaggctatttagcttgtgtcctgaagacactctcacctataatgttctttctcgtgtgtagtcagtgtccagaggggtacacctgtgtgaaaattggcagaaaccctgattatggctacacgagctttgacactttcagctgggccttcttagccttgtttaggctaatgacccaagattactgggaaaacctttaccaacaggtgagtaccaagagaaacatgcattgtatttttgaatggcatatgtacctggtgtatgttaagagcctgtattaggaggttttttatttatttgagaatggaggaaactctattatcttattaccaaaattataagtgttttactcccatcactaataatatgggtagttctcgaatcataaataattttcttatcttttctgggcatatttaatctctagatctctcgccttcctgagtatatcatcccatggggtcctagttaagtaccaggacatactgtaatgttttctcaagctgctcctgcacatggactgttttccatgcgtgcttgggatggcctgaagagaagaggctctctgatgttcacctgctcaccatgttttatttttgaactcctatttaaagttaaagatgcaattcatttctctaagtttttctctcccaaatcaattgctcccaactgtagacttccatgattttttgtattttcttccatttatatcattgatctcttgtattgcaagtggtaattcatgtggctatttcctctaacttgaatgtgaacctcttggggctgtggtctccatttacttgtatagtctcagcagccaagccaaaaggaaggagaccaaaatgattattctatagtacacataacaatgattcacttttctcagaatagctgcagatctgtttttttttcttttttggcctattttatatttactttatttccgagaatattataaatgaattcggaaattatgtaagatgcactttacagcaaaaatgggaatgcaaatattttttaaatatgcaagaagtggtcacctttactcaaaattaaagaaacacaaataaaaaaattaaaataatttttcatcagaggtaagacttatttttcatttaatatcagttgatactatcctaaattcactttaaaatgctttgtatgataaaagtatattacattatacaattattaatagaaatgttaattatcttataaatttgtttaagttacttgtagattcttaatattagacctttgtcagatgggtagattgccaaatttttctcccattctatagtttgcctgttcactctgatgttagtttcttttgctgtgcagaagctctttagtttagttatatcccatttgtcaattttggctttggttgcaattgcttttggtatttttgtcatgaagtctttgcccatgcctatttcctgaatggtgttgcctaggttttcttctagggttttcatggtttggggttttacattaagtctttaatccatcttgagttaatttttgtataaggtgtaaggaaggagcccagtttcagttttctgcatatggctagccagcttccccagcaccatttattatatagggaatcctttccacattgcttgtttttgtcaggtttgtcgaagatcagatgcttgtagaagtgtggtgttatttctgaggtctctattctattccattggcctatatgtctgtttgggtaccactaccatgctgttttggttactgtaacctcatagtatagtttgaagtcaggtaggctgatgcctccagctttgttctttttgcttagaattgtctttgctatatgtgctctttcctggtttcatatgaaatttaaagtagttttctaattctgtgaagatgtcaatggtaatttgatgggactagcattgaatctataaattactttcggcagtatggccatttacatgacattgattcttcctatccatgagtatggaatgtttttccatttgtttgtgtcctctcttgtttccttgagtagtggcttgtagttcttgaagaggtctttcatatcctttgtaagctgtattcctaggtattttattctctttatagcaattgcaaatgggagtttattcatgatttggctctctgcttgtctattgttggtgtataggaatgcctgtgatttttgcacattgattttgtatcctgataccttgctgaagttgcttatcagtttaaagagttttggggctgagatgatcgggttttctaaatatagaatcatgtcatttgcagagacaatttgacttccggtcttcctatttgaataccctttatttctttctcttgtctgattgccctggccagaaattccaacactatattgaatagcagtggtgagagagggcatccttgtcttgtgccagtttttaaagggaatgcttccagctttttcccattcagtatgatattggctatggagttttcataaatagctcttattattttgagatatgttccatcaatacctagtttattgatagtttttaacatgaatggatgttgcattttattgaagaccttctctgcatctattgagataatcatgtggtttttgtcattggttctgtttatgtgatggattacatttattgatttgcatatattgaaccagccttgcatcccatcaaaaagtgggcaaaggatatgaacagagacttctcaaaagaagacatttatgcggccaaaaaacatgaaaaaaagctcaacatcagtgatcattaagaaacgcaaatcaaaaccacaatgagataccatctcaagtcagtcagaatggtgattattaaaaagttgagaaacaatagattctggtgaggctatgaagaaatagtagcacttttacactgttggtgggagtgtaaattagttcaacaattttgaagacagtgaggcaattccttaaggacctagaaccagaaataccatttgacctagcaatcccattactgggtatataccgaaacgaatataaatcattctactacaaagacacatgcacacatatgtttattgcagtactatttacaatagcaaagagatggaatcaactcaaatgcccatcaatgttaaattgtataaagaaaatgtggtacatatacaccatggaatactatgcagccattaaaaaagaatgagatcatgtcctttacagggacatggatgaagctggaagccatcatcctcagcaaactaatacaggaagagaaaaccaaacactgcatgttctcactcataagtgggagttgaacaatttgaacacatggacacagggaggggaacaacacacaccggggcctgttggggggtgggaggcaaggggagggagaacgttaggacaactacctaatgcatgcaggtcttaaaacctagataatgggttgataggtggagcaaaccaccatagcacatgtatacctgtgtaaccaacctgcatgttcttcacatgtatcccataacttaaacatatgaaaaaaagctcatcatcactggccattagagaaatgcaattcaaaaccacaatgagataccatctcatgccagttagaatggtgataattaaaaagtcagaaaataacagatgctggagaaaatgtggagaaataggaatgcttttaaactgttggtaggagtgtaaattagttcaaccagtgtggaagacagtgacccagcaatcccattactgggtatatacccaaaagattacatatcattctatggtaaagacacatgcacacatatgtttattgtggcactattcacaatagcaaagactgggaaccaacccaaatgcccatcaatgatagactggataaagaaaatgtggcacatatacaccatggaatactatgcagccataaaaaagaataagttcatgtcctttgcagggacatggatgaagctagaaaccatcattctcagcaaactaaaacaggaacagaaaaccaaacaacgcatgttctcactcatgagtgagaggtgggcaatgagaacacatgggcaatgagaagatcatcacacactggggcctgtcggtggtggggggcaaagggaggaatagcattaggagacatacctaatgtaggtaacaggttgatgggtgcagcaaaccaccatggcacgtgtctacccatgtaacaaacctgcacgttatgccaaaaaaaagaaaaaaagaaaaaaaaatctctgtaccttcagtttaattttcctctaatcctaaaactattttgaaaaaaaattctattttaaaaagcaaagaaagcgaaaaaaaaagttaattgaaaatacctatgagatgaataaagtaattaaataagaccatctgcctagtgttaacattcaatgatattattaatatttgatgataaagaaagaaaagatagaattttaacagtaaaagggactatggatatcacctgttcctaaaatttgaaagtgagcaaaggatttaattttatgagtaattttcagactgtaggaaaaaatgcatgatttttatactactacacttaataatcaagaatcttacatgactttagtacaagcatattacattgctatattcttcgtactgaaaatgtgtttaatatttgttttccatgaagatataacttttctctttatatttttgataacatgagattataccaaactgtcagatttgctcatgcctgtcaaattgaaataatttactaaaaaaactttttattgttcaaatgacaatttccatttttccctagacgctgcgtgctgctggcaaaacctacatgatcttctttgtcgtagtgattttcctgggctccttttatctaataaacttgatcctggctgtggttgccatggcatatgaagaacagaaccaggcaaacattgaagaagctaaacagaaagaattagaatttcaacagatgttagaccgtcttaaaaaagagcaagaagaagctgaggtactgttatttgatttaaaattcttcctagaggtagaaatgcaaacggttacaacagaggctctgcggtatatgcttggccttctccagctagattttctttaatgaatttttgtgtataaactgtatcctcatttctggtgtggaaaaaggaaccaattctgggctgagctagaattgatactttccccataatcatctcactgcatgaaaagtgatctaaggctgttcctcttaaaccttttacctacaatgacttagtgtgtgtgtgtgtatgtctatatatacatacacatatacatataaatgtgtgtgtatatatatatatatatatatatatataaacacatggaactatgtgtttttttaaattattatatcatgaggaaaaatcttttagaataattgattaaataatatagtcagatatataatatatatataatctcaatttatatatatatacaatatatatatgtatatatatacacacatacacacaatggatttccccagagtaccaattgtatacagtggtttcatgaaaattattttaacgttaaaattaacagatactcttctatggggtatgactaaaacagtgcatcatgttttgttaatgtcacaaggatttttaagataaaacatgagatattaagaaaataattttggtgtggctaatttaggttgtcatagattctcgaaagtttcctccttaatcctctttccagagagattcttcagtgtaaatgtttgagaaggcttgtactaagagaaacataatttcaaatgtactgcagttgccttaacatttgtctgaaggtttcacctagttaaatctcttcaaggagtctaatgtattaggggagggcaatcatctgcaatgcagataaattctgatgcacaaatattcagtgcagcattttaataatggtaaaaatcagaaaaaatttaaattttggaaaattagaaaataactaagaaaattatgaaaattgtatatcttaaagtattattttgccatagaactatgtgtttttttaaaattattttatcatgaggaaaaatcttttagaataatgatggattaaataatatagtccgatacaatctcaattttaaaattatacatacatataaaaagaatggaagaaatattagcaccaattatttctgggaggtaagatgactttttttctgtttttcatttatatctttatttcccatgtttttacaagaattttttagtgtttataaacaaaacattttgtaaagggatagtactaggttatcccaggtactacctagggaattcccagaagtttcatttaggccaatagtcattgaccttaccactttcttacaatattatgctttaataattcatttatttttcaaataactcaatctttattaaaccatagagtgcctgattctattgatgatatggagttaattgcctgataatcaaattgtctgtcttattgcatggtttatgcgagttttacattccaaaattaccttgtaaaactttaacatataatatttaatttgtctttactgtaagcaaattaataacatgtaatagtttaattctaaaatacaatagagctttatttcaacctgagacaaaaaccaaatatatactttgtaagcattctgatcattgtgtaaagaaaacgatcatttcatgtttaaaggacatgattatatgcaccatgttgttatgctaataggtgaaagatgtcctgtcctagggtttcctaggatttggaaatgactcatttaagtgttaacgtcttggcccaaccaggcaattgcagcggcagcggctgaatatacaagtattaggagaagcagaattatgggcctctcagagagttcttctgaaacatccaaactgagctctaaaagtgctaaagaaagaagaaacagaagaaagaaaaagaatcaaaagaagctctccagtggagaggaaaagggagatgctgagaaattgtcgaaatcagaatcagaggacagcatcagaagaaaaagtttccaccttggtgtcgaagggcataggcgagcacatgaaaagaggttgtctacccccaatcaggtaccacccaaattgctaaatgtgtatcacccgaggcagaatgctagagaaaaatgtcttattttagggtaagagaactcgggagaggatagtgagtgatttcattggtatgattttaggtaaaaacctagtatcgctaggcatttatggaaagatgtacacaggtttccatttgggttcaatcatttttgaaagaccttatagtttcagaattgtcaatatattctctaataaactaattttcagaagcaaaatgttcctgagaaccattgtagaaataaagagaccattcacactatagaataacttttatatttgtatttagtctgtgttttgtaatgcttcattggttaaaaactaatcttagactagtttctcttctttcttaaacagtataggtaatagtatcactcacccatttggaactttggagaagcctttgatatcctccacccccaaccacatcaactactaatatgtagtgccatagtctgttaattctgtttatgtggaagttctctttccctttctcttttcactgaaaccttcatctctcaggcctccctgcttataattctgcacaaatccccaagatgaaccattgcttctgttttccttaggtcttccctgttttccatcacacaaccccacaaatcacttttctaaactaaaaatctagttaaaaatctacaagattctcctgatatacacagtaaaaaaagatagagtccctgccttcaaaatttcacttgaacctaggtgatgatacagaacttggacacatatatcaataaaaataaatatgacaggagcagtccaagctggttttgaggcaaagaccttaccatactttttccattgttcacaatagttgccactttgttctttacatagtaagcactaatgtctattgaactagatccttccaaaggatttacaccctggacatgagtcatataagcatgaattgaatttttcctcctctttggtattgggcaacttctttttaaatatttaatgactttataaattaactggtgctaaaagctctctttaaatacactttaactgagctcttagtatgtaaggtggattttcaatgcaaagaaccatacagcatgatttccaatttttttgtgaagcctggggattgatatataaaaagttgaatgaccagtagcaatactaagttctttttttaagttataagttctgtgcagcaaaatgaatggcattaaaagaatggagcagtaactctaggccagaatattagagagctgtatagcaacaaataatttacctggtcctgaaaggatgaattctacaaattgtttcatcccttaagtggaagaagttttcttctcactgaattgcttcgtttttaagtatgtttcttttaagtcagtacagagccacctgccctcaggccagtgggttcagtggtatttatatgaagtgtacttctatcagtaggtgcttcagcaaccacgttttttttaatttttctgcagtcaccactcagcattcgtggctccttgttttctgcaaggcgaagcagcagaacaagtctttttagtttcaaaggcagaggaagagatataggatctgagactgaatttgccgatgatgagcacagcatttttggagacaatgagagcagaaggggctcactgtttgtgccccacagaccccaggagcgacgcagcagtaacatcagccaagccagtaggtccccaccaatgctgccggtgaacgggaaaatgcacagtgctgtggactgcaacggtgtggtctccctggttgatggacgctcagccctcatgctccccaatggacagcttctgccagaggtgataatagataaggcaacttctgatgacagcgtaaggacgttttacatagctcaggcatggctgggcccttgttcctgcatcagtcagtctctctgctgaagggccttctctggtcttgtgaatggctctgcattttgcaatactttttctataggaacacacctttgattcattagcctaatcctcattctatgtgttgtaggcatctaactcaaagataaaatcccatttttgtaaaaaggtacttgtctattaattttataattgaagattataaataatctctttagagaacaatttatgacggatacatatatagatagatatcagtacagaaaatgcaaggataatgctgatattagttaataatagatcccatttctatgaagaaatttggattacttgccagctactgctatctttttcaggtcacagcttaatatgtacctattccaaggaatcttatttgagttgttaataacagcaaacacatatttagtatttatgacaaccccatgagatgatacattattatctctgttttacaaatgggaaaattgaggcatggggaaatgaagcaacttgccccaatttactcagctaataaatggtagctcctaagttgaaacggaggctgtctgtttcagatcttgtgctcttaaccactcactctgctatctctccaatgtgcagtaggcttcttatcttgaaaaaatttagaatggctatgtattaggtcctccatataaatgaaacataactgttttcatttttaaaaaatatttcatctggagtctatgaatcaaaacaaaatatctgaaatgcaagcattgacacaaacaaacataaaagcaaaacaatcattttacagcagttccaaaggtctacgagtcactaagtggttagagaaaggtgtctgattaggcttgaaagtgagatctttctcattcctggagtttccaggatatctctttagcccactcactatgctatgctgcattaccaatatcagcaagggctttgactaacccaataagcttcaaacatatgctacatgcttatatatttatatttgcagacaatgtagtatttcaagtgagagtaaagttccatcaaatccatggccacacatatatttcaatttgaataaatgcatgtgcttttaggtaacatgtgacctctatgtgtgcatgatacagcttaagcccttctgcaaggtatttttcattattgaatgagcatgttaaatttctgtggaatcaaactttgcttgcattttgttttgcaattatgtgtgtaatatgtaaaatactatataatttatatcagattaggagattatttcatatttttaatgaagtttcttcatttaagagttttttcaccatagaaaaatcagtatcacaataaatcctaaagtgaaaactacaaaactagaggaattagtggatacttataagatacaatggaaatacagtaaacataaaattgtggcacaatatatggtttatatgaaaacactgcataactaataacgtcatctctatacctgaaacatgtctacatggaaaataatttgtttaatctgacaaattcttgtttgacagactatagcacagaaaacctggttttcgagacttttcaaaaagttgactttttacagagatctctcaaatgggtgtgtctgttcacatacacatagatgcataatacacccatgcacatatctatgcctgtatgtctgtgttttgatacacatacatgcatgactatacctatacaactatacaactttacaaatctagaaatatatatttaaaatatataccacataccacacacaagcagacaaacatgatagatctagagttgtatagttgtttgtgtatatatgtgtgtgtgagtgtgtgtgtgtatttctggcctgcatggaaccaacatagtgttttaaatttgaattagtttccaagttttaaaaataagacattttctcacaaaaataaatacacttgttatgcatgagttcatgtttttgcatggcaacaatttctagagctgaagagaggtcacctctttcagatgtagcttgcattctccatttcactaattgttcctttcaaactgagtcatttatcatcatatgctctgctcagttcatgaggtcttccttggcagtaagagacaaccattacaggagcttcattaatgttgttccctcaataaaaagtaattccagaaatttatcagagaatactgagattctacccctttagatacacagcactacagcactttcactgttttcatgtaggactgtcacatgacaaactatctgaataaattgaacactcagaaaggcagagaggtgatgatagtgatgatataacaaaaaagcagctagctttattttttatctcataaatattaattcctttaatcctcacaagaatatgaagcaagcactatgattatccccattttaccaatataaaactgagacagttaagtagctctcctatgatatacagccagtaagtagcagagccataatttgaacccagcaatctaggctctacttttaattactcactacagctatataagcaagattttatcttatacctacaatttattaggattctgttcttaagacattaattgattttttttttagggcacgaccaatcaaatacacaagaaaaggcgttgtagttcctatctcctttcagaggatatgctgaatgatcccaacctcagacagagagcaatgagtagagcaagcatattaacaaacactgtggaaggtatgtaataatcttcttttactgtacagattcttaaataaaattaacataaatctggtcaaccttaaataggcacatgaaattcttaggaagaattcagagaccataaaatggttttaaatgatggtaaagataaccaatggatactgatggaattgtttttatgtgcttaaaaatggataaaattatttgctattaaacacagacattgataacatggcaaaaatgactttgcaaaatgaaaaaaagaaaacccacaaagacagatgttgggtgatctgacctttcaaagataagtgatttaatagtaattgatgcagaaaacaaggttaacccagagctacgtgattctgtcactatataatcttacaaattacatattcgctcaaaaattgtctcttctaataagcaaagatagtcaaagggtgacaactttacaaactgtgctggcttctaccctctcccctgacaatatgccccaggtcccacctggtccctgggtctaacacttcaccccccacattcccattgtgggggcaaagtgtctactcatctcctaaataaatggtgagaagtaaatgggctaatggtgtgaaatgtctatttcatgagtcctccataaattatttctagttgcttggttctaggattgttttcacctgtgaactattacagcaagagttttttctggacagaatagtctcatcttattgttgtcatgggactaaaaacacaggtgaaaaatatgcatattggtggaaaacatacatttctctgttaaggaaacatagttctcctgtttaccatcacatgttaggtgcatttttctctgaaagatgcccacatatgtgcttgcagtcataaatttcacttccttaccctctcttatgcttcatctaggcaacgaacatgtcataatagacttacataagaggctatgctttcattgaaatagtactgttaaattttcaaaatctaaactataaaccttgtttcaatattagaatgcctgactgatttgtatctggttaggagtgaaacagacaaatggctgactccatgttctctgcttttttctcccagaacttgaagagtccagacaaaaatgtccaccttggtggtacagatttgcacacaaattcttgatctggaattgctctccatattggataaaattcaaaaagtgtatctattttattgtaatggatccttttgtagatcttgcaattaccatttgcatagttttaaacacattatttatggctatggaacaccacccaatgactgaggaattcaaaaatgtacttgctataggaaatttggtaagtctcttattgtgtgttatgtactcatagtttctctttttagttgtcatcattgtcatttcatattttttatgtcagatgaatctgcagcctcagaattatgcatatccagaactttccatatgaatggatgtgtagagtcaagtttctggttatgtcttttaaaataattgtgcagtactgttgaggtctacaaataacaatactatttctataaaaggttaccatcttgggagttgataatgattttttctgccagcatatttcttgggagcaacacttaacatttttggttctttctcacaccttcctaattacattgctgctttcttgtaagtacacacctcacatattttcagaaaacctatggatgtattgctttgacataatagtagtgaagtgtttgggtcctcagagtcccaaagtcctgggtttgaatgctgattctgcattatatgagtgatatgcccttggacaaatattttaactctgtagtatttaattttatcatttataaaataatacttcattataaaataattatttataatataattgtaactgtatttcatttatcatattcttgggaggattaaatgctttctgcttattagcctggcatatagtaaatacacaataaattaaagctatcatcatcatcaacatcattgtcatcaaaacttcaaagacttaatgaaacaagctttaaaaacaactattttgagaaaccaaaatggttgaactggacatagaagactcaaggataggcaagaaggtgatcaacagacttctggaagatataagatccaattagtaaaattgatgccagagtgatctctaaaatgtacagtgctggtcacactaactcagggtaaacaaaatgccagtttttgactatggatgtgaggcaggcaccagttcatttttagggccagagaatctcagcttatgactcttcataaaccatgaataactttgggtatctttgtcaatgtctatataagataattttgcttttgctgtggtcccaaacaaattgacatttgaagaccagaagttcttttctgagtctgtcttttcttctaaattgtctacaaatattcccaatgtttttccccatgtgctcgctgagttagcattcttcttcccttctatttatctttggaaaaaggcaggaatatctagtttctaattgttggtggagatccggatcttgcaaataagtaccttctcactgagacgactgcatccctgtgaactcagaaaggatttacctggtcacagctattaccacaacctcaagtccagctgctccctgccaatcttcttgatcctgcattatttgaaaattgactcctgcagttatagacctaaacatttcccttagaatcttctttggtcagattccttgagtcaaagtgtacccaaaggattcatttcggtgttgcctttttaagtgagtcctttgataccagtatcatgaattaatgatattttgcaatgcacttttgtgtcaaagctttagaagtattactttagctgccacattgtcaattaaatacttttttctcttctgctgataaatctttaaagagtgagtcaacatgttctttattactctaacagtagtcagatcagaaaaaaaaagactggctcaaaatttcagtatcaacttatgttttctttttatcttcttccagaacaactttttgacccacataaattctttgaaaatagtgactgtgtcaattaccaaatgttttactcttctgaaattttaatatgctattctaaaatgctgtctaaaaatgatgagcactgacaggacagttagagcttttcaaccttttgatgctgatatgggagccagggaagagcaatacctaatttacaggactaagttagataagaatatccatttataaagtatttagcaaactgtctggaacacagtaaatgctaagcaaacagtgattattatcattgtgttgatttcctgttttctaatattaacagaaaaacattatttttctcacttaggtctttactggaatctttgcagctgaaatggtattaaaactgattgccatggatccatatgagtatttccaagtaggctggaatatttttgacagccttattgtgactttaagtttagtggagctctttctagcagatgtggaaggattgtcagttctgcgatcattcagactggtaaacataaactaaggttgccattatattctataataaaggggtatttctttggttttgcattatttttaaaaacttttgggaatttgaatcaaaaggcaagattatatcttgatatctagtaaaataaaatccattcattgacaaatgcacttaaagaactaaaaccacatgaatatttttacaatttttgaagacatgctcaattctaatttgaagtacacgctaatataatataaatattttctattaatatagtacaataatttatttcagagtaagaagcttgtggcttacagaatttcaatgattatatgttcataactttatgaatttggtaattaaagatcaaattgttatcccccaaaacaattgttgtctgatcttaacaatataaaaacaaataaagactttttaaagcacattcctctaatatgctagattttatctaagtgtcggcatgagaaaatgtatgaaagtattagaaagtagaattaaggataaattatcaattctaaaagaaaaatatcacaaattatgataagaggtttctatagcaatcctgaactaaaagtacatagtttcttcataatatgtgttgtaattttttgtggcaagtggaacaaaataacagagatattgaaaattgatgaaaatgaaaaataagaatattttagctggataataatgacaatattataggacttaaagtctgctttaccctttgaacaaaaatcaagattggaaacattgaaaagattttcatagtgattaacattaaactttatatttgcttttagctccgagtcttcaagttggcaaaatcctggccaacattgaacatgctgattaagatcattggtaactcagtaggggctctaggtaacctcaccttagtgttggccatcatcgtcttcatttttgctgtggtcggcatgcagctctttggtaagagctacaaagaatgtgtctgcaagatcaatgatgactgtacgctcccacggtggcacatgaacgacttcttccactccttcctgattgtgttccgcgtgctgtgtggagagtggatagagaccatgtgggactgtatggaggtcgctggtcaagctatgtgccttattgtttacatgatggtcatggtcattggaaacctggtggtatgtaaccagatgttcatgcattttaatttctctgtggaaattattttgtgatgatgtgatctattatttttataattttataattaattatatctacaattattaattgcaaaagaatagtgataattttaatacaagaaagaacaggaaagagaacgaaattttgtattagttttcctagccaaataacccatatttatatttttcaaaatcactgcctattcattttaaggcaaaggtttctatctttttctctaattttgaaagtgatggcatatgctctcttgaagctatactagtaaataaaatataattaagttaataataacaaaaatgaagttaataatggaccaaggcaaatgaaaggactggtcccgcaccttatagccatgtgtaagactagcctctatagcaaggatcagcacctatgccccacagaccaaagccagtccaatgcctgttattgtgtggccctgggagctaagaatgatttttacatttttaagtggttaaaataatcaaaagaagaataaatgacacatgcacattatatgaaatgcaaattttagtgttcataaactttattcaaacataggaatgtcaattcatttacgtatcttctatgactgcttttacattacagtggcagaggtgaatagctgccatagaaatcgtatgcccaataaagtctaaaatatttatcatttggccctttacgaaaaagttttccagccactggcctatagcatcgcgtggacatagcaatctgcctactttttgaattagtgttttcttggcttgtactttttttttattttttttcttccttatactgattagtgaaaactttcaagcttacaagcaggagaaaattgggataaaatgctcttctatgagccactcatctcaattctcaaatctgccacgttgacttgtcacagttggtcagcttgacatcaaatctctcatcaggtatccagaaggtgggatatggtaacagccatatggtaagtgccttcacttcccactgagttgaaaaaaattactaaaatattgttgtccttgtaaccaccataaaaagaggattcagaagtcaggacagaaacttacattcatccagtcacacactaaatgatactgtgccagacatacacaaaggcatttatgcagcacaaattctagatttttactgcacaaaaaatatacatcctggattaccattctataattgttgatcaccctcaagttgtagtaataaataagcttcctagttttatatagccttacaaattccttccaatatggaatgaaatgcacattctctttgtttcttcttctctgttttgtatttataagctcctatagcaggatttgcttgtgtattttttttttttttgagatggaatctcgctttgttgcccaggctggagtgcaatgatgtgatctcggctcaccccaacctccacctcccaggttcaagcaattcttctgcctcagcctcctgagtagctgagattacaggtgcatgctaccgtgccagctaatttttgtatttttagtagagacaggttttcgtcatgttggccaggctggtctcgaactcctgacctcaggtgatccagcctccgtcgcctcccaaagtgctagaattacaggcgtgagctactgcgcccagctgtgcatttcttcttacaaaattttgacataaaatcgtgaaattgggaaaggtataacttgtgaaaacatgccacatcaaagccaataatcacaaataatttaaatgtttattatttattcaggcaaattcaaagggaaaggcattcccttggctctcaccacattttccactcaatattttttagtatcttctctgtgaaaagaaaggtatttaggtattgtttagataggaaggcattgtttaggtattatttagggtggaagaccaaaaccaattacccatggtctaatccttgtttttaagatgcttattagggcacagaaaagtggaacacaaaagttatcagacaaggaagaagtgatgagttctaccaataaggttaaataaaatgcttctgacacttaaagaaagagttctagattgagacttaagggaggtttttgaagtgggcctttgaacaatggataatataatatttgatcatggacactggtgtgagagttatgttttcctttgaaaggcaatatagatttatagtaaaaaacagaggggatatattggtgagtgtttcattcgtgtagaattagtgttaactaactcatattattattctttaattattaattcttggcaaagaaagtatacagaagaaaattcataaagttgataaccaactcttgactaatggaggaactaccaatatcatagaattaaaatttaaggaaaaaaatatattcctgatatagccttccacattagagttatatgctaaactggcttctctttttttatatttctctaatttattcttttgatcattttattgccagttaattgaaatggttaaggaccataggtttaggaaaacatttggaaaaataagttttagtcagctcgggtctatatcaggaaacttgcgaagaccattaatcaaaatgagactttgaatcaagtatacattttattaatatttcttctcttgtttaacatgataatacaaagatgatacaaatggactttattttgatttgtgatacccactaggttatagaaatattagagacttatgatttttgaaggaaagaaagttgtcacagcagctgttctcaacgaagtgagaaccgcatgagccactgcaccctgttaaaatggtttaacgcttgaacagattaaggagactaaagcagtaaaatggaccctcagatcttcaccaatattcctattaaaacaaatatacacaaacatgtatttcatagtgaaatatactttttcattgacaactattaaaccatttcagggatttttagcaattatcaaaattgttcactatgattcccagacctccgacctccagaattttctcctaaatcagacttattgagacacacaattttagcctgctgtctttacttacatctcagagaaattgttttaggtatgaccaaaatgagggatgagtaccttctcttcagttaggtcttacttcgatttttaaaacatgaggtagatgatttgggccatgaaaagacttacatcaaatgccaattcaatttccttttttgtttcctggataaaacaatgtttaaatataatttacaatttacatgtttcctgccctctgtgaacattttttttttttacaatgtggagttgacctgcacttctacttcagtgagaaaaaaaaataattaactcagaaataaaactttgcttcaagagaatagaaattgtcctgaccttttttgtaatctcttctagggcaaacataataggcaatgaagccagccaatcataatgccattattgcatgtgaatcaggttcaaggggcaggtccttttctgtgctttaaaagtcacattaagcagtattctagcacgcttcttggacagaatattttattacaggaactttgtaagtattatgtgttccatttgttagaacaagtgacatttatcttctggtaaagtgaacaaagatctttagcacaatagaaaatagtcacaattgttttgttctacctgtttctcacaggtaataaagagtaatgatagatgaatgctaggagagttttttgggttcataaaagagaacttgattaaggcactactgctacagaaatgaacttagaaatgatgtaatgataatggtgagggatcattctctctgctccccatggtaattcttcctgggtatggctgaccctctgctgctgagtctgcaggggctagtctagggatacagtgatgaggagtctaccctcgcctctgtctctctcatgtggtcttatccgtgtgtgaaaaggtgcagaaatcttcgttgctggtttgtattgtggcctatattcggaaaagtgtacttattttattttattttttatatttcctgtctccctatttctctaccccctctccccaccctgatataggtcctaaacctatttctggccttattattgagctcatttagttcagacaatcttacagcaattgaagaagaccctgatgcaaacaacctccagattgcagtgactagaattaaaaagggaataaattatgtgaaacaaaccttacgtgaatttattctaaaagcattttccaaaaagccaaagatttccagggagataagacaagcagaagatctgaatactaagaaggaaaactatatttctaaccatacacttgctgaaatgagcaaaggtcacaatttcctcaaggaaaaagataaaatcagtggttttggaagcagcgtggacaaacacttgatggaagacagtgatggtcaatcatttattcacaatcccagcctcacagtgacagtgccaattgcacctggggaatccgatttggaaaatatgaatgctgaggaacttagcagtgattcggatagtgaatacagcaaagtggtaagaatgcttcatatactttgtgtttcatattaacaattagtatgaaatgaatgaaaatttcatatttctattgtcataagttcttcttaacattgcatgtagtgatggatagtttttgtgcgtgactttacacaacagcttcctgaagattcatctggtaaagttttcttactagttcttaataattgatttaatcttacttagctgtatgagaaagctcaaaggtatctttgagccttcccatgcttcatcccatgtttcctgtcagttgaaaagttacacagtctccctccacagtgcctgtttgaatccacctctccttttcatcacctttcctttgtactcactactttttgcctaaaccatttcaagcagagtctcaactgatgcctctgcttccattttgcatttactctagtttacttatttgattattttcgctcatgcagattcagtcctaatcatttcactctcatgatgtatccatgatccgcaaagtatcgctctctctctctctctcttcttttttttaaaaaacggagacaggtctcactctgttgcccagtctggagtacagtggtgctatcatagcttactgcaaccttgaactcctgggctcaagtgatcctcctgcctccacctcctgagttgctgggactacacacccatgccacaacacccagctaatttttttaaaaaagtttgtagagacaaggtttcactatgtttcccaggctggtctcaaactactggactcaggcaattcttcaaactttttccccagttgctgagattacaggagtgagccaccacgcctggccatccccaaagtctcgaattatgtactgtgcacaaagccttcacactatggccctaaccagctttgaccagctctatctcctagtgctcacccatagatttttacctgtaccaaaccaatttccttttaggtcataaacactctccacactttgcttgggatttccttctccagtcaaaatccttcaaggcatcacccttgtatttatcatctattaaattgttaatacttaaatcgcagttgggtcagggttaaatgaattaacacgttaatacatgtaaaatgtttagaaccatactcaatgttctcaaaatgttatttatttcatccctcctaatttctccccaaatggatgtagtcccttctcatttttgaaccgcaaacaattttatacttcttcctggattcagtaatcttaaattttaattttttgagaaattatggtatgcatcaaatagtactgtaaattcattgagaacaggaactctttaaaacctttttttgtgccctcctctcccacccttacctgcatatttctagtgagtcaaggtatgtattgagttttatttttattgtgaaatataacaaccgtattacaaattacttttatgagattgctttcaagtcttaaggcatcatttaaaaataatcttttgttttctctagctcatgttagaaagtatttgacagatatttaccaaatatcttctatctccttaacttactaagactccaccctccatgaatttatagtggacttggaaaacgagattatatggaataatcatgactgatatatatatatatatatatcagtaaaatgctcaggaggctgaggcatgagaatcacttgagctccagaggcggaggttgcagtaagccaagatcgcaccgctgcactccagcttggactacagagtgagactctgtctcaaaaaaaatcaatacataagattaaattaaatacataagattaaattaaattaaaacaaaataaaataaaatgctctatatttaattgagtgaataaatgattaaataaataaatcatgatatatgtttctatgtagccaaggacattttttccaagacggtaaagtaaggaatcaaatctagtcatccctcgatatctatgggcgagtagtttgaaggccccccacagagactaaaatctgcagatgctcaattcctttatataaaatggcacaatgtttaccataaaacctacacacatcctctcatatgtacactttaaattatcttcagattacttataatgtaaaagtaaatcactgacacaatgtaaatatttgttttactatattttttatttgtactatttttattgttgtatttttatatttattttttccaattattttccatctgtagttggttgactctacagatgtgggacccagggatacagaaggccagctgtattttatttgtatattttaaaatgttctacatggaagaagtaatactcccaaaattttaaaaccgtgttcatttcagcttaaagttagaaaatcagattccacgtatgccttttaaggagcaggcatctaggtttagtatcatatggaaagggaaactaactcagtcacctatttttttctaactaactctgtcacccatttaaagaagattaaaattataaactccaattgcctggcagacagttgctctcagaataaaacttggattctaggtgttaaagcacttcaggagctggggaaagtcaaccaaagacttctgaaattatttcttaacatttatgaggaattatttcttacctgttcatttttctttcatttaaagtcaatggctgacctttcatggatctccttagctgctgacaaaattcttattcttagtatgttgagttgcaacatttttaagggaaccttatagaactttccatttagcacttcattttctaactgaaaaagcagactcattaaaaggaagtgattttattcacagttataaagctagagttatacagttataaatctagagttatagagttatacagttataaaggcagagccatgtcaagagtcctaggcttgtgacttatagctcaatggaatttctacctctcttgtcctctctttgtccatttgaaacacaaaacaaacattcctattggacttaataatttgtctcccctggggtataaagccatgcagctatagaatttatatatctgtggcatgcagatgaaggtgtgttgtcataatggttagctatcttttataactagtgattttatttgtattttataactgaaatagattattttaaccattcataaaattactctgtcacttattccatgagcccttccgaaagcgtatgaaatctagtgccctataaactgagttgtctaaaaatcatttgatgttaatattctaatattcttgattttcactaatttttttctcattacataatggttatatctatatgataaaagagtgtacattcttactctattacatttccatggtatgcacaattaaatatacaggagtttgtggctaccttagaggttgtagtaacagatacacaagctagtatttttaagtcatgtagcatagtgtttgaattagcaagccattttaaaaaaagtgtttgttgtcatcgatttctgcatcattattattacttaagaactcaggttcttagtgctactgacttctaattatgaactttgaaagtttacccttcttcataaaaaattttccatgacgtatctcaaagtatgtcatattaagtatagacaaacctacctgaaaatataagaattgagaaataatgaaggttcaagatctagaggaaatttgtttgataagatattaagctgtggtcaactataaaaaccttacatctaaggtctttctttccagcctcttcagattaaaatgtttccagtttaaattctgtcatttttgttttgttttgttttcccattcagatcactaaaatagtgtacattttatttatttgtctaaaatgtctttaatcctcctatgctaacttttcctgctctaattctcataatcttcatctcccacttgagttcccagagtcttgaactgtgaattccctcttttgcaacttttatttttttcgtatctgagatgcctttgaaatagtgatggttgtgtttgtgatgagcctggatgagccaaagaggcaaattgaattacccaatagaaagtaactcctaatttatatatttatgtcattaagtcttagccactaccttgggatatagttaagaatgccactttttaagatagaatatttgcctcaaatcttagcttttgataagaccttgagcaatttacaaaaacatttttttcagtttatttatgtgtaaaatagattgtaataaagattatatgattctcataaataattagacaaatgcttattgaaaagtgctcaatgtacaattattataagtatggaattgagtgcttatatacacactgtaagtagctattggttacagattgaatttaaaagagatagcgtgatgaagatcagtgcttcagaattttagtgtgtgaaagacatacttggggactttattacaaatgcctgttctggatcatatcctttgaattgtttgcctaaatttggatgcaacttcagaatcattaacaacgtttgcagcagctactgatcaatagagactactatgaacaatcatactccaacaaattggaaaacctaatggaaattgataaattactcaacacatacaacttgccaagactgtatcatgaagaaatggaaaactcaaacagaccaataatgagtaacaagattgaatcagtaataaaatctctcctagcaaagaaaagtccaggaccacatggcttcactgatgaattctaccaaacatttaaagaagaactaatactaattcttcttaaattattccaaaaaaatcaaaaggaaaggaattcttctaaattcattctacaaggccaatattaccccaataccagaacaagacagggacacacacacacgaaaagaaagctgaaggccactatccctgatgaacacagatgcaaaatgccccaacaaaatactagcaactcaaatccaacagcctatcaaaaagattattcaccatgctcaactggaaatcgtcccagggatgcaaagatgtttcaacatttgcagaacaataaactttctatattatatcaacagaatgaaacataaaaaccatataatcatctcaatagacagagaaaaagcattttataaaatccagtatcctttatttgataaaaattatcaacaaattaagtatagaagaaacataccttaatgtaatgaaggccatatgtaacaaacccacagctaacatgatactaaccagggaagagctgaaagctttctctccaagatctggaacaagataaggatgcccactttcactacttttattcaacatagtaatgaagttctagccagagcaatcaggcaagagaaagaaataaaggacatttaaattggaaagaaggaagtcaaattctctttgtttttatatgatctgatcttagatatagagagttctaatgaccccatgaaaaaaagtagtagatttcataaatgaattcagtaatgttggaggacacaaaatcaacatgcaaaaatcaatattatttctatatgttaacaacaaataatctgaaaataagaaagcaatcccatttacaatagctacaaaaaataaaatacctagaaataaatttaaacaaggaggtgaaatatatttacaatgaaaactataaaatattggtgagagaaattgaagaggacacaaaaagggaaagatattccatgttcatggattagaataattattatttttaaaatgtccatattacccaaagcaatctacaggtttaatgcaatccctatcaaaataccagtgacattctttacagaaaaaaaaaaaaaaagtcctaacatttgtatgaaaccacaaaagaccctaaatagcccaagtaacctggaacaaaaagaacaaagctgatggcttcacactaactgacttcaaaacatactataaagctatagtaaccaaacagaatgatactgacataaaacagacacacagagcaatacataagaatagagagcccagaaataaatctacacatatacagtcaactgagttttgacaaggttccaagtacctataatagagaaaggacagcctctttagtaaatggttctgggcaaactgaatagccacatgcaaaagaatgaaattagacctttatctcttactttatacacaagtcaactcaaaatagactacttaaatgtaagactgaaaactacacaactattagagaagaaaaaaaacagggggaaagcttcatgatgttgttctaggcaaggatttttttggataaaacctaaaaagcacaggcaacaaaagcaaatatagacaaattggattatattaaactaaaaagcttctgcacagcaaggcaaacaatcaatagagtgaagagataacctacagaatgggaaaataaatttgtaaactatgcatctgataatgggttaatatctaaaacatacaaggaactcaaacaactcaacaacgacaaaaaaaacaaataattcaaaaatggacaaaagacctgaatagatatttctcaaaagaagacaaagaaatgccaacagataaatgaaaatatgctcaacatcactaatcaccagggaaatgcaaatcaaacctatcttgaggtatctcctcaccctagttaaaatggctaccatcaaaaaatcaaaagatataagtgtttaaaggatatggagaaaagggaatccttacacactgattgtgagaatgtaaattagtaccaccattatggaaagtaatatgggggttctttaaaaaattaaaaatagaattttcatatgatccagcagtcccaatactaagtaaatatccagatgaaatgaaatcagtatgttgaagagatacagtaaacatagaatactgtactcctatatgtttattgctgcactgttcacaatagccaagttatcatattaacctaaatgatcgtaaataaataaatgcataaagaaaatgtacataagaaaaattgaacgccattcagccataagaaataatgaaatcctgttatttgtggcaacatgaatgaacctggaggacattatgttaagtgaaatcagccaggcacagaaagtcaaatatctcacacatatgtggaatctaaagggttgtcatagaagtagatagtagaatagtggttatcggaggatgggaggcttggaggagcgaggtgtggggaaagattggtcaacaggtacaaagctacagtgagatagaaagaataagttctggtgttctattgaatcgttggatgactagagttaaccataatgtgttgtatatttcaaaatagctagaagtttttaaatggtctcctcacaaagaaatggtaaatgttcaaagctatggatctgctagttaccctatttgataattatataatgtatacatgtatcaaactatcacatgtaccccataaatatgtacaattattatgtgtcaataatttttaaaataaatgatgccaacttaatccctagcttacttggattcaggtttctgagatattattttctttattcaggggctatgggtatatttccccttttgcttgtttaagaacctgcattttgagcaaggggaaaaaacatcactgacctttgatatctctccctctcttctccctacccaaaaaccctctcttttatgacctagcatcctggtttctcaacagagtttatggagaattttcagtgctctcctagtataagttttcaggttgacagatttcctcaacctgatagacagttaaaaatacactccagaatcaaatcaaaatattttacagtaaaacctcttatcagaaaataaccaaagagatgagttaaaacaaacggtttagtcaaatatcttaaacagttctgaggaaacaaatggaaatagaacaatagcacttctctgaagagatagtcagtgtcagaatcaagaaagacatgctgttatctcaaaggtacccatgagaataatactcttttcactgtttctaagaatgcaggcactgaactgatggctcttctcagagtcagccttctcctgaagtgcaatgaagccctccaagcacactccaaacattaacaacacactacacaggcttttttaccactggcagtgaagtgcttcctcattagaagccttaaaggaagtatgaaatgcacccatttgtcaataaatatttattacaccttactatgctctaagcactgtgctaagaattgctgagggaaaaaagatcatgttctcacacagctgcaacaatttaatatcgggattaacttgaaagttaaaatataagggctcagggtctagatatataagattgtgagctgatccaaggatgtattagtttcttacggctgttgtaacaaattagcaaaactcagtggcttacaacaacagaaatttattcttttgccagttctggaggctagaagtctgaaatcaaggtctgagcaaggccatgatttctctaaaggctctagtaagtatcctgccttgctgctttctaactttggaggttgttggcaatccttgcagcttgcagctgcatcatcccaatctctggcccggtactcacgtagttttctccctgtgtgtctcggtcttttcttcttataaggaccccagttatattggatttggacccacacaatgacctcatcttcattgtgtctgcaaagaccatatttccaaataagatcacactcaaacatgccagggttaggacttcaacataacttttggaggattacaattcaacccacaacaaagaattttgacttagaagactagatggttattgatacaattaagaatacactgatggaggagaaaattgaggaggtaggggaaatacagagtttaatttggtggcatcataaatttgaactacttgcagtacaggcagtgaacctgaacaggaggtatatggaaagataaatcttaaggaaagaagaaaagtcagagttgtagataggttgggattttgtatgctaactttcattaatcacgggattgtgtaggggtcagttccctgggaaaccaatcctgagagagaaatctgagatttactgagaagtgttttcaggaacgaaacgtaacatctgtaaaaagtgaggcatgcaaaattattgggcagagagagaagttgaactgttaaaattgcaacagagatttcggctgatcctattggcatctctgatgctgtgacaaccctctggagtagttccaaaacaaagcaagagggtcaggcctttcttctccacaccattcattggatgcaggctctccctttacctttccaggggagaagggtataaccttgggggaagcacctcccttctgccacgataaggcccaaagagggattcagatgtgagccttcagcatccaacacttttggcagctgggggaatgagcagcttgctggaaagcaggatgtggacagtgccttagagcatctacttcatgacagaaccacagacagcagctgagaacagcggatcaaacacatttgagaggtggatgcaaaagaagaaagcacgcacacacacacggataaaatgtatacagtagcccctttacacacattatataattttcgtctttgtgacctccttaggaagtaaatataatgcatctatatttgcagatgactgcgattctaaaagattaagaaaagatttggttagtcatggacgtatctgggggtctttttaaatttaaaccccaagctgtaatcattatactacatgccatagaaaggcaagaaaatgagtagtagtataattgttttctgctatacctctgtaatggtgtaattcagggtgaaattttctgttgtaatggtattgtacatttgctgattcattgggattctgatattgtgcttctaatccaccttaatgctgactgattaggttatagtctaccttggtaataatgtgtgatttttatattttaggtgtttaaatttgtttcttcaaaactatcttattcccttctttattgaaaagtaaaactgattttacttctagaatatatttctttctcaagactacataaccctttcctttccatttattcagtattaaatgcatattttatacattatggtatttcatgtgtatctgtatatttatttttgcatatattaatgtgtctattttttcatgcaaattattgataaatatgacaaactcatctagaccaaattcaagttcttgtagcttctgcctgatacattcattcttctctaggtgaaaaccttgctgaggtaactatgaggttcttagtgctactaagacttatccagctctccctggcttgttgatgaaattggcaagtgactcagaaatatcactaaagtccagatcattgcctttaaagcatctcctttgtcatgacttattatgttattacagaaaagtattaaatcaatttggcataattttttctacacatcactttgctctctttgaacctttgtgttctctcaggacaatgtattaagtagaattgatttattattatattatctttcacattaaggctatattaggggctagggagtacacttactgccacaccaaggtacacatgttaagatcttaactatttgaataaacctattttacttaagggtacatgggtaatccctatttttttgtgtgtgtgttttgttatttttggctatttctgagtcaggactctgtttaactgcatgcaacagagacttaactacattggcataactggatagaacattctgttttcatattttaaaacatccacagatcagcagtccacgtggcctccatgtcctaggctccatctcttctcccttctgccaatcttcacatgtgttttttcttctatattaatgcctatgattagaggtgactgttccacctacagctttatatccaatacccaggtagagaaagaaatagaacgaagcagaagtaatgactatcccacttgcctgaggaagggcaaaactttcccagagatccttatccaactttggcttatgtcttattggcaagaacagtgtgacatggccacccctaacaacaaggtagcctggctgaggcaggacatttatctgggcatattgctaccctgaataaaatctggattctcttggtaagataccttggaaggtattaacaaggtcaggcatgcttctccaatattactactgctactaattattattattcctttatgaaaatgtcaaaagaattgaaggatggtaattttctattttaagagatgcagttactaggcataatttggaataatatgcatcaattgtaatgttatactttgaaacatattgtataaaattaccaaaaaacaaaagttaatttttacttattgaattagaaaatatatatgaaaataagaatattttaagttaaataataaaaaactaaatatccaaccatttttaatctgcaaaaactagaaatttcatgtggctcaacataatacaaaggcataagtaaaatataaacttcattctacctgtataatcttatttcttaattacataaatattatgttactgaacacagatattttattcatttcttttatttgtgcatagtagagaacacattaaaattttctatgactctctgcccagaattataataggcatagagtttttctctcctaaattacactcatgaaatatttttggccccctataaatattgagtcccccaaaaaccacactaaaaacttcatgtacttcaaaggtaaagggtgattgatcattgacaacaaaaagtatacactctaatagcttgcaccttaggtaaatatacaaaaaattataacacagaacaaagcaaagcacaatccgtaacaggacactaaactagtccattgaggatgcattgagtaaggggctgtatctggctgacttcacatgaaaggaggtggcacttagaccagaccttacagtatggagaaaatttcactgggcagatgtggtacagaaggaaatctcaggcagaggaacaagcattcatattgttcacctgattcactcattcattcatctatccacccattcatctattcagtgttcagtttacaaagtcattatctttggagatacttaaatatgcttattattatgaaatgtagtcattttgttgacctgataaaaagttgtttcaatatttgcagttcccaaattaaatatttaatgtccttttactatgtggtggttttaattacactgttatcaagactttctctctttagatttttaatcaaagttttagatttaccttctgcttattactacactttagaaaatataataatcttttaaaaatgcatttgactttgaaagatttagggattagttacagttatgctgtttcgtaaaattggcatttgattctatattttatgcatagattttttttaaaagcactcttctgtagaattgcacttagaccatttgtttttttcaggttgtttccaaagaatttgaaaaatctttccatcttcattggatttgtttacgtgcctaaaaacaatgaaatatttgtctgacatattcaaatcaatgtgtcttattctttatattttgataaatttggaaacttcacttttttgcaatgctgtttaaaatgtcaaaattaattaaataaaaataagtcaatcaagaaatgctaattttagaatatgctttttaaagctgcatactcttactgccacctcaccccctatactgagaatcacttatatttaaatgttgcttgcatttaaggaaaaatagaatgcaaaacattgcattagcacattatagtatttaaattcaatttttaaatagtagagtagaattatgtaactgtttaattgattgtttttaagtctctgttgaatgacatttaatcttagtactgtgattcaaatatattgcttaacgttgagtatatagcatgtactttcaaatatgtaactaatttctacttacaggtatatgactttaaacattttaaaacacagttgactaaactctttcatcatgctgcatttctaaaattcagtacctaaccttcttctcacaggggtaatatcaaactggatcagtcaaggatgcagctctgtgcttttaaccaggcagagacatatgtcaccacttttacacaggttacctaaaaaaatcatacatacgatattttttcaatgtcttgcatgctgttcagctacaaaacctttctgcctgacccactgcaagaaagtagaaacatatacagttgttcaatagataaagtgacatcacagcatgtcaccagggcaatttgattatttttttgtgattctcttgatgacttcttgatgtaaccttcatgtgcttaagtgatttcaagtgcatgatctgtagatttttattgaattgattataaaagtaaaatgcagaatgtaacagtagccattgaagctacatttaatttgaaaacctctatcaacttcccttcctgatttaatctacataaaaatatggagagatgtatatcacattaaattgtcacatttaaattaagtagatcaaaactctaaaaaattgtggaagaagtgtttctggcataaatataaaatctcagagcaggaaaagatcattgtaattctagctttgtatttaataattctggttatgtatttatggataagcaaattgagactcatgctttcttagtacacatctagaaagttggggtcaaagccaaagtaccctgcttctagtataaactttgaatacattttgaaatggttttatgcactgttagttatgggactttatttttttaaatcaactcagtaattaacatttgattttatttgctcctatataaaatgcatcagttaaaagttgataggccttgcctatgaatgaataagtgataagtgttcaaaatgtttaattttattccagaagtaatattactcattgataagaataagtttctgattatatacataatcagatcagtctctagaaactaagaggtttagatagagcatattataaatcctatctatcaataacacatagaacacttttactgaaattagatacagattaatatgtattgccttattgaatatttctaaaatgtattaaaatacatactaagatatattttaattttagcatactttgttgaatgactgaactttaatagttctaggatgggggcaggaatctctgccaataagaatcttgactcctttttataatctagttcatattttgaatttctctgtcattgttgagatgtttgtattattgttatctattgtgcaatgatgaaagcaaaactttatcctgaaattgacacaccttagagataatattgctaacagtttcaaatgaaaaattagttattttagtgttccagtttatttgaagataattttaaaggtggcctaagtaaaagatatatttttctatttcaaatttggtggggatttagaggcaatattctcatagaaaactattttattccagaatatggtgaattttcttcaactagagcctaaataaggatgcatatacacccaatattatctatataggtatagataaaacatataatttttgagaaagctatttctagaaatatatatacatgcatagacatataaacatatgcatatatatgttttgaattcataaattcttattacttttttaccaagctgttagaaaagatgaggtacatgttaatgaatggtcatcctcttaagaaaaaaactgaatattcagttaagaatgacttgaaatatggcactttagcaaaagctgcctacataaaggatatagttattttatccatgtaaaatttctatttatattttcaatattagaaatccacatgtttaaaaagctagtaattattccattcattaaaattcacttttccttgccctccttcattgtgggtagtattagtagttaacagaaatgattcagatatctaggtagcttactttgtgtttgtcattcttgtttgtttcaaaattatagataatatttacgaagcttgagtctgcgtatctacactttgcctttatgtttgctaaggttccaatccttctttacctacttgttgcatttttttaagctggcataaatagcaaaattgtttaaatgtttgcttctaaagactttaagacactctgacttgacccactgtattccagctgctttctctgcaaacagtgtagcagttctgtgacagagcacagagagagcccttgaatatcaatggccactttacactcctagctctttgcttctcttcttttcattagaaagatgcttcaagatgatactataggacctggctgagaaatctgtagccagtctagccttcttacatggccaactgttgttgcttttatatgatttctttatattcatatcttatcatttagtcacgtaagttttcatgttattggatagaaagtgccctaggtagaagattctgagaaaaagaagaggtctccgtgaaatcactggttttattctgaatttctctggtggtggctgagatttgacctcattatttcaccaacagaatactatatatcatgttgaccttgtgatggaactgtgtgtttcacgctgaataagtagctattcctcccagagcctacacaatatttctctgctagcaatagacaaagaaaaataggaaaggaaaaaaaattggttttcttgccctttttttaaaaaaagagctaaagttttagttgcccataattagaatgtgagcccagaatttattgcaactagttaataaactgacaattttctgctaaattagtggaagtaacattaaaacttagacaacagaagaaaatacagttgagttttccactgatcaataatatctggagtattttttaattatttccatttttttcctgaaattcctgagcttcagtgattttttaacttgaggcattaccttttaaaaagaatcttgagaaatttcatatcaaaaaaaaaagattgtagagacctcaaggggcccagaaatctaaatcatgttaagaaatgtgattacactcaagatgaaaagcactaatagtaatatcaataatattaatgacctccaattttaaaaactctataagaggataatgttcacatttctccagaaggaaaaaaataaaggccctgaatgattgcaagagagaagaaaggttatttttgttctacaccagaagagagtatttaaacatctagttgtcctgtaacataatgggaaacttggaaaatattggactcttatgccacatagtaactatgtgtggatttcttctcaaaggatcctttgatcctttaattgaaatttactattttactccttaatgattcaagaacacaattatagatattgaccgtaaaactacacaattaaattaaaagatggataatagtaacttgaaactaattttaaaatgtctttcttgcttattaacagataatggagcttagtttaccactgtctgccataatttttcttctcttttatacccatctcctccttttgatttgcttctctcaactttcttactgtcttttgtgagtattttaaggctcaggtgaaataaccattctaggctctgctccttaaagaatatctttttttttgttgtgtgaagcaaacagtttatagtatgtacttgaaatgtaacatttttcagttcttttcagcatcaatcattctctttgagtaaggtttttgttgagttctgtgttttgtacagtgctgctgtgtttgatagtttcttcttttgagcgttagattatcaagtcattagacactcctacctcacacctcttggaacaataaataactagcattaaccatcatccatgatagcatgaaataggcttacatcgttaatgtttatcttaggaaatgccagtgataaatttgtaagtgaacaacttaggacatgttctaatatgatacatatatttaacacaatttctactcaagcagtatagtagaaaacacagcttcaaggcaatcaaagggaaaaccatgaagaataataaagcttttaatttggacatattttcagtaattgaaaaggagcatcttaaatagctacataagtttacaatcaagataaggattgtttcacagctatagtgttgtatggatgcacactgaaaaatatggaaagatatgtctctctctctctctctctctctctcctctctctctttttctctctctcgattacctcaagggggttgaatcaaggagatagtggagggaacttttattttgcactgtaaatactattatacatttaaaatgtttgagtcaactatatcactttcatatttttaaaacacttttagaaggcacagtcaatatcatattattcaataaaaaggcaggaatgctaattatggtacacttctgattcctaatactagtgagtccaccttcttgatcctatgcttatatttatatttactttatttataactagactgccaatgatgaaaaaaattgatacttcccttttattagaaatcaaatccatgtgaaaattacagaaggtcctatttataatagcaaaagaaatttctttcatactaaagcatttatttcacttctctaatatagtcattctttcatttccaaaattacatggaaaactaaacaacagatgattgagtttaaagaaatgaagataatatagaaattaatcatataaaataatacaaataattgatatgatcttccttgtatagtaaaaaacatatattcaaagtacttaaatatggtgatgcctactgtgagaattcaatatatttcaccgcaggaaaaaaaaactttattatatatatcagcctttctatataaaggtattctcttagaaatctgctaaagaagcttatactgtagcaagtatagatttcaaaggctgaattgactagaactgtctatattgctaaagcatgcttataaacatttaattttatatacagtatggaaatagttactgtttgaatacttaatgcattcccaagaaaacattgaatgtctaagaatctattagccataataatttgtctgtaaatacaaattaattaataatagttcatgttaaatattttgataatttctgtcccattgaacaataatattgaataactttatgatgtaaaattctgtgccaagctgaattcaattgaccattaaaaaaacataaaacctgcatatgaaaaaaagtttcaacaacaagatacactatataaaatcacgtgaatttttacttgctcataatctaatttggcataaatctttgttaatgctctcttaaacagtgagaaaaccaattattgatgtttgaagatgctttcatgaatttagctaaagtcagaattctgcaaataagaatcattttatttaaaagttgttaccagctcagagaaaaaccattataactatttcctattgttaatattgtgtctaatagcaggggggtgctttgtattctttctatcagcaattcctctcactgctttacctcaaaacccaggccaataattcttttttcagaaatgaagggcctggtcttaacttcacagaataatagggccttgtctttcacttctctggtgatagttcacgtgcagccgagaggtcaggaattgaagccattaaaactgtagtcttctaaatatggtactttggattttgtggcaattggcacaaaatacatattagtaattataagtttatccaacatcgtttcctggattaatctgtattttaaaacttatatattttaaaactcattctatagtttatactttaaagcctattctatagttttatagaatattcagttataattaatttctattatcttattttaaattaaactaatttagatgaattcaatcagtttaaataacatgtttttgagtgaaattttgtactgatactttcataactgaaactgttatcaaactcctggacctggtgggaataatgcctctaggccaacatagattggaggagttctcttaataggttgttcataattatttactccagttataagaaagacattgtctgcttacatcattccttatgtacaatactttatccatgccatttttatgatgttgtatactaggtgataataatagcattgagaatttaatgtggtacccatcaagaatacttctagcttccaagtcaatctctaaaagtcaaacctttagattgggctatcatattaatacaaattcatagattgagaatcaatttatctgaaataagtattttattatttttagcacttgcacttgcttgaactactctaaaaaagataaagttgtaacttaataagagaattgtgttgtagtaactagtagctttcaatattcacatgtcatttagcaaatgagaatgaaaaattaactcttccattgatatgctgctttactcctttataaaacaatatttgggaaatttgacagagttttgctacagttgctgtatgtattttttgtaaagatttcttcgtgtgtgtgtgtgtatgtgtaaacatcatggttactcataaagctctgggtgagaaaaacctatttgcgttatgatcacttatatgatcataatgacaatgaattcattattccatgtgtaagtctgccatgtttaattttcttcgatctttgtttcagagtttgcatgtggtgcttactttagtagccactttcttgacattataaataatactgtttgatcttttctactatatactaaaatgttatatacaaaatgggtggtgttggtatggtcattggcattttgatttcaaaaccgtatttgattttgtggtcttgatcaaagaacttctatctatatcatcctgctacatatatttaaattcaagagataataaaaaatacatttggactttatcttgcacttatctttttaaaaaattttaactatcaataagttttttgccaactatttgatctgagctttcaatgatcaggcattagtaatttgacacttgtattatattgcaaatagggtataggttgtcctcaaataatgattgtcagtgtccataaatttatattaaaaatttaaaaaataagatgtggtcaatactttccaaaagtgatagttaacatcaatatattaccttatctaattttggccacgtacttgttagtataataaacataataaagtttattattttacaggatggtgtagatagaaagatcaagtatagatacatttaaggttacttgacctcaatatgtgtttatacattttttatggccatcatactattaatcttttttcataatactttgcatgagtctgattattaaataaggcttaaatatcatatttgaatgaactctaaatgaactacctggtggggtggtgaattcctttctagagattaaaccggtcaagctcctcagagtgcagcacagttgataaccctttgcctggagaaggagaagaagcagaggctgaacctatgaattccgatgagccagaggcctgtttcacagatggtaagacaaaaattgagaccttggttagcattccttaattagtgttctggggtttgtcttaacgcctaatacttaccagaggttgtcggtaagtcagaagactccattccatgctgattcagctatagctgtatgatgattaagggcaagtcactagagctctctaagcttgagtttccacatctatggcacttgactaacagtaatttcctatcactgtatgttagactgtaagcatgctactacttcttattttagagccttaattcgtattcccagatcaatggaaccaccagaatacaagttaatgaaagagtctgtactctggtccttttaatcaaaaatggaatctactcactaactctcagaaaactgtttgatactgttgactacagtctctctcaaaattcttcctttccttagtttcagaggccatagtctacctttggtcttttatttgtcttagagttctgagttcctctctctatttctttaatgagtattttttctgcacatcctgtaaataatgatttctccccacatgttcaatcttcagttatcttttcttattctgaagctctgaggtgtttcatgcatgcctgtggcttgaaaagccatttctgtgctattgactcttaacatcaatgtctttacctctaacctcactcttgagtcccagatctgcttacaattccccattggtcatcaccatctgttgtttgtaaaacattccaagtttaataagtttttttttttttgaaaagcaaaaacaaaaacaaaactttagtatggtttacagcttcctccagtctttattagtaacttcatgatctaccttaccctcaagctaaagactttgagagacccttaaagttttccttgctagtcacaagttcatcaggaactctggagatagggcccaagcccttaagaggatttgcatgcacactaaaatgtgagaatccttggcctctagggtaaagtctgaagtccttaacttgacatgtaaagttctttacatttggccttagtctcttgttttgtaacagtacctccttctactcctgtatgctttctgatatgctgaatttcttactacttctggaatttgtcagggataatgcagtattgtctgaacaagtattgtatcatctaggtaaaaatacaagcccagagtagggctgcaatgtaatacagtaatacaaagttcaaactttggagcctgataaacatatgttctaatttgccgacttatctgcctgtgagaacttgtgcacaatttaaaatcttgttcttcagatatctcatctgtaaaatggggatatcagcatccacatcacagcgatgctaagaggatcaaataagataatgattataaacttactagcacagtgcctatcttccttcttctacttttcttattcttcaaggattctgttaacaatcttatacagaagttatgatagcacagatgctaatgacttatctagtcatgggcttaccctcagcattagtattttttgaaaaatcatcgacttgctttcagtgtccaagattttctcaactggtttcttataacattcatgggttaagcaaaatgattggaaaataaagacatcagcaaactacaaagagtgcatggattttctttaaatacacaccaaatatcattttatgtaagaagcaaaggtgtacgacatccagtcaatataaattttgtgcttgctgaaattagtgctataagcaaatgcacagtttcctttaaaagtgtccatgttacagagatttaagaatttcatcaacatgtatggcctgctcctcaagaaatttattagcagtatatagacttaaatttgtattgatgctaccaagattttccctattcattttatgaatgatcatgacttaaaattctaggataactagaaagaagttgggcttatcaatcaacttaaagtttcaaaccaaaaaatgacttcaaaaaacttaaactcttacagaaagtattaatactatgctatttcagactgtaggtgctgttataataagatataatacaaagtttttttaatcctataaacatcaatgaaaatacttttgtgaatactctgagattattcattgcttccaaactgaggccagtatagaagaaatattatggaaacacattttccaatgaacctgggcttggaccattgaaatttactgagggagcatatatctctgagtcacatcccttttatgtgctccagcagagacttcatacagtctctggatctgtagcctaataaattcagtgaagcagccaatttgcttagcctttcaaccgtgtattcacataatcattctttattccagtaaacaaataattattaaatacctgtgtgccaggtccagttccaggcactggagggagatggagctgtgaacaaaacaataccccttgctgaagaaacatatcaagatgtagcaggtcagataagtgaaaagtgctatggagaaggtatatggaaaagaggaataccaagagtgagggggaagaacttactagcacataaatatgctatcaatgaatgattggtcagagaaagcctgcctataaaggtggcgtttcttccgtgatgtaaagaaaattgaagggggccgtgcaaatatatggagggaaagtattttaggcagagtaatgaaggcacatgccccaaggtgaggatatgcataacatttgggaaaaaccaagaggccaaaatagatggagaataatgaaagatgggaagaggattaaaagttaaggtcagaggctgggtgaagtggctcacacctataattccagtactttgggaggccgaggtgggcgatcacttgaggccaggagttcaagaccagcctggccaacgtagcgaaaccccatttttactgaaaatgaaaaaaaaattaattgatcttggtgatacatgcctataatcccagctacttgtgaggctgaggcacgagaattacttgaatccaggaggcagaggttgcagtgagccaagattgtgccactgcactccagcctggacgacagaggaagactctgtctccaacaaacaaacaaataaaacaaatgacattatttttaaaaagtgtagatcagagatgtagtagaggccagattataagacatttgaagaacattataaggatgtgattttaaaatctcacaatgtgcaatcttaagagtggaagggcgaatagcaatggcagaagctggaagatttcttggagtttctgcataaatccaggtgagaggtgatgggagcttagactagtatggtagagttttgtatagtgagggtgtgacatgctgagtatattttgaagttagagctgaagggatttgttgatggattgaacattgggtgcttaaaaactggaaatgcatagtttccacatgaagactgagaagcatgggcttgtcagaatgaagaatcatgaacatgtttacttacaagttggtaattggtctacaagagctgggctagaatcaatgccagttcagagcaatgtttcacctccagagatctctgctacctgaaaggtaacagaaaaaattaaatgaagtatgaaaaagaattaaatccaagtagcatgtttttactgaagataaatgttaaaacgattttgttgttcatagtctcacatttgttgattcatttcagcttcataattatcactttactaatgtatttcattgattgcatctggttcctcttattcaccttttctctccctcttatgcaaggttgttccttagctctgtgtctattactttgtatttgaagacttcagactgcttccgtatatcttgccctgttctttactatccttagttctacatgacttggcaacctatataattatgaacatgacatgactctttgggtctatgtggttttatattgtgctgcttttagattatcaaattagtaaattacataatcaggattagagttagcgaaaaataacctttttcacaaggtcaagagatcgagaccatcctggctaacatggtgaaatcccgtttctactaaaaatacaaaaattagctgagcctggtggtgagcgcctgtagtcccagctactcaagaggctgaggcaggagaatcgcttgaacccaggaggcagaggttgcagtgagctgagatcctgctactgcactccagcctggcaacaaagtgagactctgtctcaaaaaataataatggtaataatagtaatgtatttttctgttaacttggtaggagaagccaaatcatcttcctttcaaaatgttttgtattcacccccttcctataaaataactaataattaattctaactttagtaacacaataaccaactcctttgtgacttttattttttctttcttttggaaagataatgttaacaaatatttgttttagttatatttgatgttaagaaatggtgaaaaaagcttgttttcataatcaatttttaaattaattatattgatcatgctgttatggtttttaatgaatagtctgtcttttgaagtggtcagtggtttcttttttctttcctttccttttttttttttttttttttttgagagctttggtttaacatgatgaaactcagcatagactttatgtaatgcaccatgagagcacattagtgaaaccacccaaaagaaatagcatttactcactgggacccattaaaatgtttgagagattctctgttatgtttacattcaattaatagtccttttaaagagaaggtatgataacattgctaaccagcttagttggaaatctcttgggatatacctttgcacaaaaaccagacaggcgctccatcactcttgcctcaaaataagagaaagaaactctctgaggtgtaataaaccctgagggcaatatttatgaaacttcatttcagattgaaagtttactaacttttctaattaggatttttagtgtttttatttcctcaatcataagaataatgtttcccttcaattttaagattgacaaaagtaaatcccagagagcaaaatcttcttaaccaaagggataattttcttatccaataatttgcccctgagaaagataaattctttcttatatgtgatcattgtttttaagtttaagacaaattctaaagcactaagttaatattaaaaaattcatctcatttttttcgctctctctggtatttttattttccctgaaaaagattaacttagagttaatttttgaccctacccttttactctgtttttatatcattttatcgaatgttctctcttaactaaatgcatgtagataataattatttaagtataggatgatacataatttcagatctgagatcatgttcactgtaattaacattgtataaatgaagctatgtgtagattttccattcacctttttttttctttttcatccaatgtttatgtaaagtatagaattcacagagatgtaacctttcaaattatgactaactgcttagaagattatataactctttttggggaatcaagtcttttaaaaatcaactataaatgacctattaagccaagtagaggactatgatgagtactataagcacaaagagcaagcattacagactcagtttgagtcttctgggaacttagaaacttccagagagaaaaaacaggaacacataaaatatacatgataacaaaaacaccagagacatagggggagagggaaacaccttgtttgctgtatttctatagagctggaaattaaagtgctagaaaataaccttatatcaactggatcaatgcactaattagacttcagaaacatgaaaagattcattacaaaacattttctaccaggtgcatcttgtcaagaggattctagttttgttgttttagaacatacaacttatgattatctgaagttgttacttgaaaactcaatacttgttaatgaattatattgttttacagaaaaggagatcaatcttttagaaatatctaaaattcaaacttctgaaacaggtatcaaaaaagtgctgcccattcttactttcccacctcctgccttcatctaaaatgaggtttttcatctaaaattttatgaggaagaattcactgtagttactttttaaagagtctctcttttcttcagagtaattctagttgctttaataaactttaaattgattaaaatttttcaatgtttcaatcattctctttctgggcctcttatgttcatagctctgtttatcacttcttataaaactttttactctttggtttctcttttactgcaccctcttggtccttcttgttcatctctcatttcatctacttcaaagttttctcctctacctgtcctagtgatgtgtaggtaaatatttttataaactggttctccaaggaaaataaaaaatgtgtacttatatcatttactataaattttactgatattaggatatatgacacacaatttacaaataataaaatattacaactttttattgaacattctgtatatcaaattgtttctcaaagaatgctttttttgaactttgccaaacttttgcatgtataggcaacctatttttgcagctaataaacaagtatagttttagcatgaagactgcttgatatttatgttagtcagtgaaataaaaaagaaatgaagaagctatatgtcagaactttactcattcatcagtgatgtaagcaattatttgcaaatcaaaaaacagaatttcaataatgaaaggatattttttaatttttgtgctattcagagtaatggctacaaacacaatacaccttgactgtttatctgttttattggcatttcctccatcaccttcttaattctactctagaaaatcaagaagctctcatttgtagtgcttggagatttttgtaatgtaaagattcccaccatcactgatttcaaactactgatgtgaaattgctaaatgcagagttaagaagagattcacagtagctcaacagtgtgacatacttccaccatccagatagaataggccagatagaatagaccagcatccagaaagaagagttgcacagtagcccaatagtatagcatattactactgtccagagagaataatccagagagaatagaccaccgtccagaaagaagagatgcacaatagcccgacagtatgccatacttctgctattcaaacggaatagacaaataaaatacagtaaaataattaggaagtgatatgtcttagtatttattacctttccttaaggtaagttatagaattctaagtttatatagtcaaatatttaataatggctgcttttaacaacagacaagcaggaatttctgacagtttaacaacaagaattggtatggtctgctttcagcacaccattgaattctctcccttaatattgttgtttcagaatgtttcggagttggccctttttctgctcaccctaaatactctttttgtgtgacctcaaaaactgttctgacttcagcagatacatgtatgctaattattactaagaattcttttctagacaggtttttcccgtttcaggttcccacccccatttttaaaatctataggatatctgtatttaaacaattcacaggcatctcaaaatcattacctgccgagtcagctcctcctcctgtggttttcaattcactgctaacatccacctggtagtcctgggatccttcctttctcttacatttgatatccaatcaatcaccaaatcctgctgattttatctgccaaatatgtcttgaatctgttacaactctagttcctaactaatctttccctccagacaggatactggaccagaatccaaatggttttctgggtatgaaactttcttcacccctactcctctctattcacccttccaacttatcctccatgtaccgtggagacatctatttacattgcaatttgcgtatctcgttccctgcttataaccctttacttactcctcaacctcaataggatataagccaaactccttaaggtaaaggccaatgctttccataatctgaccaatttttctaagctcacctctcacccttgcctcttgtgtacattctgcttgcatcacaattctaaactggtttaaatttctatgcataacaatttttgctgcctggaattccacccaaactcttttttactgatattttgattcaacccaggctccatctcttctaggaagcctctactgatctctctttctcaagaaataagccaggtgagattgatttcttccatgctatcaaaatgaaccctgactatctgtcactttagttgccatatctagttatagtcatttatttctgtttttatctacctaaccaaaacctaacataaattcactgaggaaaggaaatcatgtacagaggtgaatacataaaagatacgtaacaagggtttgatggacaaaagaatgcgttaatgaatgaataaacctaaccatttttaagtacaattttcaattttttgtagcagtatattttgtacataaaaatttaaagtcacatttgtttttaacttagaatacttgaaacagtttcatctggatcttctaatcaagatgagtttcttttgaacttaatttcaggagtttccactaattttctcaatattgtttagaaaggtgtatttcagagggtttttaccatagaggagtggaagtatctaaaattatagactagaaaaatttatctgtcagaaaagaagaccctaaaattcatgtcgtcttcagattttagttcattcattcattcacacataaaatgaaagtggtttaaagactgtggaggagactgaagagaaattggggattacagaagacaagaatacattgtaagctaaaactaactaacaactatttagaaaacatcatgcaaagttagaagatgaaggaaggcagcatccaaattaaatctatctgaaattgcaagtataggtgttgaaattatttggttgagggagtatcacagaaagcagtttgcattaaataatgtattaaaaattttatttattctgaatatcaagatttaacatgcatgtctttcttgtcaggttgtgtatggaggttctcatgctgccaagttaacatagagtcagggaaaggaaaaatctggtggaacatcaggaaaacctgctacaagattgttgaacacagttggtttgaaagcttcattgtcctcatgatcctgctcagcagtggtgccctggtaaatgatctgacacctaagtcaatatattgattaagtcaatattctttaaaatgagctaaaagataaaaatatcatggcaagttgttttacctagaattaatccctatgattagtgccaatgacaaaattttacttgagctcagtaaatagttactgagcactcctacatgccaggcattatccttgctgccctgaatgctgttgtccaaaggaccacccagatggctataatagaaaagagaattttactggcaatatcagtttgcaaaacagggagagagtctgtggcataaactgaaagcactttccctgtagaacaaagagagaaggttagaggttttattaaaatgggaaaatgttatatattgctctttgagaaagttcattggccctagtaaggatctggggaactggtaagctccaactggcgagcaacagtcgttggcacaattagtcctagagtgtaacaggtttcctcagaagctatagataaaactaatttcaagttacaacaagcagcttcaacagtcagtcttgcagagaattataattcttggagtaatgttttgtaccctgagagcttttccccctgccttcttgactcttagttgactgttagttgagtaggacaagaactacccaatttgtatgatcaactttcacaatgcaaagataaataagatccaggtactgtttattcttaaggagctcagagcaatggagaagagaagaatacaaatgagtaactgtagagcttggatgtgtttgtaagcaaagtgtaaatgtgacaaagttctgtagcaaggagaacaactgcttgcaatttcagaaacgtaaatggtgcttttcagggtctagaaagacgaacaggaattttctagttaggcaaggcaggctgaggggaacaagtatacaatcgtaaggaggcataaaacagcatcagatattcaggaaattaaaaactgctgaactgcaagtgtggtgggaagaagaacaagtacaggtggccagagatactatgagaaagcaaggtcagcttgtgaagcatcttgtgtgttataaaaagggaaaacagatggtagttttaggataaaagtaggacaagtttctcattcttgtacacatacaagcagcttccatgagagttgaaagagatctagtagaattacaagctacctgaacctttttcttaattgtcatttttcctgctgacgctgacagagatgagccattagaagccattaggagcccagatctagctagatataccactggaagtttcattatactaaaaatagaatactcaagaactttgtaacttgcctcatagacagtcttcatgagtttggactttgattttactgtacttatatgctaataggttgatctgatactgtttcatggatgctggcagaagatatgaggtgcttgggtcagagataaaggactttgttacttatgtgtggaaagtagcatgagtttcaacatgtttctgttggttccccctttacccaggtctcatgggggcattatatgagcctgaatgagtgtgtacacatatagtggattgatgtcacagctgaggaacactgagcttagggaatgcatccttttataccaagcaacataagcaaccctgctctttgtcccagagaccctacctcatgtctcaaggttgctcactgcaaacacaaccctgaggaatgtcctaggtaaggagcaggcagagcctggcattcttggcacactccccaacaacctgcagggacccactgtggctttcctttcctatcaagtctcaactctcagaaacagaaaatgtgatcagaaaagctctattttccttacctggagtcatccaagtagaaatcatgatagtgtctaccatgcaatgtattcctagtaactaaggaggtttatcccatgctgactcacatatgtgactgagaatttcagagaggagattacaaaaaaattaaagtttggagtaattccttgttcaaaatgtctaaaacagaggaagacatttggggagggcattctcaaaagtgtattttaaataaatcatatgaggaagaaaagggaaagatatttcaagaagctggtgtttgtacttagacaggttgccaattaaactagattttttaaaagtatagcaaacccccatgacacgtgtttacctatgtaacaaaccttcacatgtaccctcaaacctaaaataaaagttaaaaagaagtaatagctctcatttagcttatacttattgtgtttcagaatcttcttttagattttctcatttaattttcaaacaaccctgtgagattatgatattttgcttattttaccaatgatgaagctgggtacagttttaaaagttgcttaatgtcccaaagctagtaaatgacagagctatcactgaattgacatctaaaatcaaagtcaccatatcttttcactgtgttgtggctcctcagaaagaagagtatacacaaaacagaaataaatgagtggtaactgaatttagtaacagtaaagcgttaagtcctaaatcagatagagctcacataaggtcattcttgcctccttgctcctacatccacaaaattggttctggaagctagaacccctgtgggctgaattctaattcctgctctgggaccagtcagagagaggttctcatcatcaagacttcctgcggctctgctctgaccctgtacaccagttctagaaacaggcttcgttttccttgtgactaagccccttccttgtctttgtatttcaaggactgagtcattgccatagtttcggtttagatggcttgcctgattcctccagaatttgggggtggagtggaggggaggaaaatcctgagggctagaagcctccctctgcacttcaattccattgaatttcagaacctctgccaactgctgggaccttctttttgcatcttagtaggtccattcagtctttctgccccaacttgtctcttttcttcaatattgataccaacagtttcccaaaactgctaaggataattttagagtattgaaagcagaacgtagaataagaagaatctgaatggcacaagttcatgactgtagacaggtaataaatataatattaaccacgaaggatacaggtctttgactttcattttatccaacagggttaatagtgtttcgatctgaaagagaaagttctaacactttagacctgttgagatcagcaatcattttcagtgtctcaatgagccttttttttttttttttttttgagacagagtcttgctctgttgcccaggctggagtgcagtggcgtgatcttggctcactgcaacaatgagcccaatttttcagtcctttcaaattctttgtcaagggacatgtggctgaagtctaaagtcaaagtcaccctgtcctttcactgtgttgtggctcctcagacagaagagcatacacaaaataggagtaaatgagtgataaccaaaattagtaacagtaaagcgtaagccctacattagatagagctcacctaatgttgtcacataatgttgtgtgtcacttactaaaatttcttggaaagcataggaagcaccgcattaatggagagaatagaacagtgatttttagagctgaagctttgaaggataatatatggatgaatagtcaaaggaaaaagcaccctctgtgttctataggcctattaagctactgcagtcattcatttagcaaatctttcctttcctgctgctgttattaacatggtactaggcactgggatgcacaagcatttgagagctgaaagacaggctccacatcctactggtggggaaaaaggggagtgtaggatgtgaaaagtttataacagtggctggccatgggatcatgtcttaaattgctaggacttatccaattttaatggagaaagaggaggaatcagatggaggtggaaatgatcaggtgtcagagatcatgagtctttttgtggattataagtaatttcattcttttgcactttctcttgacctctgaagaccagcagcagcactggtgagaaggctagaactaactgttatatgataactgtaaaccagaaaaaatgtcagctggcccatgtcaatattatgtttcagcagtcttagcattagaattgtatttttcctgttttttttaaatgaatcatgaagcttaagttgtgcatgattgaaacttgaatattatttccacaggcttttgaagatatttatattgaaaggaaaaagaccattaagattatcctggagtatgcagacaagatcttcacttacatcttcattctggaaatgcttctaaaatggatagcatatggttataaaacatatttcaccaatgcctggtgttggctggatttcctaattgttgatgtaggtacttttgagtacattttaaaagaggatttattcttactgtgtgttgtgaaaaaaccttcaccactattgcatattatcatattttaattttgttaagcttcatatattttcaggctataggtttattcttttagaaatattaacacagccagtggaaatatatctgtgaggaatattagaacagaaatcgttcaacaaatgtaaattatttctgtgacattgtaacatttataattgagttttttatgttttctgcaaaggagatgtgattttttaaaaaatatgatttgaatgtcattcttgttaagaataaaacaaaatttagaagtaggagatgataaaagggcagagacacagattaaagagaggaaatgatgattattcataatgtaaaattggaagataagagaggatgaggggcacatcaagttggctgatatgtgagctaaaatttttctaaaaatcattatacaaatacaaagttaactttgtatttggggagagagaagtcatgaaattttaaaatatctatctcagacccaattataggaaaaaccaaaatgagacacaattgtctctatctcaaatatgcgacttgcccttcatctccagaatggcagtggggcagaggagtaggcattattattaatgtaataattttagataataaattcaaagaactgttgaatacttggatgatggaaaatacataataattctttagtttacatattattttaacgaagtagaaatatttttacaggcttctgattgagatagagaagaaatcttaaaatagtcggtgagatcaaggactctcaaatagaaactaacggtacaatacccattattatcatttcattgaatgtactctaattattttatattttaataatataacttaataatatatatgacatattttgtattaattaaaatggctttcttacattattttataacctcaatctcccagtcttatttttctcagaatgatatgttctttatcttttactaattcttctagattttcagaaacctaacctttgtgtttagggaagaaacatcctataatcagtcatagtttttagagatttgttttccatatgatgcctgacataattagaggtcaacacgcaaatttgtatatgccaataagtgctttaaaatttaaccaagggtttgcattatcgagcttttaagaaaagttgaattcatacagcatcatatgtggattagaaaatcacaagtaagcatgatcatacatagtaaaatctgactgcaaaatgttaccaaattagtcatatttactcaaaagttcaatttctcaactgttattcaaagcttcataaacttttggcttctggccaggcgcggtagctcacgcctgtaatcccagcactttgagagtccgaggcaggtggatcacctgaggtcaggagtttgagaccagcctggccaacatggtgaaaccccgtctctactaaaaaatacaaaaaattagccagacatggtggcaggtgcctgtaatcccagctactcaggaggctgaggcagaagaatcgcttgaacccggtaggcagaggtttcagtgagtcgagatcgtgccactgcactccagcttgggcgacagggtgagactccatctcaaaaaaaataataataattttcgttttcttaatcacacaacatattaaatgttaaaaacaataacttttttctataatatgtaatgtgaaaaaatttttaacatagctatatattacaccttatatgtgttatcttaggtttcctctatgagaattctaacttcggatatagtaatgatttcttgctatttttcaattaatcattcatatatcaagttatctagttactgcctaaaaaatgaaaaatgataaaatgggtatttttgtaatatagtagttttgaagaaaggctgccccaaataagaagaaattatagaaggaatgtagattttcttttctcttaaactgtaaatatgacggggtctctctcttaaaaaattatacaatattttagatattttaaaatctttaaaatattatcacaaatacactcattttcatcattcagtgtaagaaatgaaggtttacaagtaaaattgaacttattttaaattaagagatatggctgtttaataatggaaattgttttatttgaaaatttaaagtgatgattctcagtgagtataacctcctgttcaggggtatgtaatgtgcagaaatcagggagataaacaactccaatttgaaatattatttaatattttggaaacgaacaaatataatgcaaagcacagagagtaaagctccacacaatattcttgctgacagagatgcaaattttgggacacagatttgggggagtggctgattttttttttttctgaaaagtatagaaacttatctacagcagttgttcttgaaggatggtctcaaaactgaaaacgttggcacatgtaggtgagaactcataagagatgcaaattctgcccccaaccccaagacatactgaatcagaaactctggggttggatgtggcaaatcattgtaacaagccctacagatgattctgttgtatattcagatttcagaactacttatattatttgctactcagagaatggacttgtctgatttctgcaggcttctgggttatttgaaacttacttgattaaacagtgttcttcagtacaggcgtatatttgtatatatacgtgtgtgtttatagaaagatgtgaatatatatgcatatatattctcagttacaaactataccatagtcttaataatatgcatatattccgtgtcataaaactcacttaacagtggcttggagaacagacctattagtattttaggggctgtatcaattataacagacatgtcttagtctaccttttattgctgtaactgaatacctgagactggttaaagtattcaataaagaaatttatttcttaaagttctggaagctgggaagtctaaggtgaagggtccacatctggtgggaactttcttgctggtgggcacaccagagagtcccaaggcagctcatggcatcacatggcgaggggtgttcactgagagccaaactgggttttataataaacccactcttgtgatatctaactcactcccatgataacccattaagcccttactctatattccattcatgaaggcagaaccttcatgacctcatcaccttttaaaggctctgcctcttaatactgttacattgggaattaagttccaacatgagtttcaaaagggacaaacattcaaaccacagcaaaacattaaaaaaaaaattttctgattaaagagggagcatacatattttcaagtgttcttaagtggaaaataatggatgtgaatgactgggcaaatttaagagtggcatgaggagtaatttattctgctaaatacgtagctttaggaatttaaaaaaaaaagccatgttacatttagtgagacatctggaaatactacatgaggatgagcactttaaaacacttcccgcttcatcagtcagtttacctgtctctgatggtaacagcaatgaactttttgtgaacaatgactttttctacgcctgaaatagataaatatataaagttggctctcatctgttcctattcttccctcgaaaaattgaatctctcagtcactaacttattcgtaatgaagaactgcattccagacatttttcttagacatcgtctcctgctacacaggatgtgaggcaaatgaatattttccatggaactaattcagagagtaaacatatttatagatccagatctaaggtcatggaggagctcattgctttgtaactaaaatgtgtattgattgtattgctgttcatgttctttcttaagataatttacgataagaactcaaatttcttttggatactggttatggtaattgtggctgatctatctaacatgatgaagctcttattatatctatattgattgtgaagccaatgagatcagctactctcattctctcttcagtacaattgtgatttttctcccatgaaaaactatttggtagatctttggtgctaatctttagaaattgtagggcagaagcaatcttgttgagaatttgccccaactcaaaatcatgagtcttcaaaatgttttttaataagatttatactaagacatagatttagttgagtgaataccacacttttagggcttagcttcagaatcacacacttcattttgacctgagttttataattaaaatgttcaccattgttctaattaaatttttagatttaataaggaattacaaagcaattgattacaaaagtgtgaaaagcatatgcatgatttctattcaaatagaataaacaacttactttgcttacattatttcttcttattttggttaaccttcttttaaatctatttccttacagaaacatccatcttaccctatttttaggcattgactacacaactccgtatataccatcttattgttcctgttgagttgcttttagtgagtttcagaattgactttttccttttatgcttcatcattttattgacacaattaatgaaaatgttatttttataggtttctttggttactttagtggcaaacactcttggctactcagatcttggccccattaaatcccttcggacactgagagctttaagacctctaagagccttatctagatttgaaggaatgagggtaagaaaatactaaactttataatgttcttattttttaatggggtttaaaatataaaaacaatgggtatgtagaaattatgagttgtttctgtttacataaatacataggctgtttatgtatgtgtgtgtacatatacatatatacatatgcatatatgtatatatgcatatgtatactatatagtatgcatttacatactatatacagtatgcatatatactatatatagtatgcatatatggtatatatatgcatattatatacactaatagtatatatatgcatattacatatactaatagtatatatgcatattatatatactaatggtatatatgcatactaatatataaatactaattatagcattaattaaatagtatattactaaaaatagtataatactcattagtatatactagtatattactaaaaaagtataatactaattagtatatactattatatattaatatatacagaatatgaaagactatatataaaatatgcatatatagtatagtatatatattatttcatttactgtacatattagtatatatatgcatactctctctctctctatatatatatatatatacacacacagtatttcagtgcatattgtttaacaagggaataagttaatcacagtaatcctgcctttatctaatattgtcaaacaatagaatgatccaaatgagtgacttttttcatattaaatggaaatcttccctgtgctaggaagtccaagtgaaaggggaaactattgttcctaacatcatctctcatacataagtaatcaaggacctacttatatcaggtgttcaaataatatttgtttaagaaactacctgggtaaggagcattagaaaagataggtaagtgagacagcatcagccatcacatttaatttcacctttaaaataacattgcaaagtaagtggaattataactaaattatatttgagaaaactaaagctcagagagtttacttaatttgcacttaggctgcagagtgcctgaaaagatactcaattagcagagacaaacaagttataatgaataacaactaccataacctagctttaaggattgtattaaaccagcaactttgtaacacacatggaggacaacaactctattttctgtaattagtccatatcctcatgtgttggtactcctctaaaatttcttctccagatttatacctgctattttatgcctctgttctggaatgcttcccctgccctgtcccgaccctaacccgtttgtatagcagacaaacacctatctgtcctttaaggctcaatttaaagttgccttgtccgtccaccaggagacataactggatattctctctctgttcagacacctaaaatcctcattatattgtattttacttatttgtttacttaactgcctccctaccttaccttctctgagcaatttaacacggagattagtccagcttactgatactttaagtgtggtttactgaccagctattagcattatctacaaacttattaaaaagacggacggatcatgaggtcaggagttcgagaccagcctggccaatatggtgaaaccccatctctactaaaaatgcaaaaaattacccgggcatagtggtgctcacctgtagtctcagctactagggaggctgaggcagaagaatcgcttgaacccgggaggcagaggttgcagtgagccgagatcatgcaactgcactccagcctggggaacagagcaagactctgtatcaaaaaaaaaaaaaaaaaaaaagcaaattcttggatctcaccccagacctactgagtcagaaactccaggatggggataagaaacttgggttttaaccagcctcaagtgaacattagagatctttcagcctaccaaatcttcactgttatccctacaaacctaattatgactgcaatctctgctattttaattatccagtgtataacggtttccaagtataaaagaaagatcatgattatacaaaaaagttcataagcacaattcaaaaatattctatctacaagttgaaaattacaagtttcattttgtgcagaaaggtggtattagaattcattgacattaacttaattttttgagcattaatagaatgttacaatctatcacaactttcccctacaaaaccacttccttcagtgattaagccagcccactatttaactctgtttgctctttgttctttatctgtattacaggtgaattggatgcaggagaatagtttagtatattttgagaggcaaatgtctatttagttacatttgttaaccatgcaaaaagagacatttattcatgccgcagggctattttgaaggataaatgaaataatgaaataatgcatttgaggcatattgtagtcagcttgtttgagtccttaggctctttagctttcccaactggcttctcatcagaccaataggtgcctgtctttctcagagacccatgattccatcattactcacattttttagaaaaacattttaaacaccaactatgtgtcaggtgttgtgatataaaaaacacagagtacagtggtacatagaccccggtccccaaagagtgagtagggtcaatggttacaagagagacagacaagggcacataatgatacaggaatatacagtagggagtggctatccggggaagtctgggaagacatcacaaggaggtgacatttcagacatttcatgactgaagagtaagccccaaatcataggcagataggcattcaaggcagtgataccacatggtagggaggcacagaatagattgtgcaaggtgtgcttggtcagtgactagatgtatcagagtgcctacgtgataaacatagtagggagggcagcaggaaatgagcatgaaaaggtaggctctattaagagctggctactttgaacttcattctctattcctattcctcctaatcttttttgaggtcacatagactgtaagaatctgatgaaagttagaattgtctttccggaaagacagcacaaccataatgtttgttatataatttcatgcccccaggaaacctctacaggtccatgaactccaaataatcattgctctggaccaaaggtcaacaaaccccagcctgtttttgtaaaaatgttcttggaacacagccataccagtttgtttatgtatgacccatgactaccatggcagagttgaatagttgcaacggagatataacccacaaagcctaaaaatatttataaaaatgtttgcaaaccttctccctagatgatctgattagtagtcagagatccttagtatttctctattttaaagcatcttgagaacctgcttaatacattgcagaaaagaaggatcccagtgctccttccctaacaggttaatctttaacacatttgtacttgaatgggtgctcaagctgaagtgtaagatgacaggtaggttttgaaagattaagtaaataagaaaatctggagagtgttaaatgccacaggaattgccctggatgaaaactggagtggcaaagacaaactggtgatcaatagcagtacaaatgctgaaaagcagaaataaagaaaggattaaagaaaaactgttcctaatttttgtcttaactgaggccagcactatatatatttagtcaaaggcttacatataaagtatttagagtaataataataaatttaaaacgattgctcttatttcgtagaaccagccaaatctaaaggatttaaaccatttaaagattctcaatccttttgtggagaatgtctcaaaaaaagagaataagaaaagaaaataattaaattaaattaaaatgatgtccagtcataattccacctacctgatatacaccaatagtttctctatgggggaatctttaatgctggtaataataggatcatgaacatttaaatgtatataaatatatatttttcatttatgtttatacatttcttttttttccagagtgtacagattttacaagtttaccttatcaataacaggttttggtttattatttttatttattttacatttatttaagctattatattatcttagttattttgtcttatattttgctaacaattttattgtgtgataatcatgtaatttgtaagcctggtgatcacagagttcatgtcttattttgtcttgcttggcacactgctgagtgtagagtaggcatttaataaacatgagtcggctggttggtttgatgtcttagatatttaagggcggcttttctaatttatttatgtctgcatggcatttctttcatgttgcctatttaacatcttactaatcctaatcatgcttttctttcttttgaatactaggtcgttgtgaatgcactcataggagcaattccttccatcatgaatgtgctacttgtgtgtcttatattctggctgatattcagcatcatgggagtaaatttgtttgctggcaagttctatgagtgtattaacaccacagatgggtcacggtttcctgcaagtcaagttccaaatcgttccgaatgttttgcccttatgaatgttagtcaaaatgtgcgatggaaaaacctgaaagtgaactttgataatgtcggacttggttacctatctctgcttcaagttgtaagtgtcccatttcatgagtgcttggtattttaatagatattggacgaaggatataagttattctttaaatagtatattaattataaattgaagataaaatactgtgtggtttttaaaacagtgccagtcttggccgggcgcagtggctcatgcctgtagtctcttcactttgggaggccaaggtgggcagatcatgaggtcaggagttcgagaccagtctggccaacatagtgaaaccccatctctactaaaaatacaaaaaaattagctgggtgtggtggcaggcgcctgtaatcctagctgcttgggaggctgaggcaggagaatcacgtgaacctgggaggcggaggttgcagtgggccaagatcgtgccactgcactccagcctgggctacagagcgagactcttgtctcaaaaaaaaaaaaaaaaaaaaaaaaaagtgttggtctttcctagaacatgttcctacatgaataagaagcatgggttctgttgcatttgagttattaatttataattgactctttggtgtcatgtggactcccatcttgcaccattcaaatattgcttcaaaaatgaacgtttttgaacttcctgcttcacactatgaagggatgagaggaacttcccacaggcatggagaaccctttaaaaaatggagaaaagatgtttcctgtcttattcataaaattttccttcacctttaaaaaataaaaggttatgtgcgaaattcacaggcaactgtaacaatttagaaataattaactgggactaagatttagaatgttacttgggaatatgtcctttttaaaaaaatctttacattaaatggacatgttgaatacagcaaaacttactagtattacttaataattgtggtaacattaaacagacaaaactctgtttatggctattttagaatatgagcttaacattcaaattctattaatgttattcttaaaggcaacttttaagggatggacgattattatgtatgcagcagtggattctgttaatgtaagtattgattatcttagcactaaactttatttttaaaagcttcttagtttatttcagtgatttccaaactataacttcatgaagcccttgggactcacatagacaatgaagaacatccacctgatagtagcctagtggttgtgagtacagcagttggagatagagcctgattcaaattctagctgtaccatatcatccaagctctacaactgacttcgggcaaattaccttaccttcttgagacttagtttatttgtctaatctataatttaatgaatctatcatttgtctaataaatgtctacaatcatccaatgcctactccatagtgttgtgaggacccaacaagaaaatagaagcagggcacttagtgcaggctggcattactaaatgctcagtaaatatgcgcttatcaaaataaaaataaaataattttaatgtatagcttttactgtttttacagtttaaaacaccttggatgagtttatgtgaaataagttccatgctaagaaactttaatgattactccctaggtaatatttttacaaaatataataaagatgtattcggaatcaaaagaaaatacatggcaaaatgtgtataaatatgataatgtctcatttacagtacttttggaaataacatatacatagtatgtcacttagttattgaattttcctaccaatatgagcacaaatatgcatatgattaagccctttgtagttatttgaagattatattatttattacataagtatgtaataaattaaataataagtatgtaatgaatacatgttgaataaattgttaagagtgattatgttaaacctcaacaatgctatggcttcttaaatcctatgtttacctaggatagcactgtttgaattacctggtcaaaccatgtgaaaaagaaaataatgaatgttactgtccatataatgctaacttttgtaaattttataggtagacaagcagcccaaatatgaatatagcctctacatgtatatttattttgtcgtctttatcatctttgggtcattcttcactttgaacttgttcattggtgtcatcatagataatttcaaccaacagaaaaagaagataagtatttcaaatatttttcattgtaatgaaagggatgaacaattattttccaaaaaagataatgtaaagtttaatttgatatcaattcatgattaattttattagaacttcatttatttttcaaataattgactagtatacaattacaaatatgcttaatcttattcatttttgcacagaaaacaattttctaaataataggttctggctaacttatgatttaggttaaacccataggttttgagcaatatatcaactattctgttttttccaatgtttcctatagaggattgaataattaagaaaaatcaatacactatttattcaaaacacattttataagtagttatttattttattttgtgtaatatatattgggttggagcttggggagaccaacgtaagatatggttcctgagctcaaggagtttatgtcatagcagaataaataacacaggtaatgaaaattaagacttgttgaatactgtaatttgagcaatcttctgaaatttttaattaattaatcttcacatcaactgtattagtttctttgacctacgagaacaggataatacaaacctgagggcttaaaacaagagaaatatattctctcacacttttggaggttagaagtccaaattcaaagtgtcagcaggtccatgttctctttaaagcctctaagaaaatatattttattgcctcttccagcttctggtagcccaggtgtttcttggtttatggcaacagaactccaatctctcccattatagtgacatgccattttccctctgtgtgtctttctcctctactagtaaggatgccagtcacactggattaagagcccactccactccagtatgacttcatcttaattacatctgcaatgacattattttcaaataaggtcacattctgaagtgctaagggttaggacttatatgtaccttcttagggcacacaatccaacccataacatcaatcacaaaagtattattacagagtctctttcacaaatgaggaaaggcacacagaggttaagtgacttacccaaggtcatatatcgagtatgtgatgaaggttagatagagtcagtagtaaattcgatgtatcttgtactctattaaactctctactatgctctatccccaaatactataatcatgataaatacaataagaaaaattaacaagcacaaaaaatatacatgacagtcagagtaaaaatagatgctgctatagaatggtttatgaaatacgtgacaattgagaaggctttggaaggtgggaaagaagacatgtagcaacggagaggcaaaagaatggctaacccacatggagtgaaacagaaatgaatgagcaaatgaattgtgttgataaaagaggcagcaattcaggggggatagcaatgctttcggttttgaagaaatttaagatttggagaaatagtaaaagatgaggttttaacctgcctgagagtagatataaaaagccgtattagaccttttttaatagcagctggtgtttcattcatatatgttgagtagggaagaatatggaagcaggagattgatgtttattaagactccatgatatgtcagatcctgtgtaaaatacttctaacataattgtagtcaaattttacaacaaatctaaaagacatagtatcctcatttaacctctcagaaaaattatcttatttgcacatggcctgacagctaaaaagtagtttcattatggatctagctccagaattgctttttctagagactgagctttacaaaaaattaatctagcagtgatgcacaaaatgactagggaaggaccaaagactatttcagaccctgcttgacctgccacctggaccacactaagtctccaaattagtcctgtgtgtcaatggagaataggaaatatgttccattaaaacattttagagggtagaatctacagtattgcttgtgatttgcaagagataaaggagtctaattctggagtttcttgtcaactaactgagatactctggttgtggtaccacctatacaaaaagagatctttggagactaaatggtagaattacaaaagagaataataattttgggtactggcttagcatctaagaagacagatgtacatgtaccaaaatgtcattagtgtgcatgattgactatttgcaagtagacaaaaatttagtaaatattgcattatttatcctgacatttacaaatacagatgtttatttttaaagatttttaaatttcatgttccattttacatttataaagtgtacactgattttcaatttatttttctcaacttaagcattatatatacatacatgtaatatataagcacacacaagtaattcctcagtcttccctgcataggataaattaagcgtgtctagaaagagcagtatgagcataaataaaacagccaacatccaattgactttaatcttcctgcttgttaatgttattcttacaacatttctactgcaggaagtaaaataggatgggttgatactatcaccaccaaagtcaggaaacagttactgatttagaagcatcaaaaactcatatcccctatcattcttccagtatttcatcttgcaatctaagtatagcttttattttctgaccttttctgaatgatttagaatttctttccactcttccctgaaaaaacatgaccaacacttcaattagttaacacatgctatgtagaagtacgtagtagatgctttgtgatgaactcatttattccaaagaaaaaaaacaaatgaaagtggtatccttttctttgcctttcctaattttaattattgtggtaaggaaacttaatatgagatctacctgcttaacaaaatttttaagtatactatatatattgttaactatagttacgaagtactacaggaagtccctagaacttattcatcttgcataattgaaactttattcccattgaatagcaattctccattctcccatcctttcatgccctggcaatcaccattcttctctctgtttccatgagtttgaccattttagttacttcaccgaagtggaattatgcaatattttttctgttacttcactcagcataaatttatccaggttcattcaagttgctgcatatggcaggatttccttctttataaggctgaatagtgtgtgtttgtgtgtgtgtgtgtacgtgcacacgcacatgcaggtatacaaataccacattttctatcccatcattcatcaatagacatttaggttgttttcatatttggatacagtaaatagtgctgaagtgaacatggaagtaaggactctgagatcctgagttcaaatttttaaaatatgctcagatgtggcattgctagatcaaatggtagttatatttttaattttttcaggaactttcatactgttttctatggcagctacatcattttacattccaacaatgtacaaaggttctaatttctccatatccttgccaacacctattatcttttgatttgggtggtttttgtttttgttttggtaatagccatcctagcaggtatgaaatgatatctctttacggttttttgttgctgtttttttttttttttttttttttttttgttgtttttttttttgagacggagtttcgctctgtcgcccaggctggagtgcagtggcgcgatctcgactcactgcaagctccgcctcccgggttcacgccattctcctgcctcagcctcccgtgtagctgggactacaggcgtgcgccaccatgcccggctaatttttgtatttttagtagagacggggtttcaccctgttagccaggatggtctcgatctcctgacctcgtgatccgcccgtctcggcctcccaaagtgctgggattacaggcgtgagccaccacgcccggccttgttgctgtttgtttgtttgtttttgaaggagtctcactctgtcaccaggctggagtgcagtggcgcgatctcagctcactgcaacctccgactccctggttcaagtgattctcctgcctcagcttcccaagtagctgggattacaggcatgtgccaccatgcccagctaatttttgtattttcagtagagatggagtttcaccattttggccaggatggtcttgatctcctgacctcatgatccacccaccttggcctcccaaagtgctgggataacaggcatgagccactgcacctggccctcattacagtttttatttgaattttctgatgattagtgatactgagcatcttttaatatacatgttggtcatttgcatgccttcttaagttgcaggagttccctatatattttggatattaaccatttattagatatatagttggaaatattttctcccattctataggttgctttttcaccctcttgaatgcttcctttttttcccaaagctttttagtttgatatggtctcaattgtctatttttgctcttgtttgaattttggagtcattgccaagaaatcattgtcaagaacagtatcatgaagctttcctgctatgttttcttctacaagttttacagttttcaggtcttaggtttaaatgtttaatctattttgagtttatttttgtgtatggtataatataagaattcaattttatttttttatatgtgaatatccagttttcccaacattatttgttgagtggcctcttttctattgtgtattcttggcacccctgttgaatatgagttaactatttatgcatgggtttatttctgggctctctatcctgtttcattggcctatatatttgttttatgccaggaccatactattttaattgttgtagctttgttatacattttgaaatgaggacctgtggggactgggtgcaatggctcatgcttataatcccagcaactcgggaggctaaggctggaggatcacttgtggccaagagtttgagaccagcctgggcaagatagactccttctttacgaagatatttgaaaagtagccaagcatgatggcacacacctatagtcctagctacttaggagcctgaggtgggaggatcactgaagcccaagagtatgaggctgcagtgagctatgatcatgccactgcactctggcctgggcaacagggtgagatcttctctctcaataaataaataaataagaaatgagaaaatgtgattcttccattttgttcttttttcttaagattgctttggctattcagagtcttttgtggttccatatgaattttaggatctttttttattttagtaaataatgctattgggattttgatatgtactgtattgaattcacagatcacttagggtagtatgggcattttaacaatattaagtcatttaattcgtgaatactgggtctttccatttatttgtgtcttctttaatttccttcatgaacattttgtagttttcagtgcatacattttcaatgccttggttaagcatatcctaagtattttatttcttttgatgttatcataaatgggatttctttcctaattttctcttcagattgtttattgttagtgtatagaaacgcaggtgatttttgtatgttgattttatatccttcaacatctagtaaatttgtttattagttctaacagattttcctgatgtgtggagtctttagggtactctccatataaaataatgtcatctgtaaacacttttttctttcagattgggatgtcttttacttgtttttcttgcctaatttctctggctaggacttctagtgctgtgttaaatacaagtggcaagagtagccatatttgtcttattcctgatcttacaggaaagtttttcagtttttcaccattgaatacgatgttagctgtgggattttcaagtagtctttgttatcatgaagtatatttcctctgttctcatccatttgtgctactataacagaataccacagactgggtaatttataatgaacataaaccttatgggatcacagtactggaggatggaaagttcatgactgaggggctgtctggtgaggtcttatttgttgtgtcataacatgctgtaaggtatcatgttatgatacacatggcacaaagagagaataagtgagaaaaggaggcagaacctgcctgtttataatgaactcaatcctgagataacagcattaatgcatttaaaagggtaaaattctcataacttaaacacctcttaaaggtctcacctcccaattctgtcacaattgcaattaaattgcaacatgagctttggaggggacaaaccttcaagttatagcaacttctatacctagcttttgttttctttttttagtttttaccatgaaaggttgttgaaatttgtcaaatgcttcttctgaatctcttctgatgatcatgtgatttttatccttctcagatcatgtggtgtatcacattattggtctgcatatgttgaaccatccttgcatcctagggacaactcccacttggtcgtagtacatgatcatttaatgggctgttgcattcagtttgctaatattttgatgaagttatttgcatctatgttcatcagggatcttgttctgtagtatgtttttttctgtttaaatatttgttcatttttaattgacaaaaattgtgcttatttatggggtacaaaacaatgttttgatctacataaacattgtagaaattttcagtcacgctaattaacataaccatcacttcacccacttatcatttttttgtggtaagaaagttaaaaagctattcttttagcaattttgaaatatacatttatttttttcatttcctattgtgtgacttttttgacttttattttaagttcaggggtacatgtacaggatgtgcagtcttgttacataggtaaacatgtcataggggtttgttgtacagattatttcatccctgaggtattaatcccagtatgcattggttatttttcctgaccctctccttcctcccacccttcaccctcccataggccccagtgtgtgttgttaccctttatgtgtccatgtgttctcatcatttagctcccacttataagtgagaacatgtggtatttggttttctgttcctgccttagtttactaatgaaaatggcctccagctccattcatgtccctgcaaaggatatgatctcgtttttttttgaatggctgcatagtattatatggtatatatttaccacattatctttatccagtctatcactgatggtcatttgagttgattctatgtctttgccattgtgaatagtgctccaataaacatatgtgtgcatgtgtctttataatagaaaaatgtatttccctttaggtatatacccagtaatgggattgctgggttgaatggcatttctgtctttaggtctttgaggaattgccacacagtcttccattatggttgaattaatttacactcccatcaacagtgtgtaggcattctttttttctccacaccctcgccagcatctgttattttttgactttttcataatagccattctgactggtgtgagatggtatctcattgtggttttgatttgcatttctctaatggtcagtgacattgagctttttttcatatgcttgtttgttgcatgtgtgtcttcttttcagaagtgtctgttcatatcctttgcccactttttaatggggttgtttatttcttgtaaactcatttaagttccttatagattctggatattagacctttgtcagatgcatagtctgcaaaaattttctcccattctgtagactgtctgcttactctgttgatagtttcttttgctgtgcagaagctctttagtttaattagatcccatttgttaatttttgtttttgttgcagttgcttttggtgtcttcatcatgaaatctttgcccatgcctatgtcctgaatggtattgcctaggctgtcttccagggtttttatagttttagattttacatttaagtctttaatccatcttaagatttattcctctactctaaatgaaaaatgtacatcctttgatcaagatttatactttctccacatcgtcttcccccagtcctatggcaaccacctttatatgctgtttctgtaatgttgacttctcacttttttattttttatttatttatttttttattttactttaagttctagggtacatgtgcacaacgtgcaggtttgttacatatgtatacatgtgccatgttggtgtgcggcacccattaactcgtcatttacattaggtatatctcctaatgctatccctcccccctccccccaccccattttacatgtaagtgagatcatgtggtatttgtctttctgtacctggctaattttgcttagcataacgtcctttagtttcattcatgttgggatgaatgacaaaatttgttcttttaaagttttgtatagtagtgcactgtgcatacctatcacattttctttattcattcacctgttgatgggtacaggttgcatgtatgggttagtgcaaaagtaattgcggttttggccattacttttaattgcagcaaaagtaatggtaaaaactgcaattacatttgcaccaacctaatatgtcttggttcttgtaaataatgctgaaatgaacatgggagtgcaggtatacctttgacagaacgattttaattcctctaaaggaattaaatatataccactatatacctagaagtggaattgctgaattatatggtaattgtatttgttttgggaacttccatgctatttttctcaaacagctgtactaatttacattttcactaacaatgtataagggttcctatttctccacatcctcatcaacacttgttatcattcatctttttgataacagccattctaggaggtgtgaggtaatatttcattgggattttaatttgcatttttctgatgattagagatgttgagcattttttaatatatctattggccattcatatctcttcttttgagaaatgtctgttcagattatttgcccattctacaattggtttattgattttctaggtgttgagttgcttgagctttttatgtataatgttggatattagccctttatcagatgtatggtttgcagatattttatttcaatccatgatctctctctccactctgttattgcttcgtttgctgtaaagaacatttttagtttgatctaatccaacttgtctcttattgtcttttgttacctgtgcttttagagtcctagccaagaaatccttgcccagataaatgtcatacagcttttcttttatgttttcttctagtagctttatagtttcatttcttatatttaagtcttttacttgtttggttttataaacatatggtatgagaaagggcccaatttcattcttttgcataaggaatattcagttttcccaacaccatttattgaagagatcgtcctttcccccattgtttattctttgcacctttgctgaaaatcaattgactataaataattgggtttatttctgggctttttatccttttccattagccaatatatctgtgtttatgccaatagcatagctttatattttgaaatgacagagtattgcacctctagctttgttctttttgctcagattgctttggccatttagcatcttttttattttattcaaatgttaggattttttactatttctgtgaaaactgacattagaattttgatagggattgcattgaatctgtagattattttgggtagtatggacattttaacaatattaattcttccaacacatgaacatgaaatatattacaatttatttgtgtcttcctcagtttttttaaatcaacttatagttttcagtgtacagatcttcaactttcctggttaaatttacttcaaagtattttatttatttattttgatgctgttttaaataggattaatttcttaatttctttgttggatacctcattgttaatgtatagaaatgctactgatttttctatgttgattttgcaaactgcaactttactgaatttatgtatcagttttaacagttctcagtagagtgttcagggttttctacatataaagatcatgtcattagcaaacagagataattttatttcttgctttcttacatggatgtattttatttctttttcctgcctaattgctatggatgggatttctagtactatgtcaaaaagaagtggtaatagtcgatatccttgtcttgttcctgatcttagaagaaaagctcccaacttttcagtttttgcataatgttagccgtgggcttgtcatatatggctttattttgttaaggtacattccttccatatctgctttgttgagattttattatgaacatatgctggattttatcaaatgctttgtctgcatctagtaagatgatcatatgatttttgttcttattttgttaatgtagcatatcacatttattgatttatgtatgtttaaccatttttgcatccctcagataaatcccaagtaatcatggtgaatgatgttttaaatgtgctagggaattcagtttgctaatatattgttgtgaatttttggatctatgttcatcaagaatattggcctgcagttttcttttctggtagcatactcttagggttttcatatcagtataatactggcctcataaaatgaatttgacctccttttcaattttttggaagaatttgagaagaattcatattacttctttaaatagaattcaccagtgaccttttccttttttttttttttggttaggagacttaatttattactgatctattttctttatccagtattggtttgttcagatgtaatatttcttgatttagtcttggtaggttgcatgtttctaaagattagttcattccttctaggttattcaatttgatggtgtatggttgttcacattaatagtttatgattccttgtatttatgtgttatcagtgttagtctcccttctttcatttcagatattgtttgagttgtcttttttcccttggttagtctagttaaaggtttttcgattttatcttttcaaaaaaacaactttttagtatttgctttgtttatttctatcattatctttgttattttcttccttctgcaaagtttggggatattctgttcttctttttctaatctctaagatgtaatattaggttgtttatttgagattttccttctttctttaatgtaggcatttattactataaatttttctctcagggcttctttttctacattccatactatttgttatcttgtgtttccattgtcattgatcccaaaatattttctgtttttccgtataatttcttctttgacccattgcctgttcaggagcatgttgtttaatttccacgtaattatgagttgttcaaggttattcttattgacttctagtttcatgtcatcatgaacagaaaacatactcgatatgattttaatcttcttaaatttgttaaaacttgtttggtggcctaacatgtgatctatcctggagagtgtttcatgtgatttgagaagaatatgtattttgttgctgttggatgaactgttctataaatatctgttagatctattggattaaagtgtagttcaagccttcctctaataagaaactgcataaattatgtcaggagcaagccagggaagaagccaccttcctgttcttggtggtagagaccttccacatcttttaaaagaaagattgtggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgtggtagcgggcgcctgtagtcccagctactcgggaggctgaggcaggagaatggcatgaacccgggaggcggagcttgcagtgagccgagattgcgccactgcactccagcctgggcgacagagcgagacagcgagactccatctcaaaaaaaaaaaaaaaaaaaaagaaagattgtagagttgggattgtctctggaggaagccagggaagaaggcgtggggaatgttattcctcttgtttaggctgaagaaggtttattcttcatttgctatgtagatttccaagtactggaagaggccagaaccacagggagcggctggaaaacgtgtagcctaatggtttccatggggaagttggaagatggtctgatcccaggaattaaagcttctgagagtgctcaggcagttcaaaaccccccatcgttttttgtacttgagagaaaactccaatggcttatctctgccattcctcaagcatggtgatctaaaagccaagctctgtgtcagggcctataaaagttgggttctacaaaatggaaaaccaaggcaattatgaagatgattttataatatagttcatctcttctctctttacactctagaaaaagcattttctagaaataaatacatttccttaaattaaaaaaaagttgggttctatatgtgtgctatagtttctcaacccttgggaagtagctgcaaatttggttttcttcccaaatgtaaggcactgtgctcagggtggggttagtgcatgagtgtctttccactttttctacccattttgatatgggtattttctgtcctctggtgtgcatgagtttttctactggattctgggtttctctggagagaactgatccatgtgtatgtagatgtttacttggtacgtccatgagagaaaaaatagtcataaacctcctattccacaatgttgctgaagtcacttccttcaaaagctgtaattttcttttcttacagtgtctttgtctggctttgatatgagaataattctggccccataaaatgactttcgaagtgttctttcctcttcaaagttttaagagagtttgagaagaattaatgttgtttcttctttaaatgtttggttggacttctccagagaagccgtctggtcctgtacatttcttttttgggagttactatcattatcattattattattattattattattattattattattggttcaatttgtctattcagttattctatttcctcatgattcagtcttggtaggttgtatatttttggaatttacctattccttctaagttatccaatttgttggtgtataattgttcatggtagtctctttgatccttcttattcccataaaatcagttgtaaattctcctctttcatttctggttttacatatttgagtcctctatcttttttcttagttatgcttgctaaggatttgtctattttatctttcgaaaaaaccaattcttagttttgttgactttttctattgcttttttattctctatttcacttacttagctctaatacttattatttccttccttttgctgatgttgggcttagtttgttctttttctagttcctagacatgtaaatctatattgtttatatgagatatttctttttataatgtagacatgtatctcaataagatttgcttttattactgcttttgctgcatctcataaattttggaaattgtgctttcatttttgtttgtttcaagatacttttaaattttcctattgattttttttctttgacccattgctttttcaagagagtgttgtttaatttccacatatttgtgaatttttcagttttccttctattactgacttccagtttccttccattgtggtcataaaagatatctggtatgatttcaacttcttaacagttgtcaaaacaaacttcatgacctgatatgtgatctatcctgagaatattctgtgcgcacttgaaaagatggtatattaagatgctgttgggtagcatattctgtgtgtttgtgatccatttggtctatagcgttgttcaaatccacagtttgtttatagattccatatctggctgttctgttcattattgcaagtcgggtattgatgtatcctactgttatggtgttgctgcatgtttatcccttcagatctgtcaatgattgctttacgtatttacatgctttgatgatggattcacatatatttataattgttatatcttcttgatgaattaactcttttatcttattttatgacctactttgtctcttgtgacagttttttatttaaaatctattttggctgatataattacagggtgtcctctgtgtctgtgggttttacatctgtagattcagccaatgctgattgaaaacactgtattcatggaatgtgaaatccacagatacagagggtcaaatttttttatccacaggtcctgcaagactgactgtgggacttaagcatcctcaattttggtatcccttggggttctgaaaccatgaccctgtagatactgaaggacgactgtatagccccattgctctcttttggttaccatttctacaaaatgtatcttttttatcaattaactttcaaactatgttttcttaaatctgaaacgagtatctggagacagcatataactggatcttgttttcataaatctatgcagccattgtaggtcttttgattgggaagtttaatccatttgtatttgaaataatcattggtaggtaatgacttactattgcctttttgttaaatttttctgttttgtaattctttttttctgtctttttctctcttaactgtcttcttttgtgattggatgatattttattgtgatatgctatgatttctcttttttatttctgtatttttacgtggattttccttgtggttatcatgaggcaatcacaaatatcttataatagtctactttaagctgataacaacttaacttcaatcatatatcaaaaatcttcacttttaatacctccctcacactttttgttatgtcacaatttacatctttttataatgtgtttctacaaacacatttttgtaattttagttattcttaattattttgacatttaacttttatattagaattataaatgatttatataatactattagagtattacagtattctctatttgtccatgtatttacctttactagtgagatttgtgctttcatatgctcttgtgttgctctttaccatgctttcgtttcaacttgaagaactcactttagcatttcctataagataaatctagtggtgatgaactccctcagcatttgcttgtatgagaaagtttttatccttccttaattttaaaggacagtttttccaggtatattattcttggtcagcagtttttttctttaagtattttgaatatatactttcactttatttgctcttttaaagtttccactgagaaatctgctgttcatcttaagagagctcttttggacatgacaattcagtttttttcttgctgctttcaaacttccctttcacttttggcaatttgatgatattatgtctctagtggagactttctttcttttttttccgagatagagtttcaccgtgttgcccaggctggagtgcagtagcatgatcttggctcactgcaacttccacctcgtgggttcaagcaattctctctgcctcagcctcccgagtagctgggattataggcacccaccaccacacccagctgatttttgtaatttttggtagagatgtggttttgccatgttggccaggctggtcttgaactcctgacctcaggtgatccactcccgctcagcccctcaaagtgctgggattacaggtgtgagccaccatgcccagccgagacttctttagatttgtcctatttgggattctttgagtttcctgaatttagatgttcatatctctccccaaatttgaacatattttagtcatttaaaaaatatttttacacccctttctgtcttctctgtgatttcataatgcatatattgttccacttaatgatgtcccatgaaccttcactcatttttttttttatttcttcttgctccactgactggataattttaaatgatctacctttttttttttttttggtgtccatcttttgagggttttttaattttattttttatttttttatttttattttttattatactttaagttttagggtacatgtgcacaacgtgcaggttagttacatatgtatacatgtgccatgttggtgtgctacacccattaactcatcatttaacattaggtatatctcctaatgttaggtatatccccctcctccccccaccccacaacaggccctggtgtgtgatggtccccttcctgtgtccatgtgttctcattgttcaattcccacctatgagtgagaaagtgcagtgtttggtttttttccttgcgatagtttgctgagaatgatggtttccagcttcatgcatgtccctgcaaaggacttgagctcatccttttttatggctgcatagtattccatggtgtatatgtgccatattttcttaatccagtctatcattgttggacatttggcttggttccaagtctttgctattgtgaatagtgccgcaataaacatacgtgtgcatgcgtctttatagcagcatgatttataatcctttgggtatatacccagtcatgggatggctgggtcaaatggtatttctagttctagatccttgaggaatcgccacacggacttccacagtggttgaactagtttacagtcccaccaacagtgtaaaagtgttcctatttctccacatcctctccagcacctgttgtttcctgactttttaatgatcaccattctaactggtatgagatggtatctcattgcggttttgatttgcatttctctgatggccagtgatgatgagcattttttcatgtgttttttggctgcataaatgtcttcttttgagaagtgtctgttcatatcctttacccagtttttgatggggttgtttttttcttggtttgttgattctttctccagtttgatcaagtctgctattgaatcctctagggagttttttagttcaattattgtattcttcaattccagagtttttactgatatttgactttttgtgtgtgtgtgtatagtttctttcccttcattgatatcctcattgtgttcatgtatcatcttcctgattttgtttagttgtctatctctgttctctggtactttgctaaacttcattaggataattattttgaattcttttttaggcaatttataaatctctatttctttagggtcagtaactgcggatttcttttatccctttttgttgtttccctaattctttgtgttccttgtgctttgcattggtgtctgtgcattggaagaatagccacctgtcagccatccgacacaatgtcacaaaggacacaaaacagccaccaagatttatggactgactttgacagggcaagacctttgccagtcagcctggctagagatactgagagtctctcaaacagctaatctaccgtccccttttcctatgtttgtagctgtatcaattgttctaatcatactggttctttcagtgctccatatgaggcaagagtgaaactggaccctagtgcagcacactgaatggcaaggacatttgatgtgctccactcctctccctcccttgaaagtggtatcttgatgaaaatgaataaattagcccctagaaaggatggataaattgtttcaaaccaagtaacaggataccagcacagttatgattgaaatgcctgtttgatttgaaaacccatatcttttccacaatgcaatgttgccacttactgctttttaacttcaataatttgatcaaagcttatggctctatcaactacccatgcctctttctaataaccaaccctattttactagtttttccaactattcatcatcctcataagtacttttttcacctctaatgatttcatgtgaaattagtttttaataatcataaaacatactactattagaacaaaagtcattaatgagttcaagttataaaaaggaagattacatgatatacaaatatataattacaaatttgagcaactatggagtaaatgttaacaataacctcatttcattaactctcatcttatcaggaagtttaatagcaccaacagtaatagtaactaaaatttaatgaagacttaccatgtgccagatattatgctaagtattcttcatgcattacctcaatttatcttctcagcaaacacaaatgcagtgactactcttataatgatgaggtggttttacttaaagaattaagaaacttacccaaggacaaaagaaaggacttgaacaaagatgatctgacaccagaagtgaagaagtctttattactatgtaatacagcctcaatgtagattgtaacacagaaaaaaactagcctgacttacagaattccttccacctaggttttaaagggatacattttgcacaaggcctaaataagctataggaaaacaaaacacccacgcaagtttctctattgaattaattcataatctcctattagttttatcccattcttcatcatattgacaaccaggatatcattaggggaaaacaaaatctattactttgattttctcttcaaagttggagttttctcctcagtttttctccagcttcttctgtcaagttcttggcatgctgtaattttcagtccccattaatgaacagtaaaggatctgggtttctcttgacattgacatgaagtctgtctctctcatgccttgccagtaggtgagatgaatatgaaaattacataaagactgcatattaaatagaggaaagctgaattaaaacactctaaggtggccggatatggtggctcacgcctgtaatcccagcactttgggaggccaaggctggcagatcacttgaggccaggagttcaaaaccagaccagccaacatggtgaaaccctgtctctactaaaaatacaaaaattagcctgatgtggtggcatgcacctgtaatcccagctacttgggaggctgagacatgagaattacttgaacccaggaggtggaggttgcagtgagccaagatggtggcactgcactccagcctgtgcctcagagcaagactccatcaaaaacaacaacaacaacaacaacaacaacaaaaacactctaaggcagaaagggaagtctggttaccattaggtgtttgatgaataagaaaattcaatataatgggaagcttcccattcaatagcaaacaattaaaaaaataagtaaataagaggaagctgaatattaccaaatccaagagaataaagggaaaccataaccaggtcatctttaattccagcaccctacaagttaagtaatgatctcagaactataaatccttgaaaataaattcttaattatatggattatactaatttatgctgtgacttaaaaaggtgaaattatagactaatgtagaatatacataaatctgtaaaacttacagctaacttaagaaattagcagagaacaaagggagcaactgaagcttctagcccccctttgtaagatttaggcttttccagtcaaaaacctcatttagatgttatgttatttactgttgaattttaaaagttatattttcttttatgtaatcaattggaaagtccttcagtttgggtatgataattgataacttcctcagtacctagaaatctatcaaggataatgttgacatccaataaagtgtttatgttgaaatcctttatttagaattaaaaaggtagtcaaagagtttcttgaaatagagatatagaaatgtatattatcattttagtttatattcattttttatgtaaaggaataatggtatattaaaatgaatataaaattaatacaattttggttattgtatgtagcccctattctttttttattattatactttaagttctagggtacatgtgcaaaacatgcaggtttgttacataggtatacatgtaccatgttggtttgctgcaccaatcaactcatcatttacattaggtatttctcctaagtaatccctcccaaagctcccaaccccctaactgcccctggtgtgtgatgttcccctccctgtgtccatgtgttctcattgttcaactcccacttatgagtgagaacatgcagagtttggttttccgtccttgtgatattttgctgagaatgatggtttccagcttcatgcatgtccctacaaaggacatgaactcatccttttttatggctgcatagtattccatggtgtatatatgccacattttctttatccagtctattattgatggacatttgggttggttccaagtctttgctattgtgaatagtgctgcaataaatatacatatgcatgtgtctttatagtagcatgatttacaatcccttgagtatatacccagtaatgagattgctgggtcaaatggtatttctagttttagattcttgaggaatcatcacactgtcttccacaatggttgaactattttacactctcaccaacagtgtaaaagcgttgctatttctccacatcctctccaacatctgttgtttcctgactttttagtgattgccattctaattggcatgaggtggtatctcattgtggttttgatttgtgtttctctgatgaccagtgatgatgagcattttttcatatgtctgttggctgcataaatgtcttcttttgagaagtacctgttcatatcctttgcccactttttgatggagttgtttgtttttttcttgtaaatttgtttaagttctttgtagattctggatattagcccttttgtcagatggataaattgcaaaaattttctcccattctgtaggttgcctgttcactctgatgatagtttcttttgctttgcagaagctctttagtttaattagatcccatttgtctgttttggcttttgttgccattgcttttggtgttttagtcatgaagtctttgcccatgcctatgtcctgaatgatattgcctagagtttcttctagggtttttatggttttaggtcttacatttaagtctttaatccatcttgagttaatttttgtacaaggtgtaaggaagggatccagtttcaactttctacatatggctagccagttttcccagcatcatttattaaatagggaagcctttccccgttgcttgtttttgtgaggtttgtcaaggatcagatggttgtagatgtgtagtgttatttctgaggcctctgttctgttctgttggtgtatgtatctgttttggtaccagtaccatgctgttttggttactgtagccttgtagtatagtttgaagtcaggtagtgtgacgcctccagctttgttctttttgcttaggattatcttggctatgcaggctcttttttggttccatatgaactttaaagtagtttttttccaattttgtgaagaaagtcagtggtagcttgatggggatagcattgaatttataaattaccttgggcagtaatgccattttcatgatattgagtcttcctatccatgagcatggaatattcttccatttgtttgtgtcctcttttatttcgctgagcagtggtttgtagttctccttgaagaggtccttcacatcccttgtaagttggattcctaggtattttattctctttgtagtaattgtgaatgggagttcactcatgatttggctccctattctttataaaagttctaaatatttgccaaatcatcaaagaagatattcattacttttaattcttacaaattttcttagcaataaaagtcacagtaagacagctagcagataaaaaagataaaagtacatagtttaaaacaacactgattagcacaccaaaagaatattgagccaatccttattatatgtaatacaaatatttatatatgcacatttaactgtgtttggagacccatgtttctacacatatgtaattatttaagatatattttatgtatagctatcatgaacataataaaatacattttatttctatattaacacattttaacctattttagtaatctatagaaagatgtagacaatgattctggttttaactacatttattttttgtttgtttctttaccttggaggtcaagacatctttatgacagaagaacagaagaaatactataatgcaatgaaaaagctggggtccaagaagccacaaaagccaattcctcgaccaggggtaaaaaaatatatatatctttagcatatagattttcaaattatttctaattcatttttaatgcacatctttaatttctggataatacttgaaaagtttactctgcattcgatattattcttatttctttgcagaacaaaatccaaggatgtatatttgacctagtgacaaatcaagcctttgatattagtatcatggttcttatctgtctcaacatggtaaccatgatggtagaaaaggagggtcaaagtcaacatatgactgaagttttatattggataaatgtggtttttataatccttttcactggagaatgtgtgctaaaactgatctccctcagacactactacttcactgtaggatggaatatttttgattttgtggttgtgattatctccattgtaggtaagaatatttatttttcagattttattttttgagtaaagctaaacttcacttatgctcaaggaagtatatgctgaattcttgaacacaaatttactcttgagtaagtcagtacaatgaaagaatgaaaaaatcctaaaaagcaaaacaaatatctgtctctcatactacagtgtttctgggtttacactacaggccccaaagccaaatatttctatctattagaaggaaattatacttgttgatatttagtttccaatattacctttgcaaaactagttaccatgtttatatatagctgtgtcttggaaaaccaatgttgaatctgaaataacttctggttttagcacatgtgagacttgtcatttgattaatgataatattaatagccagaatatctgtgaatttgtgaaaattcttccagaatttaaagtgaagttaagatctctagaaatgccagttttattttttacagcttaatacttcacaagtgtataaaatgccaatcattcctcaaccagccatggtgttcatgtctgtgaataacagcacatgattttatacttcctatacattcattgccatggttgtttttctatcttcttgtttttgttgttgttgttattttcacatatcttgcaaagtattttaggattgtatttctttttagaattataaacttttaagagagagagattatagagattactacaagtttaatcaacaggttatcaatctgaaaaggttcaaggttgcatgacttagaatcataaaattgagtgccacataacagagtaagcatttaaaatcaataattttggaaaagcatattatcaccagaagtcataccaaaattgatataagaaaatgtattaaaatttaggtgagaatttcacactaacaaaaatgattctgaaagcattcctattaagtcaggaaaaaaaaaagagtgtgcctgtatcatccctgtttttagtgttgttggccaatgcaattagataagaaaacagagaggtacatatataaaaacaatggagtaaatgatccttaatataatgaccttcttcatataatgaaagaacttgttcatatattgaaagagaatcaactaaaaataaattcaattagagttcaaaaggtacaaattttaaattacatgtgccaacactaataattatcttatacatgaaaaaaattagaaaatctgattcacaattgcaataaaaataaagtaaattatctaataataactttatcatatgagacttctgaagaaaactttaggatataaaagaaagctaaaataattgaaaggattgtcatgtacttagataagcttctctcattgtaattaagactttcaaatttattaatctataaattcaaggaaaatatcaatgagtctttttatgtagtgtgggtaataaatagacaaaaacaatataagtctaaatgattctaaattcatctggaagaaaattagggtgacaagaggtggcttattctgcaaaatattaagacatatagatgttaaataaaactgttcaaatataaaagctcagtgggtttgaagagagtccaaattaaactttaatgtataattaccaatttactgtgtgaaaagatcacatgaatcaaaatctaaagttaaaaaaaaagattctagtaggaaatatgtggaaatagacatataaccatatactcttatagtcctttctatgcaaaacacaaatttgtaaagccaaaaaaaaaaatgaagaagaaatgactgcacaaaaaaacaaaatattctttggggaaaaaatcatcataaataaaaggttgaaagacaaaaaataggtgaaatgtttacataacagttaaggccgagaaaggatcaatactttaaatagaaaattgttcttataaattaataagaatatagtaaaaatcccaatagacacatgggcaaataccaaactaagccaaaaacagaagttaaaaacaaccaataaggatacaaaataacaaaacacaaggaaaattgtggattattttcacaaaaaatgcaaataaattacaatagtagatgttttcaaatgtcatgaaaattttttggtaaaaaactataaagaagaatggtagcactcaatactgttgaatgtgtaaagtatgagcactatcatatactgccattagaaatgttaattagtacaggctttctggagataggttcagctatatattgtgtgtatatatatacacatacacacacatactatgtatatagtatagagtatgtatatatgtatactatatatatacatactctatactatatatatagtatacgcatactctatactatatatatagtatacgcatactctatactatatatatagtatacgcatactctatactatatatatagtatacgcatactctatactatatatatagtatacgcatactctatactatatatatagtatacgcatactctatactatatatatagtatacgcatactctatactatatatagtatatgcatactatatgctatatagcgtatatgcatactatatgctatatagagtatacgcatactatatgctatatatacatactatatagtatacatatactatgtactgtatatacgtatacttagtatatgtatatgtatatgtatagtatatgtatatgtatagtatatgtatactatatactgtatatagtatatatagtatgtgtatatatatagtatgtgtgtatatatagtgtgtgtatatatataaacatatatatactgttgacccaagttctattaacatattattaaaaagtactcatgcaattaggagaacatagctatttagatattcatttcagtaacaaaaatggaaacagtataaatgctagtcttcaatttgtggttattttgagagaacattataccactataataaattatttcgttgtcatttttatgaaactatctccatgacatataaagtaaataaaataggttacaggctgggcactgtggctcacgcctataatcccagcacattgggaggccgaggtgggcgaaatcacttgaagtcagaagtttgagaccagcctggccaacaaggtgaaaaccgtccctaccaaaaacacaaaaattaaccaggtgtggtggcgcatgcctgtagtcccagctactctggaggctgaggctggagaatcatttgatcccgggatgcagagattgcagtaggccaagatcacaccaccgcactccagctttggcgacagagcaagactctgtcttgaaaaataaataaataggttacaaaaaaagcttatataacatgaatacatttgtttgtgtgtgtacatgtgcttgcacagaaaaatgtctgaagtgatactcaaaaaaatattgataatagttattgttgacatttcaggtgatttatattttctttttagcatttaacggtattgcttgaattttctatgtgagtaggtatcattcttaaaatgtaaaacactattatggagggaaaaaatggcccatgaacttagtaaaaaggagaaaatgtcacaatggtaatgttagtcatttctctttggaatttcttaatccttttttaagtaattgcattcagtataaaataactgtcttggccgggcacggtggctcacgcctgtaatcccagcactttgggaggccgaggcgggcggatcacgaggtcaggagatcgagaccatcctggctaacaaggtgaaaccccgtctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaaaaaaaaaaaaaaaaaaactgtcttgaattcataagaaatgagttgacagataaaactgtttagagtcatcatttcaggtagcatacatctttaaatattttatttctattattttcctccacatacaggtatgtttctagctgatttgattgaaacgtattttgtgtcccctaccctgttccgagtgatccgtcttgccaggattggccgaatcctacgtctagtcaaaggagcaaaggggatccgcacgctgctctttgctttgatgatgtcccttcctgcgttgtttaacatcggcctcctgctcttcctggtcatgttcatctacgccatctttggaatgtccaactttgcctatgttaaaaaggaagatggaattaatgacatgttcaattttgagacctttggcaacagtatgatttgcctgttccaaattacaacctctgctggctgggatggattgctagcacctattcttaacagtaagccacccgactgtgacccaaaaaaagttcatcctggaagttcagttgaaggagactgtggtaacccatctgttggaatattctactttgttagttatatcatcatatccttcctggttgtggtgaacatgtacattgcagtcatactggagaattttagtgttgccactgaagaaagtactgaacctctgagtgaggatgactttgagatgttctatgaggtttgggagaagtttgatcccgatgcgacccagtttatagagttctctaaactctctgattttgcagctgccctggatcctcctcttctcatagcaaaacccaacaaagtccagctcattgccatggatctgcccatggttagtggtgaccggatccattgtcttgacatcttatttgcttttacaaagcgtgttttgggtgagagtggggagatggattctcttcgttcacagatggaagaaaggttcatgtctgcaaatccttccaaagtgtcctatgaacccatcacaaccacactaaaacggaaacaagaggatgtgtctgctactgtcattcagcgtgcttatagacgttaccgcttaaggcaaaatgtcaaaaatatatcaagtatatacataaaagatggagacagagatgatgatttactcaataaaaaagatatggcttttgataatgttaatgagaactcaagtccagaaaaaacagatgccacttcatccaccacctctccaccttcatatgatagtgtaacaaagccagacaaagagaaatatgaacaagacagaacagaaaaggaagacaaagggaaagacagcaaggaaagcaaaaaatagagcttcatttttgatatattgtttacagcctgtgaaagtgatttatttgtgttaataaaactcttttgaggaagtctatgccaaaatcctttttatcaaaatattctcgaaggcagtgcagtcactaactctgatttcctaagaaaggtgggcagcattagcagatggttatttttgcactgatgattctttaagaatcgtaagagaactctgtaggaattattgattatagcatacaaaagtgattcagttttttggtttttaataaatcagaagaccatgtagaaaacttttacatctgccttgtcatcttttcacaggattgtaattagtcttgtttcccatgtaaataaacaacacacgcatacagaaaaatctattatttatctattatttggaaatcaacaaaagtatttgccttggctttgcaatgaaatgcttgatagaagtaatggacattagttatgaatgtttagttaaaatgcattattagggagcttgactttttatcaatgtacagaggttattctatattttgaggtgcttaaatttattctacattgcatcagaaccaatttatatgtgcctataaaatgccatgggattaaaaatatatgtaggctattcatttctacaaatgtttttcattcatcttgactcacatgccaacaaggataagacttacctttagagtattgtgtttcatagcctttcttctttcatatccctttttgttcatagaataaccacagaacttgaaaaattattctaagtacatattacactcctcaaaaaaaacaaagataactgagaaaaaagttattgacagaagttctatttgctattatttacatagcctaacatttgactgtgctgcccaaaatactgataatagtctcttaaactcttttgtcaaattttcctgctttcttatgcagtattgtttagtcatcctttcgctgtaagcaaagttgatgaaatccttcctgatatgcagttagttgtttgaccacggtacatacttgagcagataataacttgggcacagtatttattgcatcacttgtatacaatcccgtgtttggcaagctttcaaatcatgtaatatgacagactttacacagatatgtgtttagtatgaataaaaaagcattgaaatagggattcttgccaacttgctctcttgccaccaacttactttcctaaattatggaagtaatcttttttggatatacttcaatgtatacaatgaggaagatgtcaccttctccttaaaattctatgatgtgaaatatattttgcctcaatcaacacagtaccatgggcttctaatttatcaagcacatattcattttgcattagctgtagacatctagttttttgaaaacacctattaatagtaatttgaaaagaaataaccataatgctttttttcgtgagtttatttcaggaatatgagatctttcttctataaagttattcatgcacaggcaaaaattgagctacacaggtagaatgtagttttacttagaagatttttgtgggaggttttgaagcaaatatataaaacaactttcactaatttgctttccatatttaaaaaataataaattacatttatataataaatgtttaaagcacatattttttgttgttctggcaatttaaaaagaaagaggatttaaacgtacctatagaaacaaagatttatggttaaagaatgagatcagaagtctagaatgtttttaaattgtgatatattttacaacatccgttattactttgagacatttgtcctaatctacgtataaaactcaatctagggctaaagattctttataccatcttaggttcattcatcttaggctatttgaaccactttttaatttaatatgaaagacaccatgcagtgttttccgagactacatagatcattttatcacatacctaccaagcctgttggaaataggttttgataatttaagtagggacctatacaaaatatattacatttatcagatttttaaatacattcaattaagaatttaacatcaccttaaatttgaattcaatctaccgttatttcaaactcacaaatataactgcattatgaatacttacataatgtagtaagacaagatgtttgacaggttcgtgtgtaattttctattaatgtttttacattgccttgtttttatgtaaaataaaaaatatgggcaactggtttgttaacaacacaatttcttcttagcatttcaaaaatatatataaagttgttctttttcctatttcatgaactatgtttttttttaaaataacatggttaagttttatatatatttacgtttgtttcaggaatgtctacttgtgactttttatcaattaaaaataatatttggaagaaagagcttattaagtataagcttgaagtaaaattagacctctctttccatgtagattactgtttgtactgatggtttcacccttcagaaggcactgtcatattaatatttaaattttataatcgctgaacttattacacccaacaatacagaaaggcagttacactgaagaacttaacttagaataaaatggaagcaaacaggttttctaaaaacttttttaagtgaccaggtctcgctctgtcacccaggctagagtgcaatggcatgatcatagctctctgcagcctcaactctgggctcaagcaaccctcctgcctcagcctcccaagtagctaagactacaggtacatgccaccatgcctggctaatatttaaatttttgtagataaggggtcttgctatgttgcccaggctagtctcaaactcctggcttcaagtgttcctactgtcatgacctgccaacatgctggggttacaggcatgagccaccatgccccaaacaggtttgaacacaaatctttcggatgaaaattagagaacctaattttagctttttgatagttacctagtttgcaaaagatttgggtgacttgtgagctgtttttaaatgctgattgttgaacatcacaacccaaaatacttagcatgattttatagagttttgatagctttattaaaaagagtgaaaataaaatgcatatgtaaataaagcagttctaaatagctatttcagagaaatgttaatagaagtgctgaaagaagggccaactaaattaggatggccagggaattggcctgggtttaggacctatgtatgaaggccaccaattttttaaaaatatctgtggtttattatgttattatcttcttgaggaaaacaatcaagaattgcttcatgaaaataaataaatagccatgaatatcataaagctgtttacataggattctttacaaatttcatagatctatgaatgctcaaaatgtttgagtttgccataaattatattgtagttatattgtagttatacttgagactgacacattgtaatataatctaagaataaaagttatacaaaataaaa

SEQ ID NO: 2 - DNA, 119156 bp, mfascicularis
  misc_feature (16458)..(18257): n is a, c, g, or t
  misc_feature (24073)..(24242): n is a, c, g, or t
  misc_feature (54952)..(55249): n is a, c, g, or t
ttactccaccagaaatcgcaaatcttaaactaaataccggactgatgaaaactcctgtggcctgtttatccagaagactaagggaagaggaaagaagttgtagtcacacgtaggtcctccctcatcattgcatattctttgtgtgtgttggtgaagtgtgaggctccatttttacttatgttgccctctgtcaccctgtcagcccatcatcttgccaccccactggccctgtacaataacttccaagcaaactcagatgccaactaaagctgctcttcttcaaagctataaaactagtaaaatgaaaggaagtttcacctctccaaactagaggatgaggatgaaatctgatcaaggtctcttcaacttatatttcacagaccatgatgttaagcaggttcttagtgtcttcctttaatttagcttgatcagggaaatattgcccatgcatggtaccactagcattgaccaccctttgcctctgctttttgtataaactttagtcattctggagctgtgttgagcatgtgcatgggggtcagtagagacagagacagaagagaataggaatattggaagagtcgaactgataatgggcatcggagttcacaacatctgtgtacctagatttttctcaaataattattgaaagaggtggtaccctccagttgaatgcagaatctaccaaaacaccagttattcaagccaatttttcttcaagagccacgttatgtctacactgaaagtgtagtgaggcaagttattccaacctctcacctttatgattttcaaaagtgttgaggaatccccaaagcacaagtggcaactgctttgatttgattcattgatttgtttccattttactaaatctcttcctattctgaggacaattttcacatattttatgatccaaaattattaatgcatattcgtagtagttttattgaggtataatttacacaccataaaaatcaccattttaaagtgtaccactcagtggtttttcgtatgttcaggaaggtatgcaaccatcataattacataattttagaatatttttatcacccccaaagacgtcccatactctttaaccgttaaccacataaccatcaatagtcacttcctattacacctagcctctgaaaaaaaaatactctacattttgtttctggggatttgcctactctggacatttcataagatggaatcatataatatgtgaccttttggctctgtgttccttcacagagttttcatcacatccatgttgtaacatgtacctatacttcatttgtttttattgctaactattaattcagtgtgtgaatatcccacgcttttatccatttattcattggtgagtaattaggttgtgtttactttctggctactatgaataatgctgctgaacattcatgtaaacctttttgtgtggacatatgttttaaattcccttgagtctgtacctagtagtagaattgccaagtcataacttactctttacctttcagaatcatcctacttttgaatgtgtacatgcagtgtcccttatttttcactacattctttaaaaaactttatcttaactttggtttccaacaaaatacaggcatagttcattttatggggctttgctttattgcacttcaagatactgcatttcttacaagttgaaggtttgtggcaaccctgcattgagcaagtctgtcagcataattttttcaacagcatgtgctcacttcatgcatctgtgtcacactgtggcaattctcacaatatttcaaactttgtaattgctattatttattattactatgtatgttatggtgatctatgatcagtgatctttgttgttactatggtaattgttttgaggtgccatgaaccacatccacataagacagcaaacaatcattaaatgtcatgtgttctgtttcagcattcctatctctctccctttcctccggcttccctgttccctgacacatagcaatattaaaattagttcaattagtaacccattcccatccctaatgttccagtaaaggaagagttgtgcatttctcactttaaatcagaagctgaaatgattaagcttagtgaggagggcatgttgaaagccaagacaggccaaaagctaagcctcttgcactaaatacttagccaagttgtgaatgcaaagtaaacgttcttgaaggaaatcgaatgtactactccagtgaacacacgaatgataagaaaacaaaacagccttattgcaaatatagaaaaagttttagtgattgagatagaagatcaaaccagccacaacatttccttaagccaaagcctaatctagagcaaggccataactctccgattatatgaaggctgagaggtgaagaagctgaaaaaaaagttcaaagctggcagacggtactccatgaggttgaaggaaataagccattgccataagataaaagtgcaaggtgaaacagcaagtgccgatgctgaagatgcagcaagttattcagaagatctagctaagatcattgataagagtggttacattaaaaaacagattttcaatgtaggtctaacagccttctactagaagaatatattatagctagaaaggagaaaacaatgcctaggttcaaagcttcaaagaataggctgactctcttgttaggggagaatgcagttggtgactttaaattgaagcaaatgcttatttatcattcagaaaatcttagaattatattattattcagaaaattaaacaaaaattatattatttaattattcagaaaatctcttatggattatactaaatctactctgcctgtgctctataaatagaacaaagcctggatgacagcatatctgtttacagtgtggtttattaaatattttaggcccattcttgatacctacagctcaggaaaaaaaaaaaaaagatttctttcaaaatatcactgcctattgacaatgtgcctggtcactcaagagttctaacagagatgtacaagggcgttcatgttattttcatgtctgctaaccctatccattctgtagcctatgggccaaagagtaatttcaacttttaagtcttattatttaataaatacattttataagactatagctgccatagatagtgagtcctctgatgggtctgggcaaagtaaattgaaaacttctggaaaggattcaccattctatatgttattagaccattaatgatttgtgggaggagggcaatatacaaacattaataggaaattggaatgagttgattccaactctcgtggatgaccatgaggggtttaagacctcagtgaggaaaagaactgcagatgtgggggaaatagcaagagaattagaattagaggtggagcctaaaattgtgactaatttgctgtaatcttgtggtaacttgaatgtatgaggagttgcttctgattgataacctgagaaagtgggttcttgagatggaatcttctcctggtcaagatgttatgaacattattgaaatgacagcaaaggatttagaatattacataagtttagttgataaagcagagtcatgctggagagtggtgctaccaaaggcattatcataaagactttgaaaagaggaaagttgagttgaattgccccaaaggtaaatatagatgaaaagaaggggaatttaggaaaattttttcaggaaattgtcattttttcctttaaagctgtgaagtgtaaacttcaaatttctgagtataaacatggttagaaggcatgaaaaataagtccggcctattgcctgacacatagtagtcccttaacaagctgataagtctatgaacatgttaccttttgtggtcaaaactaaaaatctgtttatgttgttattattattttttaatgtgcctttcttggcaggcaaatagttaagtcttaatttctttgtttccatccaggcctcttatgtgaggagctgaagaggaattaaaatatacaggatgaaaagatggcaatgttgcctcccccaggacctcagagctttgtccacttcacaaaacagtctcttgccctcattgaacaacgcattgctgaaagaaaatcaaaggaacccaaagaagaaaagaaagacgacgatgacgaagccccaaagccaagcagcgacttggaagctggcaaacagctgcccttcatctatggggacattcctcccggcatggtgtcagagcccctggaggacctggacccctactatgcagacaaaaaagtgagtttattttgacttcagtggtcggtttctgttgacttccttctgtataaaaattattttaaattgagattagtgaaatttagtgaataactatatcatgatgagaatgttaaattaaatatatatagaggatattcagaatatgtactttttcaaattttacattgaaactggttctggcattggcatactacttttaatttttttttacatctttcaaaattgtttagtataatgtttagtataacatagtatacattgtttagtataattgtttagcataacttttttggacatgacacagtaatctgccaaattttatagtcatacctattttttcctgctaataattaactctcgtgtgtatgcagacccaaaaatcacttaaacgtttttaatgcaaatgaaaattgctaatttcaattttaaaacaccaacctaattctttttattaaaatttcaaatacaaggtaacttgatcttgccaaaacaatgtagtaggttgtgatttttcatgttagctcggatgacctctggtttataaagtctccctggtaattttgacagtcatgtgaccctgttccctcttcactctcattcttccccgtttatatatttctgtatgatatctgtcacatcttgccttaagtagagttactttgttcttttcatttttctaggaagtaccttgaataaaaaactcatgccatatttttgttcaaactccttttcattacctgttatgatatagtttctcactaaatatgtattagttccttttttttttttttttttggagacagagtctcgctctgtcgcccaggctggagtgcagtggcgatcttggctcactttaagctgcgcctcccgggttcacgccattctccggcctcagcctcccaagtagctgggactacaggggcccaccaccacgcccggttaattttgtttttgtatttttagtagagacggggtttcaccatgtcagccaggatggtctggatctcctgaccttgggatctgcccgcctcggcctcccaaagtgctaggattacaggcgtgagccaccgcgcccggccgtattcgtccatttttacactgctataaagaactacctgagactgtgtaatttacaaagaaaagagatttaatttgctcacagttctgaatggcagaggaggcctcaggaaacctacaatcatgttttaaggcaaaggggaagcaaggcacatcttatatggttcaggagagaaagaggaaggaagtgccacacttttaaacccatcaggtctggtgagaactcactcactatcctgagaacagcactggggaaaccgcccccatgatccaatctcttccccccaggcccctcccctgacacgtggggattacaattcaaaatgagatttgggcggggacacagagccaaatcatgtaaatacaattgcagaaagaatggatgaataattttttaaaaatacagtgtactgagatttaagacaaaaagggttgtacttgaggactaggtattaattttaaaataattttcgatgtaaaagtgaattttacattaaattgtatttagatataactgatatagtgaaagggaacaaggaaataaaaagatacctgaaaataatagactctggtttgttttcttgagagaatactgccacattcaaataaaaagcctgactcacaaacttacctcccgaatacagtgagaaatgcttattgcctgaaacctactgataaccctatataaaacttagaaaatcagtgtttctcagataacacaagtcatctccctgccaatgggagaaaaatgaggcagggatttgaactttgtctttctgatttctaagccaatgctttaggttgttttaatattttgtggttactgctatttgtttttatttcttaactgaatactaaagctatggaaaaggacacttgttatggaatggctacaataacatttcaagaatatttgcttctattcatgtcattttttaaattaccatggttagtaagtgaggaatccccttaatttaagtcccttttaagagctaatattagtattataactaaaataaagaaattgaactcagtgattggaaggatttcttccaactctgacattccttaaacttaggaaaatattttttgaaagaactcatattttggaacacgtgctgctaaccatgacaattgccagcaagtattaaaattctcaaggttttatgataaaaatgatagagatagctagtttctatttgtctttcttaaaatgcaatgtaatggaagggaaagaagttgtgctttaaaataacttcaatagccacagttattgttactgttgaaagaaagtgaaaaatataaaatacagtattttgaaagttctaagtaatacatgctaacaaatagctcaatttttaaatttactatgaagaaatggacttggaattcattggccgaggtgacattgatagaccattgatgacattggaaccacattgctggtctgatggcagtgtttcttcaatatcacactgccatgtagaaaggtaaactgctgatattgatgtgaaaaattgatgttttggattctcaatttcatcctgttttctttttctgcagactttcatagtattgaacaaagggaaaacaatcttccgtttcaatgccacacctgctttatatatgctttctcctttcagtcctctaagaagaatatctattaagattttagtacactcatatccttttcaaaatgattacatccagtgttacttcatgatgtgatttttgcgattttattcaaatatagatgttacaccttttgcaccttgaaagactctggttttagtttattaatttcaatattattaatataatttgtcatgggtctcaaagaagactgggagagtggaaatattaagattttaggatatgttactcttatttattactctcttgttcaaagagacaaaatagtctacaagctcatttagaatttttttccaaggaaaagttgtaagcatttcaacaagtgtcatcctcaaatatttcaaattcacactgtcatctctttttttccttgattctaagctaccttattcagcatgctcatcatgtgcactattctgacaaactgcatatttatgaccatgagtaacccaccggactggaccaaaaatgtcgagtaagtcggtataagtacattttaatatagttttcgtattatcatttcatcctttccttttcctgctaggaagttattgcatttataaagatggggaatctttatcttcccatgttatatgcttgcttttctttcattctttatctcctctatggtattaacctgccagattagaaaaattagatgctgcagtgttttctagaagaatgaacttgtcagatgctccatcctgttcttaaacatgttacatatattttattcttgggtgtatgttttataattatatggatgtatttttaaataattgttttggtgaatttttgatagaagcaatgtaggaattttttaggcaggtctgagacctatattcttagttatttcaagtacctcttctttctgggagtgtttctgatactgacattttaacattttctggatagacctattatagtttaatagggtgtgtccgtaaaccaacagccagcctccaatatgtgtcttccattggttaattagagaaaatcaattataaggatttacatggtggttgtattcttttcacatctagtatcccaatggaatcttgtgtttaggtacacttttactggaatatatacttttgaatcacttgtaaaaatccttgcaagaggcttctgtgtaggagaattcacttttcttcgtgacccgtggaactggctggattttgtcgtcattgtttttgcgtaagtactttcagctttttgaaatggcaaacttatgaaaatctcagacagcacagtgttccaataagagcaaacatcagggtttgctccaacatggaatgtctatacttaaaggaaactggaaactatatgaaagctagtcattgataggcaaatactaaatttttcaaaaagactatctcccacgcagaggtatgtgacatcattaagattgatcatcctcctccatgctcagcacttggatggatagtctatgcagagacaattaggtttacactttttccaagttcttgtttaaaaatgaataaaacctgtaagaagcacacaaaaaactcaggcctttaaatacaagtacagtgctttttatattagccggcattattcaattatttgatatttttgtattaaaatgtaaaagtatatatttgtaaaagagctgtcactgcttactaagcctaagattaatgcattgtatgtacattacatatgcattcagtttattttcaaggaaaatattacattacaatataattttaatttattcataattgttcctcttttcccaaatgtgtttatcagaaaaaattgatttttacaatctcaaacctagatatgctattttccgttaatgactttgaattatcaaagtagtccaggttcattggagccacctagacaatacagaaggatcttaagatttttaatatctgattccctcacccctgcctaacctgtatcaggctccctccttttgaggtcacatatgatggggcagtttcatctgctacctgctgctaagaaaacattgtgcaaagaaagctacctcttctcacaggtctcctttttttcctattgatacaattgaaatactataaaattcacaattttaaagtatacaattcagtgtttttcagtatattgacaaacttgtgtgacatcaacactatctaattcgagaagtttcataaccctaaatagaaactctgtactcatcagcagtcacttcaatttccttctctccctggtcccttggaaaccactcacctatgttttatctctatggatttgtctatttggatttttttatgaatggaaatatgaaacagtatgcagacttttgtgtctgacacttagcatactattttcaatgttttataagaaattaaaaatctaaattaccataatgtcaagggtctaacaattgaagcatttaattgtttatttatgcatttaagtgtatgaatccaccttaataatatataatatctgtattttgtttgcatcagatgagtcaaatcaatgctaattaaattaaattagttatgatagaaaaaaagacttgcagaactatacatttaaaaaaaagtcccaaacgtagaaaataccttgactaaaggctcaggtaagtatcacagactctatctgaattctgaataactctgatttaattctacaggtatttaacagaatttgtaaacctaggcaatgtttcagctcttcgaactttcagagtattgagagctttgaaaactatttctgtaatcccaggtaagaagtaattggtgtgaagcattaggccactcataactccaactatttgggagttgttctttgttcgtttgtgtgtgtgtgttgtcactgtgtgtataaactcccctattacagatatgtgacagagtttgtggacctgggcaatgtctcagcattgagaacattcagagttctccgagcattgaaaacaatttcagtcattccaggtgagagtggggttaaacaccagggctgactttgatcttttgaaagaaagacataaaaaaacctttctttaagaaattttaattaaatttgatttcttctgttttccacaatcattatcaaaagtcagcctttgagtttaatcaattgcatgggtcttttaggatgaagatgactacttggctcacatctcaacttgaaatcttccctcctgtctgaggccttattttttcatttgcttacacagaaaggaaaatttcaattcattttaattgattttaaaatacttaagaaagagatcataaatatcagcatggggatattgcatgacattgttttttattttttctcagcaaaccatttctttattgaaaattattgcaaggtgtcactatatgctgcaatttgttttggtgctatttatagagaagtggtttgtttgacaagcattagaacttataaaaattatttttaaaagtggtttgaatagccgaattgttcattttgaaaacctgggtattctctgagaatatgttactatttttaagtatttgctgtaatatataaaaacaaaatatatgtaaaatagcaaacccagaaggctttttttgaattaagttacttaaggtcattgatttgatataatgcatgcctttctaggaaagcttgtgcttttgtttcaattcagaggctttatgtcattacgaacgcctttttctcccattttcaggcctgaagacaattgtgggggctttgatccagtcagtgaagaagctttctgacgtcatgatcctgactgtgttctgtctgagtgtgtttgcactaattggactacagctgttcatgggaaacctgaagcataaatgtgttcagaattcacttgtaaataatgaaacattagaaagcataatgaataccctagagagtgaagaagactttagaagtaagaatgtcctgcatttgttcgtaggttgaaataatgctgaaaacattgctctcttttaacaataaaatgttgctttcattttaatcatttcaaagctattcagatatatagaaagttgctgcagtgtaaatttggatcatgttatttatggttttcataaatcatcctggttctaagctatctagcagtactggcaaaaggaacatgggatagtgccataattcccaatttctatatcataaaaaagaatcagttttctctagagaaataaatgtagccttgaattaacattaaccattacatttaacaatgtgatagattatatattttatataattttatgatatagtacatacttatatatagtgacatattataaatttttatacttaatagatcgtactcatgactataacatcttacagttttattaatcatatacaatgataatattaattatcaaatattaaaatagcatctgtatcttgatagtattcctaaagatttgtacaaagcaaaatcatttactcggctttatcaacatccactttgtaacaaatgatgagtttcatagctgtttttaatggcttccttcaatataggctgacggcaatacatcaaagtttagtacagtagaattatttctgtaggtaccacatgagataatctgtatgcactctcagaagtctgcttacttcagaagtatcaatcagagtttcttacataaagcatgaattgaatgttctcttaaacaattatcccctagcatggtgtctctcactgtaaccattgatgaatattggatgaaaataagatcaagattgtactttctgtctgacacctggaatgtatctatctgatgttgccctcatgaatacataaaccttaatagataatttgacccaggtgtgatatggaagtcatgaaacctttcaaatatatgtgctgttcccaaaaggagatgagggaggaaaggtcaggattctccttagataataaaaagtaatatacatatttttcttattttaccaaatgtgtcctttctcactcactgtgataaataaaatccagtaacacttaaccaacatagtaaaagactataagtaaaacgacagaaaaaaatagaagccttaagaagaatactgaaagttttgaagtaaaatatttaacatatatatttttactaccaccatttgatgaggaatttagcatattttcccctcctttttatctttacccacttttccagtttctgattgttacaaatatgatttgaattttatttccaatttatggagaattttatttcttaatttatatgctgtaaaattaagatatctgataagtaacttacatttaatatcgtggattattttaccacacttatttaaacttaacttttctcactaggatcagtccttttattccgaatcttgctcctatttctgactttatactatgaatggttttcttaacctttattaaaagaaaatttttcaacaaattaaatttagcagcttatttcagcaagaaatgctatttcagcaagaaattctatttcttgctgaaataagataatagtaatgattcaggcagcattcggaaccaagagaagtttggagagctcaactcagcaacatgagcaggggtatttatagatagtaaaagggagtgacacacagaaacagcttgattgcttatagcttgacatttgccttatgtggacattgtctgataagtcagcaagctgtggttggctgaagctcagctgctgtgattggctgagacagttacttgtcataagaatatactaataaattagaatgcagtttgtttacatatgaagttagattgcagttctctatgtacagagacagcctttaatcaaatttaatttagttcatcactttctggaattgcttcatgtatatttttttcctaaaaagattaggaggagcccttgagttatgcgaattatttttatattgtgtttaaacatgaataaatgacaattagctaataatgaatcttataccacaatcctctcttccaaatctgtacattattctttattatcttctaaaatgtagttttagcaaagaggtgtttaaatcaagtctgaattttactttctgttttctatgttatagaagcctttcttgactgcataattgattcaaactttcgtttattggtgtgtttattacatggtttccctgtgctatagcaagaaacaatagcctcagcccttgctttcacagagcttaccatctagtgggaatgaaaaaatgtttgagggaacagacaaataaattaaaagtatctcaagttataataaatgccatgcaaaggaagtgagtctcaggtgctgagatatcaactagatgagaagacatctctgaggtgatggcatttaagctgagatctaaaagatagtaagaagccataaaatagtgtaagtgtgggtgtgagtctatgtgtggagggtgctgcttcagttaggaggaatagcatggccaaaggtcctgaataaagggagggtatgagtttaaagaatggaaaagcagcagcatagtttattaagagaaaggaagaatagcaacagagaaagtagcagaaaaacaggagtcatatcgtgcaggacatttcagcccatggaaaagagcttgtattttattttaaatgcagtagtttttattgctgtctgtcttgtggacaactgattgagcaagggtagatggtagatgggaataagaaagacaagttgaaggtggcttctctcatctaggcaagagaaaataaaggtttcattttctctgatggcagagaaatgaaggaaaaaaagatgagctcgacacacaaagtctgtagagcttgctgatggattaaagaagacaggaagcatcgaggatgacttctcagtttctggctgcaataactggatgggtggtgatggttaaggaggaaatggtggacacgtttgtagaggaaggtcactattagctagctaaaattaaaattagatgccttcgagatgtccaggtggatatataatatagagaattggagatttaagcatagaaatccgagggtctgctgagtttggagataaattccagagcaatcagcaaaaagaaaagacacacacatactcacacacacgtccctctagctctgaacatccctgatgtttttaagtgggtagagaatgggagacttggtatttaaagcaatctggtcctgagatctacaactctgacatttagaattcaggtagaaaagaggacccagaaaagaacacagagaaagagctgccaataaagtaagaagcaaagatataaataatgaagccaagagaagaaaatatattgtagaaacagatgtgcttgtgtggagtcatgctgcaagatcaaggattaaggatgatgaagaaagaaatacgcattaggttaagtaacagggaagtcatcagagaatttcttgagaaaatttggggaagtggtaagggttcaaggtcagaataaggtgcatacaggggtgaaaataagaggtcagaaagaaggtaatatgtgtagacaattctcccaagaaatctggatataaagggaagcagaataatggggcaatggctggcatttttttttaaaatacagatttttgtagggatgttttgctagtgatcatgaatgagtagttatatatgagtcaattcaggagagagagggagaagtcaagtcctcgtgaaagtgaaagagtaaaagagagagacagctccactaaaacaggaggaaagacagaagggaagactgcagagttatcaaaagatgattgagttcaaacccaatggtttttatttcctcaaagtataagatgggatcattgcctgagtgtgcaaatgaggaagagaaaattctacagaatatttgacaagaatgcattatatatatggtcatctcagggaatgagaaattcagtgtattttgagtgcaaatttgatgttttgagattacttaaaatataaccattcatttggttgtgagaggttctccaccaacattcagccatttggtagccagcatggaaaagacagatgctagattatgtgtagattgtggttctgtcaggttagggactgatgataatgaatgcccatggatttaagctatataaagagtgacaagaacctgagttggttgatagatcatgagaaagtactaaggcaaatgaattggcaatctttaaaagatcaaagaattataattgtaaaggcaacctacatagagaacctgagcataatctaaaagcattttctggtgcctatagcaaagtattttccgaaatgtctttcaaaattcaccatcaaataaattaactcactgtctcatctcctgctgaaaaacatacaatttgtagatttcctttggtgttgctatctactttgatgctcatcaaacactcttttatgctgctacacatgaaaatggggttcatgtgcacagtgtgtagctaaatgtgcggtattatggagaaatatactgtggttatattgggctaaaataggatattctcttcctcctatttttctaatatttgacatttaatagagaaagttttgaaaaaaacacaatgttggtcttcactgctaacaggacttttcctatcttcttcatctacagctgctttcttgtgagaaccacatttgccagagcgtagagtatcatgtgcaggccctctgcccagacctgatcccttgcactagttttctcagagttgcattatttgcccttggttgggcaaataagcccaagccatacacaattgtcataagttgtctttttttccattttgctacattaatcttgaaaattaataaaaaaaaattcacctagaatcttcattagatttaatatcaattctatatttttgttatatcaattttcattctcgccatatgtattttagtgggaagttggcatttatttacccccacccacaacctttgccattattctgcattcccttttaaaactaggtttattgaggaataatttacatgccacaaaattcctctttttaaacgtgtacaactctttttttttaaaactatattcagagttgtgcaacgatcaccactatctaattttaaatattttcatctttcccattaagaaaccctgggcccattagtagtcattccccatttcccttttcccccagtccctggcaaccactatctactttctttccctatggatttacccactctgtagatttaacataaatggaatcatataacatgtggctttttgtgactgtcttctttcacctagcataatgttttcaaaatgtatccatggtatagtatgtatctatgcttcatttttttattgctaaatgatatttcattgtatgtatatgccatattttgtttatacattcatcagttgatggacatttgggttatttccatcttttggttgtaattgtaatgaataatgttgctgtaaacattcatgtacaagtttttgtagaaatacattttcagttctcagtatatacctagtaagtggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntacaggcgcgcaccaccacacccagctagttttttgtatttatagtagagacagggtttcaccatgttagccaggaatggtctcgatctcctgacctcgtgatctgcccgcctcggcctcccaaagtgctgggattacagacgtgagccaccgcgcccgacctttttttttttttttttttttttgagacaggatctcactctgtcacccaggctggggttcagtggtgcgatctcggcttactgcagcttccacctcccaggttcaaacaattctcccacttcaccctcccgagtagctgggactacaggagcccgccaccatgcccggctaattttttgtatttttagtagagacggggtttcaccgtgttagccaggatggtctcgatctcctgacctcgtgatccacccgcctcggcctcccaaagtgctgggattacaggcgtgagccatggcgcccggccatgattcattctttatctttcagaatcatcccacttttgaatgtgcacaagcaatatttcattctttgcattcattttttccctttttaaaagacattaaccttaaatcttcttgaagccaaccatcttaataatttaatgtctagttgtttagaaattattgtaaagttttctagaaacctagagtgaactacttgcattgctttacctttcatcattcttgtactgtgttaaatacagtagtgcagcagtgtgctaatacagacagctccagagcccattcatataatttggctcctcagtggttgataaacatgaaccagagttagtttaaaggataatcctaaagtaatccatccttaataaaatacttcggatgtttatgtatgcaaaattgcatcttatcctcccccatactcactctaccaggcccatccttcccaaggttcaggagtgttttcaggaaagcttttcatgcacattttcacctacatttgcacctcaaactatgatcctttattgttttatctgtgtcttctaagtaagataaaatgatgatgtgtaagtcaatcaatagtattatctcaaattgtatactagtttaattataataactcccgtttttgagcagctagtacagaaagtgtggatcacagtatcaagtggtttacatgcaatatcctatttatactatttaacaactttataaagtaggtattcttatacatattttacagatgcagcagctaaggcttaatggcttaaagataattcacttacttaagaagggatagaagcaacattcaaatccaagaatatctgatttcatagtctttcttccatgattactccaaaagtctcctaataatcagattttttatttgacctgagcaaaataggaagtcaagttcttattaagtagaatagatatttaagagagatgacaaaaggtgttttagtcacttaaacgtttactatacaaggatagatctacctgatgtaaatgacgagttgatgggtgcagcacaccaacatgtgcaacatataatttgcacaagtatacatatgtaacaaacctacaggttgtgcacatgtaccctagaacttagtataataataattaaaaaaaaggatagatctgcctccagtcttagcaacaagccttaacagcaaatgacagaagagttgatgagaacttttactactaaaaattagcacaaagggtaacatgaaaggtgaaaaatggaaaatatacattgattacattgtcatctcctcaacacctagaattagtaggtcctaaataagtatggactggtgaatgaaaagtaagagaagttcagaaaacaaattttattcatttatatactgccttactgagtctgcctcttatggagtattttgggactcagtcaggggctctcttcctgctcttccctttccctcaactcccatttcctcctcttccctccctacccccttccttctcctctattcccctattttcccttctcttcccttccttttccttttctagctacaaactttctctccaggtaaattcatcaatcctcatggccttgatagttgtctattagatgataactctcatctttatatttccagccttgatagctctactgatgtccagaatctcatatcaacccagctgcttacttgaaaaatgtaatacccaacaatcatcccaaacaaagcatggctgaaacagaactcctggtttttctccagacctcctcttgctgcagttttcctctactcagcaataggcactaacaactccccggctgatagatcctgtgtcttctcttttgcacacaccctgcatgctctccatcagaggttcctgtccattctcctttcatccccttctctctacgtccacagctattctctgagttgtagccatgtcctgtctcactattgtcacctaactggtctccttgcctccacacctgcttctctatagcccactgcccacagagtaaccacagtgacatttttaaatgtcagtaatatcccatatttctatttcccacttctattacattgatgtaccacataacaatgttttagtcaatagtggaccacatatataatgatgatctcataagattctaatactgtattttttactgtaccttttctgtgtttagatacacactttccgttgtataacagttgcctacagtattgagtacagtagcccactctacaggtttgcagcccaggaacaataggctataccacatagcctaggtttatggtagcctataccatctaggtttgtataagtgcaccctaggaggtttgcacaaggatgaaacgtcctaatgatgcatttctcagattgtatcacagtcattatgtgacacctgactgtacctttaatcatacttaaaatccaaactcatttcaatgatatgcaaattctgacatgatctggcccctgcctacctctgccatcacaatcctccctctcaccacactgtagccacgctacactgtagccacatcttgctgggttttccacataccaacttcatctctgccttaggactttagctcttgctcttcccccagcccaaatagctcttcaccagtcttttcatggctgtcttccttgtcacatttagatcttagtttaaatatcatctccttagaaagaccttccctgacttcagcctaccacattgccttaatttattttctttagatcatttattactatctgaaaatatcttgtatatttatttatttacttaagtttttatccaaccacaacctccaccctcacctaaacataaaggaggcctcataaaactaatgattttattcatcatgtttattgtcatatttattgcctagcctataagatattagaaataaatatttgagggagaatatatgagaaaatagtaaacaaatggatgcatgttgcctggttccaggcctgaatttctagataattgtctatatcaattgatttgcctgctttatcataatcacagattaaactctgtaatgttataactgttcacattcttcttcaacagaatacttttattacttggagggatccaaagatgctctcctttgtggtttcagcacagattcagggtatgtaatatttgttttctctttagtctaaaggctgaaagagaaagaaaagaatgttcaatcagtttgcaaattagtctttgattctatattgctttttatatatttcatggttttatttgtctcattctgtcccacaacaaatccagtctttaaaatgttcacagttaatgcatactcttgaaagcaagactttgaaatagtgtttgaattaacacaagagagaagtcacataaatcttaagaaggtagaaaatgttacttctagaaaacaccaaaagttggttggatagtttcaggtctttcttctttctggaaaaagaagagtcacaaaatcaatttttctttctactcccaagttccactcattctccaattaaatgtgactcataggtgactgaatgttgtgttttggcaactctaagcaaaggtattccttggacttgggtacatatattttaaaaatattgtatatataagaatattgtatatatccaactgtccatattgctgcttctctcttacaccaacttcattttgtagcgttagctgcattagctctcgcttttccccctatgcagatatctcttcatcagtctctgtgtacactggaaacataggaataaaccggataaggttgctgttcccagaagaatctcagagtattgtgtttatttaatggcatcaggaaaaaccagtccaccaattttagaaagaaaaaagtcttaaagctacaaatttttatgaacttagataattactatattttgactattattgattctaatccattccaagaattatacagaaagaaagagctttggtagcagcctacgtatcaatatttactgatccaagaactattacaaaacgtctattgaaagtttccatgttttaatgatgataatgctaatggcagctatggtttattaaatactttctatgtgcaggaagtaagctaagtaatttgtacacattatcttatttaaacttattacaaatcttattttcttgaggtagatgttattatcctattttcacagacagacttgtgccttgaagaatttggtaaagtcactataagtagtcagtagtagagtgtgattcaaaaccagttcgtcctggctccaaatcatgtattcttaatcattattcaatgttgtccccctatagaagaaaccttgaatttgtaacttttcactacggctatttagcttgtgtcctgaagacactctcacctataatgttctttttcatgtgtagccaatgtccagaggggtacacctgtatgaaaattggcagaaaccctgattatggctacacgagctttgacactttcagctgggccttcttagccttgtttaggctaatgacccaagattactgggaaaacctctaccaacaggtgagtgccaagagaaacatgcattgtatttttgaatggcatatgtatctggtgtatgttaatagcctatattaggaggatttttatttatttgagaatggaggaaactctattatcttattactaaaattataagtgttttactctcataactaataatatggatagttcttgaatcgtaaataattttcttatcttctctgggcatatttaatctctagatctttccccttcctgagtatattatcccatggggtcctagttaagtaccgggacatactgcaatgttttctcaagctgctcctgcacatgtactgttttccatgcatgcttgggatggcctgaagagaagaggctctctgatgttcacctgctcaccatgttttatttttgaactcctatttacagttaaagattcaattcatttctttaagtttttctctcccaaatcaactgctcccaactgtagatttccatgactttttgtatattcttctgtttatatcattgatctctcgtactgtaagtgttaattcacatggccattttctctaacttgaatgtgaacctgttggggctgtggtctctatttacttgtatattctcagcagccaacccaaaaggaaggagagcaaaatgattattctatattatgcataacaatgatacacttttatcagaatagttgcagatttgtcttttttttcttttttggcctattttatatttctttatttccaagaatattataaatgaattcagaaattatgtaagatgcactttacagcaaaaacgggatgcaaatatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaggcaacaagtggtcacctttactcaaaattaaagaaacacaagtaaaaaaattaaaataatttttcatcagaggtaagacttatttttcatttaatatctgtttataatattctaaattcactttaaaatgctttgtatgataaaagtatattatattatacaattattaatataaatgctaattatcttgtaaatttgtttaagttacttatagattctcaatattagacctttgtcagatgggtagattgccaaatttttctcccattctatagtttgcctgtttactctgatgttagtttcttttgctgtgcagaagctctttagtttagttatatcccatttgtcagttttggctttggttgcaattgcttttggagtttttgtcatgaagtctttgcccatgcctatttcctgaatggtattgtctaggttttcttctagggtttttatggtttggggttttacatttaagtctttaatccatcttgagttaatttttgtataaggtgtaaggaaggagcccagtttcagttttctgtgtatggctaaccagtttccccggcaccatttattaaatagggaatcctttccacattgcttgtttttgtcaggcttgttgaagatcagatgcttgtagaagtgtggtgttgtttctgaggtctccgttctattccattggcctatatgtctgttttggtagctgtacatgttgttttggttactgtaaccttatagtatagtttgaagtcaggtagggtgatgcctccagctttgttctttttgcttagaattgtcttggctatatgtgctctttcctggtttcatatgaaatttaaagtagtttttttctaattctgtgaagatgtcaatgatatttttgatgggaacagcattgaatctataaattactttcagcagtacggccattttcatgataatgagtcttcctatccatgagtatggaatgtttttccatttgtttgtgtcctatcctgtttctttgattagtggcttgcagttcttcttgaagaggtctttcataacctttgtaagctgtattcctaggtattttattctctttatagcaattgtgaatgggagttcattcatgattttgctctctgcttgtctattgatggtatataggaatgcttgtgatttttgcacattgattttgtatcctgagaccttgctgaagttgcttatcagtttaaagagtttgggggctgagatgattgggttttctaaatatagaatcatgtcatttgcagagacaatttgacttcctgtcttcctattcgaataccctttatttctttctcttgtctgattgctctggccagaaattccaacactatgctgaataggagtggtgagagagggcatccttgtcttgtgccagtttttaaagggaatgtttccagctttttccgattcagtatgatattggctatggatttttcataaatagctcttattattttgagatgtgtttcatcaatacctactttattgatagtttttaacgtgaatggatgttaaatttcactgaagaccttttctgcatctattgagataatcatgtggtttttgtcattggttctgtttatgtgatggattacatttattgatttgcatatattgaaccagcctctcatcccatcaaaaagtgggcaaaggatatgaacagacacttctcaaaagaagatatttatgtggccaaaaaacatgaaaaaaagctcaacatcggtgatcattaagaaatgcaaaccaaaaccacagtgagctaccatctcaagtcagtcagaatggtgttgttaaaaagtcaagaaacaatagatgctggtgaggctgtgaagaaatagtagtgcttttacactgttggtggaagtgtaaattagttcaacagttttgaagacagtggggcaattcctcaaggacctagaaccggaaatatcatttgacctagcaatcccattactgggtatatacccaaatgaatataaatcattctactacaaagacacatgcacatgtatgtttattgcagtactatttgcaatagcaaagagatggactcaacccaaatgcccatcaatgttaaactgtataaagaaaatgtggtacacatacaccatggaatactatgcagccataaaaaaagaatgagatcatgtcctttgcagggacacggatgaagctggaagccatcatcctcagcaaactaaaacaggaagagaaaaccaaacactgcctgttttcactcataagtgggagttgaaaaatttgaacacatggacacagggaggggaaaaacacacactagggcctgtcggggggtgggaggcaaggggagggagaacattaggacaactacctaatgtatgcaggtcttaaaacctagataatgtgttgataggtgcagcaaaccgctatagcacatgtatacctatgtaacaaacctgcatgttctgcacatgtatcccggaacttaaagtaaacatgtgaagaaaagctcatcatcattggccattagagaaatgcaattcaaaaccacaatgagataccatctcatgccagttagaatggtgatcattaaaaagtcaggaaaaaacagatgctggagaagatgtggagaaataggaatgcttttaaactgttggtgggagtgtaaattagttcaaccattgtggaagacaatgtggcagttcctcaaggatctagaactagaaataccatttgacccagcaatcccattactgggtatatacccaaaagattatatatcattctactgtatagacacatgcacacgtatgtttattgtggcactattcacaatagcgaagacttggaaccaacccaaatgtccatcaaagatagactagataaagaaaatgtggcacatatacaccatggaatactatgcagccatagaaaagaataagttcatgtcctttgcagggacatggatgaagctagaaaccatcattctcagcaaactaaaacaggaacagaaaaccaaacaccgcatgttctcactcataagtgagaattgggtaatataacagatggtcacagggagaggatcatcacacactggggcctgtcggggttggggggtaaggggaggaatagcattaggagacataactaatgtaggtaacgggttaatgggtgcagcaacccaccatggcacatgtaacatgtaacatgtaacaaacctgcacgttatgccaaaaaaaaaaaaagaaaagaaaagaaaaatctctgtaccttcagtttaattttgctctaatcctaaaactattttgaaaaatcaaatctattttaaaaagcaaagaaaaccaaaaaaagttaattgaaacacctatgagatgaataaagtaattaaataagactatctccctagagttaacattcaatgatattattagcatttgatggtaaagaaagaaaagatagaattttaacagtaagagggactatagatatcacctgttcctaaaatttgaaagtgagcaaaggatttgattttatgagtaattttcaggctgtaggaaaaaatgcatgatttttatactactacacttaataatcaagaatcatacatgactttagtacaagcatattacattgctatattctttgtactgaaaatgtgtttaatatttgttttccatgaagatataatttctctttatatttttgataacatgagattatacaaaactgtcaaatttgcccatgccagtcaaattgaaataatttactaaaaaacttcctgttgttcaaatgacaatttccatttttccctagacgctgcgtgctgctggcaaaacatacatgatcttctttgtcgtagtgattttcctgggctccttttatctaataaacttgatcttggctgtggttgccatggcttatgaagaacagaaccaggcaaacattgaagaagctaaacagaaagaattagaatttcaacagatgttagaccgacttaaaaaagagcaagaagaagctgaggtactgttatttgatttaaaattcttcccagaggtagaaatgcaaaaggttacaacagaggctctgtggtgtatgttcggccttctccagctagattttctttaatgaacttttgtgtataaactgcatcctcatttctggtgtggataaaggatccagttttggacggagctagaattgacactttccccataatcatctcactgcatggaaagtgatctaaggctgttcctcctaaaccttttacctacaatgacttagagtgtgtgtgtgtgtgtgtgtgtgtgtgtttatatatgcatatatatatatatatgtatatgtacacacactcaaaatggatttccccagagcaccaattgtatacagtggtttcaggaaacttattttaatgttaaaattaacaggtactgttctatggggtatgaataccacagtgcatcatgttttgttaatgtcacaaggatttttaagataaaacaggagatattaagaaaataataattttgatgtggctacttcaggttgtcacagatccttgaaagtttcctccttaatcctctctgcaaagagattcttcagtgtaaatgtttgagaaggcttgtacgaagagaaacataatttcaaatatactgcaggtgtcttaacatttgtctgaaggtttgacctagttaaatctcttcaaggaatctaatgtattaggggagggccatcatctgtaatgcagataaattctgatgcacaaatattcagtgcagtgtttgaataatggtaaaaatcagaaaacatttaaattttggaaaattagaaaataactaagaaaattatgaaaactgtgtatctcagagtattattttgccatagaactatgtgttttttaaaaattatgatgtcatgaggaaaaatcttttagaataatgatggattaaattctgtagtcagatacaatctcagttttaaaattatacagacatataaaaagaatggaagaaatatttgcaccaattatttctgggatgtaagatgactttttttctgtttttcatttgtatctttatttcccatgtttttacaagaatttttttagtgtttataaacaaaacatttttgtaaaaggatagtactgggttatcccaggtactacctagggaatccccagaagtttcattcaggccaatactcattgaccttaccactttcttacaatattgtgctgtaataattcatttatttttcagataactcaatctttattaaaccatagagtgcctgattctattgatgatatggagttaattgcctgatcatcaaattgtctgtcttattacatggtttatgtgagttttacgttccaaaattaccttgtaaaacattaacatataatatttaatttgtctttactgtaagcaaattaataacatgtgatagtttaattccaaaacacaatagagctttatttcaacctgagacaaaaaatatatatatactttgtaagcattctgatcactgtgtaaagaaaacgatcatttcatgtttaaaggacatgattatatgcatcatgttgttatgctgataggtgaaagatgtcctgtcctagggtttcctaggatttggaaatgactcatttaagtgttaacgtcttggcccaaccaggcaatcgcagcggcagcagctgaatatacaagtattaggagaagcagaattatgggcctctcagagagttcttctgaaacatccaaactgagctctaaaagtgctaaagaaagaagaaacagaagaaagaaaaagaatcaaaagaagctctccagtggagaagaaaagggagatgctgagaaactgtccaaatcagactcagaggagaacatcagaagaaaaagtttccaccttggtgttgaagggcataggcgagcacacgaaaagaggttgtctacccccagtcaggtaccacccaaactgctcaatgtgtaccaccagaggcagaatgatagagaaaaatgtcttattttagggtaagagacctcaggagaggatagtgagtgacttcattggtatgattttaggtaaaaaccaagtatcgctgggcgttaatggaaagatgtacacaggttttcatttgggtttgatcatttttgaaagatcttatagtttcagaattgtcaaaatattttctaataaactaattttcagaaacaaaatattcctgagaaccattgtagaaataaagagactattcacactatagactagttgcgcttctttctttctttctttcttttttttttttttttttgagacggagtcttgctgtgtaacccaggctgcagtcggctcactgcaagctccgcctcccgggtttacgccattctcctacctcagcctcccaagtagctgggactacaggtgcctgccaccacgtccggctaattttttgtatttttagtagagacggaggtttcaccatgttagccaggatggtctcaatctcctgacctcgtgatctgccctcctcggcctcccaaagtgctgggattacaggcgtgagccaccatgcctggccttgcttctttcttaaccattatagttaatagtattacctgagtatcactcacccatttggaactttagagaagcctttgatatcctccacccccaaccacatcaactactaatatgtaatgccatagtccgttaattctgcttatgtggaagttctctttccctttctcttttcactggaaccttcatctctcaggtctccctgcctataattctgcacaaatccctaagaagaaccattgcttctattttccttaggacttccctgttttccatcacacaaccccacaaatcatttttgtaaactaaaaatctagttaagaatctgcaagattctgcctgatatacacaataataaaaggtagagtccctgccttcacaatttcacttgaacctaggcgatgatacggaacttggacacatatatcaataaaaataaatatgacaggagcagtccaagctggttttgaggcaaagaccttgccatactttttccattgttcacaatatttgccgctttgttctttacatagtaagcactaatgtctattgaactagatccttccaaaggctttgcatcctggacatgagtcatataagcatgaatttttcctcctctttcgtattaggcaactactttaaaaatatttaatgactataaattaactggtgttaaatggtctttttaaatacactctaactgagctcttagtacgtaaggtggattttcaatgcaaagaagcagacagcatgatttccagttttttgtgaagcttggggattgatatataaaaaagtgaatgaccggtaacaatactaagttctttttttaaatgatatattctgtgcagcaaaatgaatggcattaaaagaaaggagcagtaactctagaccagaatattagagagctgtacagcaacaaataatttacctggtcctgaaaggatgagttctgcaaatcatttcatcccttcagtggaataagttttttttctcactgaattgctttgtttttaagtgtgtttcttttaagtcagtacagagccacctgccctcaggccagtgggttcagtggtatttatatgaagtatacttctattagtaggtgcttcagcaaatacatgttattttgtaatttttctgcagtcaccactcagcattcgtggctccttgttttctgcaaggcgaagcagcagaacaagtctttttagtttcaaaggccgaggaagagatataggatctgagactgaatttgccgatgatgagcacagcatttttggagacaatgagagcagaaggggctcactgtttgtgccccacagaccccaggagcgacgcagcagtaacatcagccaagccagtaggtccccaccaatactgccggtgaacgggaaaatgcacagtgctgtggactgcaacggtgtggtctccctggttgatggacgctcagccctcatgctccccaatggacagcttctgccagaggtgataatagataaggcaacttctgatgacagcgtaaggacgttttacatagctcaggcatggctgggcccttgttcctgcatcagtcggtctctctgctggagggccttctctctggtcttgtgaatggctctgcattttgcaatactttttctatagaaacacacctttgattcattaccttaatcctcattctatgtgttgtaggcatctaactcaaagataaaatcccatttttgtaaaaaggtacttgtctattaattttataattgaagattataaataatctatttagagaacaatttatgaaagataaatatatagatagatatcagcacagaaaatgcaatgataatgctgatattagttaataatagatcccatttctatgaagaaatttggattacttgccagctactgctatctttttcaggtcacagcttaatatgtacctattccaaggaatttcaagaaatcttatttgagttgttagtaacagcaaacacgtatttagtatttacgacaaccccatgagatgacatattattatctctgctttacaaatgggaaaattgaggcatggggaaataaagcaacttaccccaatgtacttaggtaataaacggtagctctgaagttgaaacagaggctgtctgtttcagagcttgtactcttagccacccactctgctatctctccaatgtgcagtagaacgcttaccttgaaaaaatttagaatgactatgtcttaggtcctctacataaatgaaacataactgttttcctttttaaaaaatatttcatcaggagtttgtgaatcaaaacaaaatatctgaaatgcaagcattgatacaaacaaacataaaagcaaaacaacaattttacagcagttccaaaggtctaggagtcactgagtggttagagaaaggtgtctgattaggcttgaaagtgagatctttctcattcctggagtttccaggatatctctttagcccactcactatgctatgctgcattactaatgtcagcaagggctttgactagcccaataagcttcaaacatatgctacatgcttatatatttatatttgcagacagtgtagtattttaaatgagaataaagttccatcaaatccatggccacacatatatttcaatttgaataaatgcatgtgcttttaggtaacatgtgacctcattgtgtgcatgatacagcttaagcccttctctcaaggtatttttccttattgaatgagcatgttaaatttctgtggaatctaactttggttgcattttgtttttcaattatgtgtgtagtatgtaaaatattatataatttatatcagatttggagattatttcacattttttaatgaagtttcttcatttaagagttttttaaccataaaaaatcagtatcacaataaatcctaaagtgaaaactagaaaactagggaaattagtggatacttataagatacaatcataatacaataaacataaaattgtggcacaatatatggtttatatgaaaacactgcataacaaataatgtcatctctatacctgaaacatatctacatggaaaagaatttgtttaatctgacaaattcttatttcagactatagcacagaaaacatggtttcaagacttttcaaaaagttgactttttacagagatctctcaaatgggtgtgtctgttcacatacacatatatgtataatacacccatgcacatatttatgcctgtatgtgttttgatacaccacatacatgtatgactatacctatacaactatacaacttcacaaatctagaaatagatatttatatatttaaatatatgtatttaaaatatataccacataccacacacaagcagacaaacatgatctagggttgtatagttttatgtgtatatatgtgtgtgtgagtatgtgtgtgtatttctggcctgcacggaatcaacatagtgttttaaatttgaattaattttcaagttttacaaataagacattttctcacaaaaataaatatacttggtatgcatgaattcatatttttgcatggcaacaatttctagagctgaaagagtggtcacctgtttcagatgtagcttgcattctccatttcactaattgttcttttcaaactgagtcattgatcatcatgtactctgctcagttcatgaggtctttcttggcagtaagagacaagcattacagtagtttcattaatgttgtttcctcaatacaaagtaattccagaaatttatcagagaacactgagattctacccctttagacagcactacagcactttaactgttttcatgtaggactctcacatgataaaactatctgaataaattggacacccagaaaggtagagaggtgatgatagtgatgtaacaaaaaagcagctagctttattatttatctcctaaatattaattcctttaatcctcacaagaatatgaagcaagtactatgattatccccattttaccaataataaaactgagacagttaagtaactctcctgtgatatacagccagtaagtagcggagccataatttgaacccagcaatctaagctctacttttaattactcaccacagctatataagcaagattttatcttatacctacaatttattaggattctgttcttaagacgttaattgatctttttttagggcacaaccaatcaaatacacaagaaaaggcgttgtagttcctatctcctttcagaggatatgctgaatgatcctaacctcagacagagagcaatgagtagagcaagcatattaacaaacactgtggaaggtatgtaataatcttctttttaccggatagattcttaaataaaattaacataaatctggtcaaccttaaatagacatgtgaaattcttagcaataattcagaggccataaaatagttttaaatgatggtaaagataaccaatggatattgatggaattggttttatgtgcttaaaaatggataaagttatttgctcttaaacacacacattgataacatggcaaaaatgactttgcaaaaatgaaaaagaaaaccacaaagacagatgttgggtgatctgacctttcaaagacaagtgatttgatggtaattgatgcagaaaacaaggttaacccagagctatgtgattttgtccctatccaatcttacaaattacatattcgctcaaatttgtctcttctaataagcaaagatagtcaaagggtgacaactttacaaactgttctggcttctaccctctcccctgacaatgtgccccaggtcccacctggtccctggtctaacacttcacccccacattcccatcatgggagcagacaaagtgtctacccatcccctgaatgaatgatgagaagtaaatgggctggctaatggtgtgaaatatctatttcatgagtcctccataaattatttctagttgcttggttctaggattgttttcacccatgaactattacagcaagagttttttctggatagaatagtctcgtcttattgttgtcatgggactaaaaacacaggtgaaaaatatgcatattggtggaaaatatacatttctctgttaaggaaacattagttctcctgtttaccatcacatgttaggtgcatttttctctgaaagatgcccacatatgtgcttgcagtcattaatttcacttccttaccctcacttatgcttcatctaggcaacaaacatgtcataatagacttacgtaagaggctatactttcattgaaaacaatactgttaaattttcaaaatctaaactacaaaccctgtttcaatattagagtgcctcactgatttgtatctggttaggagtgaaatatacaaatggctgactccatgttctctgtttttttctcccagaacttgaagagtccagacaaaaatgtccaccttggtggtacagatttgcacacaaattcttgatctggaattgctctccatattggataaaattcaaaaagtgtatctattttattgtaatggatccttttgtagatcttgcaattaccatttgcatagttttaaacacattatttatggctatggaacatcacccaatgactgaggaattcaaaaatgtacttgctataggaaatttggtaagtctcttattgtgtgttgcatactcatagtttctctctttagttgtcatcactgtcatttcatatttttttatgtcagatgaaattgcagcctcataattatgcatatcaagaagttaccatatgaatggatgcgtagagccaaatttctggttatgtctttcaaaataattgcgcagtagtgttgaggtctataaataacaatactatttctataaaaggttaccatcttgggagttgataatgattttttcttccagcatatttcttgggagcaacacttaacatttttggttctttcttataccttcctaattacattgctgcttttttgtaagtacacatctcacatattttcagaaaacctgtggatgtattgctttgacataatagtggtgaagtgtttgggtcctcagagtcccaaagacctgtgtttgaatgctgattctgcattatatgagtgatatggccttggacaaatattttaactccatattatttaatttcatcatttataaaataaaacttcattataaaatgataatttttatttataatataattataactatatttcatttatcatattgttaggtggattagtttctacttattagccttgcatatagtaaatacacaataaattaaagctatcatcaccatcaacatcattatcatcaaaacttcaaaagcttaatgaaacaagctttaaaaacaactattttgagaaaccaaaatggttgaactggacatagaagtctcaaggataggtgagaaagtgatcaacaggcttctagaagatataagatccaattaggaaaactgatgccagagtgatctctaaaatgtacagtgttggtcacactaactcatagtaaacaaagggccagtttttgactatggatgtgaggcacgcaccagttcatttttagggctagagaatcccagcttatgactcttcgtaaaccatgaataactctgggtatctttgtcaatgtctacataagataattttgcttttgctgtggtcccaaacaaattgatatttgaagaccagaagttcttttctgggtctgctttctcttccaaattgtctacaaatattcccagttacaatgtttttccccatgtgctcactgagttggcattcttcttcccttctatttatctttggaaaaaggcaggaatatctagtttctaattgttggtggagatctggatcatgcaaataagtaccttctcactgagatgactacatccctgtgaactcagaaaggatttacctggtcacagctattaccacaacctcaagtccagctgctccctgccaatcgtcttgatcctgcattatttgaaaattgactcctacaattatagacctaaacatttcccttagaatcttctttgatcagattccttgagtcaaagtgtacccaaaggattcatttcggtgttgcctttttaagtgagtcctttgataccagtatcatgaattaatgatattttgcaatgcacttttgtgtcaaagctttagaagtcttactttagctgccatattgtcaattaaatacttttttctcatctgctgataaatctttaaagagtgagtcaacatgttctttattactctaacagtagtcagatcagaattttttttttttctggctcaaaatttcagtatcaatttatgttttctttttatcttcttccagaacaactttttgacccacataaattctttgaaaacagtgactgtgtcaattaccaaatgttttactttgctgaaattttaatatgctattctaaaacgctccgtaaaaatgatgagcactgacaggacagttagagcttcaaccttttgatacttatatgggtgccagggaagagtgatacctagtttataggactaagttgataagaatatccatttataaagtatttagcaaactgtctggaacacagtaaatgctaagcaaacagtgattattatcattgtgttgatttactattttctaatattaacagaaaaacattatatttctcacttaggtctttactggaatctttgcagctgaaatggtattaaaactgattgccatggatccatatgagtatttccaagtaggctggaatatttttgacagccttattgtgactttaagtttagtggagctctttctagcagatgtggaaggactgtcagttctgcgatcattcagactggtaaacataaactaaggttgccattatattctataataaagggatatttatttggttttgcattattatttaaaactttttggaatttgaatcaaaaggcaagattatatcttgatatcaagtaaaataaaatccattcattgacaaatgcacttaaagaactgaaaccacttgaatatttttacaatttttgaggacatgctcaattctaatttgaagtacacactaatataaatattttctattatattcatatagtacaataatttatttcacagtaagaagcttatggcttacagaatttcaatgattatatgttcataactttatgaatttggtaaagatcaaattgttatccctcaaacaattgttgtctgatcttaacaatataaaaacaaatagagactttttaaagcacatttctctaatatgctagattttatctaaatgtcagcatgaaaaaatgtataaaagtattagaaaatagaattaaggataaattatcaattctaaaagaaataatatcacaaattataagagatttccatagcaatcctgaactaaaagtacatactttcttcataatatctgttgtcattttttggggcaagtagaacaaactaacagaaatattgaaaatagatgaaaatgaaaaataagaatagtttattttagccagataataatgacaatattataggacttaaagtcagctttaaactttgaacaaaatcaagattggaaacattgaaaaaattttcataatgattaatattaaactttatatttgcttttagctccgagtcttcaagttggcaaaatcctggccaacattgaacatgctgattaagatcattggtaactcggtaggggctctaggtaaccttaccttagtgttggccatcatcgtcttcatttttgctgtggtcggcatgcagctctttggtaagagctacaaagaatgtgtctgcaagatcaatgatgactgtacactcccacggtggcacatgaacgacttcttccactccttcctgattgtgttccgcgtgctgtgtggagagtggatagagaccatgtgggactgtatggaggttgctggtcaagctatgtgccttattgtttacatgatggtcatggtgattggaaacctggtggtatgtaaccagatgttcttacattttaatttctctgtggaaattattttgtgatgatgtgatctattatttttataattttataattatatctaaagtcattaattacaacagaatagtgataattttaattcaagaaagaacaggaaagagaacaaaatgttgttttagtttccctagccaaataatccatatttatatttttcaaaatcactgcctattcattttaatgcaaagctttctatctttctctctaatcttgatagtaatggcatatgctctcttgaagctatactaataaataatatataattaagttaataataacaaaaatgaagttaataatggaccaaggcaaatgaaaggactggtcccgcaccttatagccatgtataagactagcctctatagcaaggatcagcacctatgccccacagaccacagcaagtccagtgcctgttattgtgcggccctgggagctaagaatgatttttaaatttttaagtggttaaaataatcaaaagaagaataaatgacacatgcacattatatgaaattcaaattttagtgttcataaattttattcaaacatgggagtgttaattcatttacgtatcttccatggctgcttttacattacaatggcagagatgaatagctgccatggaaatcctatgtccaatagcatctaaaatatttatcatttggccctttacagaaaaagttttccaaccactggcctatagcatcgcgtagacatagcaatttgcctattttttgaattagtgttttcttgacttgtacttctttttatttttttcttccttatactgattggtgaacactttcaagcttacaagcaggagaaaattgggataagatgctcttctatgagccactcatctcaattctcaaatctgccgtgttgactagtctcagttggtcagcttgacatcaaatctctcatcaggtatccagaaggtggggtatggtaacagccatatggtaagtgccttcacttcccactgagttgaaaaaagttactaaaatattgttgtccttgtaaccgccataaaaaaaaggactcagaagtcaggacagaaacctacattcatccaatcacacactaaatgatactgtgccagacatacacaaaggcatttatgcagcacatattctggatttttactgcacaaaaaatatatatcatggattaccattctgtaattgttgatcaccctcaagttgcagtaataaatgagctttctagttttatacagccttacaaattccttccaatatggaatgaaatacacattctctttgtttcttcttctcagttttgcatttataagctcctatagcaggatttgcttgtgtattttttctttttttttttttttctttgagatggagtctcactttgttgcccaggctggagtgcaatgatgcaatctcggctcgccccaacctccgcctcccgggttcaataggttcacctgcctcagcctcctgagtagctgagattacaggtgcatggtaccgtgccagctaatttttgtatttttagtagaggtgggttttcatcatgttggccaggctggtctcgagctcctgacctcaggtgatccagcctccttggcttcccaatgtgctggaattagaagcatgagtcaccacgcccagccgtgtacttcttcttaaaaaattttgacattaaaaaaaaaaatttttttttgacataaaatcatgaaattgggacaggtataacttgtgaaaacatgccacatcaaagccaatgatcacaaataatttaaatgtttattatttattcaggcaaattcaaagggaaaggcattcccttggctctcactttatccactcaatatttttcagtttcttctctgtgaaaagaaaggtatttaggtatagtttagataggaaggcattgtttaggtattatttagggtggaaaaccaaaaccaattacccatgatctaatcgttgtttttaagatgcttattaggacacagaaaagtggtacacaaaagatatcagacaaggaaggagtgatgagttctaccaatgaggttaaaataaaatgcttttgacacttaaaagaaagagttttagattgagacttaagggaggtttttgaagtgggcctttgaagaatggataatatcatatttgatcctggacactggtgtgagagttatgttttcctttgaatgacaatatagatttatagtaaaaaacagaggagatatatcaatgagtgtttcattcatgtagaattagtgttaactaactcatattgatattctttaattattaattattggcaatgaaagtatacagaaaaaaattcataaagttgataaccaattcttgactaatgaaggaactaccaatatcatagaattaaaatttaaggaaaaaaatatattcctgatatagtcttccacattagagttatatgctaaactggcttctctttttttatatttctctaatttattgtttcagtcgttttattgccaattaattgaaatggttaaggatcacaggtttaggaaaacatttggaaaaacagattttagtcagcttgggtctatatcaggaaacttgtgaagatctttaatcacaatgagactttgaatcaagtatacattttattaatatttcttctcttgtttaacgtgataatacaaagatgatacaaatagactttattttgatttgtgatacccactaggttatagaaatattagagacttaagatttttgaaggaaagaaagttgtcacagcagctgttctcaacgaagtgagaacctcatgagccaccgcaccctgttaagatggtttaacacttgaatggattaaggagaataaagcagtaaaatggaccctcagatcctcaccaatattcctattaaaacaaatatacacaaatgtgtatttcatagtgaaatatactttttcattgacaagtattaaactattaaaccatttcagggactttttgcaattatcaaaattgttcagtatgattcccagacctcccacctccagaattttctcttaaatcagacttattgagatacaattttaacctgctctctttacttacatctaagagaaatttttttttttttttccctcgagacggagtctcactgtcacccagcctggagtgcagtggtgcgatctccgctcactacaagctccgcctcccgggttcccgccattctcctgcctcagcctcctgagtagctgggactacaggtgccgctaccacgcccggctcattttgtgtatttttagtagagacggggtttcacggtgttagccaggatagtctcgatctcctgacctcgtgatacacccgcctcggcctcccaaaatgctgggattacaggcatgagccaccgcacccggcccagaaattgttttaggtatgaccaaaatgaggggtgagtaccttctcttcagttaggtcttactttgatttttcaaacatgaggtagatgatttgggccatgaaaagacttacatcaaatgccaattcaattttcttttttgtttcctgggtaaaacaatgtttaaatataatttacatgtttcctgccctctgtgaacattttttttttttttttacaattttttttgtttgtttgtttgcttgtttgtttgtttttgagatggagtctcgctgtgttgcccaggctgaagtgcagtggtgagatctcggctcactgcaagctccgcctcctgggttcatgccattctcctgcctcagcctcccgagtagctgggactacaggcgcccgccaccacgcccagctaatttttgtatttttagtagagacggggtttcacttttttagccaggatggtctcgatctcctgacctcgtgatccacccacctcgacctcctaaagtgctgggattacaggcgtgagccaccgggcgcggccttttttttacaatgttgagttcacctgaacttctacttcagtgagaaaaaaaaataattaactcaaaaataagactttgtttcaagagaatagaaattgtcctgaccttttttgtaatctcttctggggccaacataataggcaatgaagccagccaatcataatgccattattgcatgtgaatcaggttcagggggcaggtccttttctgtgctttaaaagtcacattaagtagtattctagcatacttcttggacagaatatttcattacaggaactttgtaagtattgtgtgtttcatttgttggaacaagtgacatttatcttctggtaaagtgaacaaagatctttagcacaatagaaaatagtcacaatcgtcttgttctacttgtttctcacaagtaataaagaatgatagatgaatgctaggagagttttttgggttcataaaagggaacttgattaaggaactactgctacagaaatgaacttagaaatgacgtaatgaaaatgatgagggatcattctctctgctccccatggtagttcttcctgggtatggctgatcctctgctgccaagtctgctggggctagtctagggatacagtgatgaagagtctaccctctcctctgtctctctcatgtggtcttatccgtgcgtgaaaaggtgcagaaatcttcgttgctggtttgtactgtggcctatattcagaaaagtgtgcttattttattttttttatatttcctgtctccctatttctctaccccctctccccaccctgatataggtcctaaacctatttctggccttattattgagttcatttagttcagacaatcttacagcaattgaagaagaccctgatgcaaacaacctccagattgcagtgaccaggattaaaaagggaataaattatgtgaagcaaaccttacgtgaatttattctaaaaacattttccaagaaaccaaagatttccagggagataagacaaacagaagatctgaatactaagaaggaaaactatatttctaactatacacttgctgaaatgagcaaaggtcacaatttcctcaaggaaaaagataaaatcagtggttttggaagctgcgtggacaaatacttgatggaagacagtgatggtcaatcgtttattcacaatcccagcctcacagtgactgtgccaattgcccctggggaatccgatttggaaaatatgaatactgaggaacttagcagtgattcagatagtgaatacagcaaagtggtaagaatgcttcatatactttgtgtttcatgttaacaattagtatgaaatgaatgaaaatttcagatttctgttgccataagttcttcttaacgttgcatatagtgatggatagttttctgtgcatgactacacaacagcttcctgaagatttatctggtaaagtttagttactagttcttaataattgatttaatcttacttagctgtatgagaaagcaccaggtatctttgagccttcccattcttcatcccctgttgcctgtcagttgcaaagttacacacagtctccctccacagtgcctgtttgaatccacctctccttttcatcacctttcctttgtcctcactattttttgcctaaaccatttcaagcagagtctcgactgatgcctctgcttccattttgcatttactctagtttacttatttgattaatttcactcacgcagattcagtcctaattatttcactgccatgatgtatccatgatctgcaaaatctctctctctcttttttcttttattctccttttttaaaaaaaaagaaaaatggagacaagtctcactctgttgcccaggttggagtgcagtggtgctatcatagcttactgcaaccttgaactcctgggctcaatgatccccctacctccacctcctgagtagatgggactatacgcccatgccacaacacccagctaattttttaaaaaaaaaagtttgtagagacaaggtttcactatgtttcccaggctggtctcaaactactggattaaggcaattcttcaatctttgtccccagttgctgagattacaggaatgagccaccacacctggctatccccaaagtctcttgaattatgtattgtgcacaaagccttcacactatggccctaaccagctttgaccagctgtatctcctagtgctcacctgaagatttgtacctgtaccaaaccaatttccttttaggccataaacactgtccacactttgcttgggatttccttctccagtcaaaatccttcaaggcatcacccttgtatttatcatttattaaattgttaatacttaaatcacagttgggtaaaggttaaatgaattaacacattaatacatgtaaaacatttagaaccatactcaacattctcaaaatgttactcatttcatccctcctaatttcttcccaagtggatctagtcccttctcattttgaaccccaaacaattttatacctcttcctggattcagtaatcttaaattttaattttttgagaaattatggtatgcctctaatggtactgtaaattcattgagaacagaaactcttttaaaccttcttttgtgccctcctctcccacgcttacctgcatatttctagtgaatcaaggtatatatcgagctctattttttttgtgaaatataacaaccgtattacaaattatgtttatggtgttgttttaaagtcttaaggtatcatttaaaaataatcttttgttttctgtagctcatgttaggaagtatttgacagatagttaccaaatatcttctatctccttaacttactaagactccaccctccatgaatttatagtggcctttgaaaaaagattatatggaataatcattatatatatatatacacacacacacactatatatacacacacatatatatatgtgtgtgtgtgtgtgtgtaaaatgctcaggaggctgaggcatgagaatcacttgagccccggaggtggagcttgcagtgagccaagatcttgccactgtactccagcttggactatagagtgagattctgtctcaaaaaaaaaaaaaaaaaaaatacatatatatatatatatatatatatatgagattaaattaaattaaaacaaaataaaataaaatactctatatttagttgagtgaataaactattaaatcaataaatcatgatatatgtttctatgtagccaaggacattttttccaagatggtaaagtaaggaataaaatatagtcatcccttgatatctaggggggagtggttccaaggctccccacagagactaaaatctgcagatgttcaagtcctttatataaagtggcacaatgtttactatataacctacacacatcctctcatatgtacactttaaattatcttcagattacttataatgtaaaactaaataactaacacagtgtaaatatttgtcttactgtattttttatttgtactctattttttattttgtacaataaagttgtatttttatatttattttttccaattactttccatctgtagttcgttgactctacagatgtgggacccagggatacagagggccagctttattttatttgtatattttaaagtcttttacatggaagaagtaatactcccaaaattttaaaagtgtgttcatttcagcttaaagttagaaaaccagcttccacgtatgccttttaaggagcaggcaactagatttagtatcatatggaaagggaaactaactcagtcacccatttttttctaactaactcagtcacccatttaaagaagattaaaattataaactccaatcgcctggcagacagttgctctcagagtaaaacttggattctaggtgttaaagtccttcaggagctgggcaaagtcaaccaaatacttctgaaattatttcttaacatttacgaggaattatttcttacctgttcatttttctttcttttaaagccaatggctgacctttcatggatctccttagctgctgacaaaattcttattcttagtatgttgagttgtaacatttttaagggaaccttatagaactttccatttagcacttcattttctaactgaaaaagcaggctcattaaaaggaagtgattttattcacagttataaagctagagttgtacagttataaatccagagttatatagttgtacagttataaaggtagagccatgtcaagagtcccagtcttgtgacttacagctcaatggaatttctacctctcttgccctctctttctccatttgaaagacaaaacaaacattcctattgaacttaatgttcaatttgtctcccctggggtataaagccatgcagctatagaattcatatatctgtggtatgcagatgaaggtgtgttgtcataatggtcagctatcttttataattagtgattttatttgtcttttgtaactgaaatcgattattttaaccatctgtaaaattactctgccacttattccatgagcccttcctaaagagtatgaaatctagtgtcctataaactgagttctctaaaaaccatttgatgttaatattttaatattcttgattttcactattttttctcattacataatggtcatatctatgtgataaaagagtgtacattcttattctattacatttctatggtatacacaatcaaatatacaggggtttgaggctaccttagaggttgtagtaacgggtacacaagccagtatttttaagtcatttagcatagtatttgaattagcaagctattaaaaaaatttttgttatcatcgatttctgcatcaatattattacctaagaactcaggttcttagtgctactgacttctaattatgaactttgaaagtttacccttcttcataaaaaattttccatgacatatctcaaggtatgtcatcttaagtatagacaaatctacctgaagatgtaagaattgagaaataatgaaggttcaagatctaaaggaaatttgttagataagatattaagctgtgggcaactataaaaaccttatgtctaaggtctttctttccagcctcttcagatgaaaatgtttccagtttaaattttgtcatttttgttttgttttgttttcccatttggatcaccaaaatagtgcacattttctttatttgtctaaaatgtttctaatcctcctatgctaacttttcctgctctaattctcataatcttcatctcccacttgagttcctagagtcttgaactgtgaatcccctcctttgcaacttttgtttttttcatatctgagatgcctttgaaatagtgatggttgtgtttatgatgagcctggatgagccaaaaaggcaaattcgattacccaatagaaagtaattcctaatttatatatttatgtcattaagtcttagccactaccttgggatatgattaagaacgccacttttaatttacaagaagaaaacaaacaaccccatcaaaaagtgggcaaagaatatgaacagacacttctccaaagaagacattcatatagccaacagacacatgaaaaaatgctcattatcactcgccatcagagaaatgcaaatcaaaaccacaatgagataccatctcacaccagttagaatggcaatcattaaaaagtcaagaaacaacaggtgctggagaggatgtggagaaataggaacacttttacactgttggtgggactgtaaactagttcaaccattgtggaaaacagtgtggcgattcctcaaggatctagaactagaaataccatttgacccagccatcccattactgggtatatacccaaaggatcataaatcatgctgctataaagacacatgcacacgtatgtttattgcagcactattcacaatagcaaagacttggaaccaacccaaatgtctatcagtgacagactggattaagaaaatgtggcacatatacaccatggaatactatgcagtcataaaaaaggatgaggtcgtgtcctttatagggacatggatgcagctggaaaccatcattctcagcaaactattgcaagaacagaaaaccaaacaccacatattctcactcataggtgggaattgaacaatgagatcacttggacacagaaaagggaatatcacacactggggcctcctgtggggagggggaaggggggagggatagcattaggagatatccctaatgtacatgacgagttaatgggtgcagcacaccaacatggcacatgtatacatgtgtaacaaacctgcacattgtgcacatgtaccctagaacttaaagtataataataaaaaaaagaatgtcactttttaagatagaatatttgcctcaaatcttagcttttgatatgacctcgagcaatttacaaaaacctttttttcagtttacttatgtgtaaaatagattgtaataaaaattacatgattctcataaacaattagaaaatgcttactgaaaagtaagtgctcaatgtacaattattataagtatggaattgagtgcttacatacacagtgtaagtagctgttggctacagattgaatttaaaagagatagcatgatgaaaatcagtgcttcagaattttagtgtgtgaaagacatacttggggactttattacaaatgcctgttctggatcatatcctttgaattgtctttgcctaaatttggatgcaacttcagaatcattaacaatgtttgcagcagctactgatcattagagactactatgaacaagcatacaccaacaaattggaaaacctaatggaaattgataaattactcaatacatacaacttgccaagattgtatcatgaagaagtggaaaactcgaacagaccaataatgagtaacaagattgaatcagtaattaaaactcctagcaaagaaaagtccaggactacatggcttcgctgatgagttctaccaaacatttaaaaaagaactaatattaattcttctcaaattattccaaaaaaatcaaaaggaaaggaagtcttctaaactcattctacaaggccagtattaccccaataccagaaccaggtaaggatacacacaaaaaaagaaagctagaggccactatccctgatgaacacagatgcaaaatgccccagcaaaatactagcaactcaaatccaacagcctatcaaaaagattattcaccatgctcaaccggaaatcgtcccagggatgcaaagatgtttcaacatttgcagaacaataaacttgctacattatatcaacagaatgaaatgtaaaaaccatgtggtcatctcaatagacagagaaaaagcattttataaaatttagtatcctatgtttgataaaaattatcaacaaattaggtatggaagaaacataccttaatgtaatgaaggccatatgtaacaaacccacagctaacatgatactaaccagggaagagctgaaagctttctttccacgatctgaaagaagacaaggatactcactttcactacttttattcaacatagtaacgaagttcgagccagagcaatcaggcaagagaaagaaataaaggacatttaaattggaaagaaggaagtcaaattctctctgtttttatataatccgatcttagatatagagaattctaatgaccgcgtgaaaaaaaatatttcataaatgaattcagtaatgtttgaggacacaaaatcaacatgcaaaaatcagtattatttctatatgttaacaaccaataatctgaaaataaatcaaggatgcaatcccatttacaatagctacaaaaaataaaatacctagaaataaatttaaacaaggagatgaaatatatttacaatgaaaactataaaatattggtgagagaaattgaagaggacacaaaaaggaaaagatattccatgttcatggattagaataattaatattgttaaaatgtccacattacccaaagcaatctatagatttagtgcaatccctatcaaaataccaatgacattctttacagaaataagacaaaaacagtcctaacatttgtgtgaaaccacaaaagaccctaaatagcccaagtaacctgaaacaaaaagaacaaagctggtggcatcaccctaactgacttcaaaacacactataaagctatagtaaccaaacagaatgatactgacataaaacagacatatagagcaatacataagaatagagagtccagaaataaatctacacatatacagttaactgagttttgacaaagtttccaagtacatataatagggaaaggacagtctctttagtaaatggttctgggaaaactgaatagccacatgcaaaagaatgaaattagacctttatctctaaatttttatacaagtcaactcaaaatagactaactaaaatgtaagacccacaattacatgactatcaagaaaaaaaaaatggggaaagtttcatgatgttggtcttggcaaggatttttttttatataaaacctcaaaagcacaggcaataaaagtaaatatagacaaattggattatattaaaaagcttctgcacagcaaggcaaataatcaatagagtgaagagacaacctacagaatgggaaaatatatttgtaaactatgcatctgataatgggttaatatctaaaacatataaggaactcaaacaactcaacaacaacaacaaaaaacaaataattcaaaaatggacaaaagacctgaatagatatttctcaaaagaagacatagaaatgccaacagatatatgaaaatatgctcaacatcactaatcatcagggaaatgcaaatcaaacctatattgaggtatctcctcactctagttagaatggctaccatcaaaaaatcaaaagatgaaagtattaaaaggatatggagaaaagggaatccttacacactgattatgagaatgtaaattagtaccaccattatggaaagtaatatgggggttcttaaaaaaattaaaaatagaattttcatatgatccagcagtcccaatactaggtaaatatccagatgaaatgaaatcaatgtgttgaagagatagagtaaacatagaatactatactcttatatgtttattgctgtactgttcacaataaccaagttatcatattaacctaaatgatcataaagagataaatgcataaagaaaatgtagtatataagaaacattgaacatcattcagccataagaaataatgaaatcctgttatttgtggcaacgtgagtgaacctggaggatattatgttaagtgaaatcagccagacacagaaagtcaaatatctcacacatatgtggaatctaaagagttgtcatagaagtagatagtatagaatagtggttatctgaggatgcgaggcttggaggagcgaggtatggagacagatttggtcaataggtacaaagctgcagtgagatagaataagttctggtgttctatggaatagttggatgactagagttaaccataatgtattgtatatttcaaaatagctagaagtttgtaaatgatctcctcacaaagaaacgataaatgttcaaagctatggatctgctggttaccctacttgatagttacatagtgtatacatgtatcaaactatcacacgtaccccataaatatgtacaattattatgtgtcaaaaataatttttaaaataaatgatggtaatctaatccctagcttacttgggttcaggtttctgagatactactttctttattcaggggctatggatatatttccccttttggttgtttaagaacctgcattttgagtaaggggaaataacatcactaacctttgatatctctccctctcctctccctacccaaaaaccctctcttttatgacttagcatcctggtttctcaatatagtttatgcagaattttcaatgctatcctagtataagttttcaggttggcagatttcctcatcctgatagatagttaaaaatacactccagaatcaaatcaaaatattttacagtaaaacatcttatcagaaaataaccaaagcgatgagttcaaacaaatggtttagtcaaatatcttaaacagttctgagtaaacaaatggaaatagaacaatagcacttctctgaagatacagtgtcagaatcaagaaaggcatgctgttatctcaaaggtacccatgagaataatactcttttcactgtttctaagaatgcaggcactgaactgacgtctcttctcagagtcagtcttttcttgaagtgcaatgaagccctccaagcacgctccaaacattaacaacacactacacaggtttttttttttttttttttttttaccattggcagtgaagtgcttccttgttataaaccttaaaggaagcatgaaatgcatccattcatcaataaatatctattgcaccttactatgctgtaagcactctgctaagaactgctgagggaaaaaaagttcatgttctcacacaactgcaataatttaatatctggattaacttgaaagttaaaatataagagcccagcgtctagatatataagactgtgagctgatccaaggatgtattagtttcttagtgatgtaacaaattagcacaaactcggtggcttacaacaacagaactttattcttttgacagttctggaggctagaagtctgaaattaaggtctgagcaaggccatgatttctctaaaggctgtagtgactatacttccttgctgctttctaactttggaggttgttggcaatccttgcagcttgcagctgcatcatcccaacttctggcccggaacccacatagttttctccctgtgtgtctctgtcttttcttcttataaggaccccagttacattggatttggacccacactaatgccctcatcttaattgtatctgcaaagaccatatttccaagtaagatcacattcaaacatgccagggttaggacttcaacataacttttggaggattacaattcaacccataacaaagaattttgacttagaagactggatggtttttttttttttttttttttttttgagacggagtctggctcagtcgcccaggctggagtacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttagccaggatggtctcaatctcctgacctcgtgatccacccgcctcagcctcccaaagaggtgggattacagggactggatgtttattgatgcaattagggatatactgatggaggagaaaattgaggaggtaggggaaatacagagtttaatttggtggcatcataaatttgagctacttgcagtacaggcagtgaacctgaacaggaggtatatggaaacataaatcttaaggaaagaagaaaagtcagagttgtagataggttggaattttgtatgttaacttttatttatcacaggattgtgtagggagtcagttccctgggaagccaatcctgagagagaaatctgtttgtctaagatttactgagagctgttttcaggaacgatacctaacatctgtaaaaagtgagggatgcaaaattattgggcagagagagaagttgaactgttacaattgcaacagagatttcggctgatcctattggcatccctggtgccgggacgaccctttggagtagtcccaaaacaaagcaagagggtcaggcctttcttctccataccatgccttggatgcaggctttccatttacccttccaggggagaggggcataaccttgggggaagcacctcccttctaccacgataatgcccaaagggggattcggatgtgagccttcagcagccaacacttttggcagctgggtgaatgagcagctcactggaaagcaggatgtgcacagtgccttacagcatgtaccgcacgacagagccatagacagaagctgagaacagtggatcaaacacatttgagaggtgggtgcaaaagaagaaagcacacacacacactacacacacggataaaatgtatacagtagcccctttacacacattatataattttcccttttgtgacctccttaggaagtaaatgtaattcatctatatttgcagatgactgagattctaaaagattaagaaaagattcgtttagtcacagatgtgtctgggggtatttttaaatttaagccccaagctttaatcgttatattacatgccatagaaaggcaggagagtgagtaggaatacaattgttttctgctatacctctgcaatggtgtaattcaggacgaaatttctgttgtaatggtattgtacatttgctgattcattgggattctgatattgtgcttctaatccaccttaatgctgactgattaggttatagtttaccttggtaataatgtgtgatttttatattttaagtgtttaaatttgtttcttcaaaactatcttactcacttctttattggaaagtaaaactgattttacttctagaatatatttctttctcaagactacattaccctttcctttccatttattcaatattaaacgcatatttcatatattatagtatttcatgtgtatctgtatatttatttgcatatatattaatgtatctctattttttcatgcaaattattgataaatatgacaaactcgtcttgaccaaactcaagttcttgtggcttctgcctgatatattcattcttttctggtgaaagccttgctgaggtaactattaggttcttaatgctactaagacttatccagctctccctggcttgttgatgaaattggcaagtgactcagaaatatcactaaagtccagatcactgcctttaaagcatctcctttgtcatgacatattatgttattacagaaaagtattaaatcaatttggcataattttttctacacatcgctttgctctctttgaaactttgtgttctctcaggacaatgtattaagtagaattgatttattattatattatcttccacattaaaactatattaagggctagggaatacacttactgccacaccaaggtacacatgttaagatcttaattatttgaataaatctattttacttaagggtacatggataatccctatttttttttgtattatgttatttttggctatttcggagtcaggactctgtttaactgcatgcaacagagacttaactatatcagcataagtggatagagcattctattttcatattttaaaacttccacagatcagcagtccacgtggcctccatgtcctaggctccatctcttctcccttctgccagtcttcatatgtgttttttcttctatcttaatgcctataattagaggtgactgttccacctacagctttatatccaatactcaggtaggaaagagaaagaaatagacagaagcagaagtaatgactatctcacttgcctgaggaaaagcaaaactttcccagagatctttatccaactttggcttatgtcttattggcaagaacagtgtgacatgaccacccctaacaacaaggaagcctggctgaggcaggacatttatccaggcatattgctgccctgaataaaatctggattatcttagtaagatacattggaagacattaacaaggtcagacatgcttctccaatattattactgctactaattattattattcctttatgaaaatgtcaaaagaattgaaagatggtaattttctattttaagagatttagttactaggcatagtttggaataatgtgcatcaactataatgttatactttgaaacatattgtataaaattaccaagaaacaaaatttaatttttatttactgaattagtatatatgaaaataagaatatattaagttaaataataaaaaactaaatatctaaccatttttaatatgcaaaaagtagaaatttcacgtggctcaacatagtataaagacataagtaaaatataaactttattctacctgtataatcttatttcttaattacataaatgtgttactgaacacagatattttatttattttttgtatttgtgcatagtagagaacacattaaaattttctatgactatctgcccagaattgtaataggcatagagtttttctctcctaaattacactcatgaaatatttttgaccccctataaatattgagtcccccaaaaaccacactaaaaacttcatgtacttcaaaggtaaagggtgattgatcattgacaacaaaagtttacagtctaatagttgcaccttagataaatacacaaaaaattataacacagaacaaagcaaagcacaatccataacaggacactaaactagtccattgaggatgcattgagtaaggggctatatcaggctgacttcacatgaaaggaggtggcacttaagccagaccttacagtatggagagaatttcactgggcagatgtggtacagaaggaaatctcaggcagaggaacgagcattcatgttgttcacctgattcactcattcattcgtctgtccacccattcatctattcagtgttcagtttacaaagtcattatctttagagagacttaaatatgcttattattatgaaatgtagtcattttattgacttgataaaaagttgtttcaatatttgcagttctcaaagttaaatatttaatgtccttttactatgtggtggttttaattatattgttatcaagactttctctcgttagattttttaatcaaagttttttccttaatttagatttatgttctgcttattactacactttagaaaatgtaataatcttttaaaaatgcatttgactttgaaaaatttagggattagttacagttatgctgttgggtaaaactggcatttgattgtattttttatgcatggattttttaaaaagcactcttctgtagaattgcacttagaccatttctttttttcaggttgtttccaaagaatttgaaaaatctttccatcttcatttgttgatgtgcctaaaaacaatgaaatatttgtctgacatattcaaatcaatgtgtcttgttctttatattttgataaatttggaaacttcacttttttgcaatgctgtttaaaatgtcaaaattaattaattaaaaataaaagtcaatcaagacatgttaattttagaatatgctttttaaagctgcatactcttactcccacttcaccccctatactgataatcacttatatttaaatgttgcttgcatttaaggaaaaatagaatgcaaaacattgcattagcacattatagtaaattcaattcctaaataatagaattatgtaactgtttaattgattgttttaaagtctctgctgaatgacatttaatcttagtattgtgattctaatatattgcttaacattgagtatatagcatgcactttcaaatatgtaactaatttctacttataggtatatgactttaaacattttaaaacacagttgactaaactttcatcatgctgcatttctaaaatttggtacctaaccttcttacttctcacaggggtaaaatcaaactggatcaatcaaggatgcagctctgtgcttttaaccaggcagagacatatgtcaccacttttacacaggttacctaaaaaaatcatacatacgatattttttcaatgtcttgcatgctgttcagctacaaaatctttctgcctgacccattgcaagagagtagaaacatatacagttgctcaatagataaagcgacatcacagcatgtcaccaggacaattctattatattttttgtgattctcttgatgacttctttgatgtaaccttcatgtgcttaagtgatttcaagtgcatgatctgtagatttttattgaattgattataaaagtaaaatgcagaatgtaacagtagccattgaagcaacatttgatttgaaaacctctatcaacttcccttcctgattaaatctacataaaaatatagagatgtttatcacattaaattgtcacatttaaattaagtagatcaaaactctaaaaaattgtggaagaaatgtttctggcataaatataaaatctcagagcaggaaaggatcattgtagttctagctgtatatttatggataagtaaattgagactcatgctttcttagtacacatctagaaagctggggtcagagccaaagcaccctgcttctagtataaactttgaatacattttgaaatggttttaagcactgttagttatgggactttatttttttaaaatcaactcagtaattaatttgattttatttcctcctatataaaatgcatcagttaaaagtcgataggccttgcctatgaatgaataagtgatacgtgttcaaaatgtttaattttattccagaagtaatattactcattgatgagaataagtttctgattacatacctaatcagagtggcctctagaaactaagaggtttagatagagcacattatgaagtgaacactaaacctatctatcaataacacatagaagacttttactgaaattagatacagattaatatgtattgccttattgaatatttctaaaaggtattaaaaatacatactaagatatattttaattttagcataacttgttgaatgaactttaatagtactaggatgggggaaggaatctctgtcaataagaattttgactattttttataatctagttcatattttgaatttctctgtcattgttgagatgtttgtattattgttatcttttgtgcaatgatgaaagcaaaactttatcctgaaattgacataccctagggtgtgtcattgcagtttcaaatgaaaaattagttatttttgtgttccagtttattcaaagataatcttaaaggtggcctaagtgaaagatttatttttctatttcaaatttggtggggatttagaggcaatatactcatagaaaactattttattccagaatatggtgaattttcttcaactagagcctaaataaggatgcatatacgtccaatattatctatataggtatagataaaacatacaatttttgagaaagctatttctagaactatatatacatgcatagacatataaacatatgcatatatatgttttgaattcataaattattattacttttttaccaagctattagaaaagatgaggcacatgttataaatagtcatcctcttaatcaagaaaaaactgaatattcagttaagaatgacttgaaatatggcactttagcaaaagttgcttacataagggatagagttattttattcatgtaaaatttctatttacattatattttcaatattagaaatccacatgtttaaaaaactagtaattattcagctcattaaaattcacttttccttgccctccttcattgtgggtagtattagtggttaacagaaatgattcagattcctaggtagcttactttgtgtttgtcattcttgcttgttttaaaattatatataatatttacaaagcttgagtctgagtatctacactttgcctttgtgtttgctgaggttccaatccttctttacctacatgttgcattttttaaagctggcatgaatagtaaaattgtctaaatgtttgcttctaaagattttaagatactctgacttgacccactgtgttccagctgctttctctgcaaacagtatagcagttctgtcatgcgcctctgtgtgaagagaccaccaaacaggctttgtgtgagcaacatggctgtttatttcacctgggtgcaggcgggctgagtccgaaaagagagtcagtgaagggagattagggtggggctgttttataggatttgggtaggtaaaggaatattatagtcaaagggaggttgttctctggcgggcaggagttgggggtcacaaggtgctcagtggtggagctttttgaaccaggatgagccaggaaaaggaatttcacaagataatgtcatcacttaagacaaggactggccatttgcacttcttttgtggtggaatgtcgtcagttaaggcggggcagggcattttcacttcttttgtgattcttcagttacttcaggccatctgggcctatacgtgcaagtcacaggggatgcaatggcttggcttgggctcagaggcctgacaagttctgtgacagagcacagagacaacccttgaatatcaatggccactttacactcctagctcttactagtaaatgcttctgttcttttcattagaaaaatacttcaagatgatactataggacctggctgagaaatctgtagccagtccagccttcttatatggccaagtgttgttgcttttgtatgatttttaaaatattcatatcttatcatttagtcatgtaagtgttcatgttactggatagaaagtgccctaggtagaagattctgagaaaaataagagctctccatgaaatcattggttttattttgaatttctctggtggtggctgagatttgacctcattatttcaccaacagaatactatatagcatgttgaccttgtgatggaactgtgtgtttcatgctgaatgagtagctgttcctcccagagcctacacgatatttctctgctagcaatagacaaagaaaaataggaaaggaaaaaaatcggttttattgcctttttttaaaaaaaagagctaacgttttagttgtccataattagaatgtgagcccagaatttattgcaactagttaataaactgacaattttctgctaaattagtgaaagtaacattaaaacttagacaacagaagaaaatacagttgagttttccactgatcaataatatctggagtatttttaaattatttccattttttcctgaaattcctgagcttcagttattttttaacttgaggcattacctttcaaaaagaatcttgagaaatttcatatcaaaaacaatattgcagagaccttgaggggcccagaaatctaaatcatgttaagaaatgtcattacactcgccgggcgcggtggctcacgcctgtaatcccagcactttgggaggccgaggcgggcggatcacaaggtcaggagatcgagaccacggtgaaaccccgtctctactaaaaatacaaaaaattagccgggcgcggttgtgggcgcctgtagtcccagctactcgggaggctgaggcaggagaatggcgtgaacccgggaggcggagcttgcagtgagccgagatcgtgccactgcactccagcctgggcgacagagcgagactccgtctcaaaaaaaaaaaaaaaaaaaaaaaaaaaagaaatgtcattacactcaagatgaaaagcacaaatagtaatatcaataatattaatgacctccaattttaaaaactctataaaaggataatgttcacacttctccagaaggaacaaaataaaggccctgaacaattgcaagagagaaggaaggttatcttggttctgcaccagaagagggtatttaaacaactagttatcctgtaacataatgggaaattggaaattggactcttatgccacatagtaactatgtgtggaattcttctcaaagggtcctttgatcctttaatcgaactttactattttactccttaatgattcaagaacacaattatagatattgatggtaaaactatgcagttaaattaaaagatggataacagtaacttgaaactaattttaaaatgtctttcttgcttactaacagataatggcgcttagtttaccatgaatgtctgccataatttttcttctcttttatacccatctcctccttttgattttcttctctcaactttcttactgtcttttgtgagtattttaaggctcaggtgaaataaccattctaggctctgctccttaaagaatatctttttttgttgtgtgaagcagagtttatagtatgtactttaaatgtaacacttttaggttcttttcagcatcaatcattctctttgagtaagatttttgttgagttctgtgttttgtacagtgccgctatatgtttgatagtttcttcttttgagcgttagattatcaagtcattagatattcctacttcacacctcttggaacaataattaactaaatagcgttaaccaccatccatgatagcatgaaataggcttgcattgttaatgtttatcttaggaaatgccagtgataaatttgtaagtgaacaacttaggacatgttctaatgtgatacatatatttaacgtaatttctactcaagcagtatagtagaaaacacagcttcaaggcaatcaaagggaaaaccatgaagaataataacgcttttaatttgaacatattttcagtaattgaaaggagcatcttaaatagctacataagtttacaatcaagataaggattatttcacagctatagtgttgtatggatgcacactgaaaaatatggaaagatatgccaatttctctctctctctctcctctctctctctcccctctctctctttctctctctcttgattacctcaggggggttgaatcaaggagatagtggaggaaactttacttttgcactgtaaatattatacatttaaaatgtttgagtcaactatatcactttcatatttttaaaaagacacttttagaaggcacttacagtcaatatcatattattcaatgaaaaggcaagaatgctaattatggtacacttctgattcctaataccaatgagtccaccttcttgatcctatgcttacatttatatttattttatttataagtagactgccaatgataaaaaattgatatttcccttttattagaaatcaaatccatgtgaaaattacagaaggtcctatttataatagcaaaagaaaattttttcatactaaagcatttatttcacttctctaatatagtatagtcattctttcatttccaaaattacttggaaaactaaacaacagatgattgagtttaaagaaatgaaagtaatatagaaaataatcatataaaataatacaaataattgatatgatcttccttatatagtacttaaatatggtgatgcttactgtgagaattcaataaatttcaccacaggaaaaaaactttattatatatatcagcctttctatataaaggtattctcttagaactctgctaaagaagcttatactatagcaagtgtagaattcaaaggtcaaattgattaaaactgtctatattgctaaagtgtgcatataaatatttaattttatatacagtattgaaatagttactgtttgaatacttaatgcattcccaagaaaaccctgaatgtgtaagaatctattagccataataatttgactacaaatacgaattaataatagttcatatcaaatattttgataatttctgacccattaaataataatatttaataactttaatttatgacataaaattatgtgccaagctgaattcaattgaccataaaaaaatataaaacctgcatgtgaaaaaaaaagtttcaatggcaagatacactacgtaaaatcactgaaatttttacttgctcataaactaatttggcataaatctttgttaatgctctcttaaacagtgagagaaccaattactgatgttgaagatgctttcatgaatttagcctaagtcagaattctgcaaataagaatcattttatttaaaagttgttaccagctcagagaaaacaattataactatttcctattgttaatactgtgtctaatagcagggaggtgctttgtattctttctatcagcaattcctcccactgccttacctcaaaacccaggccaataattcttttttcagaattgaaggacttggtcttaacttcacagaataatagggccctgtctttcagttccctgatgatagttcacgtgtagccgagaggtcaagacttgaagccgttcaaactgtagtcttctaaatatggtactttggattttgtggcaattggcacaaaatacatattagtaattataagtttatccaacatcatttcctggattaatctatattttaaaacttgtcatatatactttaaaactcattctatagtttctattttaaaacttgttctatagttttatagaatattcagttataattaatttctattttctaattttagattaaactaatttagatgaattcaatccgtttaaagaacctgtttttgagtgaaattgtgtactaacactttcataactgatactgtcatcaaactcatggacctggtgggaataatgcctctaggccaacgtagactagaggagttcacttaacagtttgttcataatttttactccagttataagaaagacttggtctgcttacatcattccttatgtacaatactttatcaatgccatttttatgatgttgtatactaggtgataataatagcattgagaatttaatgtggtacccatcaagaatacttccagcttccaagtcaatctctaaaaatcaaacctttggattgggcctatgatattaatacagattcatagatcaagaatcaatttatctgaaataagtactttattatttttagcacttgcacttgcttgaactactctaaaaaagataaagttgtaacttaataagagaatcgtgttgtagtaactagcagcttccaatattcatgtgtcatttagcaaatgagaatgaaaaattaattcttccattgatatgctgctttactcctttataaaacaatatttgggaaatttgacagagttttgctacaattgctgtatgtattttttgtaaagatttattcgtgtgtgtgtatgtgtaaacatcatggttactcataaagctctgggtgggaaaaacctatttgtgttatgatcagttatatgatcataatgacaatgaattcattactccatgtgtaagtctgccatgtttaattttcttcagtctttgtttcagagtttgcatgtggtgcttactttagtagccactttcttgacattataaataatactgttgtttaatcttttctacgatgtactaaaatgttatatacaaaataggtagtgttggtatggtcattggcattttgatttcaaaaccgtatttgattttgtggtcttgatcaaagaacttctatctatatcgtcctactacatgtatttaaatccaagagataataaaaaatacatttggactttatcttgaactcatctttttaaaaaattttaactattaagttttttgccaactatttgatctgagctttcaattatcaggcattagtaatttcacacttgtatattgcaaatagggtataggttgtcctcaaataatgattgtcagtgtccataaatttatattaaaaattaaaaaaaagtaagatgtggccaatactttccaaaagtgatagttaacatcaatatattaccttatctaattttggccacataactgttactgtaataaaataataaagtttattattttacaggatggtgtagatagaaagatcaagtatagatacatttaaggttacttgacctcaatatgtgtttatacatttttatggccatcatactattaatctttttttcataatactttgcatgagtctgattattaaataaggcttaaataccagatttgaatgaactctaaatgaactacctggtggggtggtgaattcctttctagagattaaaccaatcaagctcctcagagtgcagcacagttgataaccctttgcctggagaaggagaagaagcagaggctgaacctatgaattccgatgagccagaagcctgtttcacagatggtaagacaaaaattgagaccttggttagcattcctttattagtgttttggagtttgttttaatgcgtaatacttaccagagtttgttggtaagtcagaagactccattccatgctgattcagctatagctgtatgatgattaagggcaagtcactagagttctctaaatttcagtttccacatctatggcacttgactaacaataatttcctatcactgtatgttagactgtaagcacgctactgcttcttattttagagccttaattaatatttccagatcagtggaaccactagaatacaagttaatgaaagagtttatacttttgtccttttaataaaaaatggaatctactcactaactctcagaaaactgtttgatactgttgaccacagtctctctcaaaattcttcctgtccttagtttcagaggccatagtctacctttggtcttttatttgtcttagaattctgagttcctctctctatttctttaatgagtgttgtttttctgcacatcctgtaaataatgatttctccccacatgttcaaacttcagttatcttttcttattctgaagctccaaggtgtttcatgcatgcctgtggcttgaaaagccatttctgtgctattgactcttaacatcagtgtctttgcccctgacctcactcttgagtcccagatctgcttacagttccccattggtcatcaccatctgttgtttttaaaacattccaagtttaataagttttttttgaaaagggcaaaaacaaaaacaaaactttagcatgttttacagcttcctccagtctttaattagtaacttcatgatctatcttatcctcaagctaaagactttgagagagccttaaagttttccttgctagtcacaagttcatcaggaactctggagatagggcccaagccctcgagaggattttcatgcacactaaaatgtgtgaatcctttgccttcggggtaaagtctgaagtccttaactttacatgtaaagttctttacatttggccttagtcttttattttttaacggtacttctttctactcctgtatgctttctgatgtactgaatttcttactacttctggaatttgccagggataatacagtattgtctgaacaagtattgtatcatctaggtaaaaatacaagcccagcatagggctgtaatgtaatacaataacacaaagttcaaactttggagcctgataaacatacattctaatttgcctacttattatctgcctgtgagaactagtacacaatttaaaatcttgttcttcagatatctcatctgtaaaatggggatatcagcatccacatcacagcgatgctaagaggatcaaataagataatgattataaacttactagcacagtgtctatcttccttcttctacttttcttgttcttcaaggattctgttaacaatcttataaagaagctatgatagcacagatgctaatgacttatttagtcatgggcttaccctctgcattagtattttttgaaaaatcatcgacttgctttcagtctccaagattttctcaactagtctcttataacactcacacgtgaagcaaaatgattggaaaataaagacatcagcaaactacaacgagtgcatggtttttctttaaatgcacaccaaatatcattttatgtaaggagcaaaggtgtatgacatccagtcaatatacattttgcgcttgctgaaattagtgctataaggaaatgcagagtttcctttaaaagtgttcatgttatagggatttaagaatttcatcaacacgtatggcctgttcctcaagaaatttattagcagcatatagacttaaatttgtactgatgctaccaagattttccctattcattttatgaatgatcatcacttaaaattctaggataactagaaagaagttgggtttatcaatcaaattaaagtttcaaaccaaaaaacaacttcaaaaaacttaaagtcttacagaaagtattaacactgtgctatttcagactgtaggtactgttatactaagatataaagttttttaaatcctataaacatcaatgaaaatacttttgtgattactttgagaatgttcattgcttccaaattgaggccagtgtagaagaaatattatggaaacacattttccgatgaacctgggcttggaccattgaaatttactgagggagtactggtctctgagtcacatcccttttatgtgctccagtagagacttcatacagtctctggatctgtagcctaataaattcaatgaagcagccaatttgtttagcctttcaaccatgtattcacataatcattcttattcccgtaaacaaataattattaaatacctgtgtgccaggtccagttccaggcactggagggagacagagctgtgaacaaaacaatgccccttgctgaacaaacatatcaagatgtagcaggtcagacaggtgaaaagtgctatggagaaggtatatggaaaagaggaataccaagagtgagggggaagaacttactggcacataaatatgctatgaatgaatgattgatcagagaaagcctacctataaaggtgatgtttcttctgtgacataaagaaaattgaaggggggcatgcaaatatatggaagaaaagtattttaggcagagtaatgaagtcacatgccccgaggtgaggatatgcaaaacatttgggaaaaaccaaggggccaatatagatggagaataatgagaaatgagaagagtattaaaagttaaggtcagaggctaggtgaagtggctcacacctataattccagtactttgggaggccgaggtgggcgatcacttgaggccaggagttcaagaccagcctggccaacatagcgaaactccatttttactgaaaatgaaaaaaaaattagttgatcatggtggtacatgcctataatccctgctacttggaaggctgaggcacgagaatcacttaaatccaggaggcagaggttgcagtgagccaagattgtgccactgcactccagcctggatgacagaggaagactctgtctccaacaaacaaacaaacaaacaaaacaaatgatgttattttaaaaaagtatagatcaaagatgtagtagaggccagattataagacatttcaagaacattataaggatgtgattttaaaatctcacaatgtgcaatcttaagagtggaagggtgaatagcaatggcagaagctgggagatttcttggagtttctgcataaatccaggtgagaggtgatgggagcttagactagtatggtagagttttgtatagtgagggtgggacattctgagtatattttgaagttagagctgaagcaatttgttgatggattgaacatggggtgctcaaaaactagaaatgcatagtttccacatgaagactgagaagtatgggcttgtcagaatgaagaatcatgaacatgtttacttacaagttggtaattggtctacaagagctgaactagaatcaataccaattcagagcaatgtttcacctccagagatctctgttacctgaaaagtaatagaaaaaattaaagaaagtatgagaaacaattaaatccgagtagcatgtttttactgaagacaaatgttaaaatgattttgttgttcattgtctcacatttgttgattcatttcagcttcataactatcactttgctaatgtatttcattgattgcatctagttcctcttattcaccttttctcctccctcttaggccaggttgttccttagttccatgtcttttactttgtatttcaagacttcagactccttctatcttgccctgttctttactatccttggttctacatgacttggcaatctatataattatgaatatggcatgactctttgggtctatgtggttttatattgtgctgcttttagattatcaaattagtaaattacataatcaggattagagaaaaataacctttttcacaaggtcaagagatcaagaccatcctggccaacatggtgaaactccatctctactaaaaatacaaaaattagctgggcctggtggtgagcacctgtagtcccagctactcaagaggctgaggcaggagaatcgcttgaacccaggaggcggaggttgcagtgagctgagatcgtgccactgcactccagcctggcaaacaagtgagactccgtctcaaaaaataatgatgataataacaataatgtacttttctgttaacttggtaggaggagccaaatcaccttcctttcaaaatgttttgtattcacccacttcctataaaatgactaatcattaattctaactttagtaacacaataaccaactcctttgtgacttattttttctttctttggaaagataatgttaacaaatattgttttggttattttagacattaagaaatggtgaagaaagcttgttttcataatcaatgtttacattaattatattgatcatgctgttatagtttttaatgaatagtctgtctttgaaatggtcaatggtttcttttttcttttctttttctttttttttttttttgagagcattggtttaacatgatgaaactcagcatagactttatgtaatgcaccatgagagcacattagtgaaaccacccaaaagaaatagcatttactcactgggacccattaaaatgtatgagagattccctgttatgtttacatttaattaatagtccttttaaagagaaggtgtgatagcattgctaaccagcttggttggaaatctcttgggatatacctttgcacgaaaaccagacaggcactccatcactcttgcctcaaaataagagaaagaaactctctgaggtgtgataaaccctgagggcaatatttatgaaacttcatttcagattgaaagtttactaacttttctaattaggatttttagtgtttttatttcctcaatcataagaacaatgtttcccttcaactttaagatggacaaaagtaaatcccagagagtaaaatcttcttaaccaaagggataactttcttatccaataagttgtacctgagaaagatagattctttcttgtatgtgctcattgtttttaagtttaagacaaactctagagcactaagttaatattaaaaaattcatctcatgttttcactctcgtatttttatttgcccagaaaaagattaacttagagttaatttttgaccctacccttttactctgtttttatatcatttaattgaatgttctctcttaactaaatgcatttagataattatttaagtacaggatgataaataatttcagatctgagatcatgttcactgtaattagcattgtataaatgaagctatgtgtagattttccatcaccttttttttttctttttcatccaatctttatgtaaagtatagaattcacagagatgagacctttcaaattatggctaactgcttagaagattatataactctctttggggaatcaagtcttttaaaaatcgactataaatgacctattaagccaagtagaggacaacgatgagtaccgtaagcacagacagcaagcattagagactcagtttgagccttctgggaacttagaaacttccagagagaaagaacaggaacacataaaatatgcatgataacaaaaacaccagagacattgtagggggagagggaaacaccttgtttgctgtatttctatagagctggaaattaaagtgctagaaaataacctcatatcaactggatcaatgcactaattagacttcagaaacatgaaaaaatccattacaaaacattatctaccaggtgcatcttgtcaaaaggattctagttttgttgttttagaacatacatcttatgattatctgaagttgttccttgaaaggctcaatacttgttaatgaactatattgttttgcagaaaaggaaatcaattttttagaaatatctaaatttcaaacttctgaaacaggtatcgaaaaagtgctgcccattattactttcccacctcctgccttaatctaaaatgaggtttttcgtctaaaattttatgaggaggaattcattgtagttagtttttaaagagtcgctcttttcttcagagtaattctagttgctttaataaactttaaattgatttaaatttaatttttcaatttttcaatcattctctttctgggcctcctacgttcatagctctgtttatcactgcttgtaaaacgttttactctttagttcttcttgttcatctctcatttcatctacttcaaagttttctcctctacctgtcctagtgatgtataggtaaatatttttataaactggttctccaaagaaaataaaatatgcatacttatatcatttgttataaattttactgatattaaggatatatgacacacaatttacaaataataaaatattacaactttttattgaacattctgtatatcaaattgtttattaaaaaatgctttttttgaatcttgccaaacttttgcatgtataggcaacctatttttgcagctaataaacaagtatagttttaatatgaagattgcctggtatttatgttagtcagtgaagtaaaaatgaaatgaagaagctatatgtcagatactcatttatcagtgatgtaagcaattatttgcaaatcagaaaacagaatttcaataatgaaaggatttttaaaaaaatttttgtgctattcagtgtaatggctacaaacacaatacaccttgactgtttatctggtttattagcattttctccatcatcttcttaattctactctagaaaatcaagaagttctcatttgtagtgcttggagatttttatagtgtaaagattcccaccatcaccaatttcaaactactgatgtgaagttgctaaatgcagagttaagaagagattcacagtagttaaacaatgtgacatactcccaccatccagatacaataggccagatagaatagaccagcatccagaaagaaaagttgcacagtaatccagcagtaaagcatattactactgtcaagagagaataatccagagagaatagaccaccgtccagaaagaagagatgcgtgatagccccacagtatgccatacttctgccgctcagacagaatagacatatattgcccaaagagcataaataatagtaaaatacaggaaaataactaggaagtgatatgttttagtatttattacatttctttaaagtaagttatagaattctaagtttatatagtcaaatatttaataatggctgcttttaataacagactaacaggaatttctgacagtttaacaatgagaattggtatgggctgctttcagcacaccactgaattctctcccttaattttgttgtttcggaatgttttggaattggccctttttcctcctcaccctaaatactctttttgtatgacctcaaaaactgttctgacttcaacagatacatgtatgctaattattactaagtattcctttctagacaggcttttcccctgtttcagattcccacccccattttaaaaatctataggatatctgtatttaaacatttcacaggcctctcaaaatcattacctgccaagtcagctcctgctcctgtggttttcaattcactgctgacatccacgtggtagtcctgggctccttcctttctcttacatccaatatccaatcaatcaccaaatcctgctggttttatatgccaaatatgtcccgaatctgttacagctctagttcctaactagcattgccctgatcttcccctcctgacagggataccggaccggaatccgaaagtttttctgggtatggaactttctttacccctactcctctctattcacctcatccaacttatcctccatgtaccatggaggcatctatttacattgcaatctgtatatcttgttccctgcttataaccctttacttactcctcaacctgtataggatataaaccaaactccttaaggtaaaggccaatgctttccatagtctgaccaatttctctaaactcatctctcacccttgcctcttgtgtacgttctgcttgcatcaccgttctaaactggtttaaatttctatgcataacaatttttgctgccctcttggcctggcattccacccaaactcttttttactgatattttggttcaacccaggctccatctcttctaggaagcctctactgatctctctttctcaagaaataagccaggttagattgacttcttccatgctatcaaaatgaaccctatctgtcactttagttgccgtatctagttatattcatttatttctgtgtttatctacctaaccaaaacttaacataaattccctgaggaaaggaaatcatgtacagaagtgaatacatagaagatatgtaacaagggtttgatggacaaatgaatgagttaatgaatgaataaacctaaccatttttaagtacaattttcaaatttttgtagcggtatattttgtacatgaaattttaaagccagatttgtttttaacttagaatactggaaacaatttcatctggatcttctaatcaagatgactttcttttgaacctaatttcaggactttacactaattttctcaatattgtttagaaaggtgtatttcagagggtttttatcatagaggaattgaagtatcatagactagaaaaatttatctggcagaaaagaagaccgtaaaattcatttcatcttcggattttagttcattcattcattcacacatcaaatgaaagtggtttaaagcctgtggaggagactgaagagaaactggggattacagaagacacaaatacatagtaagctaaaactaactaacaagtattaagaaaacatcatggaaagttagaagatgaaggaaggcagcattcaaattaaatatatctgcaactggaagtataggtgttgaaattatttggttgagggagtatcacagagaacactttgcattaaatcgtgtattaaaaattttatttattctaaatatcaagatttaacatgcatgtctttcttgtcaggttgtgtacggaggttctcttgctgccaagttaacatagagtcagggaaaggaaaaatctggtggaacatcaggaaaacctgctacaagattgttgaacacagttggtttgaaagcttcattgtcctcatgatcctgcttagcagtggtgccctggtaaatgatctgacacctaagtcaacatattgattaagttgatattctttaaaatgaactaaaagataaaaatatcatggcaagttgttttacctagaattaatccctatgattagtgccaatgaaaaaattttacttgggctcagtaaatagttactgaacactcctacatgccaggcattatccttgctgcccagaatgctgttgtccaaaggaccacccagatggctatcatagaaaagagagttttactggcaatatcagtttgcaaaacagggagagagtctctggcataaactgaaagcactttatctgtagaacaaaaagagaaggttagaggttttattgaaaagggaaaatgttatgtattgctctttgagaaacttcattggccctagtaaggatttggggagctggtaagctccaattggcaagcaacagtggtgggcaaaattagtcctagagtgtagcaggtttcctcagaagctatagataaaactaatttcaagttacaacaagcagtttcaacagtcagtgttgccgagaattacaattcttggagtaatgttttgttccctgagagcttttcctcctgccttcttgactcttagttgactgttagttgagtatgtcaagaatgacccaatttgtatgatcaactttcacaatgcaaagataaatgagatccaggtagtatttattcttaaggatctaagagcaatggagaagagaagaatacaaatgagtaaatgtagaacttgcatgtgtttgtaagcaaagtgtatatgtgataaagttctgtagcaaggagaacaatcatctgcttgcaatttcagaaacgtaaatggtgcttttcagggtctagagagacgaataggaattttctagttaggcaaggcaggctgaggggaacaagtatacaatggtgaggaggcacaaaacagcatcagacattcaggaaattaaaagttgctgaactgtagcaagtgtggtgggaagaagaataagtacaggtggccagagatactatgagaaagataagcagggtcagcttgtgaagcatcttgtgagttataaaaagggaaaacagatggtagttttaggataaaaagtaggacaagtttctcatttttgtacacatacaagcagcttccatgggagttgaaagagatctggtagaattacaagcttcctgaacctttatcttaattgtcatttttcctgctggcgcggacagacatgagccattagaagcctttaggagcccagacccagctagatataccactggaagtttcattatactaaaaatagaatactcaagaactttgtaacttgccacaaagacagtcttcatgaatttggaatttgattttactgtacttagaggctaataggttgacctgttactgtttcatggatgctggcagaagatatgaggtgcttgggtcagagataaaggactttattacttatgcatggaaagtagcatgagtttcaacatatttctgttggttccccctttacccaagtctcatgagggcgttatatgagcctgaatgagtgtgtacacatatagtggattgatgttacagctgaggaacactgagcttagggaacacatctttttataccaagcaacataagtaaccctgccctttgtcccagagaccctacctcatgtctcaaggttgctcactgcaagcacaaccctgaggaatgtcccaggtacggagcaggcagtgtctggctttcttggcacactccccaacaacctgcagggacccactgtggctttcctctcctaacaagtctcaactctcagaaacagaaaatgtgatcagaaaagctctattgtccttatctggagttgtccaagtagaaatcttgatagtgtctaccatacaatgtattcctaataactaaggaagtttatcccatgctgactcacatatgtgactgagaatttcagagaggagattacaaaaaaaaaaaaaaaaattaaagaacagtttggagtaattccttgttcaaaatgtctaaaacagaggacaacatttggggagtgcattctcaaaactttattttaagtaaattgtatgaggaagaaaagggaaagatatttcaagaagctggtatttgtacttagagaggttgccaattaaactagattttttaaaagtatagcaaacccccatgacacgtgtttacctatgtaacaaaccttctcatgtaccctcaaacctaaaataaaagttaaaaagaagtaataactctcacttagtttatatttattgtgtttcagaaccttcttttaaatgttctcatttaattttcaaacaaccctgcaagattatggtattttgcttattttaccaatgataaagctgggtacagttttaaaagttgcttaatgtcccaaagccagcaaatgacagagctatcactgaattgatgtctaaagtcaaagtcaccatatcttttcactgtgtcatggctcctcagaaagaagagtacacacaaaatagaaataaatgagtggtaactgaatttagtaacagtaaagtgtaagtcctaaatcagatagcgctcacataaggtcattcttgcctcctcgctcctacatccacaaaattcattctggaagctagaacccctgtgggctgaattctaattcctgctctgggaccactcagacagaggttctcattatcaagacttcctgtgactctgctctgaccctgtacaccagttctagaaacaggcttctttttccttgtgactaagccccttccttgtctttgtatttaaaggactgagtcattgccatagtttctgtttagatggcttgcctgattcctccagaacttgggggtggagtggcagggaggaaaatcctgagggatagaagcctccctctgcacttcaattccattgaatttcagaacctctgccaactgctgggaccttctttttgcatcttagtaggtctgttcagtctttctgccccaacttgtctcttttcttcaatattgataccaacagtttcccaaaactgctaaggataattttagagtattgatagcggaacttagaataagaagaatctgaatggcacaagttcgttagtatagacagtcaataaatataatattattcacgaaggatacaggtctttgactttcattttatccaacagggttaatggtgtttcaatcagaaagagaaaattctaacactttagacctgttgagatcagcaatcattttcagtgtctcaatgagccctttttttttttttgagatggagtctcactctgtcccctaggctggagtacagtggcatgatcctgactcactgcaacaatgagcccaatttttaagtccattcaaattctttgtcaagggacatgtggctgaagtccaaagtcaaagtcaccctatcttttcattgtgatgtggctcctcagaaagaagagcatacacaaagtagaagtagatgagtgataaccaaaattagtaacagtaaagcgtaagccctaaatcagatagagctcacctaatgtcacataatattgttgtgtgtcacttactaaaatttcttggaaagcataggaagtaccgcattcatggagagaatcgaacagtgattttcagagctgaagctttgaaggataatacatggatagactgtcaaaggaaaaagcacgctctgtgttctataggcctattaagctactgcagtcattcgtttagcaaacctttcctttcgtgctgctgttattaacatggtgctaggcactgggatgcacaagcatttgagaactaagagaggctccacatcctactggtggggaaaaaggggagtgtaggttgtgaaaagtttataacaggggctgattatgggatcatgtcttaaattgttaggacttatccaattttaatggagaaagaggaggaatcagatggaggtggaaatgatcaggtgtcagagatcatgagtctttttgtggattacaagtaatttcattcttttgcactttctcttgacctctgatgaccagcagcagcactggtgagaaggctagaactaagtgttatgtggtaactgtaaacccgaaaaaatgtcagctggcccatgtcaatattatgtttcagtgttcttagcattagaattgtatttttcctggttttgtatgaatcataaagcttaagttgtgcatgactgaaacttgaatattatttccacaggcttttgaagatatatatattgaaaggaaaaagaccattaagattatcctggagtatgcagacaagatcttcacttacatcttcattctggaaatgcttctaaagtggatagcatatggttataaaacatacttcaccaatgcctggtgttggctggatttcctaattgttgacgtaggtacttttgagtacattttaaaagatttattcttactgagtgttgtgaaaaaaccttcaccagtgttgtatattattatattttaattttgttaagcttcatatactttcaggttataggtttattcttttagaaatattagcacagccagtggaaatatatctgtgaggcatattagaacagaaatcattcaacaaatataaattatttctgtgacatgctaacatttataattgagttttttatgttttctgcaaaggagatgtgatttaaaaaatatatatatatatgatttgaatgtcattcctgttaagaataaaacaaaatttagaagtaggaggtgataagagaaaagggcagagacacagattaaagagaggaaatgatgactattcacaatataaaattggaagataagagaggatgaggggtatatcgagttggctgatatgtgagctaaaatttttctaatttaagtaaagttaaactttgtattttttaaagttaacttcgtttttggggagggcgaaggcatgaaattttaaaatatctatctcagacccaattataggaaaaaccaaaacgagacacaattgtctctatctcaaatatgcaacttgcccttcatctccagaatggcagtggggcagtggagtaagcattattattaatgtaataattttacataataaattaaaagaactgatgaatacttgcatgatggaaaatacataataattctttagtttacatataagcctgtaaaaaaagcacaaatatttttacaggcttctgactgagatagggaataaatcttaaaacagtcggtgagatcaaggaatctcaaatagaaactgatggtacaacacccattgttatcatttcattgaatatactctattttatattttaataatataatttaataatatatatgacatattttgtattatttaaaatggcgttcttacattattttataacctcaatctcccagtcttatttttctcagaaggatatgttctttatcttttactaattcttctagatattcagaaacctaacctttgtgtttagggaagaaacatcctataatcagtcacactttttagagatttgtttacatataatgcctgacataattaggggtcaacacgcaagtttttatatgccaataagtgctttaaaatttaaccaagggtttgcattattgagcttttaagaaaagttgaattcatagagcatcatacgtggattagaaaatcacaagtaagcatgatcataaatagtaaaatctgactgcagaatgttacccaaattagtcatatttactcaaaagttcagtttctcaactgttatttaaagcttcataaacttttggcttctggccaggcgcggtggctcacgcctgtaatcccagcactttgggaggccgaggcgggtggatcacctgaggtcaggagtttgagatcagcctggccaaaatggtgaaaccctgtcactactaaaaaatacaaaaaagtagccggacatggtggcaggtgcctgtaatcccagctactgaggaggataaggcaggagaatcgcttgaacccactaggcagaggttacagtgagccaagatcgcaccactgcactccagcttgggcaacagggtgagactccatctcaaaataataataactttcgttttcttaataacttgttttctataatatgtaagttatgtgaaaaaattttttaacatagttatatattacacctcatatgtgttatcttgggtttcctctgtgagaactctaacttttgatatagtaatgatttcttgtttttttttcaattaaccattcatgtatcaagttatctagttattgcctagaaaatgaaaaatgataaaatggatatttttgtaatatagtagttttaaagaaaggctgccccaaataagaagaaattatagaaggaatgtaggttttccttttctcttaaactgtaaatatgatggggtctttctcttaaaaaattatacaatattttagatattttaaaatctttaaaatattatcacaaatatactcactttcatcattcagtgtaagagataaaggtttacaagtaaaattgaacttattttaaattaagagatatggctgtttaataatggaaattgttttatatttgaaaatttaaagtgataattctcaatgagtataacatcctgttcagggatatgtgtgcagaaatcagggagataaacaactccaatttgaaatattatttaatattttggaaacaaacaaatataatgcaaagtacagagagtaaagctccacacaatattcttgctgatatagatgcaaatttcaggacgcagatttgggggagtggctgactttttttttcctgaaaagtatagtaacttatctacagcagttgttctccaagcatggtctcaaaactgaaaacgttggcacatgtaggtgagaactcataaggggtgagttcctccaagacctactgaatcagaaactctggggttggatgcagcaaatcgttgtaacaagtcctacagatgattctgttgtatattcaggttacagaactacttatattatttgctactcagagaatggacttgtctgatttctgcaggcttctgggttatttgaaacttacttgattaaacagtgttcagcagtgcaggcacatatttgtatatatacacatgtgtttatagaaagatatgaatatatatgcatatatattctcagttacaaactataccgtggtcttcttaataatatacatatattccatgtcataaaactcacttaacagtggcttggagaacagacctatccgtattttaggggctgtatcaattatagcagacatgtcttagtctaccttttactgctgtaactgaatacctgagactgggtaaagtattcactaaagaaatttatttcttaaagctctggaagctgggaagtctaaggtggagggtccacatctaatgagaactttcttgctggtggggacactggagagtcctaaggcagtgcagggcatcacatggcgagggatgttcactgacagccaaactgggttttataataaacccacttttgtgatatctacctcactcccgtgataacccattaagcccttactccatattccattcatgaaggcagagcctttatgatctcatcaccttttaaaggctctgcctcttaatactgttacattgggaattaagtttcaacatgagtttcagaagggacaaacattcaaaccacagcaaaacattttaaaattttgttttttttctgattaaagagggagcatacatattttcaagtgttcttaagtggaaaataatggatgtgaatgactgggcaaatttaagagtggcatgaggagcaatttattctgttaaatatatagcttaaggaattaaagcagtattacatttagtgagacatttggaaataccacatgaggacgagtactttaaaacacctcctgcttcataaatcagtttacctgtctctgatggtaacagcaatgaacttgttgtgaacaatgactttttctactcctgaaatagataaatatataaaaagttggctctcatgtgttcctattcttcccttgaaaacctgaatctctcagtcactaacttaattcgtaatgaagaactgcatttcagacatttttcttagacatcgtctcctgctacacaggatgtgaggcaaatgaatattttccatggaactaattcagagagtaaatgcatttatagatccagatctaagatcattgaggagctcattgctttgtaactaaaatgtgtattgattgtattgctgttcatgttctttcttaagataatttacggtaagaactcaaatttcttttggatactggttatggtaattgtggctgatctatctaacatgatgaagttcttattatatctatattgattgtgaagccaaggagatcagctactttcattctctcttcagtacaattgtgatttttctcccatgaaaaattatttggtagatctttggtgccaatcctgagaaattgtagggcagaagcaatcttgttgagaatttgccccaactcaaaatcatgagtctacaaaatgttttttaataagatgtataccaaggcatcgatttagttgagtgaataccacacttttagggcttagcttcagaatcacacacttcactttgacctgagttttataattaaaatgttgaccattgttctaatgaaatcttcagatttaataagcaattacaaaacaattgattacaaaagtgtgaaaagcatatgcatgatttctactcaaatagaataaacaacttactttgcttacattatttcttcttattttggttaaccttcttttaaatctatttccttatagaaacatccaccttaccctgtttttaggcattgactgtacaactccatatataaaatcttattgttcctgttgagttgcttttagtgagtttcaaaattgactttttccttttatgcttcatcattttattgacacaattaatgaaaatgctatttttataggtttctttggttactttagtggcaaacactcttggctactcagatcttggccccattaaatctcttcggacactgagagctttaagacctctaagagccttatctagatttgaaggaatgagggtaagaaaatactaaactttataatattcttatttttttaatggggtttaaaatataaaaacaatgggtgtgtagaaattatgagtggtttctatttacataaatatgtgagctgtttatgtatgtgagtgtacatacatatacatatatgtatgcatatatgtatacattatgtatataatagtatgcgtatgtatactatatatagtatgcatttacatactatatacagtatgcatatatactatatatactatgcatatatataccatatatggtatatatatgcatattatatatactaatagtacatatatgcatgttacatatactaatagtatatatgcatattatatatgctaatggcatatatatgcatactaatatataaatgctaattatagcattaattaaacagtatactactaaaatagtataatactaattagtatatactagtatatattaatatataccaaatatgaaatactatatataaaatatgcatatatagtatatgtatatatcatagttactgtacatattagtgtatatatatgcatactacatatatatatacacacacacagtatttcagcacatattgtttaacaagggaataagttaatcacagtaatcctgcctttatctaatattgtcaagcaatagaatgatccaaatgagtgactttttccatgttaaatggaaatcttccctgtgctaggaagtcccagtgaaaggggaaactattagtcctaacgtcatctctcatacataagtaatcaaggacctatttatatcaggtgttcaaataatatttgtttaaggaactacctgggtaaggagcattagaaaagatagggaagtgagaccacatcagccatcacatttaatttcacctttaaaataacattacaaagtaagtggaattataactaaattatatttgagaaaactaaaggtcacagagtttacttgatttgcacttaggctgcagagtgcctgaaaagatactcaattagcagagacaaacaagttataatgaataacaactaccataacctagctttagggattgtattaaactagcaactttgtaacacacatgaaggacaactttattttctataattagtccatatcctcatgtgttggtactcttctaaaatttcttccccagatttatacctgcttttttatgcctctgttctgaaatgcttcccctgccctgccccgaccctaacccgtctgtacagcaaacacctatctgtcctttaaggctcaatttaaagttgccttgtctgtccaccagcagactaaactgcatattctctctctgttcagacacctaaaatcctcattatattgtattttacttatttgtttacttaactacctccctaccttaccatctctgagcaatttaacatggagattagtctagcttactgatattttaagtgtggtttactaaccagctattagcattatctacaaacttgttaaaaagatgggcggatcatgaggtcaggagttcgagaccagcctggccaatatggtgaaaccccatctctactaaagatacaaaaaattacccgggcgtggtggtgctcacctgtagtcccagctactccggaggctgaggcagaagaattgcttgaacctgggaggcggaggatgcagtgaactgagatcatgcaactgctgtccagcctggacaacagagcgagactctgtatcaaaaaaaaaaaaaaaaagcaaattcttggatctcaccccagacctactgagtcagaaactccaggatggggacgaaatttgggtgaacattagagatctttcagcctaccaaatcttcactgttacccctacaaacctaattatgactgcaatctctgctattttaagtatccagtgtataatggcttccaagtatgtcagaaagaacatgattatataaaaagttcataagcacaattcaaaaatattctatctacaagttgaaaattacaagtttcattttgtgcagaaaggtggtattagaattcattgacattaacttaattttttgagcattaatagaatgctacaatctacccagaactttcccctacaaaatcacttccttcagtgactaaatcagcctattattttactctgtttgcactttgttctttatctgtattacaggtgaattggatgcaggagaatagtttagtatattttgagggacaaatgtctatttagttacatttgttaaccatgcaaaaagagacatttcttcatgctgcagggctattttgaaggataaatgaaataatgaaataatacatttgaggcatattgtagttagcttgagtccttaggctctttagctttcccaactggcttctcatcagaccaatatgtgcctgtgtttctcagagacccatgattccaacattactcatgttttttgaaaaacattttaaataccaactatgtgtcaggtgttgtggtataaaaaacacagagtacagtggtacatagaccccagtccccaaagagtgagcagagtcaatggttacaagagagacagacaagggcacataatgatacaggaatatacaggagggagtggctatctggggaagtctgggaagacatcacaaggaggtgacatttcagatatttcatgactgaagagtaagccccaaatcataggcagataggcattcaaggcagagataccacatggtagggcagcacagaacagagtgtgcaaggtgtacgtggtcagtgactagacagatcagtgtgcctaggtgataaacgtagcagggagggcagcaggaaatgagcatgaaaaagtagactctactaggaggtggccactttgaacttcattctctattcctgttcctcctaatcttttttgaggtcacatggactatgaaaatctcatgaaagtaagaattttctttccagaaagagagcacaaccataatgtttgttatgtaatttcatgcccccagggaacctctacaggtccatgaactccaaataatcattgctctggaccaaaggtcaacaaaccccagcctgtttttttcttggaacacatccttaccagtttgtttatatgtgacctatgactaccatcctagagttgagtagttgcaacggagatataacccacaaagcctaaaaatatttataaaaatgtttgcaaacgttctccctagatgatctgattagtagtcagagatccttagtatttctctattttaaagcatcctgagaacctgcttaatacattgcagaaaagaaggatcccagtgctacttccctaacaggttaatctttaacacatttgtacttgaatgggaactctgaagtgaatgggagctgaagtgtgagaagacaggtagattttaaaggattaagtaaatgagaaaatctggagagtgttaaatgctacagtaattgccctggaggaaaactggagtggcaagacacactggtgatcaatagcagtacaaatgctgaaaagccgaaataaagaaaggattaaagaaaaactgttcctaatttttgccttcactgaggtcagcactatctatatttagtcaaaggcttacatataaagtatttagagtaataataataaatttaagaagattgctcttattttgtagaaccagccaaatctaaaggattcaaaccatttaaagactcaatccttttgtagagaagaatgtctcaaaaaaagagagtaagaaaataaaataacaaaattaaattaaaatgatgtccagtcataattccacctgataaacactaatagtttctctatgggggaatctttaatgctggtaataataggatcatgaacatttaaatgtatataaatatatatttttcatttatgtttataaatttcttttttttccagagggtacagattttacaagtttaccttatcaataacaggttttggtttattatttttatttattttacatttatttaagctattacattatcttagttattttgtcttatagtttgctaataattttattgtgtgatagtcatgtaatttgtaagcctggtgatcacagaattcctgtctcattttgtcttgcttggcacactgctgagtgtagagtaggcatttaataaacgtgtcggttggtttggtgtcttagatatttaagggtggcttttctaatttgtttatgtctgcatggcatttctttcatgttgcctatttaacatcttactaatcctaatcattcttttctttcttttgaatacaaggtggttgtgaatgcactcataggagcaattccttccatcatgaatgtgctacttgtttgtcttatattctggctgatattcagcatcatgggagtaaatttgtttgctggcaagttctatgagtgtattaacactacagatgggtcacggtttcctgcaagtcaagttccaaatcgttctgaatgttttgcccttatgaatgttagtcaaaatgtgcgatggaaaaacctgaaagtgaactttgataatgtcggacttggttacctatctctgcttcaagttgtaagtgtcccctttcatgaatgcttggtattttgatagatattggacaaaggatataagttattctttaaatagtctgttaattataaattgaagataaaatactgtatggtttttaaaacagtgccagtcttggccaggcttggtggctcatgcctgtaatctcttcactttgggaggccaaggtgggcagatcacgaggtcaggagttcgagactagtctggccaacatagtgaaaccccatctctactaaaaatacaaaaaattagccgggtgtggtggtaggctcctgtaatcctagctacttgggaagctgaggcaggagaatcgcgtgaacccggaaggtggaggttgcagtgggccaagatcgcgccactgcactccagcctgggctacagagcgagactccgtctcaaaaaaaaaaagtgctggtctttcctagaacatgttcctacatgaataagaagcataggttctgttgcatttgagttattaatttataattgactctttggtgtcatgtggactcccatcttgtaccattcaaatattgcttcaaaaatgaacatttttggacttcctgcttcacactatgaagggatgagaggaacttcccacaggcgtggagaacccttaaaaaaatgaagaaaagatgtttcctgtcttattcataaaagttcccttcacctttaaaaaataaaaggttatgtgcaaaattcacaggcaaatgtaacaatttggaaataattaactagaactaagatttagcatgttacttgggaatatgtccttttaaaaaaaaatctttatattaaatggacatgttgaatacagcaaaacttactagtattgcttagtaattgtggtaacattaaacagacaaaactatgtttatggctattttagaatatgagtttaacattcagattctattaatattattcttaaaggcaacatttaagggatggacgattattatgtatgcagcagtggattccgttaatgtaagtattgattatcttagcactaaactttatttttgcaagcttctgagtttatttcagtgatttccaaactataacttcatgaagcccttgggactcacacagacaatggagaacatccacctgatagtagcctagtggttgtgagtacagcagttggagatagaacccgactcaaattttagctgtaccacatcatccaagctctacggctgactttgggcaaattaccttaccttcttgagacttagtttatttgtctaatctataatttaatgaatctatcctttgcctaataaatgtctataatcatccaatgcctactccatagtgttgtgaggacccagcaagaaaatagaagcagagcacttagtgcaggctggcattactaaatgctcagtaaatattcgcttatcagataaaaataaaataattataatgtataacttttactctttttacattttaaaacaccttggatgagtttatgtgaaataagttccatgctaagaaactttaatgattactccctaggttatatttttacaaaatctaataaagacatatttggaatcaaatgaaaatacatggcaaaatttatataaatatgataatgtctcatttacagtacttttggaaataacatatacacagtatgtcacttagtttttgaattgtcctaccaatatgagcacaagtatgcatgtgattaagccctttgtagttatttgaagattatattatttattacataagtatgtaataagttaaagaagtatgtaatgaatacacattgaataaattgttaagagtgattatgttaaacctcaacaatgctatagctccttaaatcctgtttacctaggatagcactgtttgaattaccttgtcagaccatgtgaaaaagaaaatgaggaatgttattgtctatataatgctaacttttgtaaattttataggtagacaagcagcccaaatatgaatatagcctctacatgtatatttattttgtcatctttatcatctttgggtcattcttcactttgaacttgttcattggtgtcatcatagataatttcaaccaacagaaaaagaagataagtatttcaaatatttttcattgcaatgaaagggatgaacaattattttccaaaaaagataatgtaaagtttaatttgatatcaattcatgattagttttattagaacttcatttatttttcaaataattgactagtatacaattacaaatatacttaatctcattcatttttgcacagaaaacaattttctaaataataggttctggctaacctatgatttaggttaaacccataggttttgagcaatatatcaactattctgttttttccaatgtttcctatagaggattgaataattttaaaaaatcaatacactatttattcaaaacacattttataagtagctatttattttattctgtgtgatacatactgggttggagcttgggggagaccaacataagatagggttcctgagctcaaggagtttatatcgtagcagaataagtaatacaggtactgaaaattaagatttgatgaatactgtaatttgggcaatcttctgaaatttttaattaattaatcttcacatcaactgtattagtttctttgacctgtgagaacaggataatacaaacctgatgacttaaaacaagagaaatgtattctctcacacttttggaggttagaagtccaaattcaaagtgtcagcaggtccatgttctctttaaagcctctaaggaagtacattttattgcatcttccaacttttggtagcccaggtgtttcttggtttatggcaacagaactccaatctctccctttttagtgacatgccattttccctctgcgtgtgtttctcctctactagtaaggatgtccgtcacactggattaagagtcaactccactccagtatgacttcatcttaattacatcttcaatgacattattttcaaataaggtcacattctgaagtgctaaggcttaggacttatatgtaccttcttggggcacacaattcaacccataacatcaatcacaaaaagtattattacagagtctatttcataaatgaggaaaggcacagagagattaagtgacttacccaaggtcatatatcgagtatgtgatgaaggttagatagagtcagtagtaaattcgatgtatcttgtactctattactttagactctctactatgctctatccccaaatactataataatgataaatacaataagaaaaattagcaagaacaaaaaatgtacatgacagtcggaataaaaatagatgctgctatagaatagtttatgaaatatgtgacaattgagaaggctttggaagatgggtaagaagatatgcagcaactgagaggcaaaagaatggctaacccacatggagggaaacagaaatgagtgaggaaatgaattgtgttgataaaagaggcaacaattcaggggggatagcaatgccttcagttttgaagaaatttaagatttggagaaatagtaaaagatgaggttttgacctgcttgagagtagatataaaaagccatattagaccttttttagtagcagctggtgtttcattcatatatgttgagtagggaagaacatggaagcaggagattgatgtttattaagactcaatgatatgtcagatcctgtgtaaaatacttctaatgtaattgtagtcaaattttacaacaaatccaaaggacatagaatcctcatttaacctctcagaaaaattttcttatttgcacatggcctgacagctaaaacgtagtttcattatggatctagctccagaattgctttttctagagtctgagacagagctttaaaaaaaaattaatctagcagcgatgcccaaaatgactagggaaagaccaaagactatttcagaccctgcttgacctgccacctggcccatacttactctccaaattagtcctgtgtggcaatggagaataggaaatatattccattaaaacattttagagggtagaatctacagtattgcttgtgatttgcaagagataaaggagtctaactgtggagtttcttctcaactaactgagatactctggttgtagtaccacctatacaaaaagagacctttggagactaaatggtagaattacaagagaataatcatttcgggtactggcttagcatctaacaagacagatgtacatgtatcgaaatgtcattagtgtgtgtgattgactattcacacgtagacaaaaatttagtaaatattgcattatttaccctgacgtttccaaatacagatgtttatttttaaagagttttaaatctcatgttccattttacattttataaagtataccctgattttcaattgatttttctcaacttaagcattatatatacatacatttaatatataagcacacacaagtaattcctcagtcttccctgcataggataaattaaatgcatctagaaagagcagtatgagtatgaataaaacagccaaagtccaattgactttaatcttcctgcttgttaatgttattcttacaacatttctactgcaggaagtaaaatagaatgggttgatactatcaccactaaagtcaggaaagagttactgatttagaagcatcaaaaactcatatcccctatcattcttccagtatttcatcttgaaatctaagtatagcatttattttctgaccttttctgaatgatttagaaattctttccactcttccttgaaaaaacatgaccaacacttcaattagttaacacatgctattagaagtaggtagtagatgctttgtgatgaactcatttattcgatagaagaaaaaaaatgaaagtggtatccttttctttgcctttcctatttttaattattgtggtaaggacacttaatatgagatctacctgcttaacacaatttttaagtatactatatatattgttaactataggcatgaagtaccgcagcaagtccatagaacttattcattttgcataattgaaactttattcctgttgaatagcaattctccatttcactatcctttcatgccctggcaatcaccattcttctctctgtttccatgagtttgactattttagttacttcatagaagtggaattatgaaatattttttctgtgacttcatttagcataaatttctccaggttccttcaagttgttgcatatggcaggatttccttttttataaggctgaatactgtgtgtgtgtgtgtgtgtgtgtgtgtgcgcacacacacacgcacacacatgcaggtatacaaatatcacattttctatcccatcattcatcaatggacatttaggtcattttcatatttggatataataagtagtgctgaaatgaacacggaagtgtggcccctgagatcctgagttcaaatttttaaaatatgctcagatgtggcattgctagatcatatggtagttatatttttaattttttcaggaagtttcatactgttttctatggcagctacatcattttacattccaacaatatacaagggttctaatttctccatatccttgccaacacctattatcttttgatttgggtggtttttggttttgtttttggtaatagccatcctagcaggtgtgagatgatatcccattacaatttctgtttgtttgtttttgaaggagtctcactgtgtcacgaggctggagtggagtggcgtgatcttggctcactgcaacctccgactcctgcctcagcctcccaagtagctgggactataggcacgtgccaccatgcccagctaatttttgtatttttagtagagatggggttttaccatgttggccaggatggtcttgatctcctgacctcatgatccacccgccttggcctcccaaagtgctgggataacaggcatgagccactgcacccagtcctcattacagttatttgaattttctgatgattagtgatattgaacatcttttcatatacctgttggccatttgtatgccttcttaagttgtaggagttccttatatattttggatattaaccatttattagatatatagtttggaaatattttctcctattctgtaggttgctttttcaccctcttgattgcttccttttttttttttttttaccaaagctttttagtttgatatggtctcccttgtctatttttgcttttgttggttgaatttttgagtcatttccaagaaatcattgtcaagatcagtatcatgaagctttcctgctatgttttcttctaggagttttacagttttcaggtcttaggtttaaatgtttaatctattttgagttgatttttgtgtatggtataagaaaagaattcaattttattttttatatgtgaatatccagttttcccaacatcatttgttgagtggcgtcttttctattgtgtattcttggcacccctgttgaatacgagttaactatttatgcatgggtttatttctgagatctctatcctgtttcattggcccatatatttgttttatgccaggaccatactattttaattgttgtagttttgttatatattttgaaatgaggacctatggggactgggtgcagtggctcatgcttataatcacagcaactggggaggctaagactggaggatcacttgtggtggagagtttgagaccagcctgggaaagatagaaagactccttctctacaaagatatttaaaaattagccaggcatgatggcacacacctatactcctagctacttaggagcctaaggtaggaggatcactcaagcccaagaatatgaagctgcagtgagctatgatcatgccactgcactctggcctgggcaacagggtaagatcttatgtctccgtaaataaataaataaataaataataaatgagaaaatgtgattcatccattttgttcttttttcttaagattgctttggctattcagagtcttttgtagttccatgtgaattttaggattttttttaaattttagtaaataatgctgttgggattttgatatggactgtattgaattcacagatcacttagggtagtgtgggcattttaacaatattaagtcatttaattcatgagtaccagatctttctatttatttgtgtcttctttaatttctttcatgaacattttatagttttcagtgcgtacattttcaatgccttagttaagcatatcctaagtattttatttattttgatgctatcataaatgggatttcttttttaattttcttttcagattattgttagtgtatagaaacacaggtgatttttgtatgttgattttatatccttcaacatttagtaaatttgtttattagttctaacagattttcctgacgtgtggagtctttagggtattctccatataagataatgtcatctgtaagcacttttttctttcaggttgggatgtcttttacttgtttttcttgcttaatttctccggctaggacttctagtgctttattaaatacaagtggcaagagtaggcatatttgtcttattcctgatcttacaggaaagtttttcagtttttcaccattgaatatgatgttagctgtgggattttcatgtagtctttgttatcatgaggtaaatttcctctgttctcatccatttgtgctactataacagaacatcacagactgggtaatttattatgatcacaaaccttatgggatcacagtactggagaatggaaaattcatgattgaggggctggtgaggtcttatttgttgtgtcataacgtgctgtaaggtatcacatggcacaaagagagaataagtgagaaaagggggcagtacctgcctgtttataatgaacccaatcctgagataacagcattaatgcatttaaaagggtaaaattctcataacttaaatacctcttaaaggtcccacttcccgattctgtcatgatgtcaattaaattgcaacatgagttttggaggggacaaacgttcaagttatagcaacttctatacctagcttttgttttcttttttagcttttaccatgaaagggtgttgaaatttgtcaaatgcttcttctgaatctcttttgatgatcatgtgatttttatccttctcagatcatgtggtgtatcacattatttgatctgcatatgttgaaccatccttgcttgctatcgtaaatgggatttctttcttaattttcttttcagattattgttagtgtatagaaactcaggtgatttttgtatgttgattttatacccttcaacatttagtaaatttgtttattaggaataactcccacttggtcatagtacatgatcatttaatgagctgttgcattcagtttgctaatattttgatgaagttatttgcatctatgttcatcagggatcttgccctgtagtatgcttttttctttctaaaaatttgctcatttataattgacaaaaactgtgtttattaatggggtacaaaacaatgttttgatctatataaacattgtagaaagattcaatcacgctaattaacataaccatcacatcacccacttatcatttttttgtggtaagaaagttaaaaagttattcttttagcaattttgaaatatacaatacatttatttttttcatttcctaatgtgtgacttttttgacttttaagttcaggggtacatgtacaggatgtgcaggcttattacataggtaaacgtgtcatgagggtttgttgtacagattatttcatcactgaggtattaatcccagtatgcattggttatttttcctgatcctctccctcctcccacccttcatcctccaataggcaccagtgtgcgttgttcccctttatgtgtccatgtgttcttatcatttagctcccacttataagtgagaacatgtggtatttggttttctgttgctaccttagtttgctaatgaaaatggcctccagctccattcatgtccctgcaaaggatatgatctcattctttttaatggctgcatagtattacatggtatgtatttaccacattgtccttatccagtctatcattgatggtcatttgaggtgattccatgtctttgccattgtgaatagtgctccaataaacatatgtgtgcatgtgtctttataatagagcaatgtatttccttttaagcatatacccagtaatgggattgctgggttgaatggtatttccgtctttaggttttgaggaattgccacagagtcttccattatagttgaattaatttacactcccatcaacagtgtgttagcattctttttttctccacaccctcgccagcatctgtgatttttgatctttttaataatagccattctgactggtgtaagatagtatctcattgtggttttgatttgcatttctctaatgatcagtaacgttgagctttttttcatatgcttgttggttgcatgtgtgtcttcttttcagaagtgtctgttcatgtcctttgcccactttttaacaaccatggggttgtttatttcttgtaaacttgtttaagttccttatagattctggatattagacttttgtcagatgcatagtctgcaaaaattttctcccattctgtaaactgtctgcttactctattgatagtttcttttgctggcagaagctctttagtttaattagatcccatttgttaatttttgtttttgttgcagttgcttttggtgtcttcatcatgaaatccttgcccatgcctatgtactgaatggtattgcctagattgtcttccagggtttttacagttttagattttacatttaagtctttaatccatcttgagatttattcctctactctaaatgaaaactgtacaccctttgatcaacatttctcctttcttcacatcctcttcctccagtcctatgataaccacctttatacactgtttctgtgatgttgactttttaaaattgtacgtgtaagtgagatcatatgatatttatctttctgtacctggctaattttgcttagcataacatcctttagttgcattcatgttgggatgaatgacaaaatttgttcttttgaagttttgtatagtagtccactgtgcatacctatcacattttctttattcattcacctgttgatgggtacaggttgtgtctatgggttagtgcaaaagtaattgtggtttttgccattattttaattgcagcaaaagtaatggtaaaaactgcaattacatttgcagcaacctaatatgtcttggttctcataaataatgctgaaatgaacacgggagtgcaggtatacctttgacataaggattttaattccgctaaaggaattaaatatatactactatgtacctagaagtggaattgctgaattatatggtaattgtatttgtttttggaacttccatgctatttttctcaaacagctatcgtaatttacattttcactaacaatgtataagggttcctttttctccacatcatcatcaacacttgttatcatttgtctttttgataatagccattctaagaggtgtgaggtaatatttcattgggattttaatttgcattttcctgatgatcagagatgttgagtattttttaatatatctattggtcattcatatatcttcttttaagaatgtctgttcagattatttgcccattcttcaattggtttattgattttctaggtgttgagttgtttgagtttcttgcatatgttagatattagccctttatccgatgtatggtttgcagatattttatttcaatccatgagctctctctctccactctgttaattgttttgtttgctgtaaagaacgtttttagtttgatctaatccaacttgtctctcgttatcttttgttacctgtgctttgagagtcctagccaagaaatccttgcccagacaaaagtcatagagcttttcttttatgtttccttctagtagcattatagtttcatttcttatatttaatacttttacttgtttggatttttttaaaatgtggtattataaaagggtccaatttcattctttcgcataaggaatattcagttttcccaacaccatttattgaagagatcgccctttcccccattgtttattctttgcacctttgttgaaaatcaattgactataaataattgggtttatttctgggctttttctcctgttccattagccaatatatctgtgtgtatgccaataccatagctttttattttgaaatgagagaatattgtacctccagctttgttctttttgctcagattgctttggccatttggcatcttttttattttattcaaattttagcatttttgactatttctgtgaaaactgacattagaattttgatagagattgcattcattttaacaatattagttcttccaacacatgaacatgaaatatattacaattgatttgtcttcctcagttgttttttttttttaaatcaaggtatagttttcagtatacagatctttgaccttcctggttaaatttacttcaaagtattttatttatttattttgatgctgttttaaataggattaatttcttaatttctttgttggaaacctcattgttaatgtatagaaatattactgatttttctatgttgattttgcaaactgcactttactgaatttatgtatcagttttaacagttctcagtacagtgttcagagttttctacatataagatcatgtcattagcaaacagagataattttatttcttgttttcttacatggatgtcttttatttctttttcttgcctaattgctatggatgggacttctagtactatgtcaaaaagaagtagtaacagtcaatatccttgtcttgttcctgatcttagaagaaaagctcccaacttttcacagtttttgtataatattagctgtgggcttgtcatatatggctttattttgttaaggtacattccttccatatctattttttgagattttcttatgaacatatgctagattttatcaaatgctttgtctgcatctagtaagatgatcatatgatgtttgttcttattttgttaatgtagcatatcacatttattgatttatgcatgtttaaccatttttgcatccctcagataaatcccaaataatcatggtgactgattttttaagtgtgctagggaattcagtttgctaatattttattgaggatttttggatctatgttcatcaagaatattggcctgcagttttcttttctggtagtatactcttatggttttcatatcagtgtaatgctgtcctcataaaatgagtttgacctcctcttcaatttttggaagaatttgagaagaattcatattacttctttaaatagaattcaccagtgaccttttcctttttttttttttttttttttttgattggagacttaatttgttactgatctattttctttatctagtattggtttgttcaaatgtaatatttcttgctgatttagtcttggtaggttgcatgtttctaaagattagttcatttcttctaggttattcagtttgatggtgtatggttgttcacagtaatagtttatgattccttgtatgtatgtgttatcagtgttagtctcccttctttcatttcagatattgtttgagttgtcttttttccctttgttagtctagttaaaggtttttcaattttcttttttcaaaaaaacaactttttagtatttgctttgtttatttctatccttatctttgttattttcttccttctgcaaagtttggggatattttgttcttctttttctaatcactaagatgtaatattaggttgtttatttgagattgtccttctttctttaatgtaggcaattattactaccaatttttctctcagtgcttctttttctacattccatactatttgttatcttgtgtttccattgtcattgatctcaaaatattttctgcttttccatataattttctctttgacccattggctcttcaggagcatgtcatttaatttccacgtaattatgagttgttcaaggttattcctcttattgacttctagtttcatgtcatcgtgaacagaaaaggtactcgatatgattttaatcttcttaaatttgttaaagcttgttttgtggcctaacatgtgatctatcctggagagtgtttcatgtgatttgagaagaatttgtattttgttgctgttggatgaactcttctataaatatctgttaggtccattggattaaagtgtagttcatgccttcctgtaatgagagactgcagaaattatgtcaggagtaagccagggaagaagtcactctcctattcttggtggtagagaccttccacctctattaaaagaaagattgtagagttgggattgtctctgtaggaagccagggaagaaggctgggcaatgctattcctcttgtttaggctgaagaaggtttattcttcatttgctatgtagatttccaagtactggaagaggccagaaccacagggagcggctggaaaatgtgcagcctaatggtttctatgggaaagttggaagatggtctgatcccaggaattaaagcttctgaaagggctcaggcagttcaacaccccccattgttctttgtacttcagagaaaactccaatggcttatctctgccatttcacaagcatggtgatctaaaagccaaactctgttgcagggactataaaagttgggttctacaaaatggaaaactaaggcacttatgaagatgattgtataatatagttcatatcttctctctttccactctagaaaaatcattttctagaaataaatacatttcctcaaattaaaaaaaaaaaaaaaaaaggttgtgttctatatgtgtgctatagcttctcaacccttgggaagcagctgcaaattcagttttcttcctaaatgtaaggcactgtgctcagggtggggttagtgcatgagtgtctttccactttttctacacattttgatatggatattttctgtcctctggtgtgtttgagtttttctactggattatgggtttctctgcagagaactgatccatatgtatgtagatgtttacttggtatgtccgtgagataaaaaaaatagtcataagcctcctatttcacaatgttgttaagtcacttccttcaaaagttgtaattttcttttcttacggtgtctttgtctggctttggtatgagagtaattctggccccataaaatgactttcaaagtgttctctcctcttcaaagttttaagagtttgagaagaattaatgttgattcttctttaaatgtttggttagacttctcctgtgaagccatctggtcctgtacatttcttttttgggagttactattattattattattattattattggttcaatctgtagcatatcctgtgtgtttgttatccatttggtctatagcgttgttcaaatccacagtttgtttatacattccatgtctggctgttctgttcattattgcaagtcaagtattgatgtatcctactgttatggtattgctgtatgtttatcccttcaaatctgtcaatgattgctttatgtatttacatgctttgatgatggattcacatatatttataattgttatatcttcttgatgaattaactcttttatcttatttaatgacctactttgtctcttgtgacagttttttttttttttttttttttacctaatgtctattttggctgatataattatagtcagtcctctgtgtctgtgggttttacatcatagattcagtcaatgctgattgaaaacactgtattcatggaatgtgaaatccagatacagagggtcaattttttttatccacaggtcctgcagggctaactgtgggacttaagcatcctcaattttggtatcccttggggttccagaaccatgaccctgtagatactaaaggacaactgtatagccccattactctcttttggttaccgtttgtacagaatgtatcttttttgactgggcacggtggctcatacctgtaatcctagcactttgggaggccaaggcaggtggatcacaaggtcaggagatcgagaccatcctggctaacatggtgaaaccctgtctctactaaaaacacacacacaaaaaattagccaggcgtggtcgtgggcgcctgttgtcccagctactcgggaggctgacgcaggagaatggtgtgaacctgggaggcagagcttgcagtgagctgagatcgcaccactgtactccagactgggtgacagagtgagactctgtctcaaaaaaaaaagaaagaaaagaaaaagaaaaacagaatttatcttttttatcaattaactttcaaactatgttttcttaaatttgaaatgagtatctggaaacagcatataacgatcttgttttcataaatctatgcaggcattgtaggtcttttcattgggaagtttaatccatttgtatttgaaataatcattggtaggtaaggacttactattgcctttttgttgaatttttctgttttgtaattcttttttctgtctttttctctcttgactgtcttcttttgtgattggttgatattttactgtgatatgctatgatttctgatttctcttttttatttctgtatttttacatggattttccatgaggcaatcacaaatataatagtctactttaagctgataacttaacttaaatcatgtaccaaaaatctttacttttaatacctccctcacactttttgttataaatgtcacaatttacatctttttataatgtgtttctacaaacatatttttgtaattttaattattcttattttgacatataacttttatattagaattataaatgatttacataatactatttagaaaaaataatagcctgacttacagaatatcttccacctaggttttaaaggggtgcattttgcacaaggcctatataagctatagggaaacaaaacacccacacaagtttctctattgaggtaattcataatcttctattagttttatcccactcttcatcatattgacaaccagggtatcattaggggaaaacaaaatctattactttgattttctcctctaagttagagttttctcctctgcagaagtttttctgcagcttcttctgttaagttcttggcaagctgtaatattcagtccccattaatgaacaataaaggatctgggcttctcttgacattgacatgaagtctctctctctcatgcctttccagtaggtgagatgaatatgaaaattacataaagactgcatattaaatagaggaaagctgagttaaaacagtctaaggtggctgaatatggtggctcatgcctgtaatcccagcactttgggaggcaaaggcaggcagatcacttgaggccaggagttggaaaccagaccagccaacatggcgaaactctatctctactaaaaatacaaaaattagcctggtgtggtggcatgcacctgtaatcccagctacttgggaggctgagacatgagaatcacttgaacccaggaggtggaggttgcagtgggccaagatggtggcactgcactccagcctgtgcctcagagcaagactccatcaaaaacagcagcaacaacaaaaacactctaaggcagaaagggaagtctggttaacattaggtatttgatgaataagaaaattcagtataataggaagcttcccattcaatagcaaaccgaaataaataaataaataaataaataaataaataaataaataaataagagaaagctgaatattaccaaatccaagagaataaagggaaaccataaccagatcatctttaactccagtaccctgcaagttaagtaatgatctcagaactagaaatccttgaaaataaattcttaattatatggattatactaatttatgctgtgactaaaaaagatgtaattacagatcaatgtagaatatacataaatctgtaaaacttacagataatttaagaaattagcagagaatgaagggagcaactgaagcttctagccctctttgtgagatttaggcttttccagtcaaaaacctcatttagatgttatgttattttgtgttgattttaaaagttatattttcttttacataattaattggaaagtccttcagtttgggtctgataattgataacttcctcagtacccagaaatctatcaagcataatgttgacatccaataaagtgtttatgttgaaatcctttatttagaattaaaaaggtagtcaaagagtttcttgaaacagagatatagaaatgtatattatctttttagtttatattcatttttctgtaaaggaataatggcatattaaaatgaatataaaattaatacaattttggttattgtatgtagcccctattcttttttgttgttatactttaagttctagggtccatgtgcacaatgtgcaggtttgttacatacgtatacgtgtaccatgttggtttgctgcacccatcaacttgtcatttacattaggtatttctcctaacgcaatccctcccacagatcccaaccccccagcaggccccggtgtgtgatattccccttcctgtgtccttgtgttctcattgttcaactcccacttatgagtgagaacatgcagagtttgattttccgtccttgtgatattttgctgagaatgttggtttccagcttcattcatgtccctgcaaaggacatgaactcatcattttttatggctgcatagtattccatggtgtatatatgccacattttctttatccagtctattattgatggacatttgggttggttccaagtctttgctattgtgtatagtgctgcaataaacataagtgtgtgtgtgtctttatagtagcatgatttataatcctttgggtatatatccagtaatgagattgctgggtcaaatagtattactaattctagatctttgagggatcgccacactgtcttccacaatgattgaactaatttacactctcaccaacagtgtaaaagtgttcctatttctccacatcctctccagcatctgttgtttactgactttagtgatcgccattctaattggcgtgagatggtatctcattgtggttttgatttgtctttctctgacgaccagtgatgatgaacatttttttcataggtctgttggctgcataaatgccttcttttgagaagtgtctgttcatatcctttgcccactttttgatggagttgtttgtttgttttcttttcttgtaaatttgtttaagttctttgtggattctggatattagccctttgtcagatggatagattgcaaaaattttctcccattcagtaggttgcctgttcactctgatgacagtttcttttgctgtgtagaagctctttggtttaattagatcccatttgtctattttggcttttgttgtcattgcttttagtgttttactcatgaagtctttgcccacgcctatgtccctgaatggcattgcctagggtttcttctaaggtttttatggttttaggtcttacatttaagtccttaatccatcttgagttaatttttgtttaaggtgtaaggaagggatccagtttcaactttctacatatggctagccagttttcccagcaccatttattaaatagggaatcctatccccattcatcatttttgtcaggtttgtccaagatcagatggttgtagatgtgtggtgctatttctgaggtctctgttctgttccattggtctgtatatctgtgttggtaccgtaccatgctgatttggttactgtagctttttagcatagtttgaagtcaggtagtgtgatgcctccagcttggttatttttgtttgggattatcttggctatgcaggctctttgttggctccacatgaaatttaaagtagttttttccaattctgtgaagaaagtcaatgggagcttgatgaggatagcattgaatctataaattaccttgggcagtatggccattttcacgatattgatttttcctatccatgagcatggaatattcttccatttgtttgtgtcctcttttattttgttgagcagtagtttgtagttctccttgaagaggtccttcacatcccttgtaagctggattcctaggtattttattctctttgtagtaattgtgaatgggagttcactcatgatttggctccctattctttatcaaagttctgaatattagccaaatcatcaaagaggatattcattacctttaattcttataacttttcttagcaataaaattcatggtaagacagctagcagataaaaaatgtacaagtacatagtttaaaacaacactgattagcacaccaaaagaaaattgagccaatccttgttatatgtaatacaaatattttatatatgtacatttagctgtgtttggagacccacgtttctacacatatataattatttaagacatagtttatgtatagctaccatgaacacaataaaatacattttatttctatattaacacattttaacctatcttagtgatctgtagaaagatctagacaatgattctggttttaactacatttcttttttgtttctttaccttggaggtcaagacatctttatgacagaagaacagaagaaatactacaatgcaatgaaaaagctggggtccaagaagccacaaaagccaattcctcgtccaggggtaaaaaaaaatatgtatctttagcatatagattttcaaattatttctaattcatttttaatgcacatctttaatttctagatactacttgaaaaatttactctgcattcaatattattctcgtttctttgcagaacaaaatccaaggatgtatatttgacctagtgacaaatcaagcatttgatatcagtatcatggttcttatctgtctcaacatggtaaccatgatggtagaaaaggagggtcaaagtccatatatgactgacgttttatattggataaatgtggtttttataatccttttcactggagaatgtgtgctaaaactgatctccctcagatactactacttcactataggatggaatatttttgattttgtggttgtgattatctccattgtaggtaagaatatttattattcagattttattttttgagtaaagctacacttcacttatgctcgaggaagtatattctgaattcttgaatgcagatttattcttgagtaagtcagtataatgaaagaatgaaaaaatcctaaaaagcaaaataaaaatatctgtgtctctcatactacagtgtttctgggtttacgctacaggccccaaagccaaatatttctatctgttagaaggaaattatacttgttgatatttagtttccaatattacctttgcaaagctagttaccatgtttatatatagctgtgtatatagctgtgtcttggaaaaaccagtgttgaatctgaaatcacttctggttttagcacatgtgagacttgtcatttgattaatgataatatcaatagccagaatatctgtgaatttctgaaaattcttccagaatttaaagtgaagttaaagtctctagaaatgccagttttattttttaaggcttaatacttcacaagtgtataaaatgcgaatcattcctcaaccagccatggtcatgtctgtgaataacagcacatgattttatacttcctacatattcattatcatggctgtttttctatcttcttggggtttttttcattattttcacatatcttgcaaagtattttaggattgtatttctttttagaattataaacttttaatagagattatagagattatttactacaaacttaatcaacaggttatcagtctgaaaaggttaaaggttgcataacttagactcataaaactgagtgccacagaacagaataagcatttaaaatcaatatttttggaaaaggatattatcaccagaagtcataccaaaattgatataggaaaatcaattaaaatttagatgagaatttcacactaacaataataattctgaaagcattcccattaagtcagggaaaaaaaggtgtccctgtatcatccctgtttttaatagtgttggccaatgcaattagataagaaaacaaagaggtacatatatccaaacaatggagcaaacgatccttatcttaatataatgaccttcttcatataatgaaagaagttcttcatataatgaatgaccttcttcatataatgagagaatcaactaaaaataaattcaattagagttgaaaaggtacaaattttaaattacatgtgccaacactaataatcttatacatgaaaaaaatagaaaatctgattcacaattgcaataaaaataaagtaaattatctaacaataactttatcatatgagacttctgaagaaaactttaggatataaaagaaagctgaaataattgaaaggattatcatgtacttagataagcttctatcattgtaattaagacttttttatttattaatctacaagttcaaggaaaatatcaatgagtctttttatgcagtgggggtaataaattgacaataacaatgtaagtctaaatgattctaaattcatcggaagaaaattagggtgataagaggtggcttattctgcaaaatattaagacttataatattgatgttaagtaaaactgttcaaatacaaaagctcaatgagtttgaagagagagtccaaattaaactttaatgtataattaccaatttactgagtgaacggatcacatgaatcaaagtctatagttaaaaaaagagattctagtaggaaatatgtggaaatagacatataaccatatactcttatagtcctttctattcataacacaaatttgtaaagctaaaaaaaaaaacgaagaagaaatgactgcacaaaaaaacaaaatattctttgaggaaaaaatcatcataaataaaaggttgaaagacaaaaaataggggaaatgtttatgtaacagttaaggctgagaaaggatcaatactttaaatagaaaattgttcttataaattaataagaatatagtaaaaatcccaatagacacatgggaaaataccaactaagccaaaaacagaagttaaaaacaaccaataaggatacaaaatgacaaaatacaagacaaattgtggattattttcacaaaaaatgcaaataaattacaatagtagatgttttcaaatgtcatgaccattttttggtaaaaagttataaagaagaatggtagtactcaatactggtgaacgtgtaaggtatgagcacattcatatactgccattagaaatgttaattagtacaggctttctggagataggttcagctatatatagtgtatttatatatacacatacatactatatatagtatatattatgtatatatatagcatgtgtatatatatatggtatatgtgtatacatagtatgtgtgtatttatataactgtatatatgctgttgaaacaagttctgttaatatattattaaaacgtactcatgcaattaggagaacatacctatttagatattcatttcagtaacaaaaatggaaacagtataagtgctagtcttcaatctgtggttattttaagagagcattataccactataataagttattttgttgtcatttttatgaaactatctccatgacacataaataaattaaaataggttacaggctgggaaccatggctcatgcctataatcccagcacattgggaggccgaggtgggtgaaatcatttgaagtcagaagtttgagaccagcctggccaacatggtgaaacccgtccctaccaaaaatataaaaattaaccaggtgtggtggcgcatgcctgtagtcccagctactctggaggctgaggctggagaatcacttgatcttgggatgcggagattgcagtgagccaacatcacgccactgcactccaactttggtgatagagcgagaccctgtcttgaaaaataaataaaaaataggctacaaaaaaagcttatataacatgaatacatttgtttgtgtgtgtacatatgcttgcacagaaaaatgtctgaagtgatactcaaaaaaatattgataatagttattgttgttgacatttcaggtggtttatattttcttttagcatttaatggtattggttgaattttctatgtgagtaggtattattcttaaaatttaaaacactattatggaggaagaaaatggcccatgaacttagtaaaaaggagaaaatgtcagaatggtaatgtcagtcatttttctttggaattgcttaatccttttttaagtaattgcattcagtataaaataactgtcttgaattcataagaaacgagttgacagataaaactgtttagagtcatcattttatgtagcatagatatttagatattttatttctattattttcctccacacataggtatgtttctagctgatttgattgaaacatattttgtgtcccctaccctgttccgagtgatccgtcttgccaggattggccgaatcttacgtctagtcaaaggagcaaaggggatccgcacgctgctctttgctttgatgatgtcccttcctgcattgtttaacatcggcctcttgcttttcctggtcatgttcatctacgccatctttgggatgtccaactttgcctatgttaaaaaagaagatggaattaatgacatgttcaattttgagacctttggcaacagtatgatttgcctgttccaaattacaacctctgctggctgggatggattgctagcgcctattcttaacagtaagccacctgactgtgatccaaaaaaagttcatcctggaagttcagttgaaggagactgtggtaacccatctgttggaatattctactttgttagttatatcatcatttccttcctggttgtggtgaacatgtacattgcagtcatactggagaattttagtgttgccactgaagaaagtactgaacctctgagtgaggatgactttgagatgttctatgaagtttgggagaagtttgatccagatgcaactcagtttatagaatataacaagctctctgattttgcagctgccctggatcctcctcttctcatagcaaaacccaacaaagtccaactcattgccatggatctgcccatggtcagtggtgaccggatccattgtcttgacatcttatttgcttttacaaagcgtgttttgggtgagagtggggagatggattctcttcgttcacagatggaagaaaggttcatgtctgcaaatccttccaaagtttcctatgaacccatcacaaccacactgaaacggaaacaagaggatgtgtctgctactgtcattcaacgtgcttatagacgttaccgcttaaggcaaaatgtcaaaaatatatcaagtatatacataaaagatggagacagagatgatgatttactcaataaaaaagatatggcttttgataacgttaatgagaactcaagtccagaaaaaacagatgccacttcatccaccacctctccaccttcatatgatagtgtaacaaagccagacaaagagaaatatgaacaagacagaacagaaaaggaagacaaagggaaagacagcaaagaaagcaaaaaatagaacttcatttttgatatattgtttacagcctgtgaaagtgatttatttgtgttaataaaactcttttgaggaggtctatgccaaaatcctttttatcaaaatattctcaaaggcagtgcagtcactaaccctgatttcctaagaaaggtgggcagcattagcagatggttatttttgcactgatgattctgtaagaatcgtaagagaactctgtaggaattgttgattatagtaaacaaaggtgattcagttttttggttttcaataaatcagaagaccacatagaaaacttttacatctgccttgtcatcttttcacaggattgtaattattcatatttcccatgtaaataaacaacacacacacacacagaaaaatctattatttatctattatttgaaaatcaacaaaagtattttgccttggctttgcaatgagatgcttgatagaagtaatgaacattagtaatgaatgtttagttaaaatgcattattagggagcttgactttttagcaatgtacagaggttattctatatattgaggtgcttaaatttattctacattgcattagaaccaatttatatgtgcctataaaatgccatgggattaaaaatatatgtaggctattcgtttctacaaatgcttttcattcatcttgactcacaagccaacaaggataatacttacctttagagtattgtgtttcatagccttctttcatatccctctttgttcatagaataaccacagaacttgaaaaattattctaagtacatattacactcctcaaaaaataaagataactgagaaaaaaagtaattgacggaagttctatttgctattacttatatagcctaacattgggctgtgctgcccaaaatactgataatagtctcttaaacttttgtcaaattttcctgctttctcatgcggtattgtttagtcatcctttagctgtaagcaaagttgatgaaatccttcctgatatgcagttagttgtttgatcatggtatatgtttgagcagataataaccttggcacagtatttattgcatcacttgtatacaatcctgtgtttggcaagctttcaagtcatataatatgacagactttcaacacagatatatgtgtttagtatgaataaaaaagcattgaaatagggattctttccaactcgctctcttgccaccaacttactttcctaaattatggaagtaatcttttggacctacatacttcaatgtatacaatgaggaagatgtcaccttctccttaaaattctattatatgaaatatattttgcctcaatcaacacagtaccatgggcttctaatttatcaagcacatattcattttgcattagctgtatacatctagttttttgaaaacacctattaatagtactttgaaaagaaataactgtaatgctttttttcatgagtttatttcaggaatatgagatctttcttctataaagtcattcatgcacaggcaaaagttgagctacatagatagaacatagttttatttagaagatttttgtgggaggttttgaagcgaatatataaaacatctttcactaatttgcttttcatatttaaaaaataataaattacatttatataataaatgtttaaagtacatatttttgttgttctgaaaattgaaaaagaaaggatttaaatgtacttatagaaacaaacggttatggttaaagaatgagacaggaagtctagaatgtttttaaattgtgatatattttacaacatccgttatgactttgagacatttgttctaatgtacgtataaaactatatctagggctaaagattttttataccatcttaggttcattcatcttaggttatttgaaccactttttaatttaatatgaaagacagcatgcagtgttttccgagactatatagatcattgcatcccatacctgccacgcctgttggaaataggttttgataatttaagtagggacctacaaaaaatatattacagtcatcaaattttttaatacattcaattaagaatttaacatcaccttaaatttgaattcaatctatcattatttcaaactcacaagttaactgcattatgaatacttatataatatagtatgaaaagatgtttgacaggtttgtgtgtaattttctattaatgtttttacattgccttgtttttatgtaaaataaaaaatatgggcaactggttcgttaacacaatttcttcttagcttttcaaaaaaacatataaagttgttctttttcctatttcatgaactatgattttttaaaaaataacatggttaagttttatatatatttatgtttgtttcagaaatgtctacttgtgactttttatcaattaaaaacaatatttggaagaaagagcctattaagtataaccttgaagtaaaattagacttctctttccgtgtggattactgtttgtactgattgtatcacccttcagaaggcactgtcatattaatatttaaattttataatcactgaacttattacacccaacaatacagaatggcagttacactgaagaatttaatttagaataaaatggaagcaaacaggttttctaaaaattttttttaactgaccaggtcttgctctgtcacccaggctagagtgcagtggcatgatcatagctctctgcagcctcaactctgggctcaagcaaccctcttgcctcagcctcccaagtagctaggactacaggtgcatgccaccatgccagctaatatttaaatttttgtagataaggggtcttgctctgttgccccaggctagtctcaaactcctggcttcaagtggtcctacagccatgacctgccaacatgctggattacaggcatgagccaccatgccccagacaggtttgaacacaaatctttcgaatgaaaattagagaatctaattttagcattttgatagttacctagtttgcaaaagatttgggtgacttgtgagctgtttttaaatgctgattgttgaacatcacaaatatttagcatgattttatagaattttgatagctttatttaaaagagtgaaaatacaatgcatacataaataaagcagttctaaatagctatttcagagaaatgttaatagaagcactgagagaagggccaactaaattaggatggccagggaattggcctgggtgtaggacctatgtataaaggccaccagtttttaaaaaatatctgtggtttattatgttattatcttcctgaggaaaacaatcaagaattgcttcatgaaaataaataaatagccatgaatataataaagctgtttacataggattctttacaaattttatagatctatgaatgctcaaaatgtttgagtttgtcataaattatattgtagttatacttgagaccgacacattgtaatataatctaagaataaaagttatgc

SEQ ID NO: 3 - DNA, 14 bp, Homo sapiens
ctacacgagctttg

SEQ ID NO: 4 - DNA, 17 bp, Homo sapiens
tgaaatggtattaaaac

SEQ ID NO: 5 - DNA, 18 bp, Homo sapiens
tgaaatggtattaaaact

SEQ ID NO: 6 - DNA, 18 bp, Homo sapiens
gaaatggtattaaaactg

SEQ ID NO: 7 - DNA, 17 bp, Homo sapiens
aatggtattaaaactga

SEQ ID NO: 8 - DNA, 19 bp, Homo sapiens
tgaaatggtattaaaactg

SEQ ID NO: 9 - DNA, 18 bp, Homo sapiens
aaatggtattaaaactga

SEQ ID NO: 10 - DNA, 18 bp, Homo sapiens
aatggtattaaaactgat

SEQ ID NO: 11 - DNA, 19 bp, Homo sapiens
aaagggaataaattatgtg

SEQ ID NO: 12 - DNA, 19 bp, Homo sapiens
aagggaataaattatgtga

SEQ ID NO: 13 - DNA, 19 bp, Homo sapiens
agggaataaattatgtgaa

SEQ ID NO: 14 - DNA, 18 bp, Homo sapiens
gggaataaattatgtgaa

SEQ ID NO: 15 - DNA, 20 bp, Homo sapiens
aaaaagggaataaattatgt

SEQ ID NO: 16 - DNA, 19 bp, Homo sapiens
aaaaagggaataaattatg

SEQ ID NO: 17 - DNA, 19 bp, Homo sapiens
aaaagggaataaattatgt

SEQ ID NO: 18 - DNA, 20 bp, Homo sapiens
aaaagggaataaattatgtg

SEQ ID NO: 19 - DNA, 18 bp, Homo sapiens
aaaagggaataaattatg

SEQ ID NO: 20 - DNA, 20 bp, Homo sapiens
aaagggaataaattatgtga

SEQ ID NO: 21 - DNA, 18 bp, Homo sapiens
aagggaataaattatgtg

SEQ ID NO: 22 - DNA, 18 bp, Homo sapiens
agggaataaattatgtga

SEQ ID NO: 23 - DNA, 18 bp, Homo sapiens
tagaaagagcagtatgag

SEQ ID NO: 24 - DNA, 17 bp, Homo sapiens
tagaaagagcagtatga

SEQ ID NO: 25 - DNA, 16 bp, Homo sapiens
atgggttgatactatc

SEQ ID NO: 26 - DNA, 17 bp, Homo sapiens
atgggttgatactatca

SEQ ID NO: 27 - DNA, 17 bp, Homo sapiens
tgatttagaagcatcaa

SEQ ID NO: 28 - DNA, 14 bp, Artificial sequence
  Synthetic oligonucleotide
caaagctcgtgtag

SEQ ID NO: 29 - DNA, 17 bp, Artificial sequence
  Synthetic oligonucleotide
gttttaataccatttca

SEQ ID NO: 30 - DNA, 18 bp, Artificial sequence
  Synthetic oligonucleotide
agttttaataccatttca

SEQ ID NO: 31 - DNA, 18 bp, Artificial sequence
  Synthetic oligonucleotide
cagttttaataccatttc

SEQ ID NO: 32 - DNA, 17 bp, Artificial sequence
  Synthetic oligonucleotide
tcagttttaataccatt

SEQ ID NO: 33 - DNA, 19 bp, Artificial sequence
  Synthetic oligonucleotide
cagttttaataccatttca

SEQ ID NO: 34 - DNA, 18 bp, Artificial sequence
  Synthetic oligonucleotide
tcagttttaataccattt

SEQ ID NO: 35 - DNA, 18 bp, Artificial sequence
  Synthetic oligonucleotide
atcagttttaataccatt

SEQ ID NO: 36 - DNA, 19 bp, Artificial sequence
  Synthetic oligonucleotide
cacataatttattcccttt

SEQ ID NO: 37 - DNA, 19 bp, Artificial sequence
  Synthetic oligonucleotide
tcacataatttattccctt

SEQ ID NO: 38 - DNA, 19 bp, Artificial sequence
  Synthetic oligonucleotide
ttcacataatttattccct

SEQ ID NO: 39 - DNA, 18 bp, Artificial sequence
  Synthetic oligonucleotide
ttcacataatttattccc

SEQ ID NO: 40 - DNA, 20 bp, Artificial sequence
  Synthetic oligonucleotide
acataatttattcccttttt

SEQ ID NO: 41 - DNA, 19 bp, Artificial sequence
  Synthetic oligonucleotide
cataatttattcccttttt

SEQ ID NO: 42 - DNA, 19 bp, Artificial sequence
  Synthetic oligonucleotide
acataatttattccctttt

SEQ ID NO: 43 - DNA, 20 bp, Artificial sequence
  Synthetic oligonucleotide
cacataatttattccctttt

SEQ ID NO: 44 - DNA, 18 bp, Artificial sequence
  Synthetic oligonucleotide
cataatttattccctttt

SEQ ID NO: 45 - DNA, 20 bp, Artificial sequence
  Synthetic oligonucleotide
tcacataatttattcccttt

SEQ ID NO: 46 - DNA, 18 bp, Artificial sequence
  Synthetic oligonucleotide
cacataatttattccctt

SEQ ID NO: 47 - DNA, 18 bp, Artificial sequence
  Synthetic oligonucleotide
tcacataatttattccct

SEQ ID NO: 48 - DNA, 18 bp, Artificial sequence
  Synthetic oligonucleotide
ctcatactgctctttcta

SEQ ID NO: 49 - DNA, 17 bp, Artificial sequence
  Synthetic oligonucleotide
tcatactgctctttcta

SEQ ID NO: 50 - DNA, 16 bp, Artificial sequence
  Synthetic oligonucleotide
gatagtatcaacccat

SEQ ID NO: 51 - DNA, 17 bp, Artificial sequence
  Synthetic oligonucleotide
tgatagtatcaacccat

SEQ ID NO: 52 - DNA, 17 bp, Artificial sequence
  Synthetic oligonucleotide
ttgatgcttctaaatca